(19) United States
(12) Patent Application Publication
(10) Pub. No.: US 2022/0096450 A1
(43) Pub. Date: Mar. 31, 2022

(54) Compounds and Methods for Treating or Preventing Heart Failure

(71) Applicant: DREXEL UNIVERSITY, Philadelphia, PA (US)

(72) Inventors: Marilyn S. JORNS, Philadelphia, PA (US); Michael R. JACKSON, Crossville, TN (US); Kristie D. COX, Charleston, SC (US); Patrick Y. S. LAM, Philadelphia, PA (US); Simon D. P. BAUGH, Ringoes, NJ (US)

(21) Appl. No.: 17/421,821

(22) PCT Filed: Jan. 9, 2020
(86) PCT No.: PCT/US20/12938

§ 371 Date: Jul. 9, 2021

Related U.S. Application Data
(60) Provisional application No. 62/790,038, filed on Jan. 9, 2019.

(51) Int. Cl. A61K 31/444; C07D 405/14; C07D 401/04; C07D 401/14; C07D 239/34; C07D 213/64; A61P 9/04; A61K 31/505; A61K 31/5377; A61K 31/496; A61K 31/4418

(52) U.S. Cl. CPC A61K 31/444; C07D 405/14; C07D 401/04; C07D 401/14; C07D 239/34; A61K 31/4418; A61P 9/04; A61K 31/505; A61K 31/5377; A61K 31/496; C07D 213/64

(57) ABSTRACT

The present invention relates to the discovery of compounds that can be used to treat and/or prevent heart failure in a subject. In certain embodiments, the compounds of the invention are sulfide:quinone oxidoreductase (SQOR) inhibitors. In other embodiments, the compounds of the invention increase physiological levels of H₂S in the subject. In yet other embodiments, administration of the compounds of the invention treats, ameliorates, and/or prevents hypertension, and/or atherosclerosis, and/or pathological cardiac remodeling that leads to heart failure in the subject.

### CROSS-REFERENCE TO RELATED APPLICATIONS

This application claims priority under 35 U.S.C. § 119(e) to U.S. Provisional Application No. 62/790,038 filed Jan. 9, 2019, which is incorporated herein by reference in its entirety.

### STATEMENT REGARDING FEDERALLY SPONSORED RESEARCH OR DEVELOPMENT

This invention was made with government support under grant number R01 GM107389 and 1R41 HL134435 awarded by the National Institutes of Health. The government has certain rights in the invention.

### BACKGROUND OF THE INVENTION

Heart failure is a highly lethal cardiovascular syndrome, with a 5 year mortality in treated patients (˜50%) that is worse than that of many malignancies. Heart failure currently affects 6.5 million adults in the U.S. This figure is expected to increase to over 8 million by 2030, owing to the aging of the population, increasing prevalence of risk factors, and improved myocardial infarction survival. More than 1 million heart failure-related hospitalizations occur each year, and ˜25% of heart failure patients are readmitted within 1 month of discharge. Hospitalizations are the biggest driver of the costs associated with heart failure, which are currently estimated at $30.7 billion each year in the U.S. By 2030 total overall costs are expected to more than double to nearly $70 billion. Treatment and prevention of heart failure has become a burgeoning public health problem reaching epidemic levels, especially for the rapidly expanding elderly population. There is a critical need for potent new drugs to treat heart failure that improve patient survival and decrease hospitalizations.

Heart failure is a complex clinical syndrome that results from any structural or functional impairment of ventricular filling or ejection of blood. The cardinal manifestations of heart failure are shortness of breath (dyspnea) and fatigue, which may limit exercise tolerance, and fluid retention, which may lead to pulmonary, abdominal, and/or peripheral edema. Some patients have exercise intolerance, but little evidence of fluid retention, whereas others complain primarily of edema, dyspnea, or fatigue. There is no single diagnostic test for heart failure because it is largely a clinical diagnosis. Thus, a careful history, physical examination, and various diagnostic tests are essential for the assessment of patients with symptoms that suggest heart failure.

Pharmacologic therapy is a major component of ACC/AHA guidelines for the treatment of patients with symptomatic (NYHA Class II-IV) heart failure (Ponikowski el al., 2016, Eur. J. Heart Fail. 18: 891-975; Yancy et al., 2013, J. Am. Coll. Cardiol. 62: e147-e239; Yancy et al., 2016, J. Card. Fail. 22: 659-669). Angiotensin-converting enzyme inhibitors (ACEIs) and beta blockers are recommended first-line drugs, unless contraindicated. Mineralocorticoid/aldosterone receptor antagonists (MRAs) are recommended in patients who remain symptomatic despite treatment with first-line drugs. Diuretics are recommended in patients with signs and/or symptoms of congestion. An angiotensin II type I receptor blocker (ARB) is recommended in symptomatic patients unable to tolerate an ACEI (who should also receive a beta blocker and an MRA). Angiotensin receptor neprilysin inhibitors (ARNIs) are new combination therapeutics that contain an ARB and a neprilysin inhibitor in a single substance. Entresto (sacubitril/valsartan), which is a first in class ARNI, is recommended as an ACEI replacement in ambulatory heart failure patients who remain symptomatic despite optimal treatment with an ACEI, a beta blocker and an MRA. Hydralazine and isosorbide dinitrate should be considered in self-identified black patients who also remain symptomatic, despite treatment with the same three drugs. Patients with persistently severe symptoms despite maximum guideline determined medical therapy may be eligible for advanced treatment strategies, such as cardiac resynchronization therapy, an implantable cardioverter-defibrillator or transplantation. Cardiac transplantation is the current gold standard for the treatment of refractory heart failure but is restricted by the availability of organ donors and requires lifelong immunosuppressant drugs. There is a compelling need for new drugs that reduce the major risk factors for myocardial damage and mitigate the pathological remodeling triggered by injury that leads to heart failure. Over the past 15 years, the endogenous gaseous transmitter hydrogen sulfide (H₂S) has become recognized as a crucial signaling molecule in the cardiovascular system where H₂S is produced primarily by cystathionine γ-lyase (CSE). H₂S is a key regulator of blood pressure and protects against the development of hypertension and atherosclerosis, which are major causes of heart failure. Patients with essential hypertension exhibit a marked decrease in plasma H₂S levels. In the setting of myocardial injury, exogenously administered H₂S or overexpression of CSE reduces infarct size in a myocardial ischemia/reperfusion (MI/R) mouse model and protects against pressure overload-induced, volume overload-induced, and ischemia-induced heart failure. Conversely, myocardial injury is exacerbated by pharmacological inhibition or genetic deficiency of CSE. Patients with coronary heart disease or congestive heart failure exhibit a pronounced decrease (up to 3-fold) in plasma H₂S levels that appears to be correlated with the severity of the disease. The finding of reduced H₂S levels has been replicated in several experimental rodent models of heart failure where the decrease could be prevented by exogenous administration of H₂S.

After myocardial insult or injury, the left ventricle undergoes morphological changes that are initially adaptive but ultimately become maladaptive, leading to the development of heart failure. H₂S mitigates pathological remodeling by regulating multiple critical cellular processes including anti-hypertrophy, anti-oxidant response, anti-apoptosis, anti-fibrosis, pro-angiogenesis and anti-inflammation. Exogenous H₂S or overexpression of CSE prevents cardiac enlargement and preserves left ventricle function in animal models of heart failure. When vascular growth cannot keep pace with pathological myocyte growth the heart rapidly progresses from a compensatory hypertrophic state to decompensated failure. H₂S mitigates cardiac remodeling by promoting angiogenesis via upregulation of vascular endothelial growth factor (VEGF) and activation of the VEGF receptor. Oxidative stress, a prominent factor in left ventricle remodeling, results from excessive production of reactive oxygen species (ROS), which can overwhelm the antioxidant defense system and cause irreversible mitochondrial injury and release of apoptotoic signaling molecules. H₂S induces nuclear translocation of Nrf2, a master regulator of the anti-oxidant response, and consequently increases expression of Nrf2-targeted genes that detoxify pro-oxidative stressors. H₂S also mitigates apoptosis and preserves mitochondrial function by reducing ROS production and by activating prosurvival signaling cascades. Anomalous fibroblast proliferation and transformation into myofibroblasts are hallmarks of cardiac fibrosis, which result in excessive extracellular matrix deposition and loss of tissue compliance. Exogenous H₂S attenuates fibrosis in several models of heart failure by decreasing oxidative and proteolytic stresses. H₂S reduces inflammation, a key player in MI/R injury, by limiting neutrophil adhesion and activation and by suppressing the release of proinflammatory cytokines and free radicals.

There is a need in the art for novel compositions that are useful for the treatment or prevention of heart failure in a subject. The present invention addresses this unmet need.

### BRIEF SUMMARY OF THE INVENTION

The present invention provides a compound of formula (IA), (IB), (II), or (III), or a salt, solvate, stereoisomer, geometric isomer, and/or tautomer thereof, as defined elsewhere herein. The present invention provides a pharmaceutical composition comprising at least one compound contemplated in the invention and at least one pharmaceutically acceptable carrier. The present invention further provides a method of treating or preventing heart failure in a subject in need thereof, the method comprising administering to the subject a therapeutically effective amount of at least one compound contemplated in the invention. The present invention further provides a method of increasing, or reversing loss of, physiological levels of H₂S in a tissue from a subject, the method comprising administering to the subject a therapeutically effective amount of at least one compound contemplated in the invention.

### BRIEF DESCRIPTION OF THE DRAWINGS

The following detailed description of certain embodiments of the invention will be better understood when read in conjunction with the appended drawings. For the purpose of illustrating the invention, shown in the drawings are specific embodiments. It should be understood, however, that the invention is not limited to the precise arrangements and instrumentalities of the embodiments shown in the drawings.

FIG. 1 is a schematic illustration of cardioprotective effects of H₂S in heart failure.

FIGS. 2A-2B are schematic illustrations of pathways for H₂S metabolism with glutathione (GS⁻) (FIG. 2A) or sulfite (FIG. 2B) as the sulfane sulfur acceptor in the SQOR reaction. SQOR, sulfide; quinone oxidoreductase; TST, thiosulfate sulfurtransferase; SDO, sulfur dioxygenase; SO, sulfite oxidase.

FIG. 3 is a graph illustrating IC₅₀ determination for compound 1 against SQOR.

FIG. 4 illustrates certain Sets of potent SQOR inhibitors, including compounds 1-3.

FIGS. 5A-5F illustrate non-limiting synthetic schemes for preparation of Set A/A′ analogs.

FIGS. 6A-6B illustrate non-limiting synthetic schemes for preparation of Set B analogs.

FIGS. 7A-7B illustrate non-limiting synthetic schemes for preparation of Set C analogs.

FIGS. 8A-8C illustrate views of the CoQ binding pocket in models of SQORf●inhibitor complexes. Except as noted, inhibitor docking was performed using GOLD (Cambridge Crystallographic Data Centre) with flexible ligand/rigid protein docking to ligand-free SQOR (PDB:ID 6M06). Carbons in amino acid sidechains are colored white. FIG. 8A: The structure of the human SQOR complex with decylCoQ (gold carbons) is compared with the model of the SQOR complex with 4-(4-aminophenyl)-2-methoxy-6-(3-methylpyridin-2-yl)pyridine-3-carbonitrile (FC9402) (yellow carbons). A hydrogen bond between the 2-pyridyl ring in FC9402 and W435:NE1 is indicated by a dash yellow line. FIG. 8B: The model of the SQOR complex with HTS12441 (compound 1) (magenta carbons) was produced using GLIDE (Schródinger) with flexible ligand and flexible amino acid sidechains within the CoQ binding pocket. Amino acid sidechains in the SQOR●HTS12441 complex (green carbons) are compared those observed in the SQOR crystal structure (white carbons).

FIG. 8C: The model of the SQOR complex with FC9402 (yellow carbons) is compared with the binding mode observed for HTS12441 (magenta carbons).

FIG. 9 shows assay conditions for assessing the selectivity of SQOR inhibitors.

FIG. 10 shows the efficacy of FC9402 in a cell-based model of cardiac hypertrophy induced by treatment of H9C2 cells with isoproterenol (100 μM, 24 h; then 50 μM, 24 h). The top panel shows confocal images of cells stained with phalloidin (actin fibers), WGA (membranes) and DAPI (nuclei). The bottom panel is a graph showing the surface area of H9C2 cells observed under different treatment conditions. Statistical significance was determined by one-way ANOVA with Tukey-Kramer post hoc test.

FIG. 11 shows the efficacy of FC9402 in a cell-based model of cardiac hypertrophy induced by treatment of rat neonatal ventricular cardiomyocytes (NVCMs) with angiotensin II (Ang II). The graph compares the surface area observed for control NVCMs with that observed for NVCMs incubated for 24 h with 200 nM Ang II in the absence or presence of 10 μM FC9402. ^{††}P<0.01 vs. Ang II, ^{††††}P<0.0001 vs. Ang II, ****P<0.0001 vs. control. Statistical significance was determined by one-way ANOVA with Tukey-Kramerpost hoc test.

FIG. 12 shows the timeline for the TAC mouse study with FC9402 (adapted from Polhemus et al., 2013, Circ. Heart Fail. 6: 1077-1086).

FIG. 13 shows Kaplan-Meir survival curves for 84 days following surgery for sham operated mice (n=8), TAC mice that received vehicle (n=4 at t>35 days post-surgery), and TAC mice that received FC9402 (n=5).

FIGS. 14A-14D illustrate gross cardiac morphology 12 weeks post-surgery. FIGS. 14A-14C show representative pictures of hearts. FIG. 14D is a graph of the ratio of heart weight to tibia length (HW:TL). ^{††}P<0.01 vs. sham, *P<0.05 vs. TAC+FC9402. Statistical significance was determined by one-way ANOVA with Tukey-Kramer post hoc test.

FIGS. 15A-15E are a set of graphs depicting the results of echocardiography studies to evaluate cardiac structure and function. Parasternal long axis images were obtained in two-dimensional B-mode and M-mode. FIG. 15A: Left ventricle ejection fraction (EF) (%); FIG. 15B: Left ventricular fractional shortening (FS) (%); FIG. 15C: Left ventricular end-systolic diameter (LVD_{systole}) (mm); FIG. 15D: Left ventricular end-diastolic diameter (LVD_{diastole}) (mm); FIG. 15E: Left ventricular volume during diastole (LVV_{diastole} (μL). Statistical significance was determined by two-way ANOVA with repeated measures and Bonferroni post hoc test.

FIGS. 16A-16D show results of cardiac histology studies demonstrating that FC9402 attenuates TAC-induced left ventricle (LV) fibrosis. FIGS. 16A-16C: Left ventricle sections were stained with Masson's trichrome to reveal deposition of interstitial collagen (blue). Images were captured at 160× magnification; scale bar=50 μm. FIG. 16D: Collagen fraction (fibrosis area, expressed as % of left ventricle area) was quantified using Image J. ^{††}P<0.01 vs. sham. Statistical significance was determined by one-way ANOVA with Tukey-Kramer post hoc test.

FIGS. 17A-17D show results of cardiac histology studies demonstrating that FC9402 attenuates TAC-induced cardiomyocyte hypertrophy. FIG. 17A-17C: Confocal images of left ventricle sections that were stained with wheat germ agglutinin (WGA)-Alexa Fluor 594 conjugate. Scale bar=50 μm. FIG. 17D: Cardiomyocyte cross-sectional area was quantified using ImageJ. Statistical significance was determined by one-way ANOVA with Tukey-Kramer post hoc test.

FIG. 18 shows a graph of the ratio of lung weight to tibia length (LW:TL). The dotted red line indicates the 95% confidence interval for sham mice.

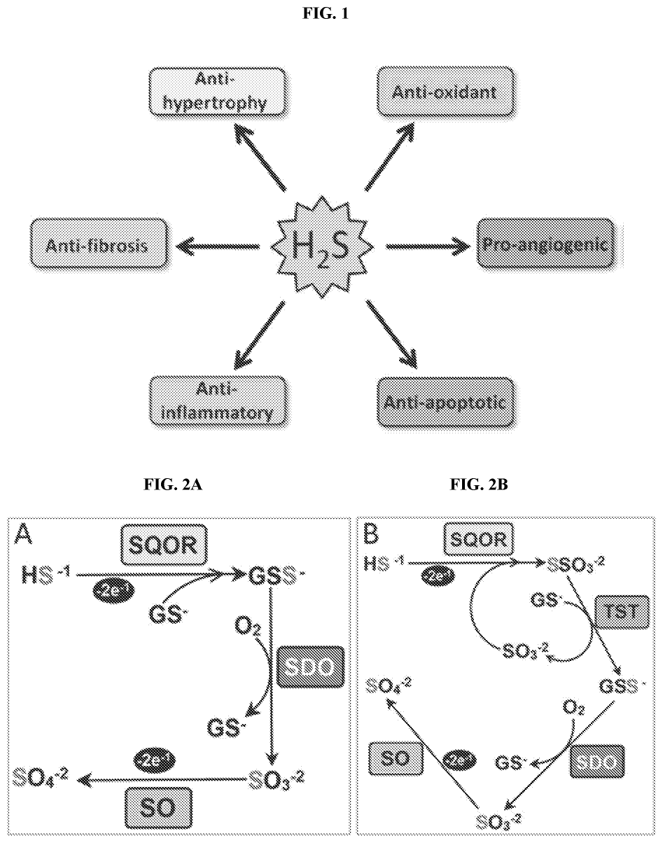

### DETAILED DESCRIPTION OF THE INVENTION

The present invention relates to the discovery of novel compounds that can be used to treat and/or prevent heart failure in a subject. In certain embodiments, the compounds of the invention are sulfide:quinone oxidoreductase (SQOR) inhibitors. In other embodiments, the compounds of the invention increase physiological levels of H₂S in a tissue of the subject. In yet other embodiments, administration of the compounds of the invention treats and/or prevents hypertension and/or atherosclerosis in the subject. In yet other embodiments, the compounds of the invention can be used to treat and/or prevent myocardial ischemia-reperfusion injury. In yet other embodiments, the compounds of the invention help relax vascular smooth muscle, induce vasodilation of isolated blood vessels, and/or reduce blood pressure. In yet other embodiments, the compounds of the invention inhibit leukocyte adherence in mesenteric microcirculation during vascular inflammation. In yet other embodiments, the compounds of the invention inhibit vascular smooth muscle cell proliferation, inflammatory signaling, and platelet aggregation. In yet other embodiments, the compounds of the invention inhibit plaque formation by upregulating anti-oxidant defenses and suppressing reactive oxygen species (ROS) production, low-density lipoprotein oxidation, and foam cell formation. In yet other embodiments, the compounds of the invention inhibit pathological cardiac remodeling and hypertrophy by promoting angiogenesis, stimulating antioxidant defense systems, inhibiting production of ROS, inhibiting release of apoptotoic signaling molecules, activating prosurvival signaling cascades, and inhibiting the development of cardiac fibrosis. In certain non-limiting embodiments, due to the well-established protective properties of H₂S in renal physiology and disease, a new class of drugs to treat heart failure based on modulation of H₂S can address the large unmet need of therapies for 40-50% of heart failure patients with coexisting chronic renal impairment. In yet other embodiments, the subject is a mammal, such as but not limited to a human.

H₂S is the only gasotransmitter that is enzymatically degraded (unlike nitric oxide or carbon monoxide), and this provides basis for a therapeutic strategy for increasing H₂S levels to treat heart failure. The first step in H₂S metabolism is performed by sulfide:quinone oxidoreductase (SQOR), an inner mitochondrial membrane-bound flavoenzyme, which catalyzes a two-electron oxidation of H₂S to sulfane sulfur (S⁰), using glutathione or sulfite as sulfane sulfur acceptor and coenzyme Q (CoQ) as electron acceptor, respectively (FIGS. 2A-2B). Human SQOR can be expressed in Escherichia coli as a catalytically active membrane-bound protein that is readily solubilized and purified to homogeneity in substantial quantities and the X-ray structure of human SQOR was recently reported at 2.59 Å resolution (Jackson et al., 2012, Biochemistry 51: 6804-6815; Jackson et al., 2019, Structure 27: 794-805). Knockout mice lacking sulfur dioxygenase (SDO), which is a downstream enzyme in the H₂S metabolic pathway with glutathione or sulfite as sulfane sulfur acceptor (FIGS. 2A-2B), exhibit ˜40-fold increase in tissue H₂S levels. Slowing H₂S metabolism by inhibiting SQOR thus can achieve the increase needed to normalize H₂S levels in heart failure patients.

Without wishing to be limited by any theory, the strategy of slowing H₂S metabolism by inhibiting SQOR affords advantages as compared with an alternate approach of using inorganic or organic H₂S-releasing donor compounds. Inhibiting H₂S oxidation stimulates signaling most effectively in tissues with high SQOR activity and high oxidative metabolism, such as the heart, whereas H₂S donors are most effective in tissues with low SQOR activity, such as the brain. SQOR inhibitors preferentially increase the concentration of H₂S in mitochondria, an important site of H₂S-mediated protection against MI/R injury. The inorganic donor commonly used in preclinical studies, NaHS, is unsuitable as a therapeutic agent, owing to the rapid release of H₂S. Statins, one of the most commonly used drugs in cardiovascular medicine, slow H₂S metabolism and increase the concentration of H₂S in the vascular cell wall. The observed effect on H₂S metabolism is, however, caused by statin-induced depletion of mitochondrial levels of CoQ and resultant organelle dysfunction.

The compounds of the invention should not be construed to be limited only to treating and/or preventing heart failure in a subject. Additional non-limiting uses of the compounds of the invention include, for example, treating and/or preventing cardiovascular disease, myocardial ischemia-reperfusion injury, cardiomyopathy, vascular abnormalities, cirrhosis, liver injury, kidney injury, vascular calcification, gastric injury induced by drug treatment (such as by non-steroidal anti-inflammatory drugs or NSAIDS), burns, lung injury, neutrophil adhesion, leukocyte-mediated inflammation, erectile dysfunction, irritable bowel syndrome, anti-nociceptive effects in post-inflammatory hypersensitivity, acute coronary syndrome, cardiac arrest, planned cardiac bypass surgery, congestive heart failure, neonatal hypoxia/ischemia, myocardial ischemic reperfusion injury, unstable angina, postangioplasty, aneurysm, trauma, diabetic cardiomyopathy, tissue ischemia following organ transplantation, and/or stroke. Disease states or conditions that result in hypertension and can be treated with the compounds of the invention include, for example, aneurysm, stroke, metabolic syndrome, liver injury, dementia, kidney injury, kidney disease, vascular calcification, angina, peripheral artery disease, and/or transient ischemic attack. Additional non-limiting uses of the compounds of the invention include, for example, promoting wound healing, or pre-treating a subject prior to an ischemic or hypoxic injury or disease insult.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof. Pharmaceutically unacceptable salts may nonetheless possess properties such as high crystallinity, which have utility in the practice of the present invention, such as for example utility in process of synthesis, purification or formulation of compounds useful within the methods of the invention. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate). Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the terms “pharmaceutically effective amount” and “effective amount” and “therapeutically effective amount” refer to a nontoxic but sufficient amount of an agent to provide the desired biological result. That result can be reduction and/or alleviation of the signs, symptoms, or causes of a disease, or any other desired alteration of a biological system. An appropriate therapeutic amount in any individual case can be determined by one of ordinary skill in the art using routine experimentation.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments can be specifically tailored or modified, based on knowledge obtained from the field of pharmacogenomics.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. A non-limiting example is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms can be optionally oxidized and the nitrogen heteroatom can be optionally quaternized. The heteroatom(s) can be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms can be consecutive, such as, for example, —CH₂NHOCH₃, or —CH₂CH₂SSCH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. A non-limiting example is (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

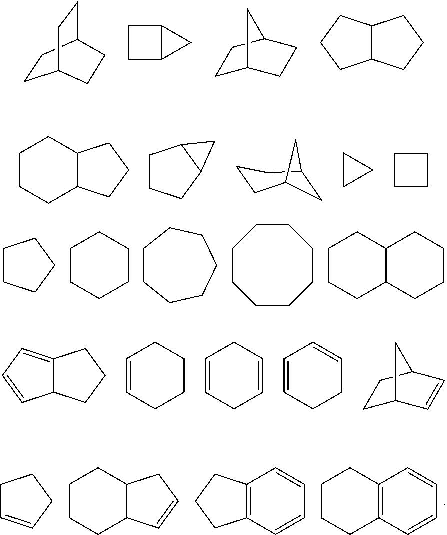

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbomane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, Sand N. In certain embodiments, each heterocycloalkyl group has from 4 to 10 atoms in its ring system, with the proviso that the ring of said group does not contain two adjacent O or S atoms. In other embodiments, the heterocycloalkyl group is fused with an aromatic ring. In certain embodiments, the nitrogen and sulfur heteroatoms can be optionally oxidized, and the nitrogen atom can be optionally quaternized. The heterocyclic system can be attached, unless otherwise stated, at any heteroatom or carbon atom that affords a stable structure. A heterocycle can be aromatic or non-aromatic in nature. In certain embodiments, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

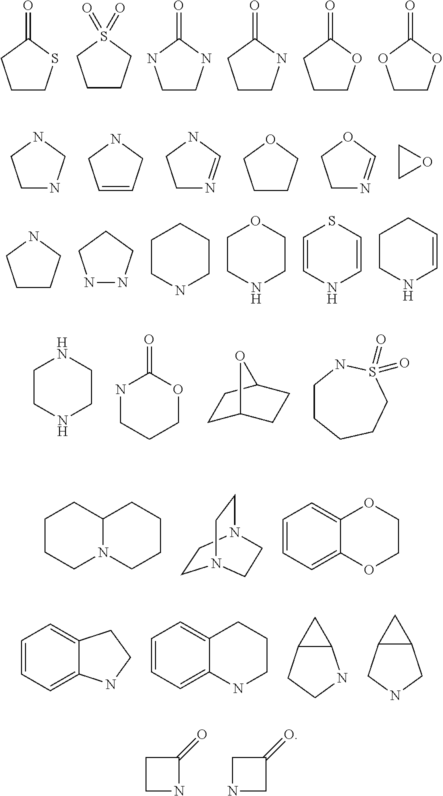

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized JI (pi) electrons, where n is an integer.

As used herein, the term “aryl,” employed alone or in combination with other terms, means, unless otherwise stated, a carbocyclic aromatic system containing one or more rings (typically one, two or three rings), wherein such rings can be attached together in a pendent manner, such as a biphenyl, or can be fused, such as naphthalene. Examples of aryl groups include phenyl, anthracyl, and naphthyl. Non-limiting examples are phenyl and naphthyl, for example phenyl.

As used herein, the term “aryl-(C₁-C₃)alkyl” means a functional group wherein a one- to three-carbon alkylene chain is attached to an aryl group, e.g., —CH₂CH₂-phenyl. Non-limiting examples are aryl-CH₂— and aryl-CH(CH₃)—. The term “substituted aryl-(C₁-C₃)alkyl” means an aryl-(C₁-C₃)alkyl functional group in which the aryl group is substituted. A non-limiting example is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. A non-limiting example is heteroaryl-(CH₂)—. The term “substituted heteroaryl-(C₁-C₃)alkyl” means a heteroaryl-(C₁-C₃)alkyl functional group in which the heteroaryl group is substituted. A non-limiting example is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

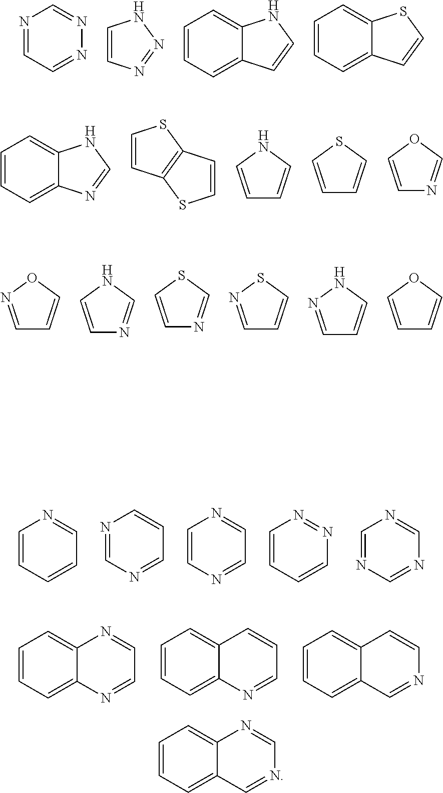

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution can be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group can be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet other embodiments, the substituents are independently selected from the group consisting of C₁₋₆ alkyl, C₁₋₆ alkoxy, halo, acetamido, and nitro. As used herein, where a substituent is an alkyl or alkoxy group, the carbon chain can be branched, straight or cyclic.

As used herein, the term “oxetanoxyl” refers to the group:

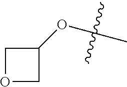 [SMILES: *OC1COC1]

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The compounds of the present invention can be synthesized using techniques well-known in the art of organic synthesis. The starting materials and intermediates required for the synthesis can be obtained from commercial sources or synthesized according to methods known to those skilled in the art.

In one aspect, the invention provides a compound of formula (IA), or a salt, solvate, stereoisomer, geometric isomer, and/or tautomer thereof:

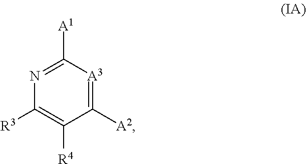

wherein in (IA):

A¹ is-C(R⁷)(R⁸)(R⁹),

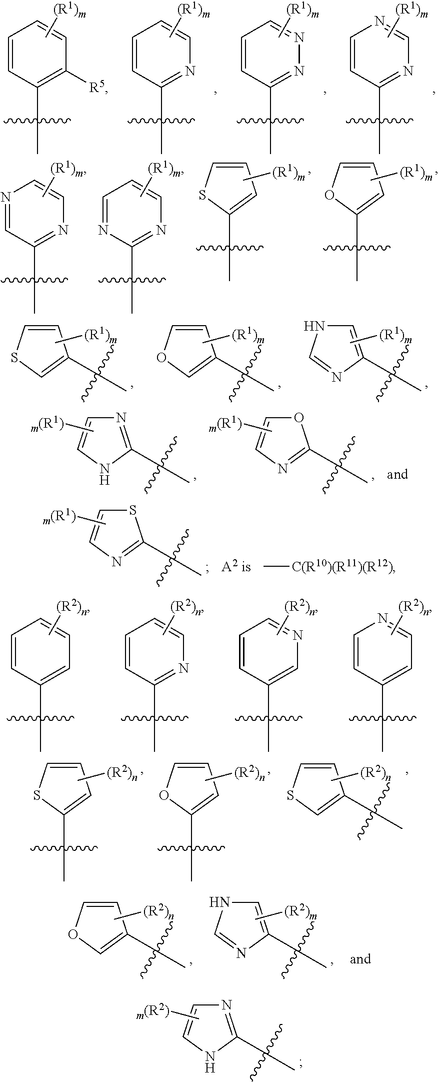

A³ is N or C—R⁶;

m is 1, 2 or 3;

n is 1, 2 or 3;

each occurrence of R¹ is independently selected from the group consisting of H, F, Cl, Br, I, —OR, —SR, —S(═O)(C₁-C₆ alkyl), —S(═O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, N¹-β-propiolactam, N¹-γ-butyrolactam, N¹-δ-valerolactam, and N¹-ε-caprolactam;
wherein, if A¹ is pyridinyl, then (i) at least one R¹ is not H, or (ii) R⁴ is not —CN;
wherein, if two R¹ groups are present in neighboring carbons, they optionally
combine to form the divalent group —O(CH₂)₁₋₃O—;

each occurrence of R² is independently selected from the group consisting of H, F, Cl, Br, I, —OR, —SR, —S(═O)(C₁-C₆ alkyl), —S(═O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, N¹-β-propiolactam, N¹-γ-butyrolactam, N¹-δ-valerolactam, and N¹-ε-caprolactam;
wherein, if two R² groups are present in neighboring carbons, they optionally
combine to form the divalent group —O(CH₂)₁₋₃O—;

R⁴ is independently selected from the group consisting of H, —CH₃, —CN, —C≡CH, —C(═O)OR, and —C(═O)NHR;

R³, R⁵ and R⁶ are selected such that:
(a) R⁵ and R⁶ combine to form the divalent group —O— or —CH₂—, and
   R³ is selected from the group consisting of H, C₁-C₆, alkyl, (C₃-C₆)alkoxy, (C₁-C₆)haloalkyl, (C₁-C₆)haloalkoy, dialkylamino(C₁-C₆)alkoxy, —NR₂, N¹-β-propiolactam, N¹-γ-butyrolactam, N¹-δ-valerolactam, oxetanoyl, oxetanoxyl N¹-ε-caprolactam, (C₂-C₆)alkenyloxy, —O(CH₂)₂₋₄O(CH₂)₁₋₄C(═O)OR, —O(CH₂)₁₋₄NRR, —O(CH₂)₁₋₄(heteroaryl), —O(CH₂)₁₋₄CN, —O(CH₂)₁₋₄(pyrrolidin-2-one), —O(CH₂)₁₋₄C(═O)OR, —O(CH₂)₁₋₄C(═O)NRR, —O(CH₂)₁₋₄NRC(═O)R, N⁴-morpholinyl, —O(CH₂)₂₋₄(N⁴-morpholinyl), —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), —O(CH₂)₂₋₄(N¹-δ-valerolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, and —C(═O)OR;
(b) R⁵ and R⁶ combine to form the divalent group —O—, —CH(C₁-C₆ alkyl) or —C(C₁-C₆ alkyl)(C₁-C₆ alkyl)-, and
   R³ is selected from the group consisting of H, —OH, C₁-C₆ alkyl, (C₂-C₆)alkoxy, (C₁-C₆)haloalkyl, (C₁-C₆)haloalkoy, benzyloxy, —NR₂, N⁴-morpholinyl, N¹-piperidinyl, N¹-β-propiolactam, N¹-γ-butyrolactam, N¹-δ-valerolactam, N¹-ε-caprolactam, oxetanoxyl, —SH, —S(═O)(C₁-C₆ alkyl), —S(═O)₂(C₁-C₆ alkyl), (C₂-C₆)alkenyloxy, —O(CH₂)₂₋₄O(CH₂)₁₋₄C(═O)OR, —O(CH₂)₁₋₄NRR, —O(CH₂)₁₋₄(heteroaryl), —O(CH₂)₁₋₄CN, —O(CH₂)₁₋₄(pyrrolidin-2-one), —O(CH₂)₁₋₄C(═O)OR, —O(CH₂)₁₋₄C(═O)NRR, —O(CH₂)₁₋₄NRC(═O)R, N⁴-morpholinyl, —O(CH₂)₂₋₄(N⁴-morpholinyl), —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), —O(CH₂)₂₋₄(N¹-δ-valerolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, and —C(═O)OR;
(c) R⁵ and R⁶ combine to form the divalent group —CH₂CH₂—, and
   R³ is selected from the group consisting of H, (C₁-C₆)alkoxy, (C₁-C₆)haloalkyl, (C₁-C₆)haloalkoxy, —NH(C₁-C₆ alkyl), —N(C₁-C₆ alkyl)(C₁-C₆ alkyl), N¹-β-propiolactam, N¹-γ-butyrolactam, N¹-δ-valerolactam, oxetanoxyl, N¹-ε-caprolactam, (C₂-C₆)alkenyloxy, —O(CH₂)₂₋₄O(CH₂)₁₋₄C(═O)OR, —O(CH₂)₁₋₄NRR, —O(CH₂)₁₋₄(heteroaryl), —O(CH₂)₁₋₄CN, —O(CH₂)₁₋₄(pyrrolidin-2-one), —O(CH₂)₁₋₄C(═O)OR, —O(CH₂)₁₋₄C(═O)NRR, —O(CH₂)₁₋₄NRC(═O)R, N⁴-morpholinyl, —O(CH₂)₂₋₄(N⁴-morpholinyl), —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), —O(CH₂)₂₋₄(N¹-δ-valerolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, and —C(═O)OR,
      with the proviso that R² is not H, para-F or para-OMe, and, if two R² groups are present in neighboring carbons, they do not combine to form the divalent group —O(CH₂)₁₋₃O—;
(d) R⁵ and R⁶ combine to form the divalent group —CR₂CR₂—, wherein at least one R is not H;
   R³ is selected from the group consisting of H, —OH, C₁-C₆ alkyl, (C₁-C₆)alkoxy, (C₂-C₆)alkenyloxy, (C₁-C₆)haloalkyl, (C₁-C₆)haloalkoxy, benzyloxy, —O(CH₂)₂₋₄O(CH₂)₁₋₄C(═O)OR, —O(CH₂)₁₋₄NRR, —O(CH₂)₁₋₄(heteroaryl), —O(CH₂)₁₋₄CN, —O(CH₂)₁₋₄(pyrrolidin-2-one), —O(CH₂)₁₋₄C(═O)OR, —O(CH₂)₁₋₄C(═O)NRR, —O(CH₂)₁₋₄NRC(═O)R, —NR₂, N⁴-morpholinyl, —O(CH₂)₂₋₄(N⁴-morpholinyl), N¹-piperidinyl, —O(CH₂)₂₋₄(N¹-piperidinyl), N¹-β-propiolactam, —O(CH₂)₂₋₄(N¹-β-propiolactam), N-γ-butyrolactam, —O(CH₂)₂₋₄(N¹-γ-butyrolactam), N¹-δ-valerolactam, —O(CH₂)₂₋₄(N¹-δ-valerolactam), N¹-ε-caprolactam, —O(CH₂)₂₋₄(N¹-δ-valerolactam), oxetanoxyl, —O(CH₂)₂₋₄(N¹-δ-valerolactam), N¹-piperazinyl, —O(CH₂)₂₋₄(N¹-piperazinyl), —SH, —S(═O)(C₁-C₆ alkyl), and —S(═O)₂(C₁-C₆ alkyl); wherein the 4-position of the piperazinyl group is optionally substituted with a group selected from R, —C(═O)R, and —C(═O)OR, and
(e) R⁵ is R₁, and R⁶ is H or C₁-C₆ alkyl, and
   R³ is selected from the group consisting of H, (C₁-C₆)alkoxy, (C₁-C₆)haloalkyl, (C₁-C₆)haloalkoxy, —NR₂, N¹-β-propiolactam, N¹-γ-butyrolactam, N¹-δ-valerolactam, oxetanoxyl, N-ε-caprolactam, (C₂-C₆)alkenyloxy, —O(CH₂)₂₋₄O(CH₂)₁₋₄C(═O)OR, —O(CH₂)₁₋₄NRR, —O(CH₂)₁₋₄(heteroaryl), —O(CH₂)₁₋₄CN, —O(CH₂)₁₋₄(pyrrolidin-2-one), —O(CH₂)₁₋₄C(═O)OR, —O(CH₂)₁₋₄C(═O)NRR, —O(CH₂)₁₋₄NRC(═O)R, N⁴-morpholinyl, —O(CH₂)₂₋₄(N⁴-morpholinyl), —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), —O(CH₂)₂₋₄(N¹-δ-valerolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, and —C(═O)OR,
      with the proviso that R² is not H, meta-F or meta-NH₂;

each of R⁷, R⁸, R⁹, R¹⁰, R¹¹, and R¹² is independently optionally substituted C₁-C₁₀ alkyl, wherein two or three of R⁷, R⁸, R⁹ or of R¹⁰, R¹¹, R¹² can optionally combine to form monocyclic or polycyclic groups (such as, for example, cyclohexyl or adamantly);

each occurrence of R is independently H or C₁-C₆ alkyl.

In one aspect, the invention provides a compound of formula (IA), or a salt, solvate, stereoisomer, geometric isomer, and/or tautomer thereof:

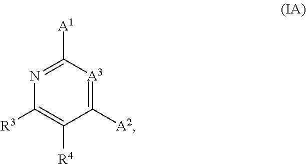

wherein in (IA):

A¹ is —C(R⁷)(R⁸)(R⁹),

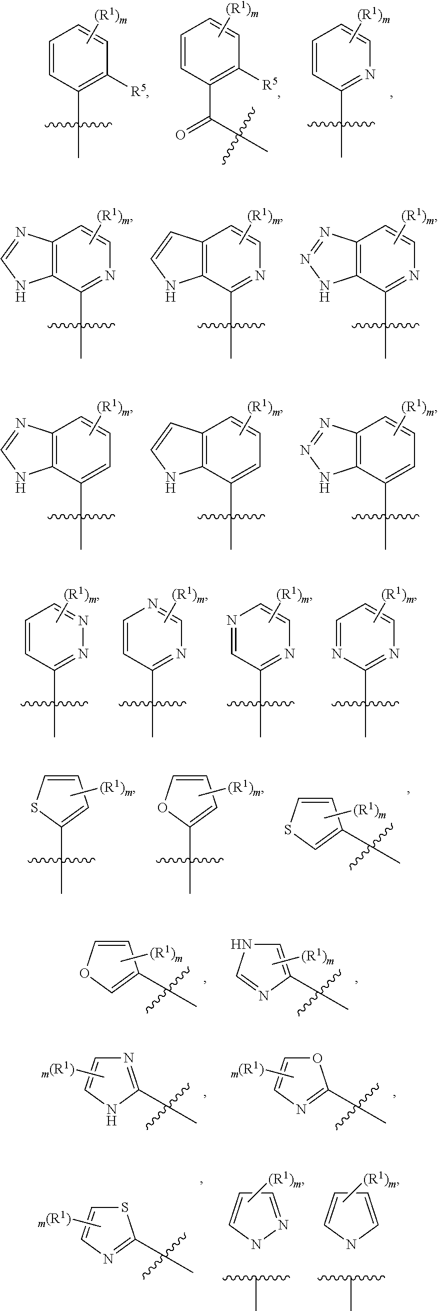

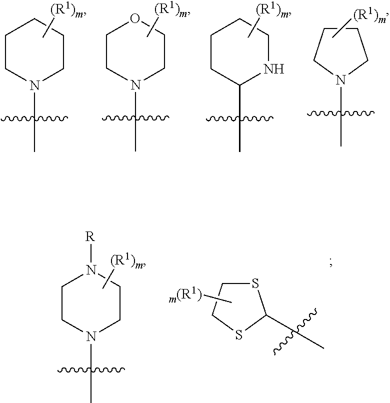

A² is —(C(R¹⁰)(R¹¹)(R¹²),

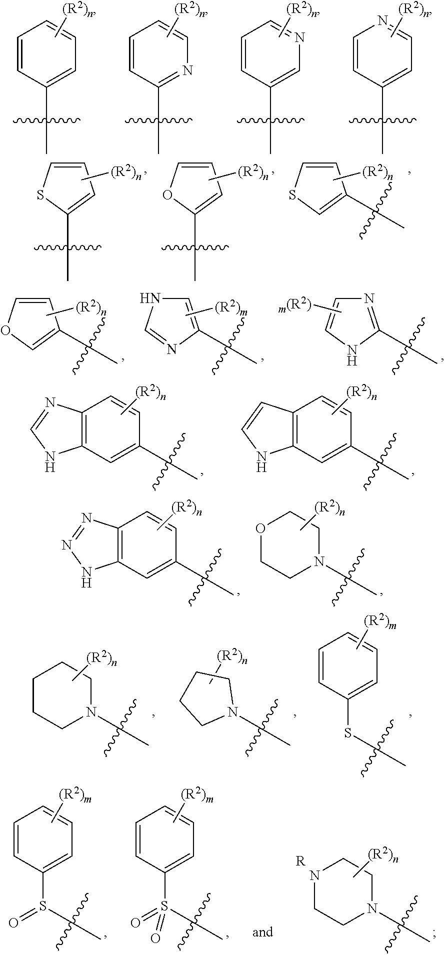

A³ is N or C—R⁶;

m is 1, 2 or 3;

n is 1, 2 or 3;

each occurrence of R¹ is independently selected from the group consisting of H, F, Cl, Br, I, —OR, —SR, —S(O)(C₁-C₆ alkyl), —S(O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R¹ groups are present in neighboring carbons, they optionally combine to form the divalent group —O(CH₂)₁₋₃O—;

each occurrence of R² is independently selected from the group consisting of H, F, Cl, Br, I, —OR, —SR, —S(O)(C₁-C₆ alkyl), —S(O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R² groups are present in neighboring carbons, they optionally combine to form the divalent group —O(CH₂)₁₋₃O—;
with the proviso that R² is not positioned ortho to the bond between the central heteroaryl ring and A²;

R⁴ is independently selected from the group consisting of —CH₃, —CN, —C≡CR, —C(═O)OR, and —C(═O)NR₂; or R⁴ can combine with R³ to form a 5-membered amino heterocycle such that the compound of formula (IA) has one of the following structures:

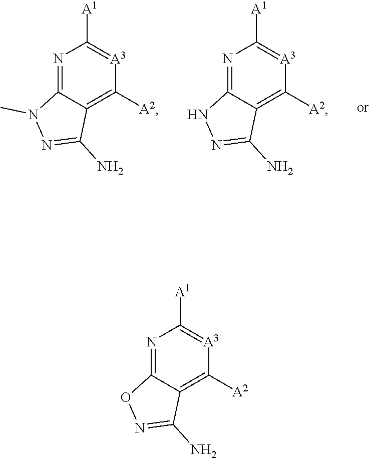

R³, R⁵ and R⁶ are selected such that:
(a) R⁵ and R⁶ combine to form the divalent group —O—, —CH₂—, —CH(C₁-C₆ alkyl) or —C(C₁-C₆ alkyl)(C₁-C₆ alkyl)-, and
   R³ is selected from the group consisting of C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆) alkyl), —S(O)(C₁-C₆ alkyl), —S(O)₂(C₁-C₆ alkyl), N¹-βpropiolactam, N¹-γ-butyrolactam, N¹-δ-valerolactam, oxetanoyl, oxetanoxyl N¹-ε-caprolactam, (C₂-C₆)alkenyloxy, (C₂-C₆)alkynyloxy, —O(CH₂)₂₋₄OR, —O(CH₂)₂₋₄O(CH₂)₁₋₄C(═O)OR, —O(CH₂)₂₋₄NRR, —O(CH₂)₁₋₄(aryl), O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, N¹-pyrrolidinyl, —O(CH₂)₂₋₄(N¹-pyrrolidinyl), —N¹-piperidinyl, —O(CH₂)₂₋₄(N⁴-morpholinyl), —O(CH₂)₂₋₄(N¹-piperidinyl), —O(CH₂)₂₋₄(N¹—β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;
(b) R⁵ and R⁶ combine to form the divalent group —CH₂CH₂—, and
   R³ is selected from the group consisting of C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆)alkyl), —S(O)(C₁-C₆)alkyl), —S(O)₂(C₁-C₆ alkyl), N¹-β-propiolactam, N¹-γ-butyrolactam, N¹-δ-valerolactam, oxetanoyl, oxetanoxyl, N¹-ε-caprolactam, (C₂-C₆)alkenyloxy, (C₂-C₆)alkynyloxy, —O(CH₂)₂₋₄OR, —O(CH₂)₂₋₄O(CH₂)₂₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl. —O(CH₂)₂₋₄(N¹-pyrrolidinyl), —N¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR,
(c) R⁵ and R⁶ combine to form the divalent group —CR₂CR₂—, wherein at least one R is not H;
   R³ is selected from the group consisting of C₁-C₆ alkyl, (C₁-C₆)alkoxy, (C₁-C₆)haloalkyl, (C₁-C₆)haloalkoxyl, dialkylamino(C₁-C₆)alkoxy, —NR₂, N¹-β-propiolactam, N¹-γ-butyrolactam, N¹-δ-valerolactam, oxetanoyl, oxetanoxyl N¹-ε-caprolactam, (C₂-C₆)alkenyloxy, (C₂-C₆)alkynyloxy, —O(CH₂)₂₋₄OR, —O(CH₂)₂₋₄O(CH₂)₁₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl. —O(CH₂)₂₋₄(N¹-pyrrolidinyl)ⱼ N¹-piperidinyl, —O(CH₂)₂₋₄(N¹-piperidinyl). —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR, and
(d) R⁵ is R₁, and R⁶ is H or C₁-C₆ alkyl, and
   R³ is selected from the group consisting of C₁-C₆ alkyl, (C₁-C₆)alkoxy, (C₁-C₆)haloalkyl, (C₁-C₆)haloalkoy, dialkylamino(C₁-C₆)alkoxy, —NR₂, N¹-(4-propiolactam, N¹-γ-butyrolactam, N¹-δ-valerolactam, oxetanoyl, oxetanoxyl, N-ε-caprolactam, (C₂-C₆)alkenyloxy, —(C₂-C₆)alkynyloxy, —O(CH₂)₂₋₄OR, —O(CH₂)₂₋₄O(CH₂)₁₋₄C(═O)OR, —O(CH₂)₁₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl. —O(CH₂)₂₋₄(N¹-pyrrolidinyl)ⱼN¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam) N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;

each of R⁷, R⁸, R⁹, R¹⁰, R¹¹, and R¹² is independently selected from optionally substituted C₁-C₁₀ alkyl, wherein two or three of R⁷, R⁸, R⁹ or of R¹⁰, R¹¹, R¹² can optionally combine to form monocyclic or polycyclic groups (such as, for example, cyclohexyl or adamantyl);

each occurrence of R is independently H or C₁-C₆ alkyl,

with the provisos that

i) when R³ is (C₁-C₆)alkoxy, R⁴ is CN and A¹ is

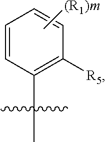

then A² is not

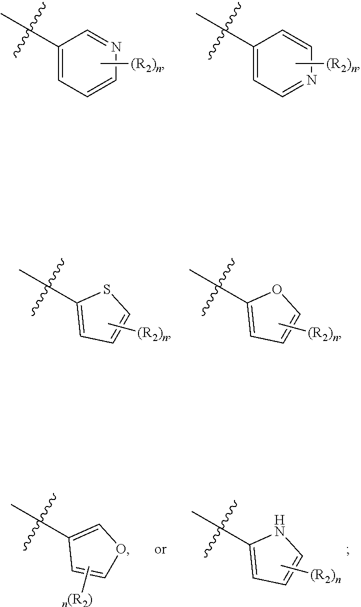

ii) when R³ is (C₁-C₆)alkoxy, R⁴ is CN and A¹ is

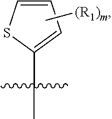

then A² is not

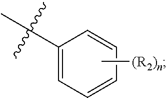

iii) when R³ is (C₁-C₆)alkoxy, R⁴ is CN and A¹ is

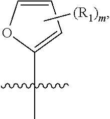

than A² is not

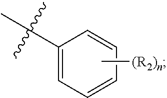

iv) when R³ is (C₁-C₆)alkoxy, R⁴ is CN and A¹ is

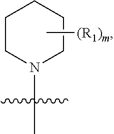

then A² is not

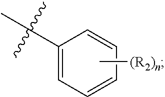

v) when R³ is (C₁-C₆)alkoxy, R⁴ is CN and A¹ is

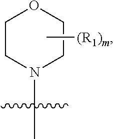

then A² is not

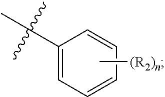

vi) when R³ is (C₁-C₆)alkoxy, R⁴ is CN, A¹ is

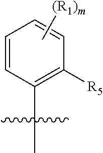

and R⁵ and R⁶ combine to form the divalent group —CH₂CH₂—,

then A² is not

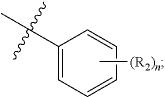

vii) when R³ is (C₁-C₆)alkoxy, R⁴ is C(═O)OR and A¹ is

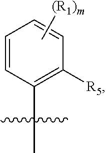

then A² is not

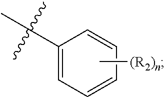

vii) when R³ is (C₁-C₆)alkoxy, R⁴ is CN and A¹ is

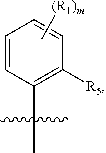

then A² is not

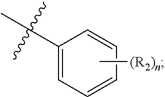

except that the compound of formula (IA) can be any of the following compounds:

| | |
|---|---|
| 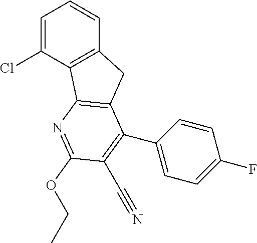 [SMILES: CCOc1nc2c(c(-c3ccc(F)cc3)c1C#N)Cc1cccc(Cl)c1-2] | 9-Chloro-2- ethoxy-4- (4-fluorophenyl)-5H- indeno[1,2-b] pyridine- 3-carbonitrile |
| 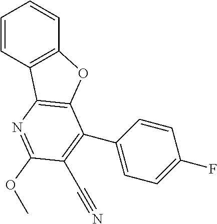 [SMILES: COc1nc2c(oc3ccccc32)c(-c2ccc(F)cc2)c1C#N] | 4-(4- Fluorophenyl)-2- methoxybenzofuro [3,2-b]pyridine-3- carbonitrile |
| 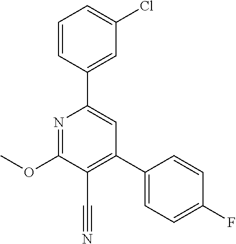 [SMILES: COc1nc(-c2cccc(Cl)c2)cc(-c2ccc(F)cc2)c1C#N] | 6-(3- Chlorophenyl)-4- (4-fluorophenyl)-2- methoxypyridine-3- carbonitrile |
| 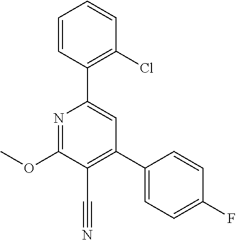 [SMILES: COc1nc(-c2ccccc2Cl)cc(-c2ccc(F)cc2)c1C#N] | 6-(2-Chlorophenyl)- 4-(4-fluorophenyl)-2- methoxypyridine-3- carbonitrile |
| 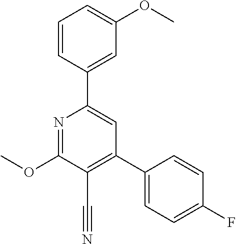 [SMILES: COc1cccc(-c2cc(-c3ccc(F)cc3)c(C#N)c(OC)n2)c1] | 4-(4- Fluorophenyl)-2- methoxy-6-(3- methoxyphenyl) pyridine- 3-carbonitrile |
| 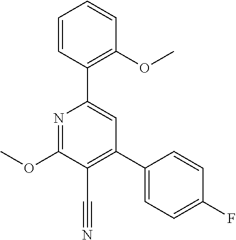 [SMILES: COc1ccccc1-c1cc(-c2ccc(F)cc2)c(C#N)c(OC)n1] | 4-(4- Fluorophenyl)-2- methoxy-6-(2- methoxyphenyl) pyridine-3- carbonitrile |
| 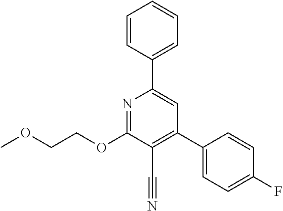 [SMILES: COCCOc1nc(-c2ccccc2)cc(-c2ccc(F)cc2)c1C#N] | 2-(2- Methoxyethoxy)-4- (4-fluorophenyl)-6- phenylpyridine-3- carbonitrile |
| 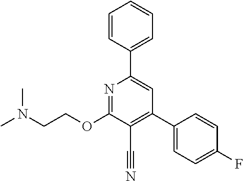 [SMILES: CN(C)CCOc1nc(-c2ccccc2)cc(-c2ccc(F)cc2)c1C#N] | 2-(2- (Dimethylamino) ethoxy)-4-(4- fluorophenyl)-6- phenylpyridine-3- carbonitrile |
| 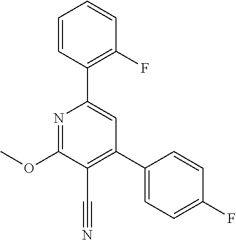 [SMILES: COc1nc(-c2ccccc2F)cc(-c2ccc(F)cc2)c1C#N] | 6-(2- Fluorophenyl)-4- (4-fluorophenyl)-2- methoxypyridine-3- carbonitrile |
| 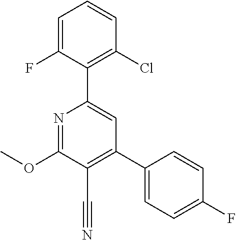 [SMILES: COc1nc(-c2c(F)cccc2Cl)cc(-c2ccc(F)cc2)c1C#N] | 6-(2-Chloro-6-fluoro- phenyl)-4-(4-fluoro- phenyl)-2-methoxy- nicotinonitrile |
| 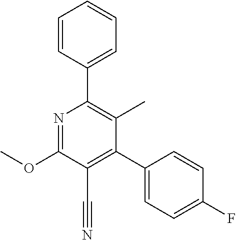 [SMILES: COc1nc(-c2ccccc2)c(C)c(-c2ccc(F)cc2)c1C#N] | 4-(4-Fluoro- phenyl)-2- methoxy-5- methyl-6- phenyl- nicotinonitrile |
| 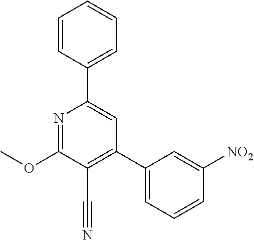 [SMILES: COc1nc(-c2ccccc2)cc(-c2cccc([N+](=O)[O-])c2)c1C#N] | 2-Methoxy-4-(3- nitrophenyl)-6- phenylpyridine- 3-carbonitrile |
| 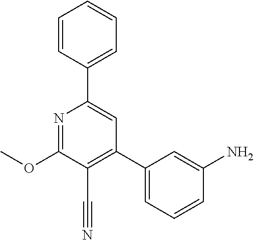 [SMILES: COc1nc(-c2ccccc2)cc(-c2cccc(N)c2)c1C#N] | 4-(3- Aminophenyl)-2- methoxy-6- phenylpyridine- 3-carbonitrile |
| 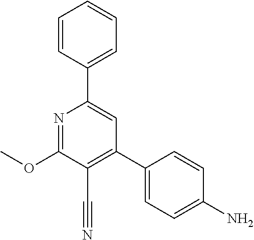 [SMILES: COc1nc(-c2ccccc2)cc(-c2ccc(N)cc2)c1C#N] | 4-(4- Aminophenyl)- 2-methoxy-6- phenylpyridine- 3-carbonitrile |
| 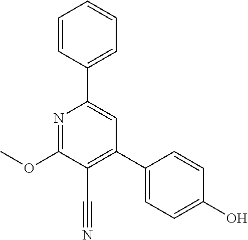 [SMILES: COc1nc(-c2ccccc2)cc(-c2ccc(O)cc2)c1C#N] | 4-(4- Hydroxyphenyl)-2- methoxy-6- phenylpyridine-3- carbonitrile |
| 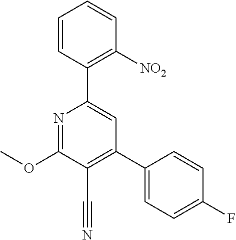 [SMILES: COc1nc(-c2ccccc2[N+](=O)[O-])cc(-c2ccc(F)cc2)c1C#N] | 4-(4- Fluorophenyl)-2- methoxy-6-(2- nitrophenyl) pyridine-3- carbonitrile |
| 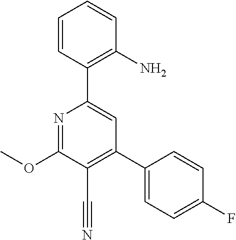 [SMILES: COc1nc(-c2ccccc2N)cc(-c2ccc(F)cc2)c1C#N] | 6-(2- Aminophenyl)-4- (4-fluorophenyl)-2- methoxypyridine-3- carbonitrile |
| 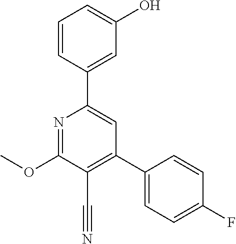 [SMILES: COc1nc(-c2cccc(O)c2)cc(-c2ccc(F)cc2)c1C#N] | 4-(4- fluorophenyl)-6- (3-hydroxy- phenyl)-2- methoxypyridine- 3-carbonitrile |
| 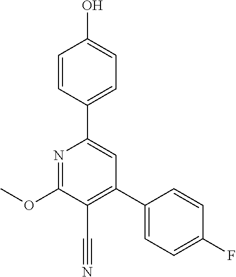 [SMILES: COc1nc(-c2ccc(O)cc2)cc(-c2ccc(F)cc2)c1C#N] | 4-(4- Fluorophenyl)-6- (4-hydroxy- phenyl)-2- methoxypyridine- 3-carbonitrile |

viii) when R³ is (C₁-C₆)alkoxy, R⁴ is CN and A¹ is

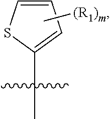

then A² is not

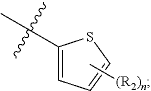

ix) when R³ is (C₁-C₆)alkoxy, R⁴ is CN and A¹ is

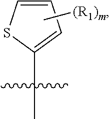

then A² is not

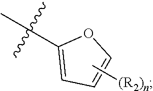

x) when R³ is (C₁-C₆)alkoxy, R⁴ is CN and A¹ is

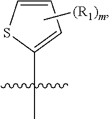

then A is not:

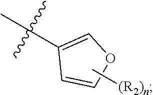

xi) when R³ is (C₁-C₆)alkoxy. R⁴ is CN and A¹ is

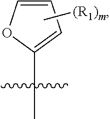

then A² is not;

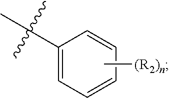

xii) when R³ is (C₁-C₆)alkoxy, R⁴ is CN and A¹ is

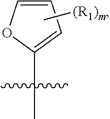

then A² is not;

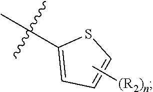

xiii) when R³ is (C₁-C₆)alkoxy, R⁴ is CN and A¹ is

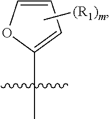

then A is not

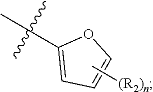

xiv) when R³ is (C₁-C₆)alkoxy, R⁴ is CN and A¹ is

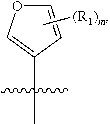

then A² is not

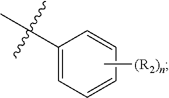

xv) when R³ is (C₁-C₆)alkoxy, R⁴ is CN and A¹ is

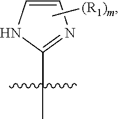

then A² is not

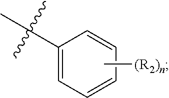

xvi) when R³ is (C₁-C₆)alkoxy, R⁴ is CN and A¹ is

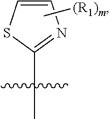

then A is not

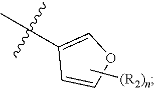

and with the proviso that the compound (of formula (IA)) is not any of the following compounds:

| | |
|---|---|
| 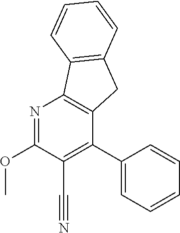 [SMILES: COc1nc2c(c(-c3ccccc3)c1C#N)Cc1ccccc1-2] | 2-Methoxy-4-phenyl-5H- indeno[1,2-b]pyridine-3- carbonitrile |
| 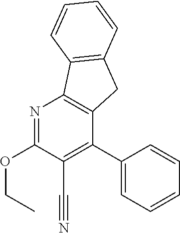 [SMILES: CCOc1nc2c(c(-c3ccccc3)c1C#N)Cc1ccccc1-2] | 2-Ethoxy-4-phenyl-5H- indeno[1,2-b]pyridine-3- carbonitrile |
| 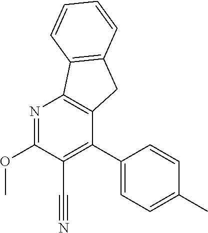 [SMILES: COc1nc2c(c(-c3ccc(C)cc3)c1C#N)Cc1ccccc1-2] | 2-Methoxy-4-p-tolyl-5H- ideno[1,2-b]pyridine-3- carbonitrile |
| 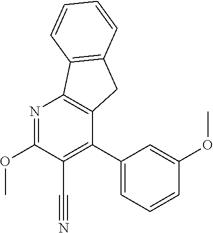 [SMILES: COc1cccc(-c2c(C#N)c(OC)nc3c2Cc2ccccc2-3)c1] | 2-Methoxy-4-(3-methoxy- phenyl)-5H-indeno[1,2- b]pyridine-3-carbonitrile |
| 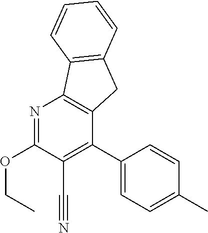 [SMILES: CCOc1nc2c(c(-c3ccc(C)cc3)c1C#N)Cc1ccccc1-2] | 2-Ethoxy-4-p-tolyl-5H- indeno[1,2-b]pyridine-3- carbonitrile |
| 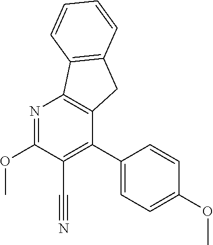 [SMILES: COc1ccc(-c2c(C#N)c(OC)nc3c2Cc2ccccc2-3)cc1] | 2-Methoxy-4-(4-methoxy- phenyl)-5H-indeno[1,2- b]pyridine-3-carbonitrile |
| 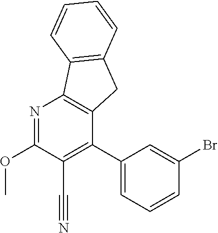 [SMILES: COc1nc2c(c(-c3cccc(Br)c3)c1C#N)Cc1ccccc1-2] | 4-(3-Bromo-phenyl)-2- methoxy-5H-indeno[1,2- b]pyridine-3 -carbonitrile |
| 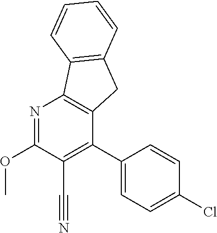 [SMILES: COc1nc2c(c(-c3ccc(Cl)cc3)c1C#N)Cc1ccccc1-2] | 4-(4-Chloro-phenyl)-2- methoxy-5H-indeno[1,2- b]pyridine-3-carbonitrile |
| 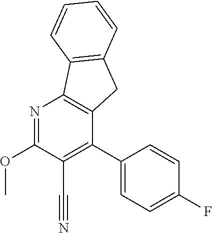 [SMILES: COc1nc2c(c(-c3ccc(F)cc3)c1C#N)Cc1ccccc1-2] | 4-(4-Fluoro-phenyl)-2- methoxy-5H-indeno[1,2- b]pyridine-3-carbonitrile |
| 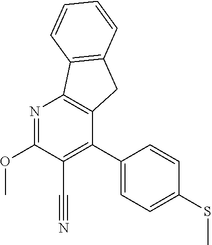 [SMILES: COc1nc2c(c(-c3ccc(SC)cc3)c1C#N)Cc1ccccc1-2] | 2-Methoxy-4-(4- methylsulfanyl-phenyl)-5H- indeno[1,2-b]pyridine-3- carbonitrile |
| 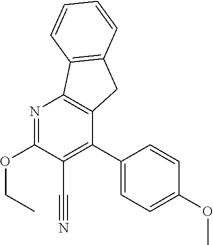 [SMILES: CCOc1nc2c(c(-c3ccc(OC)cc3)c1C#N)Cc1ccccc1-2] | 2-Ethoxy-4-(4-methoxy- phenyl)-5H-indeno[1,2- b]pyridine-3-carbonitrile |
| 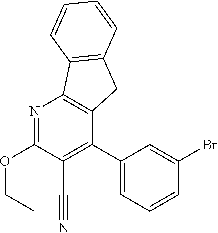 [SMILES: CCOc1nc2c(c(-c3cccc(Br)c3)c1C#N)Cc1ccccc1-2] | 4-(3-Bromo-phenyl)-2- ethoxy-5H-indeno[1,2- b]pyridine-3-carbonitrile |
| 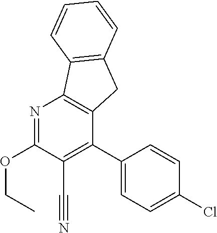 [SMILES: CCOc1nc2c(c(-c3ccc(Cl)cc3)c1C#N)Cc1ccccc1-2] | 4-(4-Chloro-phenyl)-2- ethoxy-5H-indeno[1,2- b]pyridine-3-carbonitrile |
| 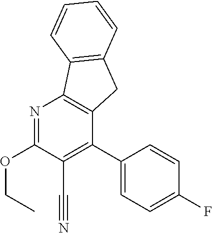 [SMILES: CCOc1nc2c(c(-c3ccc(F)cc3)c1C#N)Cc1ccccc1-2] | 2-Ethoxy-4-(4-fluoro- phenyl)-5H-indeno[1,2- b]pyridine-3-carbonitrile |
| 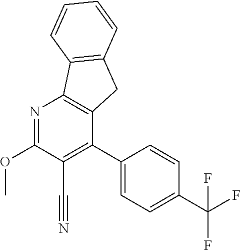 [SMILES: COc1nc2c(c(-c3ccc(C(F)(F)F)cc3)c1C#N)Cc1ccccc1-2] | 2-Methoxy-4-(4- trifluoromethyl-phenyl)-5H- indeno[1,2-b]pyridine-3- carbonitrile |
| 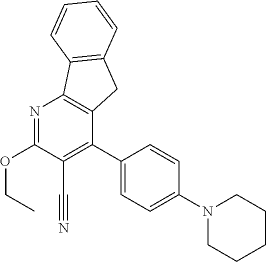 [SMILES: CCOc1nc2c(c(-c3ccc(N4CCCCC4)cc3)c1C#N)Cc1ccccc1-2] | 2-Ethoxy-4-(4-piperidin-1- yl-phenyl)-5H-indeno[1,2- b]pyridine-3-carbonitrile |
| 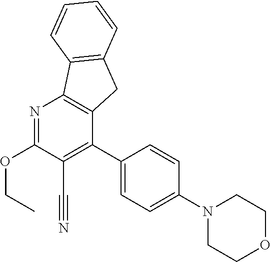 [SMILES: CCOc1nc2c(c(-c3ccc(N4CCOCC4)cc3)c1C#N)Cc1ccccc1-2] | 2-Ethoxy-4-(4-morpholin-4- yl-phenyl)-5H-indeno[1,2- b]pyridine-3-carbonitrile |
| 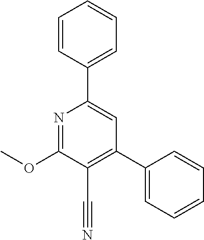 [SMILES: COc1nc(-c2ccccc2)cc(-c2ccccc2)c1C#N] | 6-Methoxy-4-phenyl- [2,2′]bipyridinyl- 5-carbonitrile |
| 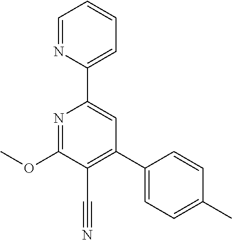 | 6-Methoxy-4-p-tolyl- [2,2′]bipyridinyl- 5-carbonitrile |
| 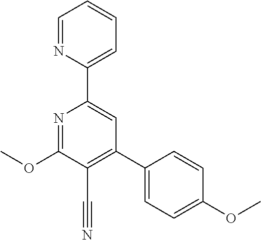 | 6-Methoxy-4-(4-methoxy- phenyl)-[2,2′]bipyridinyl- 5-carbonitrile |
| 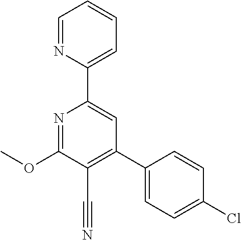 [SMILES: COc1nc(-c2ccccn2)cc(-c2ccc(Cl)cc2)c1C#N] | 4-(4-Chloro-phenyl)-6- methoxy-[2,2′]bipyridinyl- 5-carbonitrile |
| 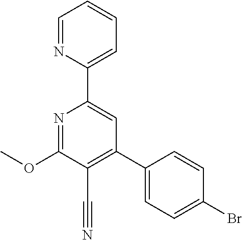 [SMILES: COc1nc(-c2ccccn2)cc(-c2ccc(Br)cc2)c1C#N] | 4-(4-Bromo-phenyl)-6- methoxy-[2,2′]bipyridinyl- 5-carbonitrile |
| 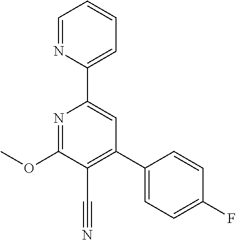 [SMILES: COc1nc(-c2ccccn2)cc(-c2ccc(F)cc2)c1C#N] | 4-(4-Fluoro-phenyl)-6- methoxy-[2,2′]bipyridinyl- 5-carbonitrile |
| 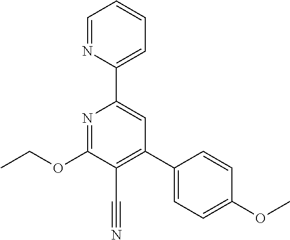 [SMILES: CCOc1nc(-c2ccccn2)cc(-c2ccc(OC)cc2)c1C#N] | 6-Ethoxy-4-(4-methoxy-phenyl)- [2,2′]bipyridinyl- 5-carbonitrile |
| 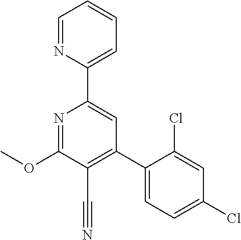 [SMILES: COc1nc(-c2ccccn2)cc(-c2ccc(Cl)cc2Cl)c1C#N] | 4-(2,4-Dichloro-phenyl)-6- methoxy-[2,2′]bipyridinyl- 5-carbonitrile |
| 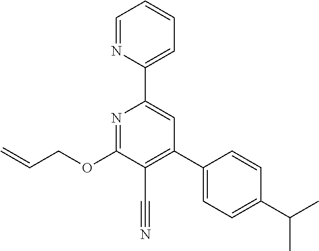 [SMILES: C=CCOc1nc(-c2ccccn2)cc(-c2ccc(C(C)C)cc2)c1C#N] | 6-Allyloxy-4-(4-isopropyl- phenyl)-[2,2′]bipyridinyl- 5-carbonitrile |
| 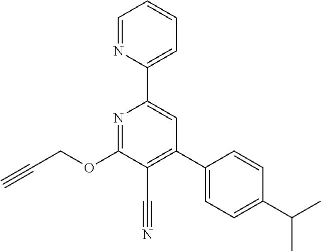 [SMILES: C#CCOc1nc(-c2ccccn2)cc(-c2ccc(C(C)C)cc2)c1C#N] | 4-(4-Isopropyl-phenyl)-6-prop- 2-ynyloxy-[2,2′]bipyridinyl- 5-carbonitrile |
| 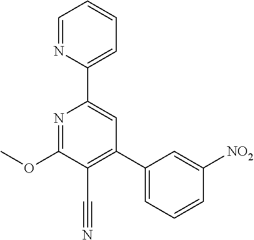 [SMILES: COc1nc(-c2ccccn2)cc(-c2cccc([N+](=O)[O-])c2)c1C#N] | 6-Methoxy-4-(3-nitro-phenyl)- [2,2′]bipyridinyl-5-carbonitrile |
| 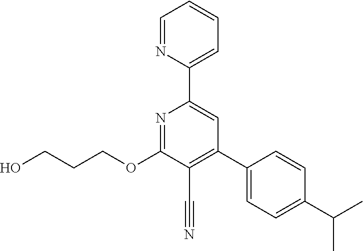 [SMILES: CC(C)c1ccc(-c2cc(-c3ccccn3)nc(OCCCO)c2C#N)cc1] | 6-(3-Hydroxy-propoxy)-4-(4- isopropyl-phenyl)- [2,2′]bipyridinyl-5-carbonitrile |
| 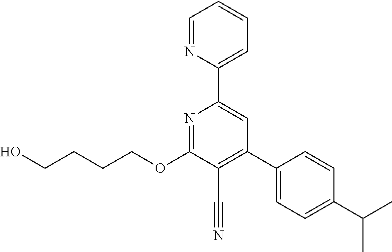 [SMILES: CC(C)c1ccc(-c2cc(-c3ccccn3)nc(OCCCCO)c2C#N)cc1] | 6-(4-Hydroxy-butoxy)-4-(4- isopropyl-phenyl)- [2,2′]bipyridinyl-5-carbonitrile |
| 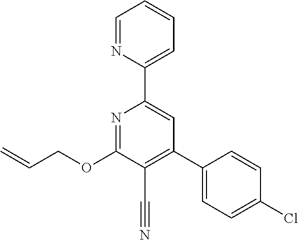 [SMILES: C=CCOc1nc(-c2ccccn2)cc(-c2ccc(Cl)cc2)c1C#N] | 6-Allyloxy-4-(4-chloro-phenyl)- [2,2′]bipyridinyl-5-carbonitrile |
| 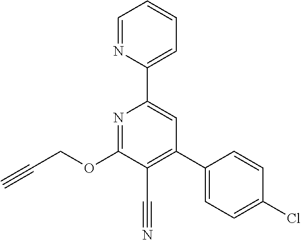 [SMILES: C#CCOc1nc(-c2ccccn2)cc(-c2ccc(Cl)cc2)c1C#N] | 4-(4-Chloro-phenyl)-6-prop- 2-ynyloxy-[2,2′]bipyridinyl- 5-carbonitrile |
| 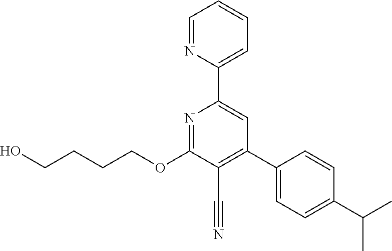 [SMILES: CC(C)c1ccc(-c2cc(-c3ccccn3)nc(OCCCCO)c2C#N)cc1] | 6-(4-Hydroxy-butoxy)-4-(4- isopropyl-phenyl)- [2,2′]bipyridinyl-5-carbonitrile |
| 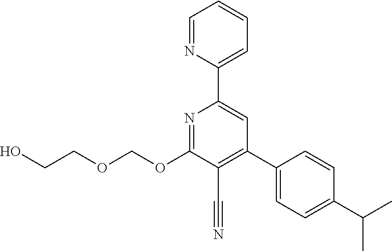 [SMILES: CC(C)c1ccc(-c2cc(-c3ccccn3)nc(OCOCCO)c2C#N)cc1] | 6-(2-Hydroxy-ethoxymethoxy)- 4-(4-isopropyl-phenyl)- [2,2′]bipyridinyl-5-carbonitrile |
| 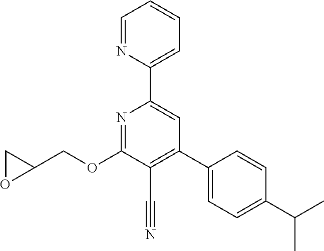 [SMILES: CC(C)c1ccc(-c2cc(-c3ccccn3)nc(OCC3CO3)c2C#N)cc1] | 4-(4-Isopropyl-phenyl)-6- oxiranylmethoxy- [2,2′]bipyridinyl-5-carbonitrile |
| 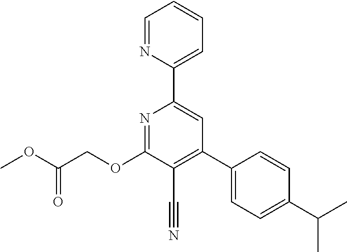 [SMILES: COC(=O)COc1nc(-c2ccccn2)cc(-c2ccc(C(C)C)cc2)c1C#N] | [5-Cyano-4-(4-isopropyl- phenyl)-[2,2′]bipyridinyl- 6-yloxy]-acetic acid methyl ester |
| 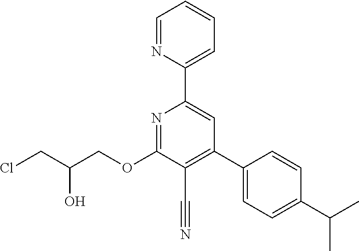 [SMILES: CC(C)c1ccc(-c2cc(-c3ccccn3)nc(OCC(O)CCl)c2C#N)cc1] | 6-(3-Chloro-2-hydroxy- propoxy)-4-(4- isopropyl-phenyl)- [2,2′]bipyridinyl- 5 -carbonitrile |
| 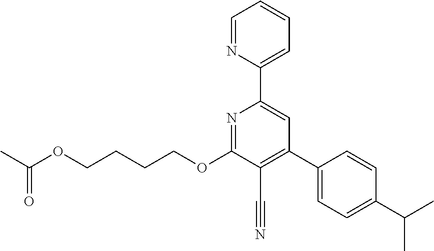 [SMILES: CC(=O)OCCCCOc1nc(-c2ccccn2)cc(-c2ccc(C(C)C)cc2)c1C#N] | Acetic acid 4-[5- cyano-4-(4-isopropyl-phenyl)- [2,2′]bipyridinyl- 6-yloxy]-butyl ester |
| 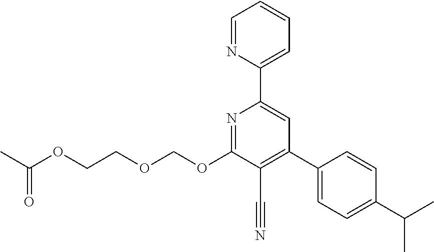 [SMILES: CC(=O)OCCOCOc1nc(-c2ccccn2)cc(-c2ccc(C(C)C)cc2)c1C#N] | Acetic acid 2-[5-cyano-4-(4- isopropyl-phenyl)- [2,2′]bipyridinyl- 6-yloxymethoxy]- ethyl ester |
| 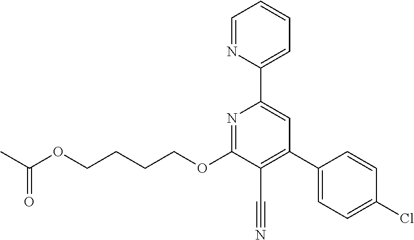 [SMILES: CC(=O)OCCCCOc1nc(-c2ccccn2)cc(-c2ccc(Cl)cc2)c1C#N] | Acetic acid 4-[4-(4-chloro- phenyl)-5-cyano- [2,2′]bipyridinyl- 6-yloxy]-butyl ester |
| 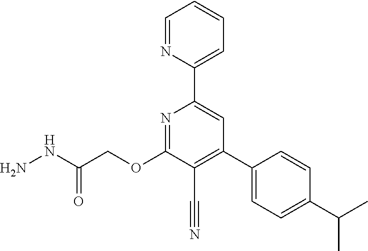 [SMILES: CC(C)c1ccc(-c2cc(-c3ccccn3)nc(OCC(=O)NN)c2C#N)cc1] | [5-Cyano-4-(4-isopropyl-phenyl)- [2,2′]bipyridinyl- 6-yloxy]-acetic acid hydrazide |
| 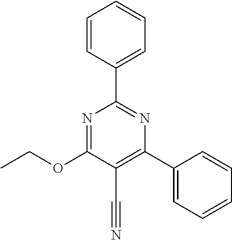 [SMILES: CCOc1nc(-c2ccccc2)nc(-c2ccccc2)c1C#N] | 4-Ethoxy-2,6-diphenyl- pyrimidine-5-carbonitrile |
| 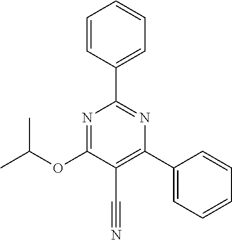 [SMILES: CC(C)Oc1nc(-c2ccccc2)nc(-c2ccccc2)c1C#N] | 4-Isopropoxy-2,6-diphenyl- pyrimidine-5-carbonitrile |
| 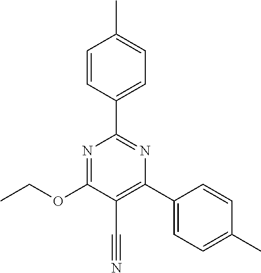 [SMILES: CCOc1nc(-c2ccc(C)cc2)nc(-c2ccc(C)cc2)c1C#N] | 4-Ethoxy-2,6-di-p-tolyl- pyrimidine-5-carbonitrile |
| 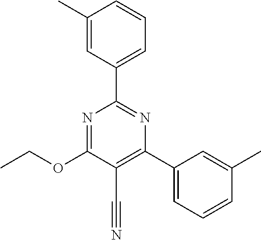 [SMILES: CCOc1nc(-c2cccc(C)c2)nc(-c2cccc(C)c2)c1C#N] | 4-Ethoxy-2,6-di-m-tolyl- pyrimidine-5-carbonitrile |
| 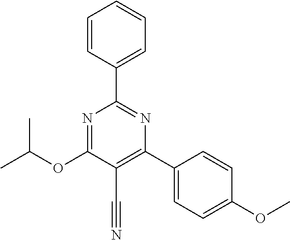 [SMILES: COc1ccc(-c2nc(-c3ccccc3)nc(OC(C)C)c2C#N)cc1] | 4-Isopropoxy-6-(4-methoxy-phenyl)-2- phenyl-pyrimidine-5-carbonitrile |
| 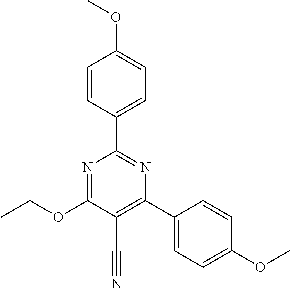 [SMILES: CCOc1nc(-c2ccc(OC)cc2)nc(-c2ccc(OC)cc2)c1C#N] | 4-Ethoxy-2,6-bis-(4-methoxy- phenyl)-pyrimidine-5-carbonitrile |
| 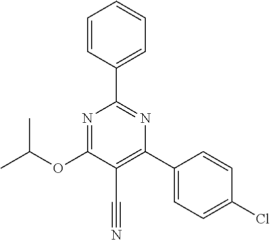 [SMILES: CC(C)Oc1nc(-c2ccccc2)nc(-c2ccc(Cl)cc2)c1C#N] | 4-(4-Chloro-phenyl)-6- isopropoxy-2- phenyl-pyrimidine-5-carbonitrile |
| 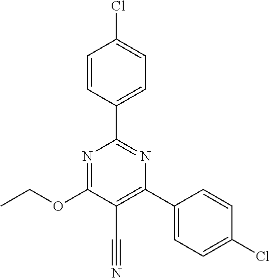 [SMILES: CCOc1nc(-c2ccc(Cl)cc2)nc(-c2ccc(Cl)cc2)c1C#N] | 2,4-Bis-(4-chloro-phenyl)- 6-ethoxy-pyrimidine-5- carbonitrile |
| 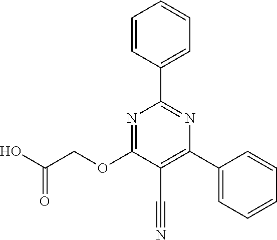 [SMILES: N#Cc1c(OCC(=O)O)nc(-c2ccccc2)nc1-c1ccccc1] | (5-Cyano-2,6-diphenyl- pyrimidin-4-yloxy)-acetic acid |
| 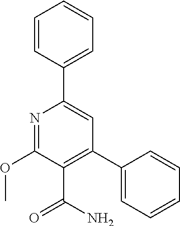 [SMILES: COc1nc(-c2ccccc2)cc(-c2ccccc2)c1C(N)=O] | 2-Methoxy-4,6-diphenyl- nicotinamide |
| 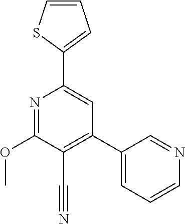 [SMILES: COc1nc(-c2cccs2)cc(-c2cccnc2)c1C#N] | 2′-Methoxy-6′-thiophen-2-yl- [3,4′]bipyridinyl-3′-carbonitrile |
| 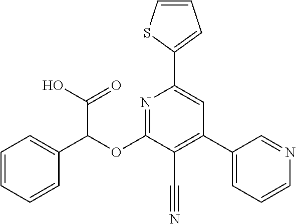 [SMILES: N#Cc1c(-c2cccnc2)cc(-c2cccs2)nc1OC(C(=O)O)c1ccccc1] | (3′-Cyano-6′-thiophen-2- yl-[3,4′]bipyridinyl-2′- yloxy)-phenyl-acetic acid |
| 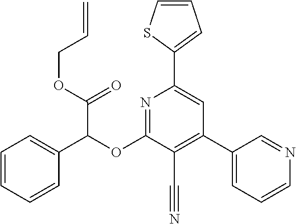 [SMILES: C=CCOC(=O)C(Oc1nc(-c2cccs2)cc(-c2cccnc2)c1C#N)c1ccccc1] | (3′-Cyano-6′-thiophen-2-yl- [3,4′]bipyridinyl-2′- yloxy)-phenyl-acetic acid allyl ester |
| 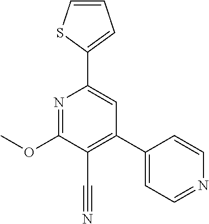 [SMILES: COc1nc(-c2cccs2)cc(-c2ccncc2)c1C#N] | 2-Methoxy-6-thiophen-2-yl- [4,4′]bipyridinyl-3- carbonitrile |
| 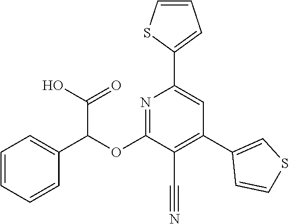 [SMILES: N#Cc1c(-c2ccsc2)cc(-c2cccs2)nc1OC(C(=O)O)c1ccccc1] | (3-Cyano-6-thiophen-2-yl-4- thiophen-3-yl-pyridin-2-yloxy)- phenyl-acetic acid |
| 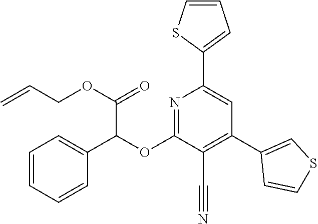 [SMILES: C=CCOC(=O)C(Oc1nc(-c2cccs2)cc(-c2ccsc2)c1C#N)c1ccccc1] | (3-Cyano-6-thiophen-2-yl-4- thiophen-3-yl-pyridin-2-yloxy)- phenyl-acetic acid ally ester |
| 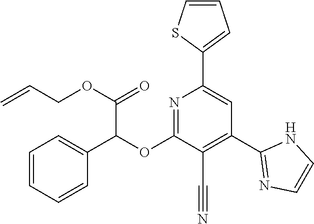 [SMILES: C=CCOC(=O)C(Oc1nc(-c2cccs2)cc(-c2ncc[nH]2)c1C#N)c1ccccc1] | [3-Cyano-4-(1H- imidazol-2-yl)-6- thiophen-2-yl- pyridin-2-yloxy]- phenyl-acetic acid ally ester |
| 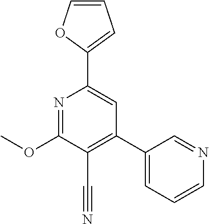 [SMILES: COc1nc(-c2ccco2)cc(-c2cccnc2)c1C#N] | 6′-Furan-2-yl-2′-methoxy- [3,4′]bipyridinyl-3′- carbonitrile |
| 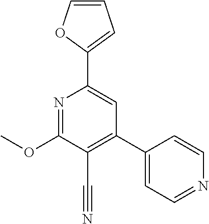 [SMILES: COc1nc(-c2ccco2)cc(-c2ccncc2)c1C#N] | 6-Furan-2-yl-2-methoxy- [4,4′]bipyridinyl-3- carbonitrile |
| 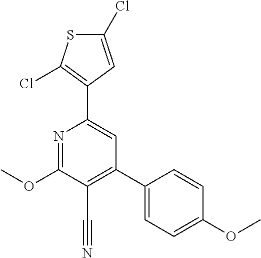 | 6-(2,5-Dichloro-thiophen-3-yl)-2- methoxy-4-(4- methoxy-phenyl)- nicotinonitrile |
| 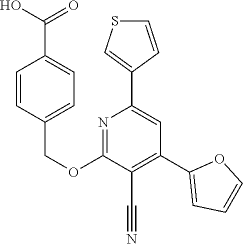 [SMILES: N#Cc1c(-c2ccco2)cc(-c2ccsc2)nc1OCc1ccc(C(=O)O)cc1] | 4-(3-Cyano-4-furan- 2-yl-6-thiophen-3- yl-pyridin-2-yloxymethyl)- benzoic acid |
| 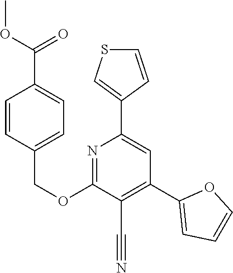 [SMILES: COC(=O)c1ccc(COc2nc(-c3ccsc3)cc(-c3ccco3)c2C#N)cc1] | 4-(3-Cyano-4-furan- 2-yl-6-thiophen-3- yl-pyridin-2-yloxymethyl)- benzoic acid methyl ester |
| 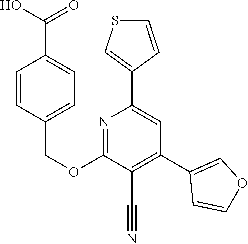 [SMILES: N#Cc1c(-c2ccoc2)cc(-c2ccsc2)nc1OCc1ccc(C(=O)O)cc1] | 4-(3-Cyano-4-furan- 3-yl-6-thiophen-3- yl-pyridin-2- yloxymethyl)-benzoic acid |
| 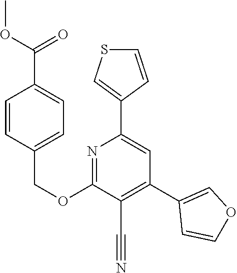 [SMILES: COC(=O)c1ccc(COc2nc(-c3ccsc3)cc(-c3ccoc3)c2C#N)cc1] | 4-(3-Cyano-4-furan- 3-yl-6-thiophen-3- yl-pyridin-2-yloxymethyl)- benzoic acid methyl ester |
| 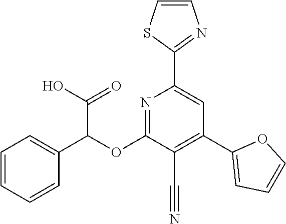 [SMILES: N#Cc1c(-c2ccco2)cc(-c2nccs2)nc1OC(C(=O)O)c1ccccc1] | (3-Cyano-4-furan-2- yl-6-thiazol-2-yl- pyridin-2-yloxy)- phenyl-acetic acid |
| 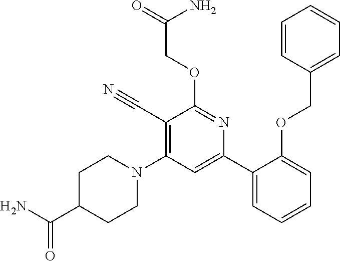 [SMILES: N#Cc1c(N2CCC(C(N)=O)CC2)cc(-c2ccccc2OCc2ccccc2)nc1OCC(N)=O] | 6′-(2-Benzyloxy- phenyl)-2′- carbamoylmethoxy- 3′-cyano-3,4,5,6- tetrahydro-2H- [1,4′]bipyridinyl-4- carboxylic acid amide |
| 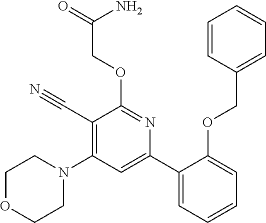 [SMILES: N#Cc1c(N2CCOCC2)cc(-c2ccccc2OCc2ccccc2)nc1OCC(N)=O] | 2-[6-(2-Benzyloxy- phenyl)-3-cyano-4- morpholin-4-yl- pyridin-2-yloxy]-acetamide |
| 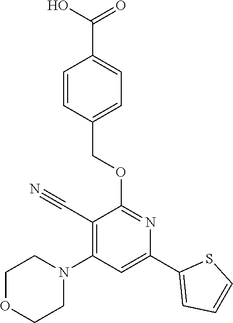 [SMILES: N#Cc1c(N2CCOCC2)cc(-c2cccs2)nc1OCc1ccc(C(=O)O)cc1] | 4-(3-Cyano-4-morpholin-4-yl-6- thiophen-2-yl-pyridin-2- yloxymethyl)-benzoic acid |
| 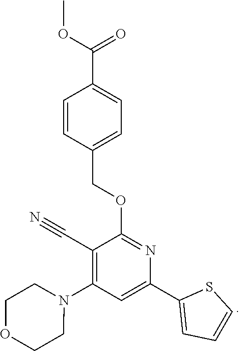 [SMILES: COC(=O)c1ccc(COc2nc(-c3cccs3)cc(N3CCOCC3)c2C#N)cc1] | 4-(3-Cyano-4- morpholin-4-yl-6- thiophen-2-yl-pyridin-2- yloxymethyl)- benzoic acid methyl ester |

In one aspect, the invention provides a compound of formula (IB), or a salt, solvate, stereoisomer, geometric isomer, and/or tautomer thereof:

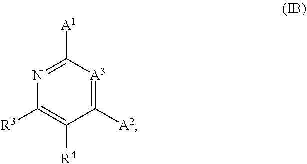

wherein in (IB):

A¹ is —C(R⁷)(R⁸)(R⁹),

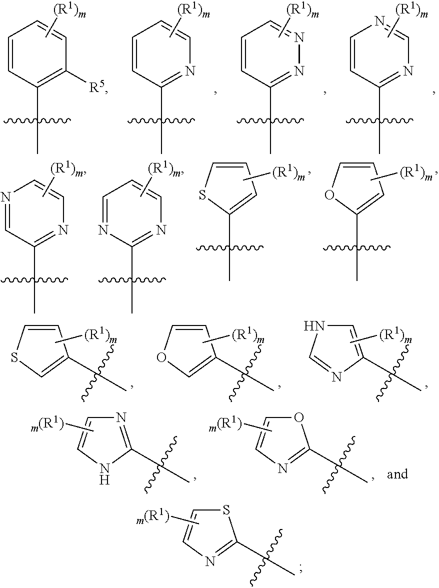

A is —C(R¹⁰)(R¹¹)(R¹²),

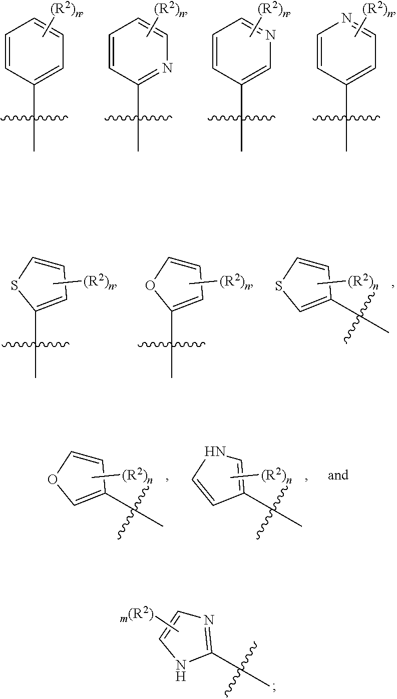

A³ is N or C—R⁶;

m is 1, 2 or 3;

n is 1, 2 or 3;

each occurrence of R¹ is independently selected from the group consisting of H, F, Cl, Br, I, —OR, —SR, —S(═O)(C₁-C₆ alkyl), —S(═O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, N¹-β-propiolactam, N¹-γ-butyrolactam, N¹-δ-valerolactam, and N¹-ε-caprolactam;
wherein, if two R¹ groups are present in neighboring carbons, they optionally
combine to form the divalent group —O(CH₂)₁₋₃O—;

each occurrence of R² is independently selected from the group consisting of H, F, Cl, Br, I, —OR, —SR, —S(═O)(C₁-C₆ alkyl), —S(═O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, N¹-β-propiolactam, N¹-γ-butyrolactam, N¹-δ-valerolactam, and N¹-ε-caprolactam;
wherein, if two R² groups are present in neighboring carbons, they optionally
combine to form the divalent group —O(CH₂)₁₋₃O—;

R³ is selected from the group consisting of H, —OH, C₁-C₆ alkyl, (C₁-C₆)alkoxy, (C₁-C₆)haloalkyl, (C₁-C₆)haloalkoxy, benzyloxy, —OCH₂C(═O)OR, —(CH₂)₀₋₅NR₂, N⁴-morpholinyl, N¹-piperidinyl, N¹-β-propiolactam, N¹-γ-butyrolactam, N¹-δ-valerolactam, N¹-ε-caprolactam, oxetanoxyl, —SH, —S(═O)(C₁-C₆ alkyl), and —S(═O)₂(C₁-C₆ alkyl);

R⁴ is independently selected from the group consisting of H, —CH₃, —CN, —C≡CH, —C(═O)OR, and —C(═O)NHR;

R⁵ is R¹ and R⁶ is H or C₁-C₆ alkyl, or R⁵ and R⁶ combine to form a divalent group selected from the group consisting of —O—, —CRR—, or —CRR—CRR—,

each of R⁷, R⁸, R⁹, R¹⁰, R¹¹, and R¹² is independently optionally substituted C₁-C₁₀ alkyl, wherein two or three of R⁷, R⁸, R⁹ or of R¹⁰, R¹¹, R¹² can optionally combine to form monocyclic or polycyclic groups (such as cyclohexyl or adamantly); and

each occurrence of R is independently H or C₁-C₆ alkyl.

In one aspect, the invention provides a compound of formula (IB), or a salt, solvate, stereoisomer, geometric isomer, and/or tautomer thereof:

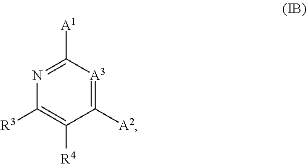

wherein in (IB):

A is —C(R⁷)(R⁸)(R⁹),

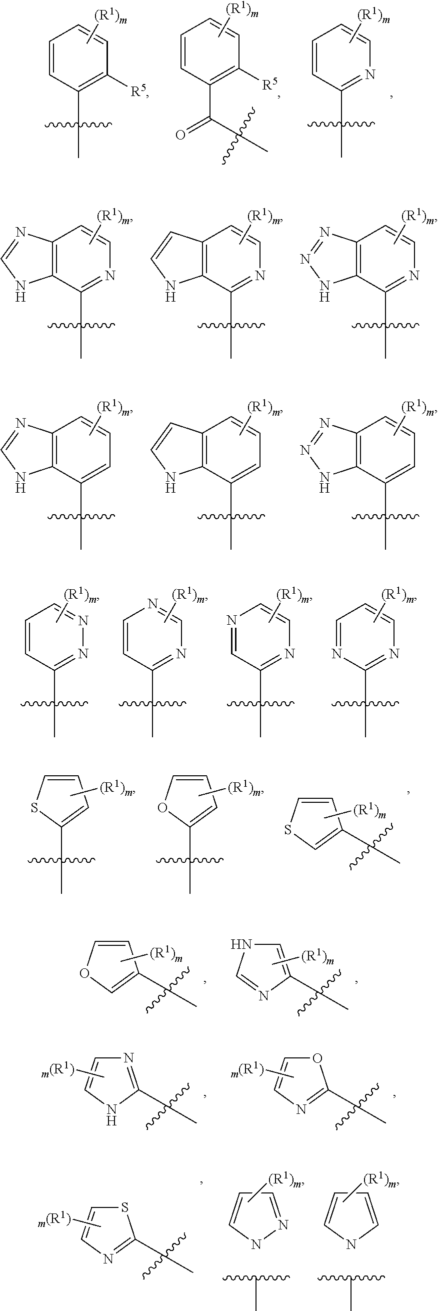

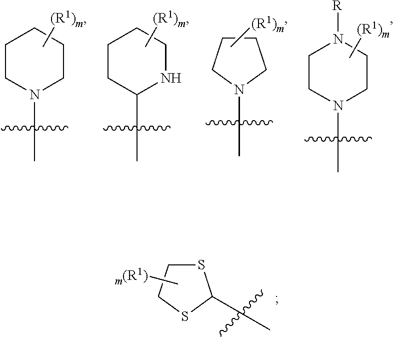

A² is —C(R¹⁰)(R¹¹)(R¹²),

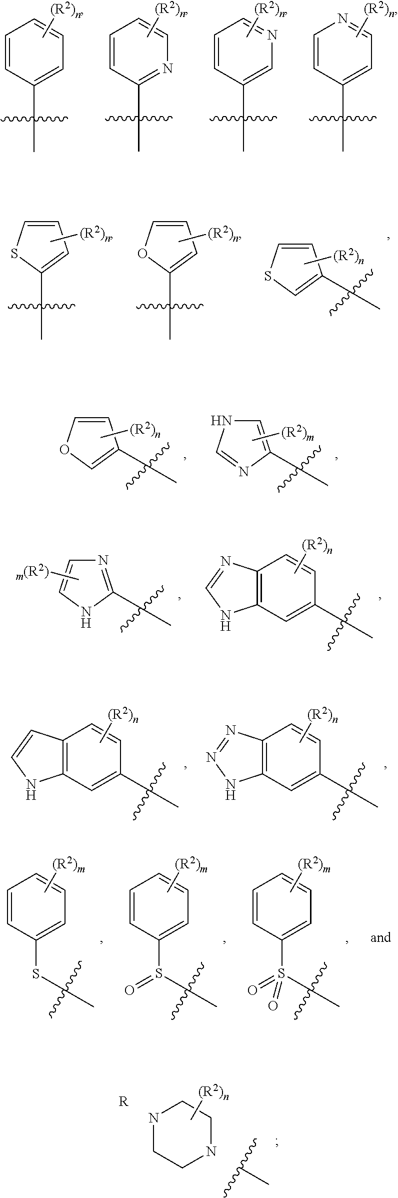

A³ is N or C—R⁶;

m is 1, 2 or 3;

n is 1, 2 or 3;

each occurrence of R¹ is independently selected from the group consisting of H, F, Cl, Br, I, —OR, —NR₂, —SR, —S(O)(C₁-C₆ alkyl), —S(O)₂(C₁-C₆ alkyl), —(CH₂)₀₋₅NR₂, —CN, —NO₂, Cᵣ C₆ alkyl, (C₁-C₆)haloalkyl, (C₁-C₆)haloalkoy, —C(═O)NR₂, —SO₂NR₂, N¹-β-propiolactam, N¹-γ-butyrolactam, N¹-δ-valerolactam, and N¹-ε-caprolactam;
wherein, if two R¹ groups are present in neighboring carbons, they optionally
combine to form the divalent group —O(CH₂)₁₋₃O—;

each occurrence of R² is independently selected from the group consisting of H, F, Cl, Br, I, —OR, —SR, —S(O)(C₁-C₆ alkyl), —S(O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R² groups are present in neighboring carbons, they optionally
combine to form the divalent group —O(CH₂)₁₋₃O—;
with the proviso that R² is not positioned ortho to the bond between the central heteroaryl ring and A²;

R³ is selected from the group consisting of H, C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆ alkyl), —S(O)(C₁-C₆ alkyl), —S(O)₂(C₁-C₆ alkyl), N¹-β-propiolactam, N¹-γ-butyrolactam, N³-δ-valerolactam, oxetanoyl, oxetanoxyl, N¹-ε-caprolactam, (C₂-C₆)alkenyloxy, (C₂-C₆)alkynyloxy, —O(CH₂)₂₋₄OR, —O(CH₂)₂₋₄O(CH₂)₂₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl. —O(CH₂)₂₋₄(N¹-pyrrolidinyl), —N¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;

R⁴ is independently selected from the group consisting of H, —CH₃, —CN, —C≡CR, —C(═O)OR, —C(═O)NR₂, and —C(═O)NHR, or R⁴ can combine with R³ to form a 5-membered amino heterocycle so that the compound of formula (IB) has one of the following structures

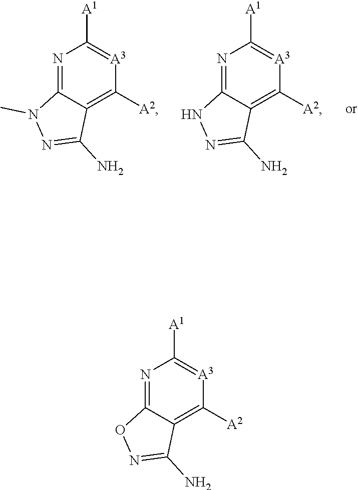

R⁵ is R¹ and R⁶ is H or C₁-C₆ alkyl, or R⁵ and R⁶ combine to form a divalent group selected from the group consisting of —O—, —CRR—, or —CRR—CRR—,

each of R⁷, R⁸, R⁹, R¹⁰, R¹¹, and R¹² is independently selected from optionally substituted C₁-C₁₀ alkyl, wherein two or three of R⁷, R⁸, R⁹ or of R¹⁰, R¹¹, R¹² can optionally combine to form monocyclic or polycyclic groups (such as cyclohexyl or adamantly); and

each occurrence of R is independently H or C₁-C₆ alkyl.

In one aspect, the invention provides a compound of formula (II), or a salt, solvate, stereoisomer, geometric isomer, and/or tautomer thereof:

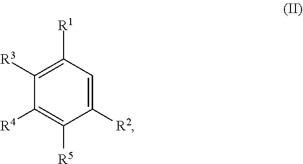

wherein in (II):

R¹ is selected from the group consisting of phenyl, N¹-pyrrolyl. N¹-imidazolyl. N¹-pyrazolyl, N¹-triazolyl, N²-1,2,3-triazolyl, N¹-triazolyl, N⁴-1,2,3-triazolyl, N¹-tetrazolyl, N¹-isoxazolyl. N¹-pyrrolidinyl, N¹-piperidinyl, N¹-morpholinyl, piperizin-1-yl, and N⁴—(C₁-C₆ alkyl)-piperizin-1-yl,
wherein the phenyl group is optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, C(═O)NR₂, N¹-β-propiolactam, N¹-γ-butyrolactam, N¹-δ-valerolactam, and N¹-ε-caprolactam; wherein, if two substituents are present in neighboring carbons, they optionally combine to form the divalent group —O(CH₂)₁₋₃O—;

R² is independently selected from the group consisting of H, piperizin-1-yl, N⁴—(C₁-C₆ alkyl)-piperizin-1-yl, phenyl, pyridin-2-yl, pyridin-3-yl, and pyridin-4-yl; wherein the phenyl group is optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, C(═O)NR₂, N¹-β-propiolactam, N¹-γ-butyrolactam, N¹-δ-valerolactam, and N¹-ε-caprolactam; wherein, if two substituents are present in neighboring carbons, they optionally combine to form the divalent group —O(CH₂)₁₋₃O—;

R³ is selected from the group consisting of H and Cl;

R⁴ is selected from the group consisting of H, —OH, (C₁-C₆)alkyl, (C₁-C₆)alkoxy, (C₁-C₆)haloalkyl, (C₁-C₆)haloalkoxy, and benzyloxy;

R⁵ is independently selected from the group consisting of H, —NO₂, —CN, —C≡CH, —C(═O)OH, —SO₂(C₁-C₆ alkyl), —SO₂NHAc, and tetrazol-1-yl.

In one aspect, the invention provides a compound of formula (III), or a salt, solvate, stereoisomer, geometric isomer, and/or tautomer thereof:

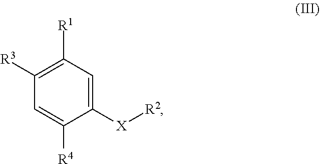

wherein in (III):

X is selected from the group consisting of O, S, S(═O), and S(═O)₂;

R¹ is selected from the group consisting of H, phenyl, —C(═O)phenyl, 1,3-dithiol-2-yl, pyrrol-1-yl, imidazole-1-yl, pyrazol-1-yl, 1,2,3-triazol-1-yl, 1,2,3-triazol-2-yl, 1,2,4-triazol-1-yl, 1,2,3-triazol-4-yl, tetrazol-1-yl, isoxazol-1-yl, pyrrolidin-1-yl, piperidin-1-yl, morpholin-1-yl, piperizin-1-yl, and N⁴—(C₁-C₆ alkyl)-piperizin-1-yl;
wherein each phenyl group is independently optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, —C(═O)NR₂, N¹-β-propiolactam, N¹-γ-butyrolactam, N¹-δ-valerolactam, and N¹-ε-caprolactam;
wherein, if two substituents are present in neighboring carbons, they optionally combine to form the divalent group —O(CH₂)₁₋₃O—;

R² is selected from the group consisting of phenyl, pyridin-2-yl, pyridin-3-yl, and pyridin-4-yl;
wherein each phenyl group is independently optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, —C(═O)NR₂, N¹-β-propiolactam, N¹-γ-butyrolactam, N¹-δ-valerolactam, and N¹-ε-caprolactam;
   wherein, if two substituents are present in neighboring carbons, they optionally combine to form the divalent group —O(CH₂)₁₋₃O—;

R³ is selected from the group consisting of H, F, Cl, Br, I, C₁-C₆ alkyl, and C₁-C₆ alkoxy; and

R⁴ is independently selected from the group consisting of H, —NO₂, —CN, —C≡CH, —C(═O)OH, —SO₂(C₁-C₆ alkyl), —SO₂NHAc, and N¹-tetrazolyl.

In certain embodiments, the compound of formula (IB) is selected from the group consisting of:

| Structure | Name | Cpd ID |
|---|---|---|
| 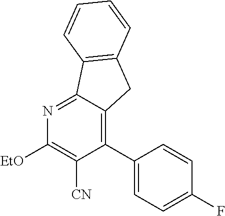 [SMILES: CCOc1nc2c(c(-c3ccc(F)cc3)c1C#N)Cc1ccccc1-2] | 2-ethoxy-4-(4-fluorophenyl)- 5H-indeno[1,2-b]pyridine-3- carbonitrile | 1 |
| 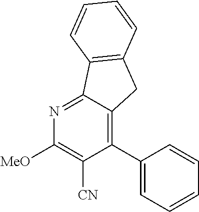 [SMILES: COc1nc2c(c(-c3ccccc3)c1C#N)Cc1ccccc1-2] | 2-methoxy-4-phenyl-5H- indeno[1,2-b]pyridine-3- carbonitrile | 4 |
| 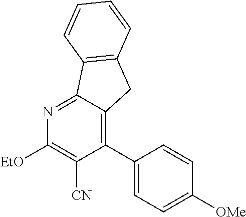 [SMILES: CCOc1nc2c(c(-c3ccc(OC)cc3)c1C#N)Cc1ccccc1-2] | 2-ethoxy-4-(4- methoxyphenyl)-5H- indeno[1,2-b]pyridine-3- carbonitrile | 5 |
| 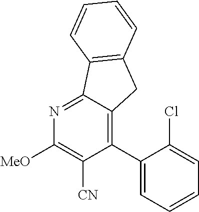 [SMILES: COc1nc2c(c(-c3ccccc3Cl)c1C#N)Cc1ccccc1-2] | 4-(2-chlorophenyl)-2- methoxy-5H-indeno[1,2- b]pyridine-3-carbonitrile | 6 |
| 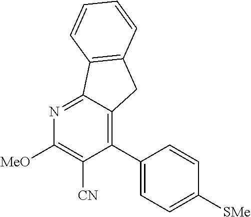 [SMILES: COc1nc2c(c(-c3ccc(SC)cc3)c1C#N)Cc1ccccc1-2] | 2-methoxy-4-(4- (methylthio)phenyl)-5H- indeno[1,2-b]pyridine-3- carbonitrile | 7 |
| 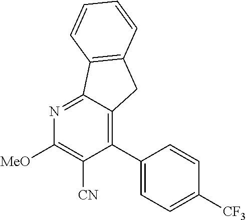 [SMILES: COc1nc2c(c(-c3ccc(C(F)(F)F)cc3)c1C#N)Cc1ccccc1-2] | 2-methoxy-4-(4- (trifluoromethyl)phenyl)-5H- indeno[1,2-b]pyridine-3- carbonitrile | 8 |
| 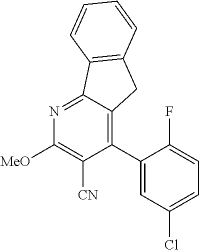 [SMILES: COc1nc2c(c(-c3cc(Cl)ccc3F)c1C#N)Cc1ccccc1-2] | 4-(5-chloro-2-fluorophenyl)- 2-methoxy-5H-indeno[1,2- b]pyridine-3-carbonitrile | 9 |
| 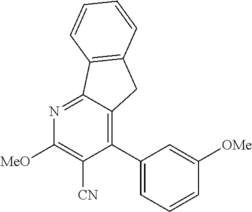 [SMILES: COc1cccc(-c2c(C#N)c(OC)nc3c2Cc2ccccc2-3)c1] | 2-methoxy-4-(3- methoxyphenyl)-5H- indeno[1,2-b]pyridine-3- carbonitrile | 10 |
| 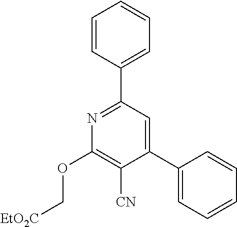 [SMILES: CCOC(=O)COc1nc(-c2ccccc2)cc(-c2ccccc2)c1C#N] | ethyl 2-((3-cyano-4,6- diphenylpyridin-2- yl)oxy)acetate | 11 |
| 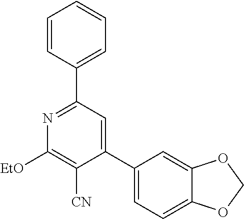 [SMILES: CCOc1nc(-c2ccccc2)cc(-c2ccc3c(c2)OCO3)c1C#N] | 4-(benzo[d][1,3]dioxol-5-yl)- 2-ethoxy-6- phenylnicotinonitrile | 12 |
| 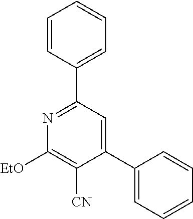 [SMILES: CCOc1nc(-c2ccccc2)cc(-c2ccccc2)c1C#N] | 2-ethoxy-4,6- diphenylnicotinonitrile | 13 |
| 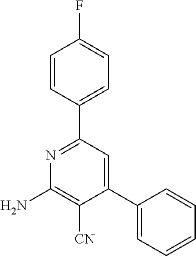 [SMILES: N#Cc1c(-c2ccccc2)cc(-c2ccc(F)cc2)nc1N] | 2-amino-6-(4-fluorophenyl)- 4-phenylnicotinonitrile | 14 |
| 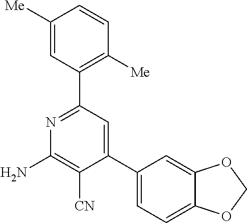 [SMILES: Cc1ccc(C)c(-c2cc(-c3ccc4c(c3)OCO4)c(C#N)c(N)n2)c1] | 2-amino-4- (benzo[d][1,3]dioxol-5-yl)-6- (2,5- dimethylphenyl)nicotinonitrile | 15 |
| 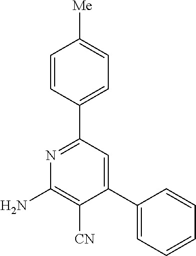 [SMILES: Cc1ccc(-c2cc(-c3ccccc3)c(C#N)c(N)n2)cc1] | 2-amino-4-phenyl-6-(p- tolyl)nicotinonitrile | 16 |
| 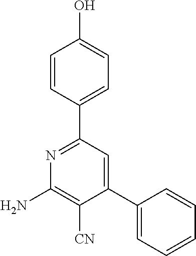 [SMILES: N#Cc1c(-c2ccccc2)cc(-c2ccc(O)cc2)nc1N] | 2-amino-6-(4- hydroxyphenyl)-4- phenylnicotinonitrile | 17 |
| 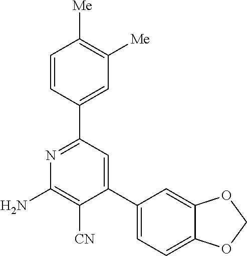 [SMILES: Cc1ccc(-c2cc(-c3ccc4c(c3)OCO4)c(C#N)c(N)n2)cc1C] | 2-amino-4- (benzo[d][1,3]dioxol-5-yl)-6- (3,4- dimethylphenyl)nicotinonitrile | 18 |
| 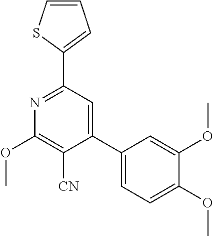 [SMILES: COc1ccc(-c2cc(-c3cccs3)nc(OC)c2C#N)cc1OC] | 4-(3,4-Dimethoxy-phenyl)-2- methoxy-6-thiophen-2-yl- nicotinonitrile | |
| 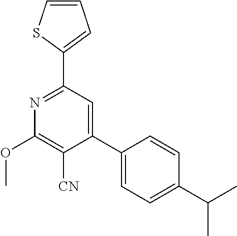 [SMILES: COc1nc(-c2cccs2)cc(-c2ccc(C(C)C)cc2)c1C#N] | 4-(4-Isopropyl-phenyl)-2- methoxy-6-thiophen-2-yl- nicotinonitrile | |
| 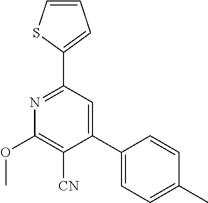 [SMILES: COc1nc(-c2cccs2)cc(-c2ccc(C)cc2)c1C#N] | 2-Methoxy-6-thiophen-2-yl-4- p-tolyl-nicotinonitrile | |
| 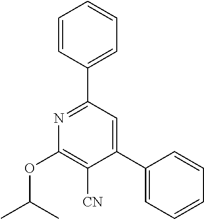 [SMILES: CC(C)Oc1nc(-c2ccccc2)cc(-c2ccccc2)c1C#N] | 2-Isopropoxy-4,6-diphenyl- nicotinonitrile | |
| 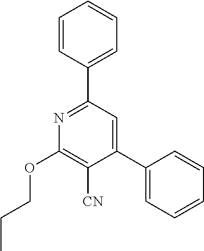 [SMILES: CCCOc1nc(-c2ccccc2)cc(-c2ccccc2)c1C#N] | 4,6-Diphenyl-2-propoxy- nicotinonitrile | |
| 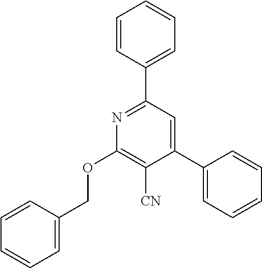 [SMILES: N#Cc1c(-c2ccccc2)cc(-c2ccccc2)nc1OCc1ccccc1] | 2-Benzyloxy-4,6-diphenyl- nicotinonitrile | |
| 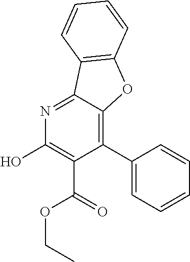 [SMILES: CCOC(=O)c1c(O)nc2c(oc3ccccc32)c1-c1ccccc1] | 2-Hydroxy-4-phenyl- benzo[4,5]furo[3,2- b]pyridine-3-carboxylic acid ethyl ester | |
| 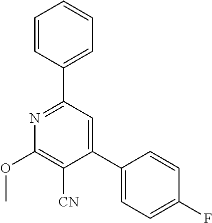 [SMILES: COc1nc(-c2ccccc2)cc(-c2ccc(F)cc2)c1C#N] | 4-(4-Fluoro-phenyl)-2- methoxy-6-phenyl- nicotinonitrile | |
| 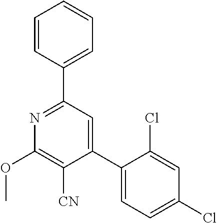 [SMILES: COc1nc(-c2ccccc2)cc(-c2ccc(Cl)cc2Cl)c1C#N] | 4-(2,4-Dichloro-phenyl)-2- methoxy-6-phenyl- nicotinonitrile | |
| 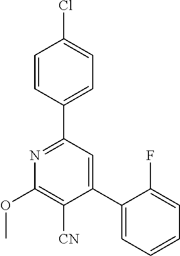 [SMILES: COc1nc(-c2ccc(Cl)cc2)cc(-c2ccccc2F)c1C#N] | 6-(4-Chloro-phenyl)-4-(2- fluoro-phenyl)-2-methoxy- nicotinonitrile | |
| 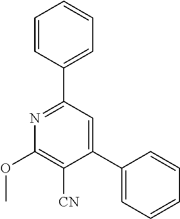 [SMILES: COc1nc(-c2ccccc2)cc(-c2ccccc2)c1C#N] | 2-Methoxy-4,6-diphenyl- nicotinonitrile | |
| 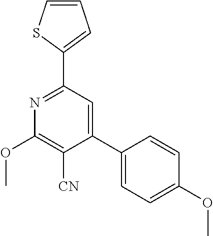 [SMILES: COc1ccc(-c2cc(-c3cccs3)nc(OC)c2C#N)cc1] | 2-Methoxy-4-(4-methoxy- phenyl)-6-thiophen-2-yl- nicotinonitrile | |
| 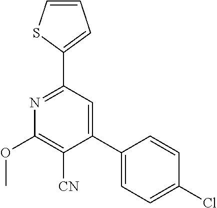 [SMILES: COc1nc(-c2cccs2)cc(-c2ccc(Cl)cc2)c1C#N] | 4-(4-Chloro-phenyl)-2- methoxy-6-thiophen-2-yl- nicotinonitrile | |
| 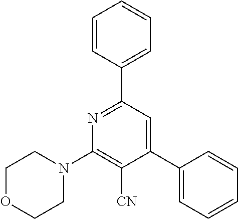 [SMILES: N#Cc1c(-c2ccccc2)cc(-c2ccccc2)nc1N1CCOCC1] | 2-Morpholin-4-yl-4,6- diphenyl-nicotinonitrile | |
| 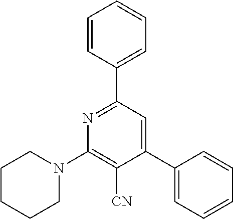 [SMILES: N#Cc1c(-c2ccccc2)cc(-c2ccccc2)nc1N1CCCCC1] | 4,6-diphenyl-2-(piperidin-1- yl)pyridine-3-carbonitrile | |
| 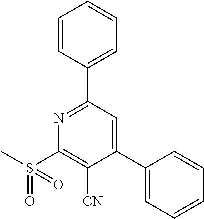 [SMILES: CS(=O)(=O)c1nc(-c2ccccc2)cc(-c2ccccc2)c1C#N] | 2-Methanesulfonyl-4,6- diphenyl-nicotinonitrile | |
| 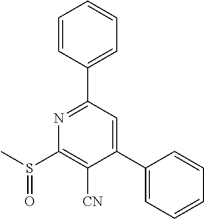 [SMILES: CS(=O)c1nc(-c2ccccc2)cc(-c2ccccc2)c1C#N] | 2-Methanesulfinyl-4,6- diphenyl-nicotinonitrile | |
| 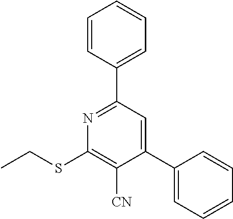 [SMILES: CCSc1nc(-c2ccccc2)cc(-c2ccccc2)c1C#N] | 2-Ethylsulfanyl-4,6-diphenyl- nicotinonitrile | |
| 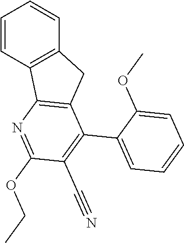 [SMILES: CCOc1nc2c(c(-c3ccccc3OC)c1C#N)Cc1ccccc1-2] | 2-Ethoxy-4-(2- methoxyphenyl)-5H- indeno[1,2-b]pyridine-3- carbonitrile | |
| 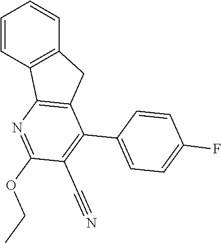 [SMILES: CCOc1nc2c(c(-c3ccc(F)cc3)c1C#N)Cc1ccccc1-2] | 4-(4-Fluorophenyl)-2-ethoxy- 5H-indeno[1,2-b]pyridine-3- carbonitrile | |
| 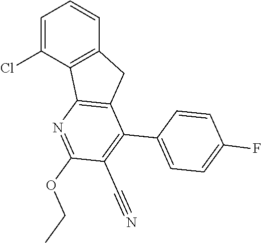 [SMILES: CCOc1nc2c(c(-c3ccc(F)cc3)c1C#N)Cc1cccc(Cl)c1-2] | 9-Chloro-2-ethoxy-4-(4- fluorophenyl)-5H-indeno[1,2- b]pyridine-3-carbonitrile | |
| 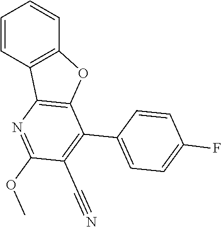 [SMILES: COc1nc2c(oc3ccccc32)c(-c2ccc(F)cc2)c1C#N] | 4-(4-Fluorophenyl)-2- methoxybenzofuro[3,2- b]pyridine-3-carbonitrile | |
| 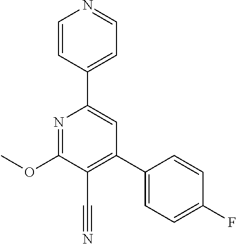 [SMILES: COc1nc(-c2ccncc2)cc(-c2ccc(F)cc2)c1C#N] | 4-(4-Fluorophenyl)-2- methoxy-6-(pyridin-4- yl)pyridine-3-carbonitrile | |
| 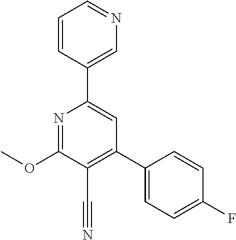 [SMILES: COc1nc(-c2cccnc2)cc(-c2ccc(F)cc2)c1C#N] | 4-(4-Fluorophenyl)-2- methoxy-6-(pyridin-3- yl)pyridine-3-carbonitrile | |
| 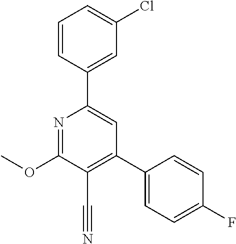 [SMILES: COc1nc(-c2cccc(Cl)c2)cc(-c2ccc(F)cc2)c1C#N] | 6-(3-Chlorophenyl)-4-(4- fluorophenyl)-2- methoxypyridine-3- carbonitrile | |
| 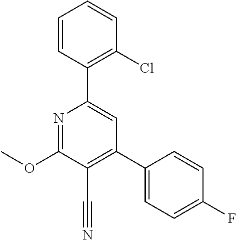 [SMILES: COc1nc(-c2ccccc2Cl)cc(-c2ccc(F)cc2)c1C#N] | 6-(2-Chlorophenyl)-4-(4- fluorophenyl)-2- methoxypyridine-3- carbonitrile | |
| 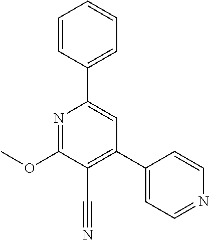 [SMILES: COc1nc(-c2ccccc2)cc(-c2ccncc2)c1C#N] | 2-Methoxy-6-phenyl-4- (pyridin-4-yl)pyridine-3- carbonitrile | |
| 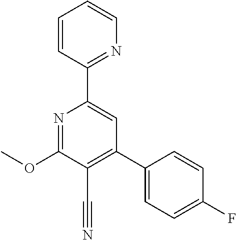 [SMILES: COc1nc(-c2ccccn2)cc(-c2ccc(F)cc2)c1C#N] | 4-(4-Fluorophenyl)-2- methoxy-6-(pyridin-2- yl)pyridine-3-carbonitrile | |
| 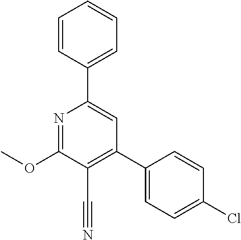 [SMILES: COc1nc(-c2ccccc2)cc(-c2ccc(Cl)cc2)c1C#N] | 4-(4-Chlorophenyl)-2- methoxy-6-phenylpyridine-3- carbonitrile | |
| 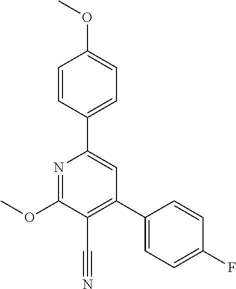 [SMILES: COc1ccc(-c2cc(-c3ccc(F)cc3)c(C#N)c(OC)n2)cc1] | 4-(4-Fluorophenyl)-2- methoxy-6-(4- methoxyphenyl)pyridine-3- carbonitrile | |
| 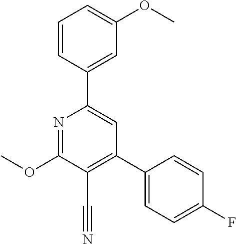 [SMILES: COc1cccc(-c2cc(-c3ccc(F)cc3)c(C#N)c(OC)n2)c1] | 4-(4-Fluorophenyl)-2- methoxy-6-(3- methoxyphenyl)pyridine-3- carbonitrile | |
| 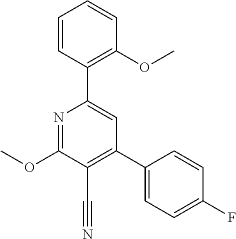 [SMILES: COc1ccccc1-c1cc(-c2ccc(F)cc2)c(C#N)c(OC)n1] | 4-(4-Fluorophenyl)-2- methoxy-6-(2- methoxyphenyl)pyridine-3- carbonitrile | |
| 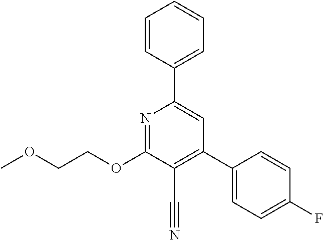 [SMILES: COCCOc1nc(-c2ccccc2)cc(-c2ccc(F)cc2)c1C#N] | 2-(2-Methoxyethoxy)-4-(4- fluorophenyl)-6- phenylpyridine-3-carbonitrile | |
| 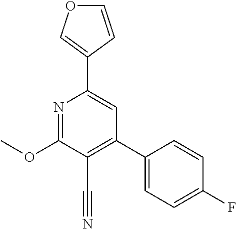 [SMILES: COc1nc(-c2ccoc2)cc(-c2ccc(F)cc2)c1C#N] | 4-(4-Fluoro-phenyl)-6-furan- 3-yl-2-methoxy- nicotinonitrile | |
| 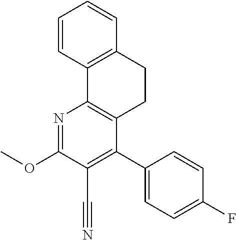 [SMILES: COc1nc2c(c(-c3ccc(F)cc3)c1C#N)CCc1ccccc1-2] | 4-(4-Fluorophenyl)-5,6- dihydro-2- methoxybenzo[h]quinoline-3- carbonitrile | |
| 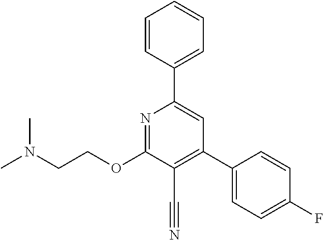 [SMILES: CN(C)CCOc1nc(-c2ccccc2)cc(-c2ccc(F)cc2)c1C#N] | 2-(2- (Dimethylamino)ethoxy)-4- (4-fluorophenyl)-6- phenylpyridine-3-carbonitrile | |
| 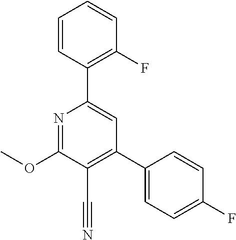 [SMILES: COc1nc(-c2ccccc2F)cc(-c2ccc(F)cc2)c1C#N] | 6-(2-Fluorophenyl)-4-(4- fluorophenyl)-2- methoxypyridine-3- carbonitrile | |
| 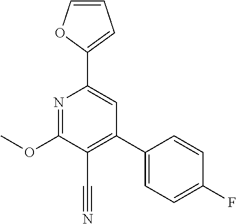 [SMILES: COc1nc(-c2ccco2)cc(-c2ccc(F)cc2)c1C#N] | 4-(4-Fluoro-phenyl)-6-furan- 2-yl-2-methoxy- nicotinonitrile | |
| 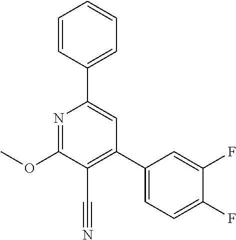 [SMILES: COc1nc(-c2ccccc2)cc(-c2ccc(F)c(F)c2)c1C#N] | 4-(3,4-Difluoro-phenyl)-2- methoxy-6-phenyl- nicotinonitrile | |
| 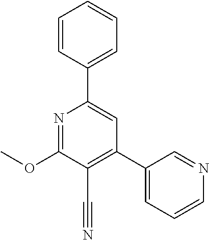 [SMILES: COc1nc(-c2ccccc2)cc(-c2cccnc2)c1C#N] | 2′-Methoxy-6′-phenyl- [3,4′]bipyridinyl-3′- carbonitrile | |
| 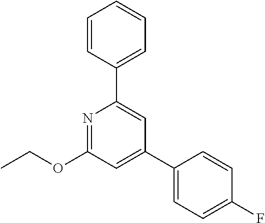 [SMILES: CCOc1cc(-c2ccc(F)cc2)cc(-c2ccccc2)n1] | 2-Ethoxy-4-(4-fluoro-phenyl)- 6-phenyl-pyridine | |
| 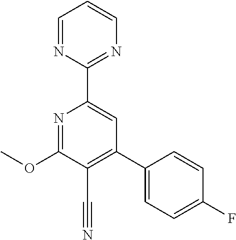 [SMILES: COc1nc(-c2ncccn2)cc(-c2ccc(F)cc2)c1C#N] | 4-(4-Fluoro-phenyl)-2- methoxy-6-pyrimidin-2-yl- nicotinonitrile | |
| 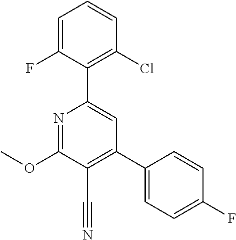 [SMILES: COc1nc(-c2c(F)cccc2Cl)cc(-c2ccc(F)cc2)c1C#N] | 6-(2-Chloro-6-fluoro-phenyl)- 4-(4-fluoro-phenyl)-2- methoxy-nicotinonitrile | |
| 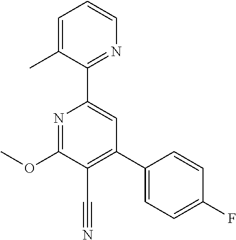 [SMILES: COc1nc(-c2ncccc2C)cc(-c2ccc(F)cc2)c1C#N] | 4-(4-Fluoro-phenyl)-6- methoxy-3′-methyl- [2,2′]bipyridinyl-5- carbonitrile | |
| 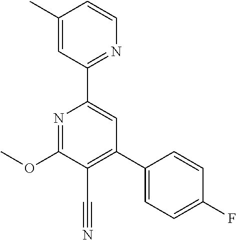 [SMILES: COc1nc(-c2cc(C)ccn2)cc(-c2ccc(F)cc2)c1C#N] | 4-(4-Fluoro-phenyl)-6- methoxy-4′-methyl- [2,2′]bipyridinyl-5- carbonitrile | |
| 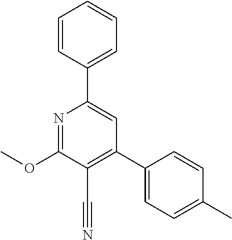 | 2-Methoxy-6-phenyl-4-p- tolyl-nicotinonitrile | |
| 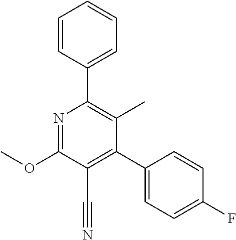 [SMILES: COc1nc(-c2ccccc2)c(C)c(-c2ccc(F)cc2)c1C#N] | 4-(4-Fluoro-phenyl)-2- methoxy-5-methyl-6-phenyl- nicotinonitrile | |
| 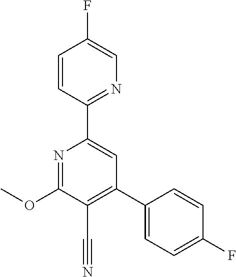 [SMILES: COc1nc(-c2ccc(F)cn2)cc(-c2ccc(F)cc2)c1C#N] | 5′-Fluoro-4-(4-fluoro-phenyl)- 6-methoxy-[2,2′]bipyridinyl- 5-carbonitrile | |
| 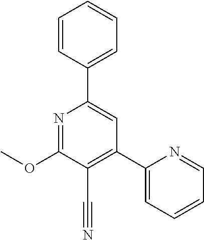 [SMILES: COc1nc(-c2ccccc2)cc(-c2ccccn2)c1C#N] | 2′-Methoxy-6′-phenyl- [2,4′]bipyridinyl-3′- carbonitrile | |
| 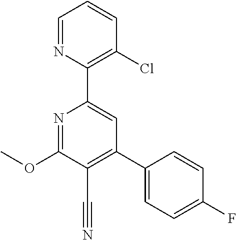 [SMILES: COc1nc(-c2ncccc2Cl)cc(-c2ccc(F)cc2)c1C#N] | 6-(3-Chloropyridin-2-yl)-4- (4-fluorophenyl)-2- methoxypyridine-3- carbonitrile | |
| 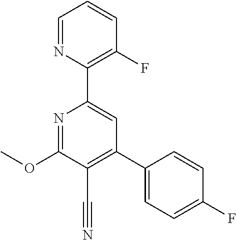 [SMILES: COc1nc(-c2ncccc2F)cc(-c2ccc(F)cc2)c1C#N] | 4-(4-Fluorophenyl)-6-(3- fluoropyridin-2-yl)-2- methoxypyridine-3- carbonitrile | |
| 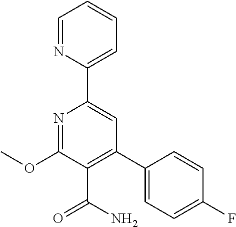 [SMILES: COc1nc(-c2ccccn2)cc(-c2ccc(F)cc2)c1C(N)=O] | 4-(4-Fluorophenyl)-2- methoxy-6-(pyridin-2- yl)pyridine-3-carboxamide | |
| 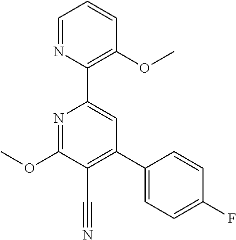 [SMILES: COc1cccnc1-c1cc(-c2ccc(F)cc2)c(C#N)c(OC)n1] | 4-(4-Fluorophenyl)-2- methoxy-6-(3- methoxypyridin-2- yl)pyridine-3-carbonitrile | |
| 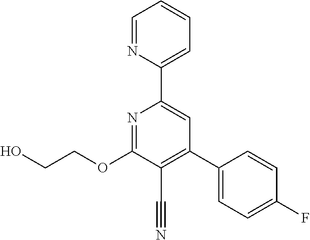 [SMILES: N#Cc1c(-c2ccc(F)cc2)cc(-c2ccccn2)nc1OCCO] | 2-(2-Hydroxyethoxy)-4-(4- fluorophenyl)-6-(pyridin-2- yl)pyridine-3-carbonitrile | |
| 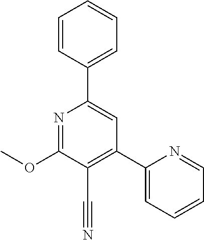 [SMILES: COc1nc(-c2ccccc2)cc(-c2ccccn2)c1C#N] | 2-Methoxy-6-phenyl-4- (pyridin-2-yl)pyridine-3- carbonitrile | |
| 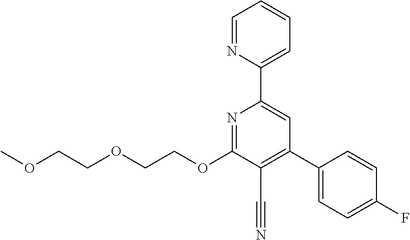 [SMILES: COCCOCCOc1nc(-c2ccccn2)cc(-c2ccc(F)cc2)c1C#N] | 2-(2-(2- Methoxyethoxy)ethoxy)-4-(4- fluorophenyl)-6-(pyridin-2- yl)pyridine-3-carbonitrile | |
| 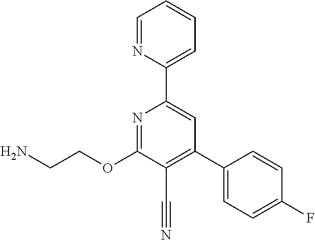 [SMILES: N#Cc1c(-c2ccc(F)cc2)cc(-c2ccccn2)nc1OCCN] | 2-(2-Aminoethoxy)-4-(4- fluorophenyl)-6-(pyridin-2- yl)pyridine-3-carbonitrile | |
| 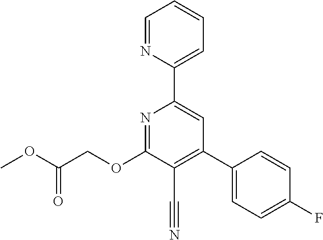 [SMILES: COC(=O)COc1nc(-c2ccccn2)cc(-c2ccc(F)cc2)c1C#N] | Methyl 2-(3-cyano-4-(4- fluorophenyl)-6-(pyridin-2- yl)pyridin-2-yloxy)acetate | |
| 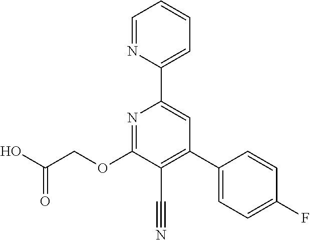 [SMILES: N#Cc1c(-c2ccc(F)cc2)cc(-c2ccccn2)nc1OCC(=O)O] | 2-(3-Cyano-4-(4- fluorophenyl)-6-(pyridin-2- yl)pyridin-2-yloxy)acetic acid | |
| 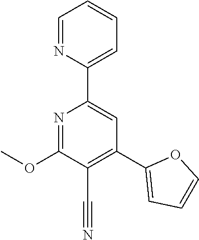 [SMILES: COc1nc(-c2ccccn2)cc(-c2ccco2)c1C#N] | 4-(Furan-2-yl)-2-methoxy-6- (pyridin-2-yl)pyridine-3- carbonitrile | |
| 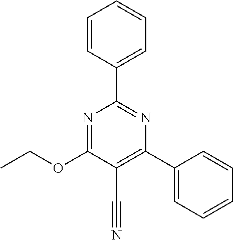 [SMILES: CCOc1nc(-c2ccccc2)nc(-c2ccccc2)c1C#N] | 4-ethoxy-2,6- diphenylpyrimidine-5- carbonitrile | |
| 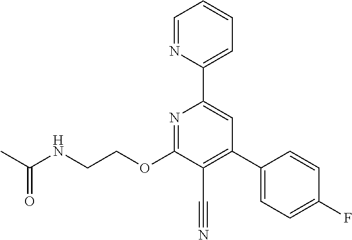 [SMILES: CC(=O)NCCOc1nc(-c2ccccn2)cc(-c2ccc(F)cc2)c1C#N] | N-(2-(3-Cyano-4-(4- fluorophenyl)-6-(pyridin-2- yl)pyridin-2- yloxy)ethyl)acetamide | |
| 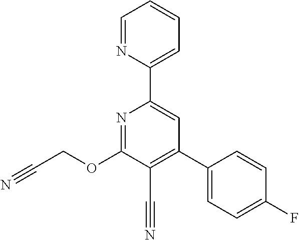 [SMILES: N#CCOc1nc(-c2ccccn2)cc(-c2ccc(F)cc2)c1C#N] | 2-(Cyanomethoxy)-4-(4- fluorophenyl)-6-(pyridin-2- yl)pyridine-3-carbonitrile | |
| 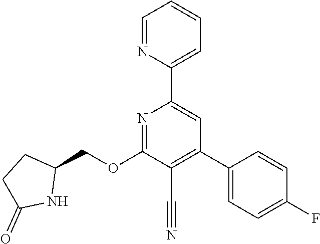 [SMILES: N#Cc1c(-c2ccc(F)cc2)cc(-c2ccccn2)nc1OC[C@@H]1CCC(=O)N1] | 2-((S)-5-(Methoxy)pyrrolidin- 2-one)-4-(4-fluorophenyl)-6- (pyridin-2-yl)pyridine-3- carbonitrile | |
| 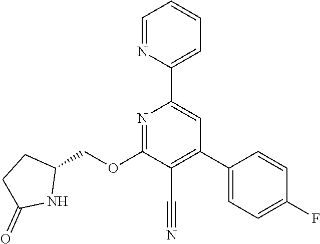 [SMILES: N#Cc1c(-c2ccc(F)cc2)cc(-c2ccccn2)nc1OC[C@H]1CCC(=O)N1] | 2-((R)-5-(Methoxy)pyrrolidin- 2-one)-4-(4-fluorophenyl)-6- (pyridin-2-yl)pyridine-3- carbonitrile | |
| 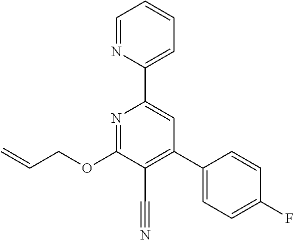 [SMILES: C=CCOc1nc(-c2ccccn2)cc(-c2ccc(F)cc2)c1C#N] | 2-(Allyloxy)-4-(4- fluorophenyl)-6-(pyridin-2- yl)pyridine-3-carbonitrile | |
| 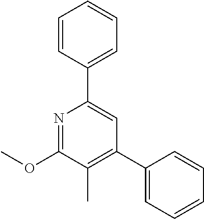 [SMILES: COc1nc(-c2ccccc2)cc(-c2ccccc2)c1C] | 2-Methoxy-3-methyl-4,6- diphenylpyridine | |
| 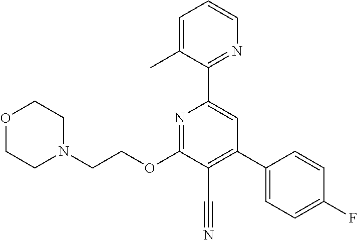 [SMILES: Cc1cccnc1-c1cc(-c2ccc(F)cc2)c(C#N)c(OCCN2CCOCC2)n1] | 2-(2-Morpholinoethoxy)-4-(4- fluorophenyl)-6-(3- methylpyridin-2-yl)pyridine- 3-carbonitrile | |
| 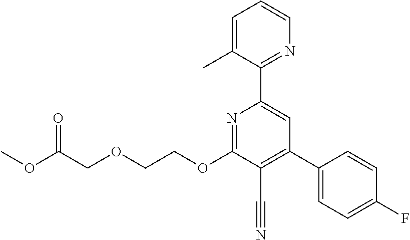 [SMILES: COC(=O)COCCOc1nc(-c2ncccc2C)cc(-c2ccc(F)cc2)c1C#N] | Methyl 2-(3-cyano-4-(4- fluorophenyl)-6-(3- methylpyridin-2-yl)pyridin-2- yloxy) 2-(2-methoxyethoxy) acetate | |
| 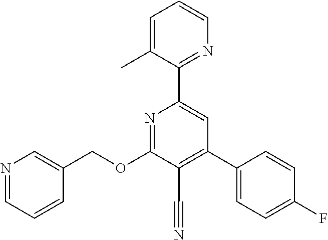 [SMILES: Cc1cccnc1-c1cc(-c2ccc(F)cc2)c(C#N)c(OCc2cccnc2)n1] | 2-((Pyridin-3-yl)methoxy)-4- (4-fluorophenyl)-6-(3- methylpyridin-2-yl)pyridine- 3-carbonitrile | |
| 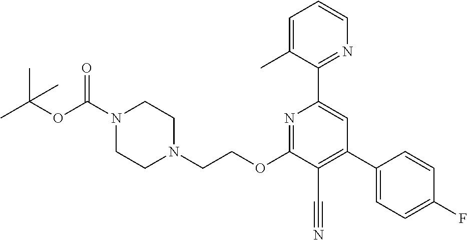 [SMILES: Cc1cccnc1-c1cc(-c2ccc(F)cc2)c(C#N)c(OCCN2CCN(C(=O)OC(C)(C)C)CC2)n1] | tert-Butyl 4-(2-(3-cyano-4-(4- fluorophenyl)-6-(3- methylpyridin-2-yl)pyridin-2- yloxy)ethyl)piperazine-1- carboxylate | |
| 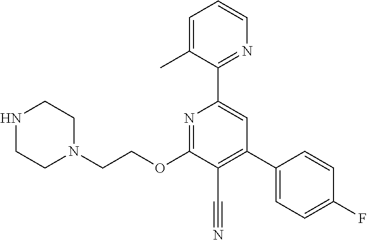 [SMILES: Cc1cccnc1-c1cc(-c2ccc(F)cc2)c(C#N)c(OCCN2CCNCC2)n1] | 2-(2-(Piperazin-1-yl)ethoxy)- 4-(4-fluorophenyl)-6-(3- methylpyridin-2-yl)pyridine- 3-carbonitrile | |
| 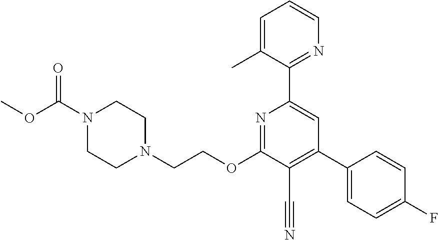 [SMILES: COC(=O)N1CCN(CCOc2nc(-c3ncccc3C)cc(-c3ccc(F)cc3)c2C#N)CC1] | Methyl 4-(2-(3-cyano-4-(4- fluorophenyl)-6-(3- methylpyridin-2-yl)pyridin-2- yloxy)ethyl)piperazine-1- carboxylate | |
| 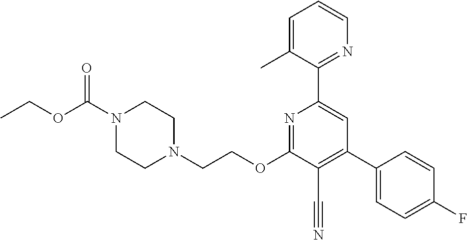 [SMILES: CCOC(=O)N1CCN(CCOc2nc(-c3ncccc3C)cc(-c3ccc(F)cc3)c2C#N)CC1] | Ethyl 4-(2-(3-cyano-4-(4- fluorophenyl)-6-(3- methylpyridin-2-yl)pyridin-2- yloxy)ethyl)piperazine-1- carboxylate | |
| 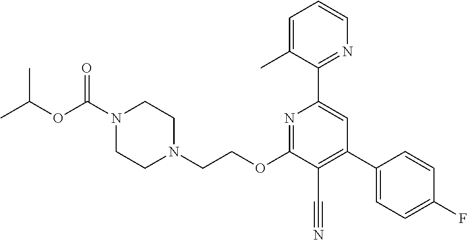 [SMILES: Cc1cccnc1-c1cc(-c2ccc(F)cc2)c(C#N)c(OCCN2CCN(C(=O)OC(C)C)CC2)n1] | Isopropyl 4-(2-(3-cyano-4-(4- fluorophenyl)-6-(3- methylpyridin-2-yl)pyridin-2- yloxy)ethyl)piperazine-1- carboxylate | |
| 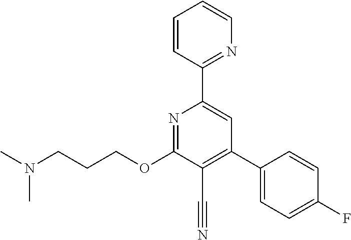 [SMILES: CN(C)CCCOc1nc(-c2ccccn2)cc(-c2ccc(F)cc2)c1C#N] | 2-(3- (Dimethylamino)propoxy)-4- (4-fluorophenyl)-6-(pyridin-2- yl)pyridine-3-carbonitrile | |
| 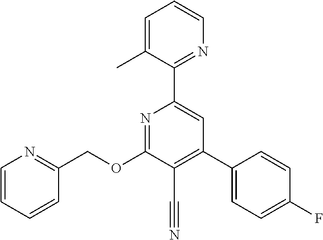 [SMILES: Cc1cccnc1-c1cc(-c2ccc(F)cc2)c(C#N)c(OCc2ccccn2)n1] | 2-((Pyridin-2-yl)methoxy)-4- (4-fluorophenyl)-6-(3- methylpyridin-2-yl)pyridine- 3-carbonitrile | |
| 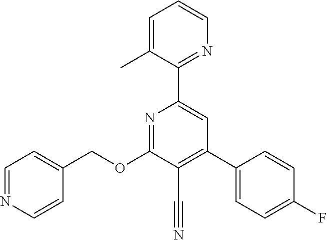 [SMILES: Cc1cccnc1-c1cc(-c2ccc(F)cc2)c(C#N)c(OCc2ccncc2)n1] | 2-((Pyridin-4-yl)methoxy)-4- (4-fluorophenyl)-6-(3- methylpyridin-2-yl)pyridine- 3-carbonitrile | |
| 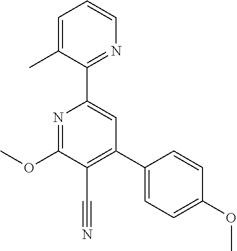 | 2-Methoxy-4-(4- methoxyphenyl)-6-(3- methylpyridin-2-yl)pyridine- 3-carbonitrile | |
| 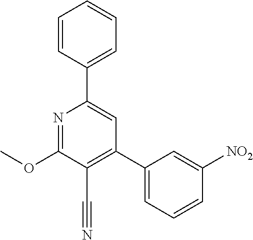 [SMILES: COc1nc(-c2ccccc2)cc(-c2cccc([N+](=O)[O-])c2)c1C#N] | 2-Methoxy-4-(3-nitrophenyl)- 6-phenylpyridine-3- carbonitrile | |
| 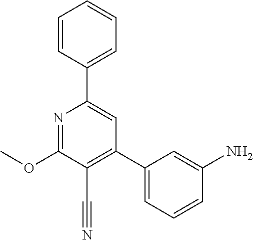 [SMILES: COc1nc(-c2ccccc2)cc(-c2cccc(N)c2)c1C#N] | 4-(3-Aminophenyl)-2- methoxy-6-phenylpyridine-3- carbonitrile | |
| 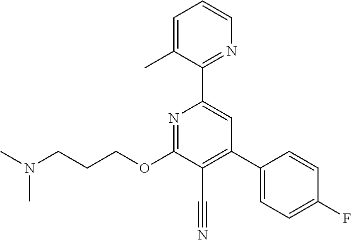 [SMILES: Cc1cccnc1-c1cc(-c2ccc(F)cc2)c(C#N)c(OCCCN(C)C)n1] | 2-(3- (Dimethylamino)propoxy)-4- (4-fluorophenyl)-6-(3- methylpyridin-2-yl)pyridine- 3-carbonitrile | |
| 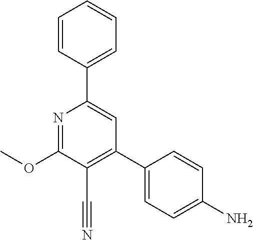 [SMILES: COc1nc(-c2ccccc2)cc(-c2ccc(N)cc2)c1C#N] | 4-(4-Aminophenyl)-2- methoxy-6-phenylpyridine-3- carbonitrile | |
| 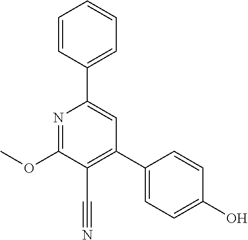 [SMILES: COc1nc(-c2ccccc2)cc(-c2ccc(O)cc2)c1C#N] | 4-(4-Hydroxyphenyl)-2- methoxy-6-phenylpyridine-3 carbonitrile | |
| 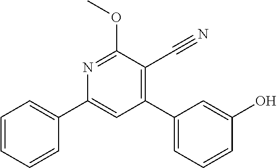 [SMILES: COc1nc(-c2ccccc2)cc(-c2cccc(O)c2)c1C#N] | 4-(3-Hydroxyphenyl)-2- methoxy-6-phenylpyridine-3 carbonitrile | |
| 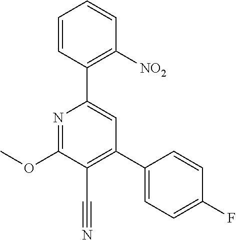 [SMILES: COc1nc(-c2ccccc2[N+](=O)[O-])cc(-c2ccc(F)cc2)c1C#N] | 4-(4-Fluorophenyl)-2- methoxy-6-(2- nitrophenyl)pyridine-3- carbonitrile | |
| 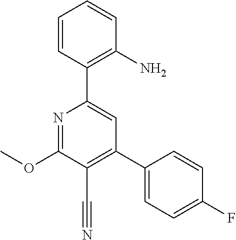 [SMILES: COc1nc(-c2ccccc2N)cc(-c2ccc(F)cc2)c1C#N] | 6-(2-Aminophenyl)-4-(4- fluorophenyl)-2- methoxypyridine-3- carbonitrile | |
| 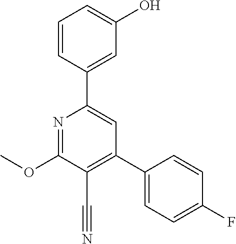 [SMILES: COc1nc(-c2cccc(O)c2)cc(-c2ccc(F)cc2)c1C#N] | 4-(4-fluorophenyl)-6-(3- hydroxyphenyl)-2- methoxypyridine-3- carbonitrile | |
| 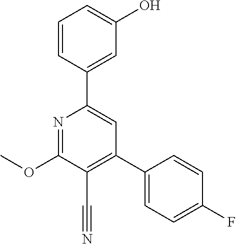 [SMILES: COc1nc(-c2cccc(O)c2)cc(-c2ccc(F)cc2)c1C#N] | 4-(4-Fluorophenyl)-6-(3- hydroxyphenyl)-2- methoxypyridine-3- carbonitrile | |
| 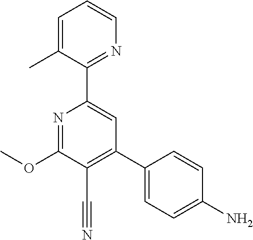 [SMILES: COc1nc(-c2ncccc2C)cc(-c2ccc(N)cc2)c1C#N] | 4-(4-Aminophenyl)-2- methoxy-6-(3-methylpyridin- 2-yl)pyridine-3-carbonitrile | |
| 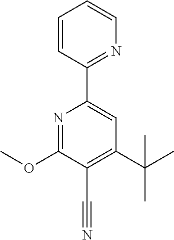 [SMILES: COc1nc(-c2ccccn2)cc(C(C)(C)C)c1C#N] | 4-tert-Butyl-2-methoxy-6- (pyridin-2-yl)pyridine-3- carbonitrile | |

In certain embodiments, the compound of formula (II) is selected from the group consisting of:

| Structure | Name | Cpd ID |
|---|---|---|
| 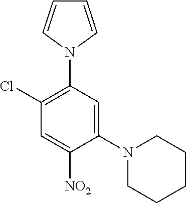 [SMILES: O=[N+]([O-])c1cc(Cl)c(-n2cccc2)cc1N1CCCCC1] | 1-(4-chloro-2-nitro-5-(1H- pyrrol-1- yl)phenyl)piperidine | 2 |
| 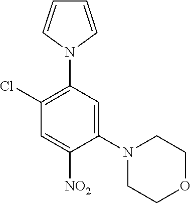 [SMILES: O=[N+]([O-])c1cc(Cl)c(-n2cccc2)cc1N1CCOCC1] | 4-(4-chloro-2-nitro-5-(1H- pyrrol-1- yl)phenyl)morpholine | 19 |
| 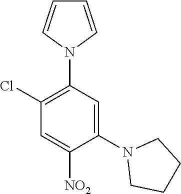 [SMILES: O=[N+]([O-])c1cc(Cl)c(-n2cccc2)cc1N1CCCC1] | 1-(2-chloro-4-nitro-5- (pyrrolidin-1- yl)phenyl)-1H-pyrrole | 20 |

In certain embodiments, the compound of formula (III) is selected from the group consisting of:

| | | Cpd |
|---|---|---|
| Structure | Name | ID |
| 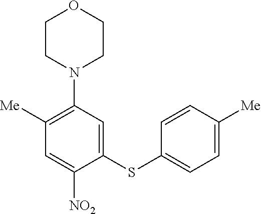 [SMILES: Cc1ccc(Sc2cc(N3CCOCC3)c(C)cc2[N+](=O)[O-])cc1] | 4-(2-methyl-4-nitro- 5-(p- tolylthio)phenyl) morpholine | 3 |
| 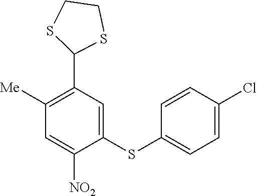 [SMILES: Cc1cc([N+](=O)[O-])c(Sc2ccc(Cl)cc2)cc1C1SCCS1] | 2-(5-((4- chlorophenyl)thio)- 2-methyl-4- nitrophenyl)-1,3- dithiolane | 32 |
| 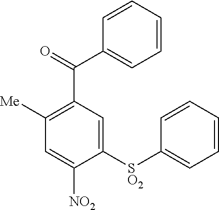 [SMILES: Cc1cc([N+](=O)[O-])c(S(=O)(=O)c2ccccc2)cc1C(=O)c1ccccc1] | (2-methyl-4-nitro-5- (phenylsulfonyl) phenyl)(phenyl) methanone | 33 |
| 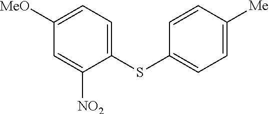 [SMILES: COc1ccc(Sc2ccc(C)cc2)c([N+](=O)[O-])c1] | (4-methoxy-2- nitrophenyl)(p- tolyl)sulfane | 34 |

The invention contemplates a pharmaceutical composition comprising at least one of the compounds recited herein.

Compounds of the invention can be prepared by the general schemes described elsewhere herein, using the synthetic method known by those skilled in the art. The examples provided herein illustrate non-limiting embodiments of the invention.

The compounds of the invention can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the invention contemplates that at least one quaternary carbon atom present in a compound of the invention can be replaced with a silicon atom. In non-limiting examples, a tert-butyl group can be replaced with a trimethylsilyl group. In certain embodiments, the replacement with silicon atom does not create a Si—O or Si—N bond.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplemental (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal. In certain embodiments, protective groups are removed by acid, base, reducing conditions (such as, for example, hydrogenolysis), and/or oxidative conditions.

### Methods

The invention includes a method of treating or preventing heart failure in a subject in need thereof. The method comprises administering to the subject a therapeutically effective amount of a compound of the invention. The method comprises, in some embodiments, administering to the subject a therapeutically effective amount of a compound of formula (IA), formula (IB), formula (II), and/or formula (III). In certain embodiments, treating or preventing heart failure comprises at least one of the following: regulating or lowering blood pressure (thus protecting against hypertension), inhibiting or minimizing atherosclerotic plaque formation (thus protecting against atherosclerosis), and/or reducing or reversing cardiac remodeling (which leads to heart failure). In other embodiments, the method further comprises administering to the subject an additional therapeutic agent that treats or prevents heart failure, high or elevated blood pressure, atherosclerotic plaque formation, and/or cardiac remodeling.

In certain embodiments, administering the compound of the invention to the subject allows for administering a lower dose of the additional therapeutic agent compared to the dose of the additional therapeutic agent alone that is required to achieve similar results in treating or preventing heart failure, high or elevated blood pressure, atherosclerotic plaque formation, and/or cardiac remodeling. For example, in other embodiments, the compound of the invention enhances the activity of the additional therapeutic compound, thereby allowing for a lower dose of the additional therapeutic compound to provide the same effect.

In certain embodiments, the compound of the invention and the additional therapeutic agent are co-administered to the subject. In other embodiments, the compound of the invention and the additional therapeutic agent are coformulated and co-administered to the subject.

The invention further includes a method of increasing, or reversing loss of, physiological levels of H₂S in a tissue of a subject. The method comprises administering to the subject a therapeutically effective amount of a compound of the invention.

In certain embodiments, the subject is a mammal. In other embodiments, the mammal is a human.

### Combination Therapies

The compounds useful within the methods of the invention can be used in combination with one or more additional agents useful for treating or preventing heart failure, high or elevated blood pressure, atherosclerotic plaque formation, and/or cardiac remodeling. These additional agents may comprise compounds that are commercially available or synthetically accessible to those skilled in the art. These additional agents are known to treat, prevent, or reduce the symptoms of heart failure, high or elevated blood pressure, atherosclerotic plaque formation, and/or cardiac remodeling.

In non-limiting examples, the compounds useful within the invention can be used in combination with one or more of the following agents: ACE inhibitors, beta blockers, neprilysin inhibitors, diuretics, aldosterone antagonists, vasodilators (such as hydralazine), angiotensin II-receptor blocker (ARB), a combination of an ARB with a neprilysin inhibitor (valsartan or sacubitril), and/or nitrates (such as isosorbide dinitrate).

A synergistic effect can be calculated, for example, using suitable methods such as, for example, the Sigrnoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations can be administered to the subject either prior to or after the onset of failure heart failure, high or elevated blood pressure, atherosclerotic plaque formation, and/or cardiac remodeling. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present invention to a patient, preferably a mammal, more preferably a human, can be carried out using known procedures, at dosages and for periods of time effective to treat or prevent heart failure, high or elevated blood pressure, atherosclerotic plaque formation, and/or cardiac remodeling. An effective amount of the therapeutic compound necessary to achieve a therapeutic effect may vary according to factors such as the state of the disease or disorder in the patient; the age, sex, and weight of the patient; and the ability of the therapeutic compound to treat or prevent heart failure, high or elevated blood pressure, atherosclerotic plaque formation, and/or cardiac remodeling. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention can be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of heart failure in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier can be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration can be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of heart failure, high or elevated blood pressure, atherosclerotic plaque formation, and/or cardiac remodeling in a patient.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use can be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets can be uncoated or they can be coated by known techniques for elegance or to delay the release of the active ingredients. Formulations for oral use may also be presented as hard gelatin capsules wherein the active ingredient is mixed with an inert diluent.

### Parenteral Administration

For parenteral administration, the compounds of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of heart failure in the patient being treated. The skilled artisan is able to determine appropriate dosages depending on these and other factors.

A suitable dose of a compound of the present invention can be in the range of from about 0.01 mg to about 5,000 mg per day, such as from about 0.1 mg to about 1,000 mg, for example, from about 1 mg to about 500 mg, such as about 5 mg to about 250 mg per day. The dose can be administered in a single dosage or in multiple dosages, for example from 1 to 4 or more times per day. When multiple dosages are used, the amount of each dosage can be the same or different. For example, a dose of 1 mg per day can be administered as two 0.5 mg doses, with about a 12-hour interval between doses.

It is understood that the amount of compound dosed per day can be administered, in non-limiting examples, every day, every other day, every 2 days, every 3 days, every 4 days, or every 5 days. For example, with every other day administration, a 5 mg per day dose can be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1: High-Throughput Screen of a Small Molecule Library

A biochemical assay (DCIP endpoint assay) to monitor SQOR activity that employs a water-soluble coenzyme Q derivative (CoQ1) was developed and found to be suitable for high-throughput screening (e.g., Z-factor=0.78). The assay is based on the ability of reduced CoQ1, which is produced in the SQOR reaction, to nonenzymically reduce 2,6-dichloroindiphenol (DCIP) (Δεox-red=20 mM−1 cm−1 at 600 nm). Reaction mixtures containing SQOR, substrates (H₂S, sulfite, CoQ1) and various concentrations of an inhibitor are incubated in 96-well plates for 60 s. The reactions are quenched by addition of formaldehyde and N-ethylmaleimide to denature SQOR and consume unreacted H₂S, respectively, and are then incubated for 10 min prior to addition of DCIP. The absorbance at 600 nm is read 30 s after DCIP addition by using a BioTek Synergy 2 plate reader.

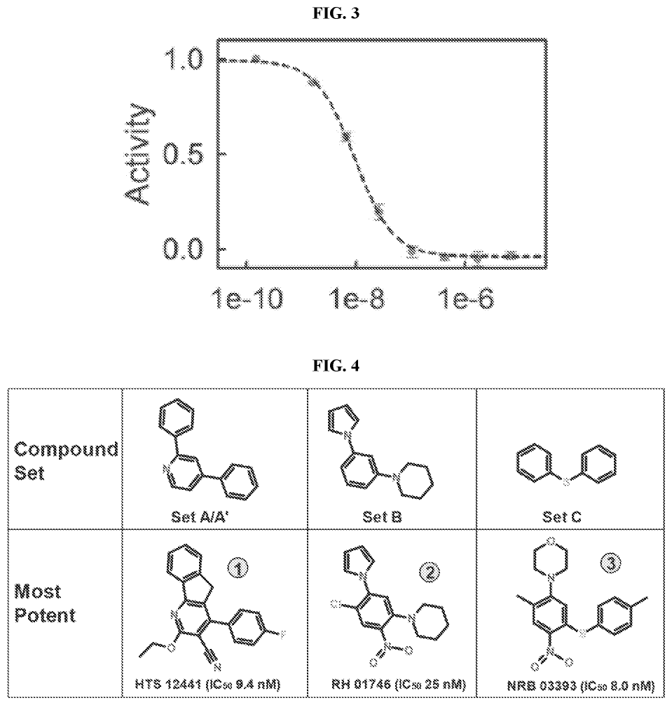

This assay was used to screen 41,000 compounds from a small molecule library. The screen identified a diverse collection of 521 compounds that inhibit SQOR with IC50 values below 20 μM and one of the most potent (Compound 1) has an IC50 of 9.4 nM (FIG. 3). The mechanism of inhibition was determined by using a continuous spectrophotometric assay in which the concentration of CoQ1 was varied in the absence or presence of several fixed inhibitor concentrations. Each compound was found to act as a competitive inhibitor with respect to CoQ1 and exhibit a Ki value 2.5-fold smaller than the IC₅₀ value determined using the DCIP endpoint assay, a difference expected because the endpoint assay is conducted at [CoQ1]=4 Km. The identification of drug-like compounds that competitively inhibit SQOR demonstrate the druggability of the CoQ site in this enzyme that catalyzes the pivotal first step in H₂S metabolism. The validated positives were further prioritized on the basis of observed IC50 values, allowing for the selection of three sets of compounds for further investigation (FIG. 4).

### Example 2: Synthesis and Biological Activity

Three sets (A, B, C) of SQOR inhibitors are shown in FIG. 4.

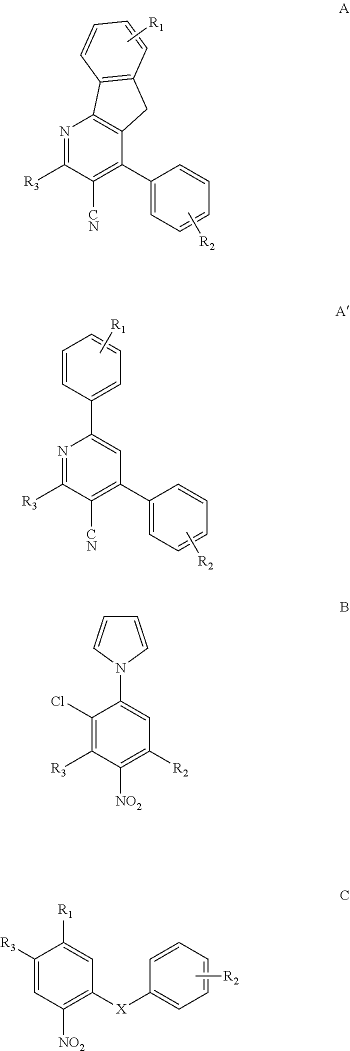

### Set A/A′:

Set A/A′ compounds and their corresponding SQOR inhibition data are illustrated in Table 1.

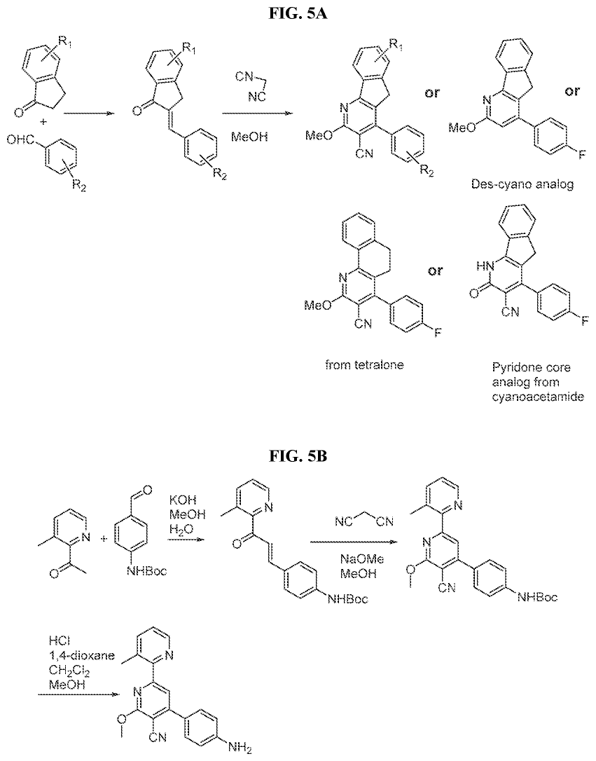

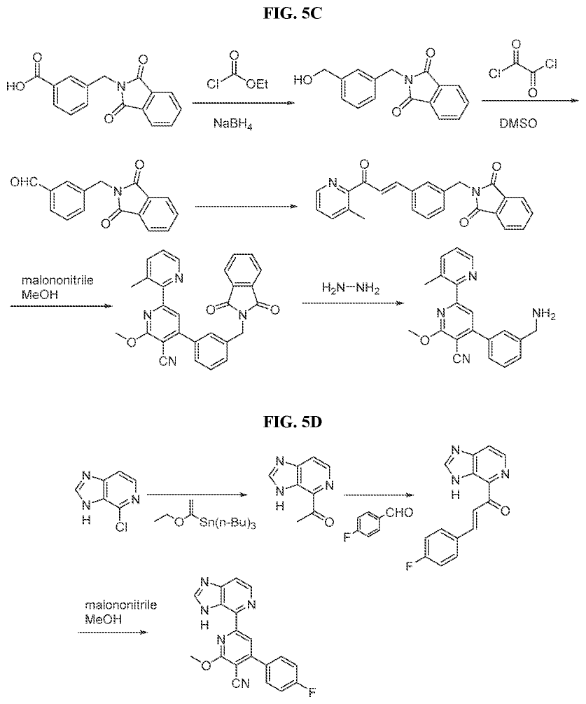

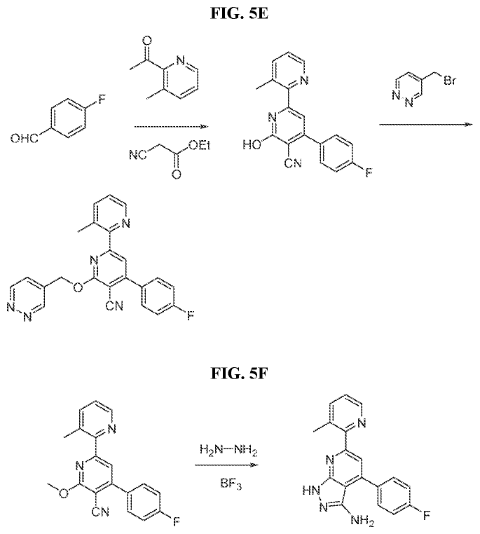

As illustrated in FIGS. 5A-5F, Set A/A′ compounds can be prepared in a straightforward manner.

As illustrated in FIG. 5A, condensation of an indalone and aldehyde, followed by cyclization with cyanoacetonitrile, provides the desired compound. Selection of various indalones and aldehydes, which may be independently commercially available or accessible through synthesis, allows for exploration of diverse substituents R₁ and R₂. Substituted acetophenones can be used to prepare derivatives of Set A′. Non-limiting general procedures are exemplified in the detailed experimentals which follow. The synthetic scheme of FIG. 5A allows for the synthesis of des-cyano analogues. Using a tetralone instead of an indalone results in a ring-expanded analogue. Further, use of cyanoacetamide, instead of using cyanoacetonile, can be used to provide a pyridone core.

FC9402 can be synthesized using a route such as that described in FIG. 5B.

As illustrated in FIG. 5C, the benzylamine analogues of FC9402 can be synthesized whereby the primary amine is protected as phthalimide at the onset of the synthesis. The phthamide protection can be removed by heating the protected compound with hydrazine at the end of the synthesis.

Azabenzimidazole analogues can be similarly prepared using a route such as that described in FIG. 5D.

The methoxy analog of set A′ can be prepared by benzylation as illustrated in FIG. 5E. As illustrated in FIG. 5F, a FC9402 analogue can react with hydrazine to give aminoindazole analogues.

### Set B:

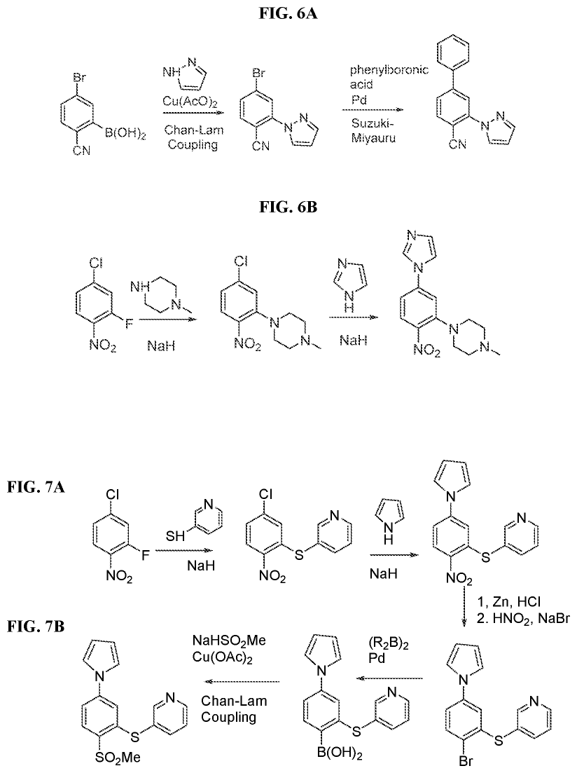

FIG. 6A illustrates the synthesis of Set B compounds involving two coupling reactions. The cyano analogue can be prepared from commercially available boronic acid by using the Chan-Lam Coupling reaction (Qiao and Lam, Synthesis 2011:829-856) in an open-flask reaction. In the second step, regular Suzuki-Miyaura Coupling [Suzuki, A. In Modern Arene Chemistry; Astruc, D., Ed.; Wiley-VCH: Weinheim, Germany, 2002, 5.] can be performed to yield the cyano analog.

As illustrated in FIG. 6B, two SNAr reactions can be performed under standard condition to give the piperizine imidazole analogue.

### Set C:

As illustrated in FIG. 7A, two SNAr can be used to yield the pyridylthio Set C compound.

For the methylsulfone (nitro replacement) analogue, the nitro compound can be converted to the corresponding bromo anlogues. Treatment with pinacol bisboronate in the presence of palladium catalyst provides the corresponding boronic acid. Again, Chan-Lam coupling can be performed on the boronic acid to yield the corresponding methylsulfone. Likewise, other heteroatom groups can be introduced, since Chan-Lam Coupling is capable of converting boronic acids into various heteroatom-containing substituents. The high-throughput SQOR biochemical assay can be used to determine IC₅₀ values for synthesized compounds.

TABLE 1
SAR of Set A/A′ and Set B

| | | R₁ | R₂ | R₃ | IC₅₀ nM |
|---|---|---|---|---|---|
| 1 | A | — | 4-F | OEt | 9.4 |
| 4 | A | — | — | OMe | 21 |
| 5 | A | — | 4-OMe | OEt | 260 |
| 6 | A | — | 2-Cl | OMe | 340 |
| 7 | A | — | 4-OMe | OMe | 880 |
| 8 | A | — | 4-CF₃ | OMe | 1,100 |
| 9 | A | — | 2-F-5-Cl | OMe | 3,500 |
| 10 | A | — | 3-OMe | OMe | 6,100 |
| 11 | A′ | — | — | OCH₂CO₂Et | 30 |
| 12 | A′ | — | 3,4-OCH₂O— | OEt | 890 |
| 13 | A′ | — | — | OEt | 19 |
| 14 | A′ | 4-F | — | NH₃ | 600 |
| 15 | A′ | 2,5-dimethyl | 3,4-OCH₂O— | NH₃ | 1,100 |
| 16 | A′ | 4-Me | — | NH₃ | 11,000 |
| 17 | A′ | 4-OH | — | NH₃ | 12,000 |
| 18 | A′ | 3,4-dimethyl | 3,4- | NH₃ | 14,000 |
| | | | dimethoxy | | |
| 2 | B | — | N-piperidine | — | 25 |
| 19 | B | — | N-morpholine | — | 440 |
| 20 | B | — | N-pyrrolidine | — | 460 |

TABLE 2
SAR of Set C

| | X | R₁ | R₂ | R₃ | IC₅₀ nM |
|---|---|---|---|---|---|
| 3 | S | N-morpholino | 4-Me | Me | 8.0 |
| 32 | S | 1,3-dithiolane | 4-Cl | — | 24 |
| 33 | SO₂ | benzoyl | — | — | 152 |
| 34 | S | — | 4-Me | MeO | 4,900 |

TABLE 3

| | IC₅₀ |
|---|---|
| Compound ID | (nM) |
| 4-(Furan-2-yl)-2-methoxy-6-(pyridin-2-yl)pyridine-3-carbonitrile | 680 |
| 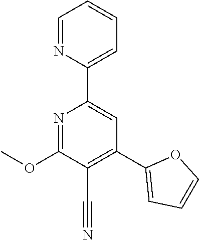 [SMILES: COc1nc(-c2ccccn2)cc(-c2ccco2)c1C#N] | |
| 4-Ethoxy-2,6-diphenylpyrimidine-5-carbonitrile | 120 |
| 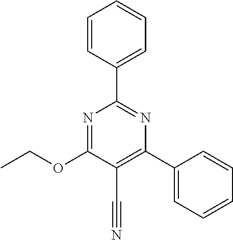 [SMILES: CCOc1nc(-c2ccccc2)nc(-c2ccccc2)c1C#N] | |
| N-(2-(3-Cyano-4-(4-fluorophenyl)-6-(pyridin-2-yl)pyridin-2- | 1,900 |
| yloxy)ethyl)acetamide | |
| 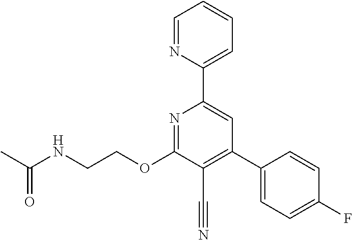 [SMILES: CC(=O)NCCOc1nc(-c2ccccn2)cc(-c2ccc(F)cc2)c1C#N] | |
| 2-(Cyanomethoxy)-4-(4-fluorophenyl)-6-(pyridin-2-yl)pyridine-3-carbonitrile | 25 |
| 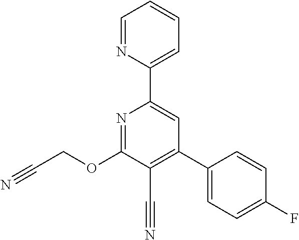 [SMILES: N#CCOc1nc(-c2ccccn2)cc(-c2ccc(F)cc2)c1C#N] | |
| 2-((S)-5-(Methoxy)pyrrolidin-2-one)-4-(4-fluorophenyl)-6-(pyridin-2- | 39,000 |
| yl)pyridine-3-carbonitrile | |
| 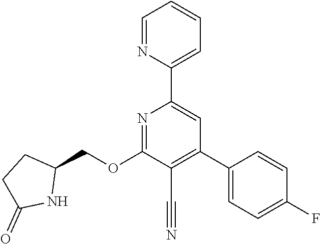 [SMILES: N#Cc1c(-c2ccc(F)cc2)cc(-c2ccccn2)nc1OC[C@@H]1CCC(=O)N1] | |
| 2-((R)-5-(Methoxy)pyrrolidin-2-one)-4-(4-fluorophenyl)-6-(pyridin-2- | 2,700 |
| yl)pyridine-3-carbonitrile | |
| 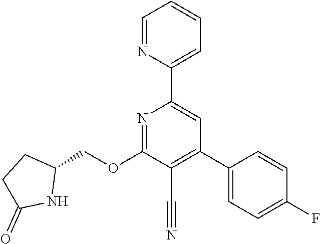 [SMILES: N#Cc1c(-c2ccc(F)cc2)cc(-c2ccccn2)nc1OC[C@H]1CCC(=O)N1] | |
| 2-(Allyloxy)-4-(4-fluorophenyl)-6-(pyridin-2-yl)pyridine-3-carbonitrile | 7.2 |
| 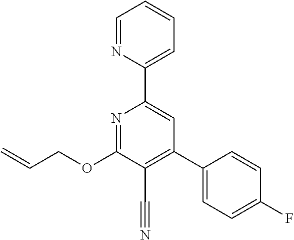 [SMILES: C=CCOc1nc(-c2ccccn2)cc(-c2ccc(F)cc2)c1C#N] | |
| 2-Methoxy-3-methyl-4,6-diphenylpyridine | 440 |
| 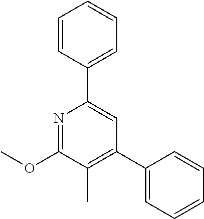 [SMILES: COc1nc(-c2ccccc2)cc(-c2ccccc2)c1C] | |
| 2-(2-Morpholinoethoxy)-4-(4-fluorophenyl)-6-(3-methylpyridin-2- | 270 |
| yl)pyridine-3-carbonitrile | |
| 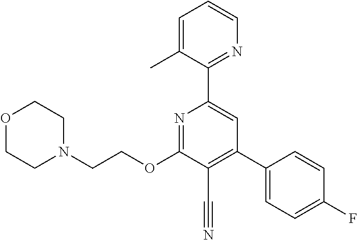 [SMILES: Cc1cccnc1-c1cc(-c2ccc(F)cc2)c(C#N)c(OCCN2CCOCC2)n1] | |
| 4-(4-fluorophenyl)-1,2-dihydro-6-(3-methylpyridin-2-yl)-1-(2-morpholinoethyl)- | 14,000 |
| 2-oxopyridine-3-carbonitrile | |
| 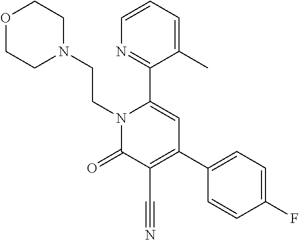 [SMILES: Cc1cccnc1-c1cc(-c2ccc(F)cc2)c(C#N)c(=O)n1CCN1CCOCC1] | |
| Methyl 2-(3-cyano-4-(4-fluorophenyl)-6-(3-methylpyridin-2-yl)pyridin-2- | 96 |
| yloxy) 2-(2-methoxyethoxy) acetate | |
| 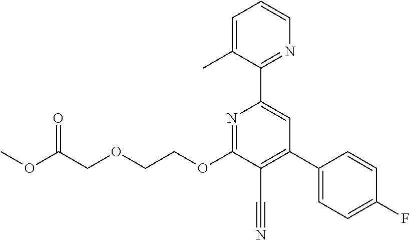 [SMILES: COC(=O)COCCOc1nc(-c2ncccc2C)cc(-c2ccc(F)cc2)c1C#N] | |
| 2-((Pyridin-3-yl)methoxy)-4-(4-fluorophenyl)-6-(3-methylpyridin-2- | 40 |
| yl)pyridine-3-carbonitrile | |
| 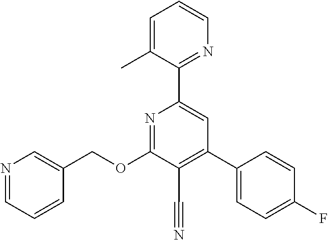 [SMILES: Cc1cccnc1-c1cc(-c2ccc(F)cc2)c(C#N)c(OCc2cccnc2)n1] | |
| 4-(4-fluorophenyl)-1,2-dihydro-6-(3-methylpyridin-2-yl)-2-oxo-1-((pyridin-3- | 10,000 |
| yl)methyl)pyridine-3-carbonitrile | |
| 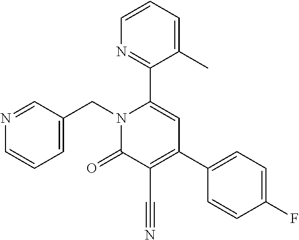 [SMILES: Cc1cccnc1-c1cc(-c2ccc(F)cc2)c(C#N)c(=O)n1Cc1cccnc1] | |
| tert-Butyl 4-(2-(3-cyano-4-(4-fluorophenyl)-6-(3-methylpyridin-2-yl)pyridin- | 43 |
| 2-yloxy)ethyl)piperazine-1-carboxylate | |
| 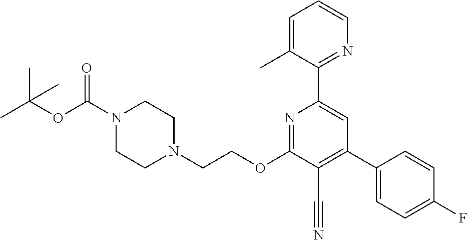 [SMILES: Cc1cccnc1-c1cc(-c2ccc(F)cc2)c(C#N)c(OCCN2CCN(C(=O)OC(C)(C)C)CC2)n1] | |
| Methyl 4-(2-(3-cyano-4-(4-fluorophenyl)-6-(3-methylpyridin-2-yl)pyridin-2- | 540 |
| yloxy)ethyl)piperazine-1-carboxylate | |
| 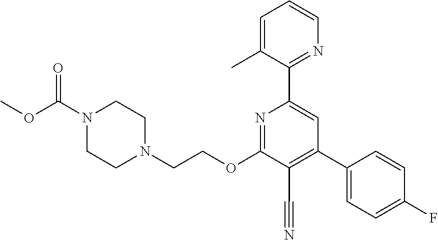 [SMILES: COC(=O)N1CCN(CCOc2nc(-c3ncccc3C)cc(-c3ccc(F)cc3)c2C#N)CC1] | |
| Ethyl 4-(2-(3-cyano-4-(4-fluorophenyl)-6-(3-methylpyridin-2-yl)pyridin-2- | 230 |
| yloxy)ethyl)piperazine-1-carboxylate | |
| 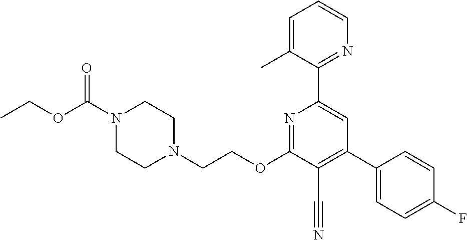 [SMILES: CCOC(=O)N1CCN(CCOc2nc(-c3ncccc3C)cc(-c3ccc(F)cc3)c2C#N)CC1] | |
| Isopropyl 4-(2-(3-cyano-4-(4-fluorophenyl)-6-(3-methylpyridin-2-yl)pyridin- | 120 |
| 2-yloxy)ethyl)piperazine-1-carboxylate | |
| 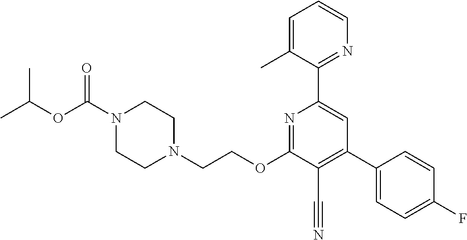 [SMILES: Cc1cccnc1-c1cc(-c2ccc(F)cc2)c(C#N)c(OCCN2CCN(C(=O)OC(C)C)CC2)n1] | |
| 2-(3-(Dimethylamino)propoxy)-4-(4-fluorophenyl)-6-(pyridin-2-yl)pyridine- | 2,200 |
| 3-carbonitrile | |
| 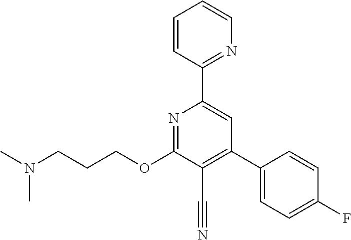 [SMILES: CN(C)CCCOc1nc(-c2ccccn2)cc(-c2ccc(F)cc2)c1C#N] | |
| 2-((Pyridin-2-yl)methoxy)-4-(4-fluorophenyl)-6-(3-methylpyridin-2- | 32 |
| yl)pyridine-3-carbonitrile | |
| 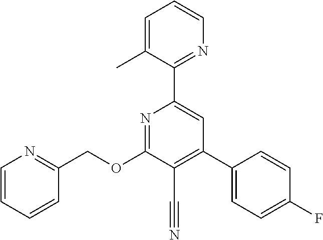 [SMILES: Cc1cccnc1-c1cc(-c2ccc(F)cc2)c(C#N)c(OCc2ccccn2)n1] | |
| 2-((Pyridin-4-yl)methoxy)-4-(4-fluorophenyl)-6-(3-methylpyridin-2- | 120 |
| yl)pyridine-3-carbonitrile | |
| 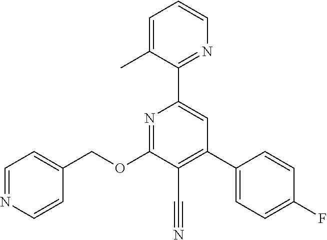 [SMILES: Cc1cccnc1-c1cc(-c2ccc(F)cc2)c(C#N)c(OCc2ccncc2)n1] | |
| 2-Methoxy-4-(4-methoxyphenyl)-6-(3-methylpyridin-2-yl)pyridine-3-carbonitrile | 670 |
| 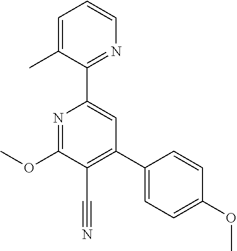 | |
| 2-Methoxy-4-(3-nitrophenyl)-6-phenylpyridine-3-carbonitrile | 48,000 |
| 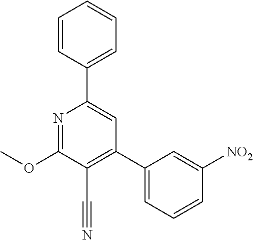 [SMILES: COc1nc(-c2ccccc2)cc(-c2cccc([N+](=O)[O-])c2)c1C#N] | |
| 4-(3-Aminophenyl)-2-methoxy-6-phenylpyridine-3-carbonitrile | 1,500 |
| 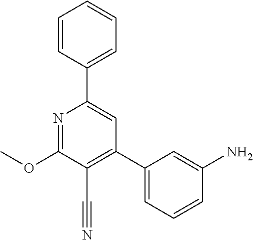 [SMILES: COc1nc(-c2ccccc2)cc(-c2cccc(N)c2)c1C#N] | |
| 2-(3-(Dimethylamino)propoxy)-4-(4-fluorophenyl)-6-(3-methylpyridin-2- | 2,700 |
| yl)pyridine-3-carbonitrile | |
| 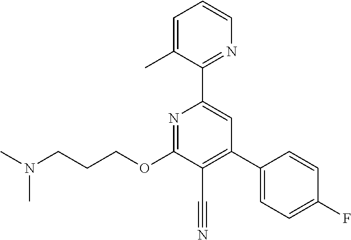 [SMILES: Cc1cccnc1-c1cc(-c2ccc(F)cc2)c(C#N)c(OCCCN(C)C)n1] | |
| 4-(4-Aminophenyl)-2-methoxy-6-phenylpyridine-3-carbonitrile | 92 |
| 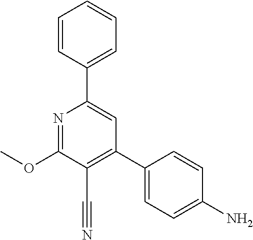 [SMILES: COc1nc(-c2ccccc2)cc(-c2ccc(N)cc2)c1C#N] | |
| 4-(4-Hydroxyphenyl)-2-methoxy-6-phenylpyridine-3-carbonitrile | 480 |
| 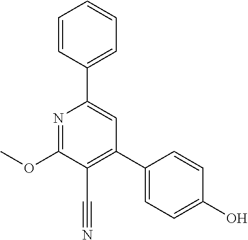 [SMILES: COc1nc(-c2ccccc2)cc(-c2ccc(O)cc2)c1C#N] | |
| 4-(3-Hydroxyphenyl)-2-methoxy-6-phenylpyridine-3-carbonitrile | 1,900 |
| 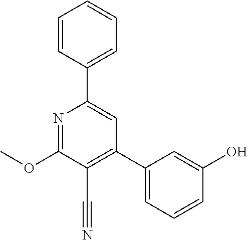 [SMILES: COc1nc(-c2ccccc2)cc(-c2cccc(O)c2)c1C#N] | |
| 4-(4-Fluorophenyl)-2-methoxy-6-(2-nitrophenyl)pyridine-3-carbonitrile | 7.0 |
| 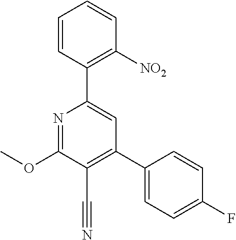 [SMILES: COc1nc(-c2ccccc2[N+](=O)[O-])cc(-c2ccc(F)cc2)c1C#N] | |
| 6-(2-Aminophenyl)-4-(4-fluorophenyl)-2-methoxypyridine-3-carbonitrile | 230 |
| 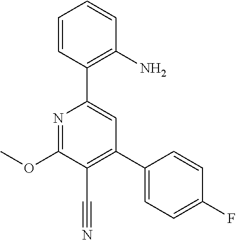 [SMILES: COc1nc(-c2ccccc2N)cc(-c2ccc(F)cc2)c1C#N] | |
| 4-(4-fluorophenyl)-6-(3-hydroxyphenyl)-2-methoxypyridine-3-carbonitrile | 230 |
| 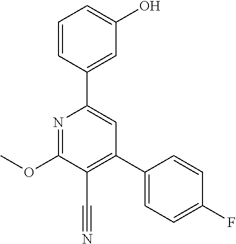 [SMILES: COc1nc(-c2cccc(O)c2)cc(-c2ccc(F)cc2)c1C#N] | |
| 4-(4-Fluorophenyl)-6-(4-hydroxyphenyl)-2-methoxypyridine-3-carbonitrile | 1,000 |
| 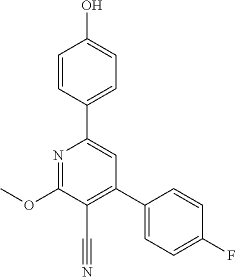 [SMILES: COc1nc(-c2ccc(O)cc2)cc(-c2ccc(F)cc2)c1C#N] | |
| 4-(4-Aminophenyl)-2-methoxy-6-(3-methylpyridin-2-yl)pyridine-3-carbonitrile | 29 |
| 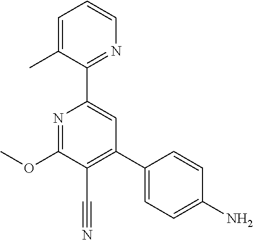 [SMILES: COc1nc(-c2ncccc2C)cc(-c2ccc(N)cc2)c1C#N] | |

### Example 3: Models of SQOR Complexes with Potent Set A′ and Set A Inhibitors

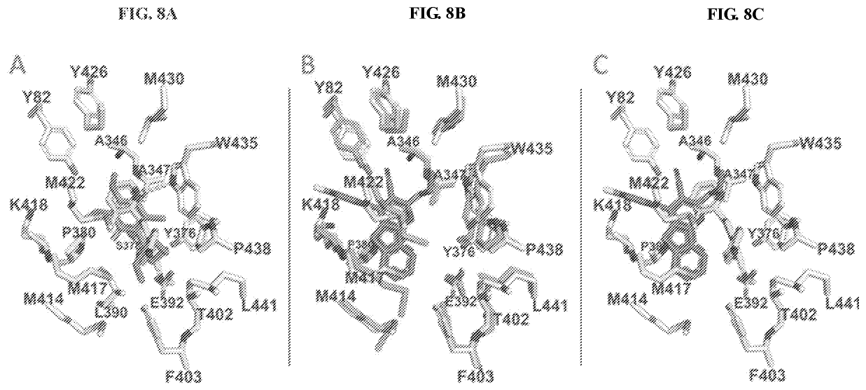

Human SQOR is an integral mitochondrial membrane protein that oxidizes H₂S using FAD and two active-site cysteines (Cys201, Cys379) as redox cofactors to catalyze the transfer of electrons from H₂S to coenzyme Q (CoQ). The X-ray structure of human SQOR at 2.59 Å resolution was recently reported (Jackson et al., 2019, Structure 27: 794-805). The observed structure reveals an internal tunnel that binds CoQ and connects the enzyme's membrane-binding surface to its hydrophilic H₂S-oxidizing active site. The entrance to the hydrophobic CoQ-binding pocket is located on the membrane-facing surface. The polar quinone ring of decyl-CoQ (DCQ) is inserted deep into the CoQ-binding pocket (FIG. 8A). The molecule of DCQ is surrounded by hydrophobic residues. The O2 carbonyl oxygen in the quinone ring of DCQ is hydrogen bonded to Trp345:NE1. The hydrophobic decyl tail of DCQ points to the outside of the protein, the region likely to be inserted into the mitochondrial inner membrane.

All SQOR inhibitors tested thus far act as competitive inhibitors with respect to DCQ. Models of SQOR●inhibitor complexes were produced using GOLD with flexible ligand/rigid protein docking to ligand-free SQOR (PDB:ID 6M06). Results obtained with 4-(4-aminophenyl)-2-methoxy-6-(3-methylpyridin-2-yl)pyridine-3-carbonitrile (FC9402) indicate that this Set A′ inhibitor binds within the CoQ-binding pocket, close to DCQ. The 2-pyridyl ring nitrogen in FC9402 and the O2 carbonyl in DCQ occupy similar positions and are both hydrogen bonded to Trp345:NE1. The aniline moiety of FC9402 points toward entrance of the pocket (FIG. 8A). A very similar binding mode is observed upon docking other potent Set A′ inhibitors.

Models of SQOR●Set A′ inhibitor complexes produced with GOLD are very similar to those obtained with GLIDE, also using flexible ligand/rigid protein docking. The same approach with either program failed to dock the most potent Set A inhibitor (HTS12441, compound 1) into the CoQ binding pocket. However, a stable SQOR●HTS12441 complex (MMGBSA dG=−80.5 kcal/mol) was obtained using GLIDE by enabling sidechain flexibility within the CoQ binding pocket. Except for Met422 and Met417, only minor differences in the position of amino acid sidechains within the CoQ binding pocket are observed in SQOR●HTS12441 complex as compared with the SQOR crystal structure (FIG. 8B). The observed displacement of Met417 in the model of the SQOR●HTS12441 complex eliminates a steric clash between the inhibitor and the position of the sidechain in the crystal structure. The predicted binding site of HTS12441 is considerably displaced from that of FC9402 (FIG. 8C) and other Set A′ inhibitors, a difference that may reflect the enforced coplanarity of the central pyridine ring and the 2-aryl substituent in the constrained Set A inhibitor.

### Example 4: Biological Activity of FC9402 in Cell-Based and Biochemical Assays

### Materials and Methods

Biochemical Selectivity Assays:

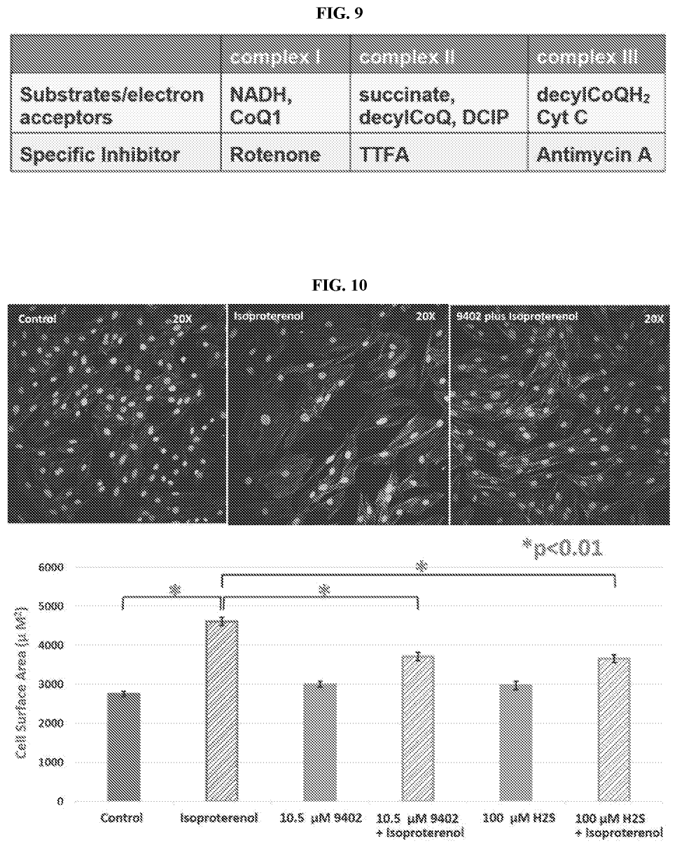

The selectivity of FC9402 for the CoQ site in SQOR was evaluated by determining whether the compound inhibited any of the three major mitochondrial CoQ-dependent respiratory complexes (I, II, III). Inhibitor selectivity was assessed using mouse muscle homogenate preparations that contain disrupted mitochondrial membranes, a feature that makes substrates accessible to each of the membrane-bound respiratory complexes. Spectrophotometric assays for each complex are conducted in the presence or absence of a target-specific inhibitor (FIG. 9) to correct for nonspecific substrate oxidation, as previously described (Spinazzi et al., 2012, Nature Protocols 7: 1235-1246). Thus, the activity of complex I is determined by monitoring the oxidation of NADH in the absence or presence of rotenone. Complex III activity is measured based on the rate of cytochrome C reduction observed in the presence or absence of antimycin A. The reaction catalyzed by complex II produces decylubiquinol (decylCoQH₂), which rapidly reduces DCIP. The activity of complex III is determined by monitoring DCIP reduction in the presence and absence of 2-thenoyltrifluoroacetone (TTFA).

Isolation of Rat Neonatal Ventricular Cardiomyocytes:

Rat neonatal ventricular cardiomyocytes (NVCMs) were isolated from the hearts of 1-2 day old Sprague-Dawley rats (Charles River) in 50-90% yield (70-95% viability) by following the protocol described by Golden et al. (Golden et al., 2012, in Cardiovascular Development. Methods in Molecular Biology (Methods and Protocols), vol 843 (Peng, X., Antonyak, M., Eds.) pp 205-214, Springer, Totowa, N.J.).

Hypertrophy Assays with Rat Neonatal Ventricular Cardiomyocytes (NVCMs) or H9C2 Cells:

NVCMs are maintained in Dulbecco's modified Eagle's medium (DMEM:F12) containing 20% fetal bovine serum (FBS), 5% horse serum, 1× GlutaMAX, and 2 μg/mL insulin. For hypertrophy studies, cells were plated onto 4-well glass chamber slides, precoated with poly-D-lysine and laminin. The maintenance medium was aspirated after incubation for 72 h. The cells were serum-starved overnight in DMEM:F12 containing 1% FBS, 1× GlutaMAX, and 2 ug/mL insulin and then incubated for 24 h in fresh serum-starved medium containing angiotensin II, as indicated.

H9C2 cells, a rat ventricular cardiomyoblast cell line were obtained from ATCC (CRL-1446) and maintained in Dulbecco's modified Eagle's medium (DMEM) containing 10% FBS and 1× GlutaMAX. For hypertrophy studies, the cells were plated onto 4-well glass chamber slides precoated with poly-D-lysine. The maintenance medium was aspirated after 24 h and the cells were serum-starved overnight in DMEM containing 1% FBS and 1× GlutaMAX and then incubated for 48 h in fresh serum-starved medium containing isoproterenol, as indicated.

For cell size determination, treated NRVMs or H9C2 cells were fixed and cell membranes stained with wheat germ agglutinin (WGA)-Alexa Fluor 594 conjugate (Biotium (29023). To visualize sarcomeric α-actinin in NRVMs, the cells were blocked and permeabilized, and then incubated with sarcomeric α-actinin rabbit primary antibody (Abeam ab9465), followed by Alexa Fluor 488 goat anti-rabbit IgG secondary antibody (Abeam ab150077). To visualize F-actin in H9C2 cells, the cells were permeabilized and then stained with phalloidin-Alexa Fluor 488 conjugate (Invitrogen A12379). Nuclei in NRVMs or H9C2 cells were stained with DAPI (EMD Millipore Corp 2965127). Images were acquired using a FV3000 Olympus Confocal Microscope and analyzed using ImageJ. Cell surface area was determined by encircling the outer membrane of 30-50 cells per field in 6-10 field images for a total of 500 cells per condition.

The Cell Counting Kit-8 (Dojindo Molecular Technologies) was used to evaluate the possible cytotoxicity of FC9402 in assays with NVCMs or H9C2 cells. Cytotoxicity assays were conducted under the same conditions used for hypertrophy assays with NVCMs or H9C2 cells, as described elsewhere herein.

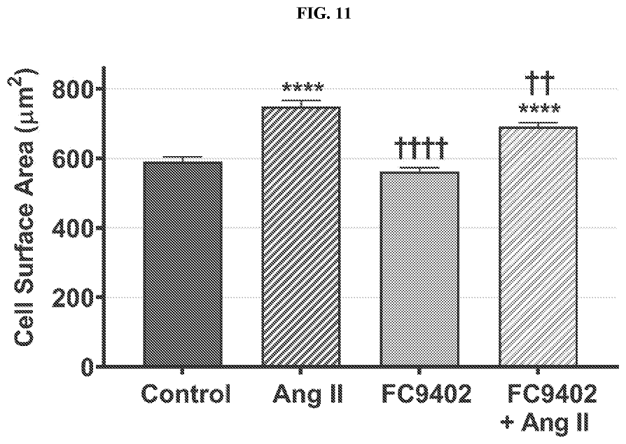

Selected results are illustrated herein. FC9402 exhibits little or no cytotoxicity in H9C2 cells (CC₅₀=56±10 μM) or in NVCMs (CC₅₀=27.9±2.6 μM). FC9402 did not inhibit any of the three major CoQ-dependent mitochondrial respiratory complexes (I, II, III) at the highest concentration tested (11 μM) in selectivity assays (FIG. 9) with mammalian mitochondrial membrane preparations. The results show that FC9402 exhibits a high degree of specificity for the CoQ binding site in SQOR (IC₅₀=30 nM). FC9402 mitigates the hypertrophic response induced by treatment of H9C2 cells with isoproterenol, an analog of epinephrine that is a non-selective β-adrenoreceptor agonist. The cardioprotective effect observed with 10.5 μM FC9402 is similar to that seen with 100 μM H₂S (FIG. 10). Further, FC9402 prevents angiotensin II-induced hypertrophy of NVCMs (FIG. 11).

### Example 5: Efficacy of FC9402 in a Mouse Model of Pressure-Induced Heart Failure

Transverse aortic constriction (TAC), which mimics human aortic stenosis, is a well-established mouse model for pressure-induced cardiac hypertrophy and heart failure with reduced ejection fraction (van Oort & Wehrens, 2010, JoVE e1729; Zhang, el al., 2013, Exp. Clin. Cardiol. 18:e115; Stansfield, et al., 2007, J. Surg. Res. 142:387-393). Patients with heart failure with reduced ejection volume exhibit a pronounced decrease (up to 3-fold) in plasma H₂S levels that appears to be correlated with the severity of the disease (Jiang et al., 2005, J. First Mil. Med. Univ. 25: 951-954; Kovacic et al., 2012, J. Card. Fail. 18: 541-548; Polhemus et al., 2014, Scientifica 2014: 768607). The finding of reduced H₂S levels has been replicated in the TAC mouse model of heart failure (Kondo et al., 2013, Circulation 127: 1116-1127; Polhemus et al., 2013, Circ. Heart Fail. 6: 1077-1086). The steady-state tissue concentration of H₂S reflects the difference between high rates of H₂S biosynthesis and mitochondrial metabolism (Vitvitsky et al., 2012, Antioxid. Redox Signal. 17: 22-31). A ˜40-fold increase in H₂S tissue levels is observed when the mitochondrial metabolism of H₂S (see FIGS. 2A-2B) is completely blocked by a genetic deficiency of sulfur dioxygenase (SDO). Slowing H₂S metabolism by inhibiting SQOR is predicted to normalize H₂S levels in the TAC mouse model and mitigate pressure-induced cardiac hypertrophy and heart failure. This prediction is validated by results obtained in the first-in-mouse efficacy study with FC9402 in the TAC mouse model of heart failure, as described below.

### Materials and Methods

Mice:

Male C57BL/6J mice 10-14 weeks of age were purchased from The Jackson Laboratory (Bar Harbor, Me.).

Transverse Aortic Constriction (TAC) Protocol:

Mice were anesthetized with isofluran or ketamine/xylazine. The mice were orally intubated and placed on a Harvard volume-cycled rodent ventilator to maintain respiration during surgery. The TAC procedure was performed by tying the aortic arch between the brachiocephalic trunk (innominate artery) and the left carotid artery against a 27-gauge needle spacer with a 7-0 silk suture, followed by immediate removal of the needle. For sham surgeries, thoracotomy was performed and the aorta was surgically exposed without any further intervention. Following surgical closure of the chest, the animals were allowed to recover from anesthesia, followed by post-operative care. The animals were euthanized 12 weeks post-surgery; hearts were harvested, weighed and tibia lengths were measured.

FC9402 Preparation and Administration:

Stock solutions of FC9402 (5 mg/mL) were prepared weekly in N-methyl-2-pyrrolidone (NMP):propylene glycol (PG): 19% (w/v) 2-hydroxypropyl-beta-cyclodextran (kleptose) in water (10:40:50), filter-sterilized through a 0.2 μm PTFE membrane into vials with snap-top caps with butyl rubber septa, and stored in aliquots at −80° C. until use. Sterile stock solutions of vehicle only [NMP:PG:kleptose in water (10:40:50)] were similarly prepared each week and stored in aliquots at 4° C. until use. For daily injections, a vial containing FC9402 or vehicle only was removed from storage, heated to 55° C. (to minimize precipitation), and then cooled to room temperature. Mice were injected intraperitoneally with 50 uL of FC9402 (10 mg/kg) or vehicle only, once a day for 12 weeks following surgery.

Echocardiography:

Transthoracic echocardiography was used to noninvasively quantify cardiac structural and functional changes during the progression to heart failure in the TAC mouse model. Measurements were conducted with animals under 1-3% isoflurane anesthesia using a Vevo® 2100 Imaging System with a high-frequency (18-38 MHz) probe (VisualSonics MS400) (Kohut et al., 2016, J. Cardiovasc. Ultrasound 24: 229-238). Echocardiography using color Doppler ultrasound was performed at 1 week after surgery to confirm the aortic constriction. The aortic constriction was further confirmed by conducting color Doppler and pulsed wave Dopier measurements at 12 weeks after surgery. Baseline echocardiography evaluations were performed 1-2 days prior to surgery. For these studies, parasternal long and short axis images were obtained in two-dimensional B-mode and M-mode. Additional images were obtained at regular intervals (2, 4, 6, 8, 10, 12 weeks) after surgery. To evaluate cardiac structure and function, the echocardiographic data were used to determine left ventricle (LV) end-diastolic diameter, LV end-systolic diameter, LV end-diasolic volume, LV ejection fraction, LV fractional shortening, and LV mass.

Cardiac Histology:

Mouse hearts were collected at 12 weeks post surgery, fixed in 10% buffered formalin, and then embedded in paraffin blocks, which were used to prepare serial 4 μm thick heart sections. To detect interstitial fibrosis, heart sections were stained with Masson's trichrome which colors collagen blue and cytoplasm red. Digital images of the slides were captured at 160× magnification using a Leica DFC290 digital camera in conjunction with a Leica MZ16F microscope. Interstitial fibrosis analysis was independently performed in a blinded manner by two observers using ImageJ. For each heart, 2 sections taken from the mid-ventricle were analyzed. Outliers were identified using Microsoft Excel and eliminated before averaging the numbers to obtain a single % fibrosis area/left ventricle measurement for each group of animals. To assess cardiomyocyte hypertrophy, heart sections were stained with wheat germ agglutinin (WGA)-Alexa Fluor 594 conjugate (Biotium). Images were acquired using a FV3000 Olympus Confocal Microscope and analyzed in a blinded manner by two observers. Five field views per mid-ventricle heart section were analyzed by measuring the cross-sectional area of 100 cells per field view using ImageJ. A total of 500 cells per heart section were measured.

Statistical Analysis:

All data are expressed as means±SEM. Statistical significance was evaluated for comparison between 3 or more means using one-way ANOVA with Tukey-Kramer post hoc test. For the echcocardiography data, obtained prior to and at two-week intervals after surgery, statistical significance was evaluated using two-way repeated ANOVA with Bonferroni post hoc test.

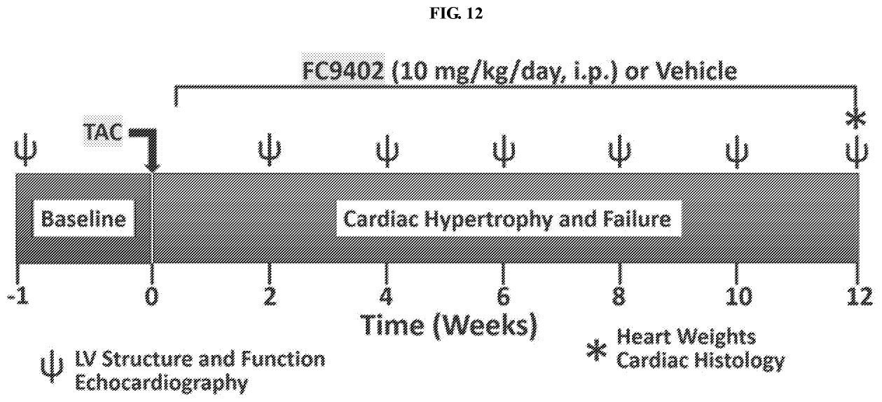

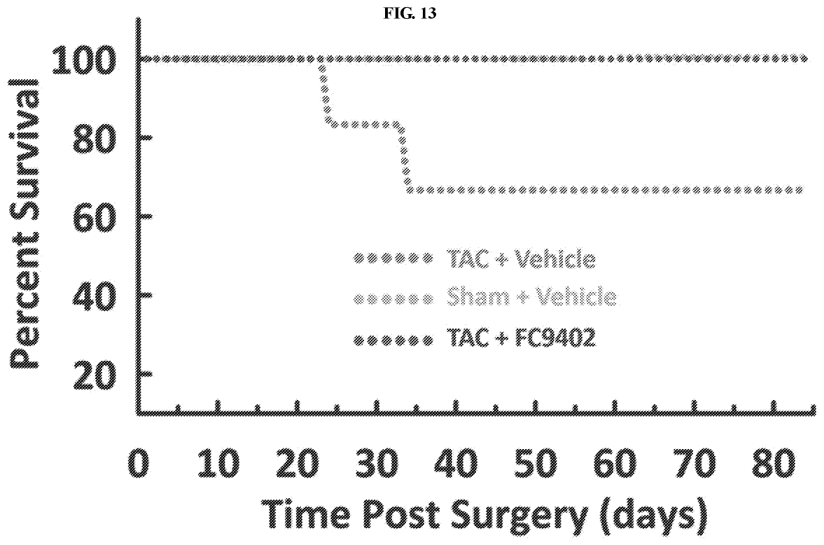

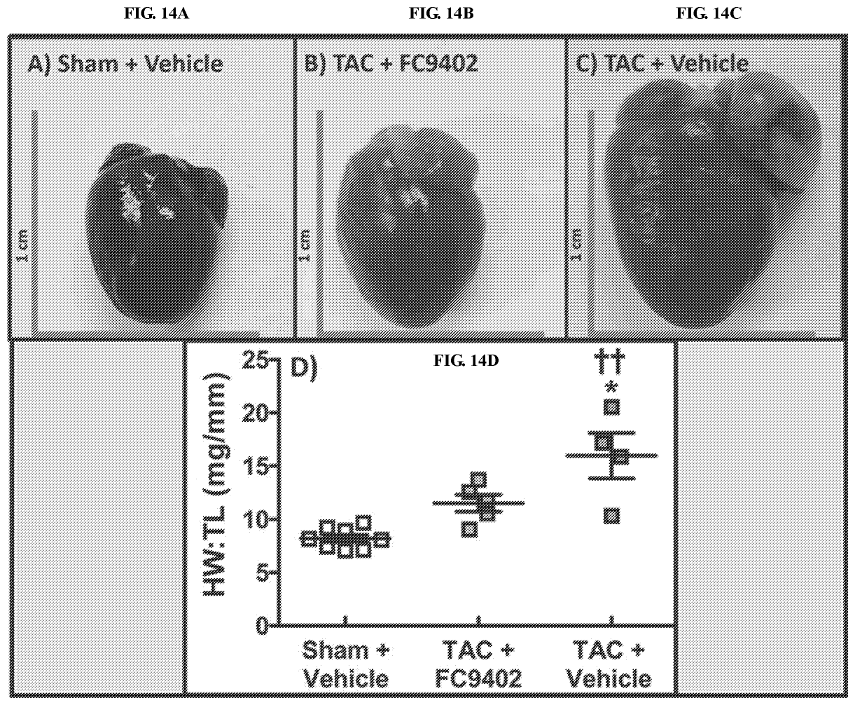

Selected results are illustrated herein. The proof-of-concept 12-week study with FC9402 in the TAC mouse model of heart failure was conducted using three groups of animals: Sham+vehicle; TAC+vehicle; TAC+FC9402. Daily i.p. injections of vehicle or FC9402 (10 mg/kg) were started on day 1 after surgery (FIG. 12). FC9402 was well tolerated by the mice, an outcome that underscores the ability of the inhibitor screening protocol to identify compounds that are highly selective for the CoQ site in SQOR and are not toxic to mammalian cells. Indeed, animals that survived TAC surgery and received daily i.p. injections of FC9402 exhibited 100% survival after 12 weeks, as observed for sham-operated mice. In contrast, only 66% survival was observed for animals receiving vehicle after TAC surgery (FIG. 13). FC9402 prevented the TAC-induced cardiac enlargement observed with animals receiving vehicle after TAC surgery, as judged by the gross morphology of hearts harvested at the termination of the study (FIGS. 14A-14C) and the observed ratios of heart weight to tibia length (FIG. 14D).

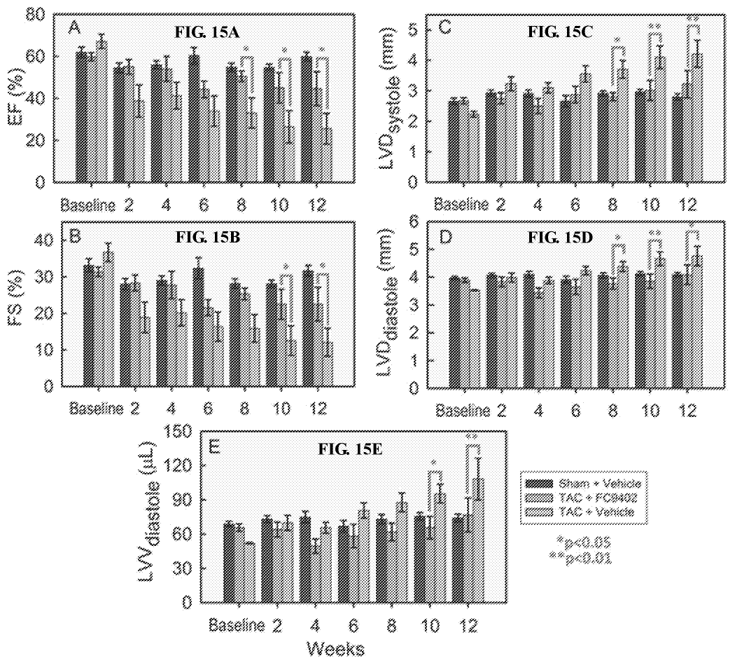

Parasternal long axis echocardiography images were acquired prior to surgery and at two-week intervals post surgery to monitor TAC-induced changes in left ventricle function and structure (FIGS. 15A-15E). TAC animals receiving vehicle exhibited a progressive deterioration of left ventricle function, as judged by observed decreases in ejection fraction (FIG. 15A), the percentage of blood ejected from the ventricle during systole in relation to the total end-diastolic volume, and observed decreases in fractional shortening (FIG. 15B), the degree of shortening of the left ventricular diameter between end-diastole and end-systole. TAC animals that received vehicle also exhibited progressive hypertrophy of the left ventricle, as judged by observed increases in left ventricular end-systolic diameter (FIG. 15C), increases in left ventricular end-diastolic diameter (FIG. 15D), increases in ventricular end-diastolic volume (FIG. 15E), and increases in left ventricular mass (2.6-fold). In dramatic contrast, FC9402 (i) preserved left ventricle function by mitigating TAC-induced decreases in ejection fraction and fractional shortening and (ii) maintained left ventricle size by mitigating TAC-induced increases in left ventricle diameter, left ventricle volume, and left ventricle mass (FIGS. 15A-15E).

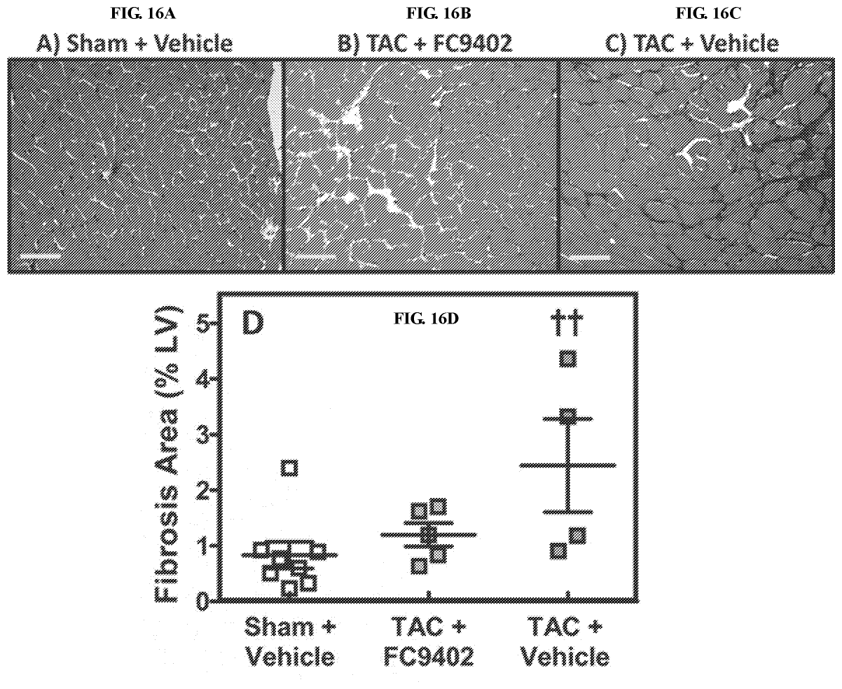

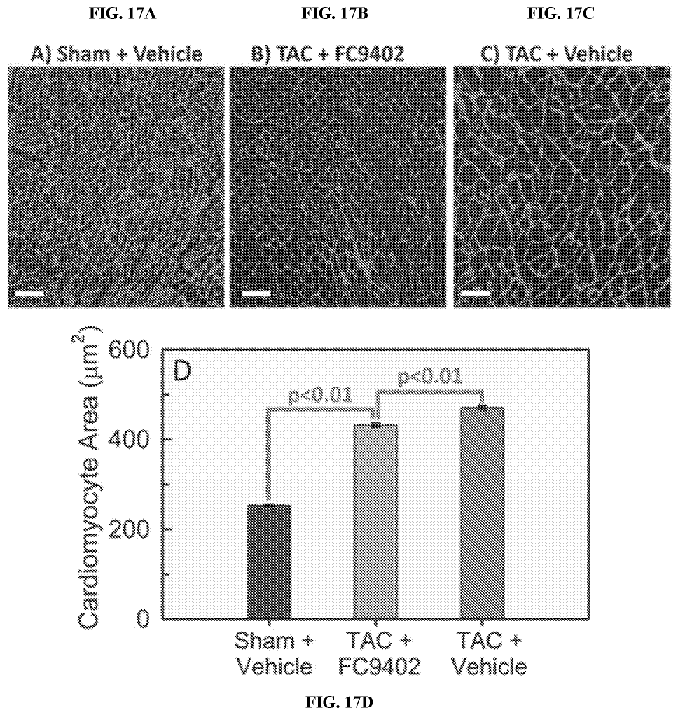

To investigate the extent of left ventricle fibrosis at 12 weeks after TAC, heart sections were stained with Masson's Trichrome, which revealed significant deposition of interstitial collagen (blue) in vehicle-treated TAC mice (FIG. 16C) as compared with Sham-operated (FIG. 16A) or FC9402-treated TAC mice (FIG. 16B). Quantitative analysis using Image J showed that FC9402 mitigated a TAC-induced 3-fold increase in interstitial fibrosis observed in animals receiving vehicle after TAC surgery (FIG. 16D). To evaluate hypertrophy of cardiomycytes in the left ventricle 12 weeks after TAC, myocyte cross-sectional area was determined by analysis of heart sections stained with wheat germ agglutinin (WGA). The results show that FC9402 attenuated a TAC-induced 2-fold increase in cardiomyocyte cross-sectional area observed in animals receiving vehicle after TAC surgery (FIGS. 17A-17D).

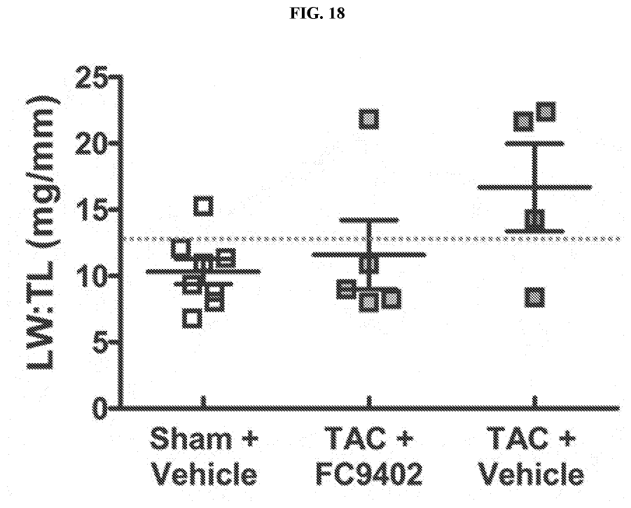

Pulmonary edema is the most severe cardinal manifestation of heart failure. TAC mice with lung weight/tibia length ratios above the 95% confidence interval observed for sham mice were regarded as having a heart failure phenotype, similar to that previously defined (Mohammed et al., 2012, Cardiovasc. Pathol. 27: 188-198). Vehicle-treated TAC mice exhibited lung weight/tibia length ratios that were on average 1.6-fold higher than sham mice and 75% had developed heart failure. In contrast, FC9402-treated TAC mice exhibited ratios that were on average only 1.1-fold higher than sham animals and just 20% developed heart failure (FIG. 18).

The results indicate biological efficacy of FC9402 in mitigating cardiac hypertrophy and progressive deterioration of left ventricle function in the TAC mouse model of pressure-induced heart failure. In certain embodiments, FC9402 exhibits similar efficacy in the treatment of ischemia/reperfusion-induced heart failure, another pathological condition associated with impaired H₂S homeostasis and heart failure with reduced ejection fraction (Wang et al., 2011, Biosci. Rep. 31: 87-98).

### Example 6: Synthesis

### General Procedure A:

### 2-(4-Fluoro-benzylidene)-malononitrile

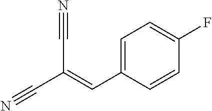 [SMILES: N#CC(C#N)=Cc1ccc(F)cc1]

To a solution of 4-fluorobenzaldehyde (2.00 g, 1.70 mL, 16.00 mmol), and malononitrile (1.06 g, 16.00 mmol) in ethanol (16 mL) was added a 10% aqueous potassium hydroxide solution (0.32 mL). The reaction was stirred for 15 minutes, then was allowed to stand for 30 minutes, followed by cooling to 0° C. The mixture was filtered, then the solid was washed with ethanol (2×5 mL), and hexane (5 mL), to give 2-(4-fluoro-benzylidene)-malononitrile (2.37 g, 86%) as a pale pink solid. ¹H NMR (300 MHz, DMSO-d₆) δ 8.52 (s, 1H), 7.90-8.11 (m, 2H), 7.47 (t, J=8.79 Hz, 2H).

### General Procedure B:

### 2-(2-Methoxy-benzylidene)-indan-1-one

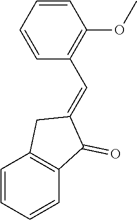 [SMILES: COc1ccccc1/C=C1\Cc2ccccc2C1=O]

A mixture of indan-1-one (0.5 g, 3.78 mmol), and 2-methoxybenzaldehyde (0.54 g, 0.48 mL, 3.97 mmol) in ethanol (6 mL) was treated with a 10N sodium hydroxide solution (0.47 mL, 4.73 mmol). The reaction was stirred for 1.5 hours, then was cooled to 0° C. The mixture was filtered, then the solid was washed with cold ethanol (2×5 mL), and hexane (2×5 mL), to give 2-(2-methoxy-benzylidene)-indan-1-one (842 mg, 89% as a pale yellow solid. MS: [M+H]⁺ 251. ¹H NMR (300 MHz, DMSO-d₆) δ 7.88 (s, 1H), 7.73-7.81 (m, 2H), 7.59-7.72 (m, 2H), 7.36-7.50 (m, 2H), 7.00-7.15 (m, 2H), 4.06 (s, 2H), 3.86 (s, 3H).

### General Procedure C:

### 2-Ethoxy-4-(2-methoxyphenyl)-5H-indeno[1,2-b]pyridine-3-carbonitrile

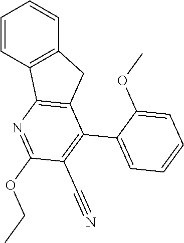 [SMILES: CCOc1nc2c(c(-c3ccccc3OC)c1C#N)Cc1ccccc1-2]

A mixture of 2-(2-methoxy-benzylidene)-indan-1-one (50 mg, 0.20 mmol), and malononitrile (13 mg, 0.20 mmol) in ethanol (0.6 mL) was treated with crushed sodium hydroxide (40 mg, 1.00 mmol). The reaction was stirred for 16 hours, then was filtered. The solid was washed with cold ethanol (2×5 mL), and hexane (5 mL). The crude material was purified by preparative HPLC, to give 2-ethoxy-4-(2-methoxyphenyl)-5H-indeno[1,2-b]pyridine-3-carbonitrile (5 mg) as a white solid. MS: [M+H]⁺ 343. ¹H NMR (300 MHz, CD₃OD) δ 7.95-8.04 (m, 1H), 7.42-7.60 (m, 4H), 7.34 (d, J=7.03 Hz, 1H), 7.20 (d, J=8.20 Hz, 1H), 7.07-7.16 (m, 1H), 4.57-4.76 (m, 2H), 3.83 (d, J=1.17 Hz, 3H), 3.64-3.75 (m, 1H), 3.50-3.63 (m, 1H), 1.51 (dt, J=1.17, 7.03 Hz, 3H).

### 4-(4-Fluorophenyl)-2-ethoxy-5H-indeno[1,2-b]pyridine-3-carbonitrile

### (i) (E)-2-(4-Fluorobenzylidene)-2,3-dihydroinden-1-one

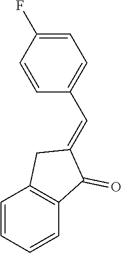 [SMILES: O=C1/C(=C/c2ccc(F)cc2)Cc2ccccc21]

General Procedure B was followed, using indan-1-one (2.00 g, 15.15 mmol), and 4-fluorobenzaldehyde (1.99 g, 1.70 mL, 15.88 mmol), to give (E)-2-(4-fluorobenzylidene)-2,3-dihydroinden-1-one (3.43 g, 95%) as a white solid. MS: [M+H]⁺ 239. ¹H NMR (300 MHz, DMSO-d₆) δ 7.84 (dd, J=5.57, 8.49 Hz, 2H), 7.77 (d, J=7.62 Hz, 1H), 7.61-7.74 (m, 2H), 7.53 (s, 1H), 7.41-7.50 (m, 1H), 7.33 (t, J=8.79 Hz, 2H), 4.09 (s, 2H).

### (ii) 4-(4-Fluorophenyl)-2-ethoxy-5H-indeno[1,2-b]pyridine-3-carbonitrile

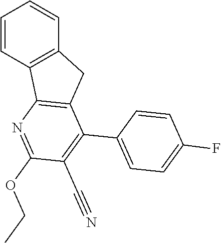 [SMILES: CCOc1nc2c(c(-c3ccc(F)cc3)c1C#N)Cc1ccccc1-2]

General Procedure C was followed, using (E)-2-(4-fluorobenzylidene)-2,3-dihydroinden-1-one (48 mg, 0.20 mmol), and malononitrile (13 mg, 0.20 mmol), to give 4-(4-fluorophenyl)-2-ethoxy-5H-indeno[1,2-b]pyridine-3-carbonitrile (3 mg, 1%) as a white solid. MS: [M+H]⁺331. ¹H NMR (300 MHz, CD₃OD) δ 7.93-8.10 (m, 1H), 7.56-7.74 (m, 4H), 7.47 (dd, J=3.22, 5.57 Hz, 2H), 7.23-7.41 (m, 2H), 4.57-4.77 (m, 2H), 3.78 (s, 2H), 1.40-1.58 (m, 3H).

### 9-Chloro-2-ethoxy-4-(4-fluorophenyl)-5H-indeno[1,2-b]pyridine-3-carbonitrile

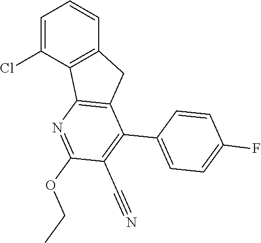 [SMILES: CCOc1nc2c(c(-c3ccc(F)cc3)c1C#N)Cc1cccc(Cl)c1-2]

A mixture of 2-(4-fluoro-benzylidene)-malononitrile (0.22 g, 1.25 mmol) and 7-chloroindan-1-one (0.21 g, 1.25 mmol) was treated with a solution of potassium hydroxide (0.25 g, 4.97 mmol) in ethanol (6 mL). The reaction was stirred for 3 days. The mixture was filtered, then the solid was washed with cold ethanol (2×5 mL), and hexane (5 mL). The solid was recrystallized from n-butanol, to give 9-chloro-2-ethoxy-4-(4-fluorophenyl)-5H-indeno[1,2-b]pyridine-3-carbonitrile (96 mg, 21%) as an off-white solid. MS: [M+H]⁺ 365; ¹H NMR (300 MHz, DMSO-d₆) δ 7.76 (dd, J=5.57, 8.49 Hz, 2H), 7.58-7.64 (m, 1H), 7.41-7.56 (m, 4H), 4.64 (q, J=6.64 Hz, 2H), 3.88 (s, 2H), 1.35-1.57 (m, 3H).

### 4-(4-Fluorophenyl)-2-methoxybenzofuro[3,2-b]pyridine-3-carbonitrile

### (i) 2-(4-Chlorobenzylidene)malononitrile

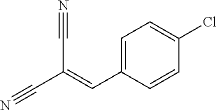 [SMILES: N#CC(C#N)=Cc1ccc(Cl)cc1]

General Procedure A was followed, using malononitrile (1.06 g, 16.00 mmol) and 4-chlorobenzaldehyde (2.27 g 16.00 mmol), to give 2-(4-chlorobenzylidene)malononitrile (2.69 g, 89%) as an off-white solid. MS: [M+H]⁺ 189; ¹H NMR (300 MHz, CDCl₃) δ 7.83-7.94 (m, 2H), 7.72-7.80 (m, 1H), 7.48-7.61 (m, 2H).

### (ii) 4-(4-Fluorophenyl)-2-methoxybenzofuro[3,2-b]pyridine-3-carbonitrile

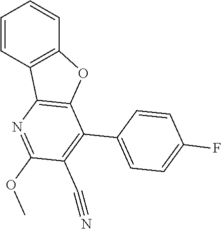 [SMILES: COc1nc2c(oc3ccccc32)c(-c2ccc(F)cc2)c1C#N]

A mixture of 2-(4-fluoro-benzylidene)-malononitrile (0.22 g, 1.25 mmol) and benzofuran-3(2H)-one (0.17 g, 1.25 mmol) was treated with a 4% solution of potassium hydroxide in ethanol (6 mL). The reaction was stirred for 16 hours. The mixture was filtered, then the solid was washed with cold ethanol (2×5 mL) and pentane (5 mL). The crude material was purified by column on silica (0-20% ethyl acetate:hexane), to give 4-(4-fluorophenyl)-2-methoxybenzofuro[3,2-b]pyridine-3-carbonitrile (16 mg, 4%) as a white solid. MS: [M+H]⁺ 333; ¹H NMR (300 MHz, DMSO-d₆) δ 8.14-8.21 (m, 1H), 7.87-7.96 (m, 2H), 7.78-7.86 (m, 1H), 7.68-7.76 (m, 1H), 7.47-7.58 (m, 3H), 4.63 (q, J=7.03 Hz, 2H), 1.46 (t, J=7.03 Hz, 3H).

### General Procedure D:

### 4-(4-Fluorophenyl)-2-methoxy-6-(pyridin-4-yl)pyridine-3-carbonitrile

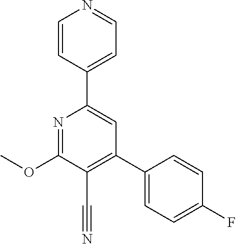 [SMILES: COc1nc(-c2ccncc2)cc(-c2ccc(F)cc2)c1C#N]

A mixture of 2-(4-fluoro-benzylidene)-malononitrile (0.22 g, 1.25 mmol) and 1-(pyridin-4-yl)ethanone (151 mg, 1.25 mmol) in methanol (4 mL) was treated with crushed sodium hydroxide (100 mg, 2.50 mmol). The reaction was stirred for 16 hours. The mixture was filtered, then the solid was washed with cold methanol (2×5 mL) and pentane (5 mL). The crude material was purified by column on silica (0-7% methanol:dichloromethane). The product was dissolved in CH₂Cl₂ (5 mL) and methanol (1 mL), then was treated with 4N HCl in 1,4-dioxane (0.25 mL). The mixture was allowed to stand for 5 minutes, then was concentrated to give 4-(4-fluorophenyl)-2-methoxy-6-(pyridin-4-yl)pyridine-3-carbonitrile as the HCl salt (45 mg, 11%) as a yellow solid. MS: [M+H]⁺ 306; ¹H NMR (300 MHz, DMSO-d₆) δ 8.84-9.25 (m, 2H), 8.72 (d, J=4.10 Hz, 2H), 8.25 (s, 1H), 7.72-8.00 (m, 2H), 7.34-7.62 (m, 2H), 4.15-4.27 (m, 3H).

### 4-(4-Fluorophenyl)-2-methoxy-6-(pyridin-3-yl)pyridine-3-carbonitrile

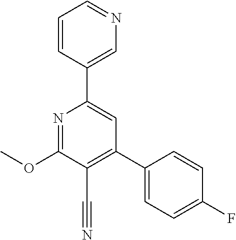 [SMILES: COc1nc(-c2cccnc2)cc(-c2ccc(F)cc2)c1C#N]

General Procedure D was followed, using 2-(4-fluoro-benzylidene)-malononitrile (0.22 g, 1.25 mmol) and 1-(pyridin-3-yl)ethanone (151 mg, 1.25 mmol), to give 4-(4-fluorophenyl)-2-methoxy-6-(pyridin-3-yl)pyridine-3-carbonitrile as the HCl salt (76 mg, 19%) as a pale yellow solid. MS: [M+H]⁺ 306; ¹H NMR (300 MHz, DMSO-d₆) δ 9.63 (s, 1H), 9.07 (d, J=8.20 Hz, 1H), 8.91 (d, J=4.69 Hz, 1H), 8.11 (s, 1H), 7.75-8.05 (m, 3H), 7.48 (t, J=9.08 Hz, 2H), 4.11-4.23 (m, 3H).

### General Procedure E:

### 6-(3-Chlorophenyl)-4-(4-fluorophenyl)-2-methoxypyridine-3-carbonitrile

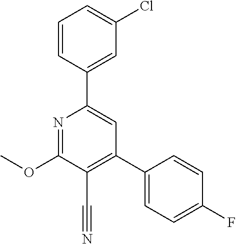 [SMILES: COc1nc(-c2cccc(Cl)c2)cc(-c2ccc(F)cc2)c1C#N]

A mixture of 2-(4-fluoro-benzylidene)-malononitrile (0.22 g, 1.25 mmol) and 1-(3-chlorophenyl)ethanone (0.19 g, 1.25 mmol) in methanol (4 mL) was treated with crushed sodium hydroxide (0.10 g, 2.50 mmol). The reaction was stirred for 16 hours. The mixture was filtered, then the solid was washed with cold methanol (2×5 mL) and pentane (5 mL). The solid was recrystallized from ethanol, to give 6-(3-chlorophenyl)-4-(4-fluorophenyl)-2-methoxypyridine-3-carbonitrile (74 mg, 18%) as a white solid. MS: [M+H]⁺ 339; ¹H NMR (300 MHz, CDCl₃) δ 8.10 (br. s., 1H), 7.97 (d, J=6.44 Hz, 1H), 7.66 (dd, J=5.57, 7.32 Hz, 2H), 7.46 (d, J=8.20 Hz, 3H), 7.16-7.35 (m, 3H), 4.22 (s, 3H).

### 6-(2-Chlorophenyl)-4-(4-fluorophenyl)-2-methoxypyridine-3-carbonitrile

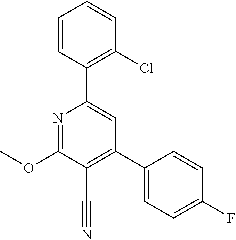 [SMILES: COc1nc(-c2ccccc2Cl)cc(-c2ccc(F)cc2)c1C#N]

General Procedure E was followed, using 2-(4-fluoro-benzylidene)-malononitrile (0.22 g, 1.25 mmol) and 1-(2-chlorophenyl)ethanone (0.19 g, 1.25 mmol), to give 6-(2-chlorophenyl)-4-(4-fluorophenyl)-2-methoxypyridine-3-carbonitrile (47 mg, 11%) as a white solid. MS: [M+H]⁺ 339; ¹H NMR (300 MHz, CDCl₃) δ 7.63-7.74 (m, 3H), 7.49-7.58 (m, 1H), 7.37-7.47 (m, 3H), 7.18-7.31 (m, 3H), 4.16 (s, 3H).

### 2-Methoxy-6-phenyl-4-(pyridin-4-yl)pyridine-3-carbonitrile

### (i) 2-((Pyridin-4-yl)methylene)malononitrile

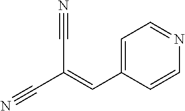 [SMILES: N#CC(C#N)=Cc1ccncc1]

A mixture of isonicotinaldehyde (0.96 g, 8.00 mmol) and malononitrile (0.53 g, 8.00 mmol) in water (4 mL) was treated with DABCO (0.90 g, 8.00 mmol). The reaction was stirred for 30 minutes, then was extracted with ethyl acetate (2×30 mL). The combined organic extracts was washed with brine (3 mL), dried (Na₂SO₄), and concentrated. The crude material was purified by column on silica (0-5% methanol:dichloromethane), to give 2-((pyridin-4-yl)methylene)malononitrile (0.42 g, 31%) as a purple gum. ¹H NMR (300 MHz, DMSO-d₆) δ 8.86 (d, J=5.86 Hz, 2H), 8.63 (s, 1H), 7.76 (d, J=5.86 Hz, 2H).

### (ii) 2-Methoxy-6-phenyl-4-(pyridin-4-yl)pyridine-3-carbonitrile

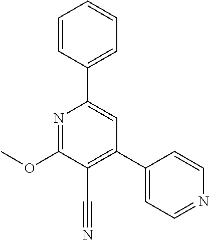 [SMILES: COc1nc(-c2ccccc2)cc(-c2ccncc2)c1C#N]

General Procedure D was followed, using 2-((pyridin-4-yl)methylene)malononitrile (0.22 g, 1.25 mmol) and acetophenone (150 mg, 1.25 mmol), to give 2-methoxy-6-phenyl-4-(pyridin-4-yl)pyridine-3-carbonitrile as the HCl salt (25 mg, 6%) as a white solid. MS: [M+H]⁺ 288. ¹H NMR (300 MHz, DMSO-d₆) δ 8.75-9.05 (m, 2H), 8.29 (dd, J=2.34, 6.44 Hz, 2H), 7.95 (s, 3H), 7.49-7.68 (m, 3H), 4.10-4.25 (m, 3H).

### 4-(4-Fluorophenyl)-2-methoxy-6-(pyridin-2-yl)pyridine-3-carbonitrile

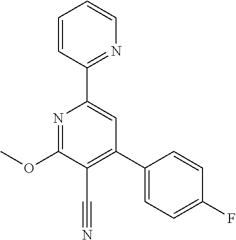 [SMILES: COc1nc(-c2ccccn2)cc(-c2ccc(F)cc2)c1C#N]

General Procedure D was followed, using 2-(4-fluoro-benzylidene)-malononitrile (0.22 g, 1.25 mmol) and 1-(pyridin-2-yl)ethanone (151 mg, 1.225 mmol), to give 4-(4-fluorophenyl)-2-methoxy-6-(pyridin-2-yl)pyridine-3-carbonitrile as the HCl salt (56 mg, 13%) as a white solid. MS: [M+H]⁺ 306. ¹H NMR (300 MHz, DMSO-d₆) δ 8.71-8.80 (m, 1H), 8.50 (d, J=8.20 Hz, 1H), 8.12-8.18 (m, 1H), 8.01-8.11 (m, 1H), 7.76-7.86 (m, 2H), 7.58 (dd, J=5.27, 7.03 Hz, 1H), 7.38-7.51 (m, 2H), 4.18 (s, 3H).

### 4-(4-Chlorophenyl)-2-methoxy-6-phenylpyridine-3-carbonitrile

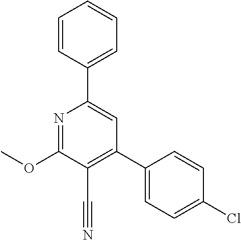 [SMILES: COc1nc(-c2ccccc2)cc(-c2ccc(Cl)cc2)c1C#N]

General Procedure E was followed, using 2-(4-chlorobenzylidene)malononitrile (0.24 g, 1.25 mmol) and acetophenone (150 mg, 1.25 mmol), to give 4-(4-chlorophenyl)-2-methoxy-6-phenylpyridine-3-carbonitrile (62 mg, 16%) as a white solid. MS: [M+H]⁺ 321. ¹H NMR (300 MHz, DMSO-d₆) δ 8.24-8.32 (m, 2H), 7.84 (s, 1H), 7.76-7.83 (m, 2H), 7.64-7.71 (m, 2H), 7.51-7.58 (m, 3H), 4.15 (s, 3H).

### 4-(4-Fluorophenyl)-2-methoxy-6-(4-methoxyphenyl)pyridine-3-carbonitrile

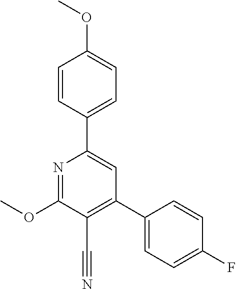 [SMILES: COc1ccc(-c2cc(-c3ccc(F)cc3)c(C#N)c(OC)n2)cc1]

General Procedure E was followed, using 2-(4-fluoro-benzylidene)-malononitrile (0.22 g, 1.25 mmol) and 1-(4-methoxyphenyl)ethanone (0.19 g, 1.25 mmol), to give 4-(4-fluorophenyl)-2-methoxy-6-(4-methoxyphenyl)pyridine-3-carbonitrile (22 mg, 5%) as a white solid. MS: [M+H]⁺ 335. ¹H NMR (300 MHz, DMSO-d₆) δ 8.25 (d, J=8.79 Hz, 2H), 7.70-7.93 (m, 3H), 7.43 (t, J=8.79 Hz, 2H), 7.08 (d, J=8.20 Hz, 2H), 4.13 (s, 3H), 3.84 (s, 3H).

### 4-(4-Fluorophenyl)-2-methoxy-6-(3-methoxyphenyl)pyridine-3-carbonitrile

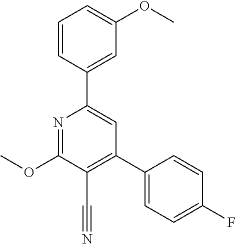 [SMILES: COc1cccc(-c2cc(-c3ccc(F)cc3)c(C#N)c(OC)n2)c1]

General Procedure E was followed, using 2-(4-fluoro-benzylidene)-malononitrile (0.22 g, 1.25 mmol), and 1-(3-methoxyphenyl)ethanone (0.19 g, 1.25 mmol), to give 4-(4-fluorophenyl)-2-methoxy-6-(3-methoxyphenyl)pyridine-3-carbonitrile (44 mg, 11%) as a white solid. MS: [M+H]⁺ 335. ¹H NMR (300 MHz, DMSO-d₆) δ 7.72-8.06 (m, 6H), 7.35-7.63 (m, 3H), 7.00-7.22 (m, 1H), 4.15 (s, 3H), 3.85 (s, 3H).

### General Procedure F:

### 4-(4-Fluorophenyl)-2-methoxy-6-(2-methoxyphenyl)pyridine-3-carbonitrile

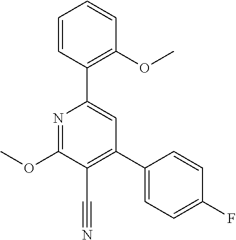 [SMILES: COc1ccccc1-c1cc(-c2ccc(F)cc2)c(C#N)c(OC)n1]

A mixture of 2-(4-fluoro-benzylidene)-malononitrile (0.22 g, 1.25 mmol) and 1-(2-methoxyphenyl)ethanone (0.19 g, 1.25 mmol) in methanol (4 mL) was treated with crushed sodium hydroxide (0.10 g, 2.50 mmol). The reaction was stirred for 16 hours. The mixture was filtered, then the solid was washed with cold methanol (2×5 mL), and pentane (5 mL). The solid was purified by column on silica (0-30% ethyl acetate:hexane), to give 4-(4-fluorophenyl)-2-methoxy-6-(2-methoxyphenyl)pyridine-3-carbonitrile (19 mg, 5%) as a white solid. MS: [M+H]⁺ 335. ¹H NMR (300 MHz, CDCl₃) δ 8.04 (dd, J=1.76, 7.62 Hz, 1H), 7.75 (s, 1H), 7.61-7.70 (m, 2H), 7.40-7.51 (m, 1H), 7.18-7.29 (m, 2H), 7.13 (t, J=7.62 Hz, 1H), 7.04 (d, J=8.79 Hz, 1H), 4.17 (s, 3H), 3.91 (s, 3H).

### 2-(2-Methoxyethoxy)-4-(4-fluorophenyl)-6-phenylpyridine-3-carbonitrile

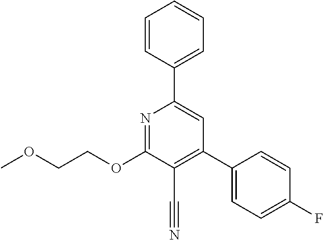 [SMILES: COCCOc1nc(-c2ccccc2)cc(-c2ccc(F)cc2)c1C#N]

A mixture of 2-(4-fluoro-benzylidene)-malononitrile (0.22 g, 1.25 mmol) and acetophenone (0.15 g, 1.25 mmol) in 2-methoxyethanol (3 mL) was treated with crushed sodium hydroxide (0.10 g, 2.50 mmol). The reaction was stirred for 16 hours. The mixture was filtered, then the solid was washed with cold methanol (2×5 mL), and pentane (5 mL). The solid was recrystallized from ethanol, to give 2-(2-methoxyethoxy)-4-(4-fluorophenyl)-6-phenylpyridine-3-carbonitrile (61 mg, 14%) as a white solid. MS: [M+H]⁺ 349. ¹H NMR (300 MHz, DMSO-d₆) δ 8.25 (dd, J=2.34, 6.44 Hz, 2H), 7.79-7.89 (m, 3H), 7.50-7.58 (m, 3H), 7.44 (t, J=8.79 Hz, 2H), 4.64-4.81 (m, 2H), 3.72-3.85 (m, 2H), 3.30 (s, 3H).

### 4-(4-Fluoro-phenyl)-6-furan-3-yl-2-methoxy-nicotinonitrile

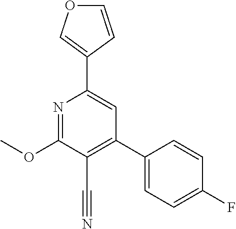 [SMILES: COc1nc(-c2ccoc2)cc(-c2ccc(F)cc2)c1C#N]

General Procedure E was followed, using 2-(4-fluoro-benzylidene)-malononitrile (0.22 g, 1.25 mmol) and 1-furan-3-yl-ethanone (0.14 g, 1.25 mmol), to give 4-(4-fluoro-phenyl)-6-furan-3-yl-2-methoxy-nicotinonitrile (41 mg, 11%) as a white solid. MS: [M+H]⁺ 294. ¹H NMR (300 MHz, DMSO-d₆) δ 8.58 (br. s., 1H), 7.70-7.92 (m, 3H), 7.58-7.69 (m, 1H), 7.38-7.55 (m, 2H), 7.16-7.27 (m, 1H), 4.01-4.20 (m, 3H).

### 4-(4-Fluorophenyl)-5,6-dihydro-2-methoxybenzo[h]quinoline-3-carbonitrile

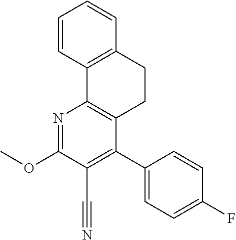 [SMILES: COc1nc2c(c(-c3ccc(F)cc3)c1C#N)CCc1ccccc1-2]

General Procedure E was followed, using 2-(4-fluoro-benzylidene)-malononitrile (0.22 g, 1.25 mmol) and 3,4-dihydronaphthalen-1(2H)-one (0.18 g, 1.25 mmol), to give 4-(4-fluorophenyl)-5,6-dihydro-2-methoxybenzo[h]quinoline-3-carbonitrile (122 mg, 30%) as a pale yellow solid. MS: [M+H]⁺ 331. ¹H NMR (300 MHz, DMSO-d₆) δ 8.23-8.32 (m, 1H), 7.47-7.57 (m, 2H), 7.36-7.47 (m, 4H), 7.32 (d, J=2.93 Hz, 1H), 4.13 (s, 3H), 2.80 (d, J=7.62 Hz, 2H), 2.56-2.65 (m, 2H).

### 2-(2-(Dimethylamino)ethoxy)-4-(4-fluorophenyl)-6-phenylpyridine-3-carbonitrile

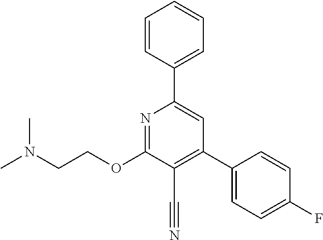 [SMILES: CN(C)CCOc1nc(-c2ccccc2)cc(-c2ccc(F)cc2)c1C#N]

A mixture of 2-(4-fluoro-benzylidene)-malononitrile (0.22 g, 1.25 mmol) and acetophenone (0.15 g, 1.25 mmol) in 2-(dimethylamino)ethanol (3 mL) was treated with crushed sodium hydroxide (0.10 g, 2.50 mmol). The reaction was stirred for 3 days. The mixture was filtered, then the solid was washed with cold methanol (2×5 mL), and pentane (5 mL). The crude material was purified by column on silica (0-7% methanol:dichloromethane). The product was dissolved in CH₂Cl₂ (5 mL) and methanol (1 mL), then was treated with 4N HCl in 1,4-dioxane (0.25 mL). The mixture was allowed to stand for 5 minutes, then was concentrated to give 2-(2-(dimethylamino)ethoxy)-4-(4-fluorophenyl)-6-phenylpyridine-3-carbonitrile as the HCl salt (97 mg, 20%) as a white solid. MS: [M+H]⁺ 362. ¹H NMR (300 MHz, DMSO-d₆) δ 10.53-10.83 (m, 1H), 8.16-8.39 (m, 2H), 7.91 (s, 1H), 7.85 (dd, J=5.57, 7.32 Hz, 2H), 7.56 (dd, J=1.76, 2.93 Hz, 3H), 7.42-7.52 (m, 2H), 4.98 (d, J=3.51 Hz, 2H), 2.91 (br. s., 6H), 2.76 (d, J=3.51 Hz, 2H).

### 6-(2-Fluorophenyl)-4-(4-fluorophenyl)-2-methoxypyridine-3-carbonitrile

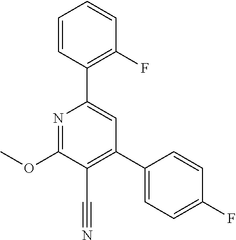 [SMILES: COc1nc(-c2ccccc2F)cc(-c2ccc(F)cc2)c1C#N]

General Procedure E was followed, using 2-(4-fluoro-benzylidene)-malononitrile (0.22 g, 1.25 mmol) and 1-(2-fluorophenyl)ethanone (0.17 g, 1.25 mmol), to give 6-(2-fluorophenyl)-4-(4-fluorophenyl)-2-methoxypyridine-3-carbonitrile (58 mg, 14%) as a white solid. MS: [M+H]⁺ 323. ¹H NMR (300 MHz, DMSO-d₆) δ 8.04-8.29 (m, 1H), 7.75-7.90 (m, 2H), 7.54-7.73 (m, 2H), 7.29-7.53 (m, 4H), 4.06-4.19 (m, 3H).

### 4-(4-Fluoro-phenyl)-6-furan-2-yl-2-methoxy-nicotinonitrile

### (i) 2-(3,4-Difluoro-benzylidene)-malononitrile

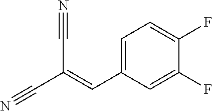 [SMILES: N#CC(C#N)=Cc1ccc(F)c(F)c1]

General Procedure A was followed, using malononitrile (1.06 g, 16.00 mmol) and 3,4-difluorobenzaldehyde (2.27 g 16.00 mmol), to give 2-(3,4-difluoro-benzylidene)-malononitrile (1.63 g, 54%) as a white solid. ¹H NMR (300 MHz, DMSO-d₆) δ 8.52 (s, 1H), 7.97 (ddd, J=2.05, 7.76, 11.28 Hz, 1H), 7.80-7.89 (m, 1H), 7.67-7.79 (m, 1H).

### (ii) 4-(4-Fluoro-phenyl)-6-furan-2-yl-2-methoxy-nicotinonitrile

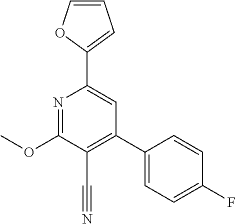 [SMILES: COc1nc(-c2ccco2)cc(-c2ccc(F)cc2)c1C#N]

General Procedure E was followed, using 2-(4-fluoro-benzylidene)-malononitrile (0.22 g, 1.25 mmol) and 1-furan-2-yl-ethanone (0.14 g, 1.25 mmol), to give 4-(4-fluoro-phenyl)-6-furan-2-yl-2-methoxy-nicotinonitrile (43 mg, 12%) as a white solid. MS: [M+H]⁺ 295. ¹H NMR (300 MHz, DMSO-d₆) δ 7.95-8.01 (m, 1H), 7.74-7.84 (m, 2H), 7.50 (s, 1H), 7.39-7.48 (m, 3H), 6.75 (d, J=1.76 Hz, 1H), 4.09 (s, 3H).

### 4-(3,4-Difluoro-phenyl)-2-methoxy-6-phenyl-nicotinonitrile

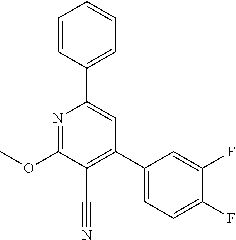 [SMILES: COc1nc(-c2ccccc2)cc(-c2ccc(F)c(F)c2)c1C#N]

General Procedure E was followed, using 2-(3,4-difluoro-benzylidene)-malononitrile (0.22 g, 1.25 mmol) and acetophenone (0.15 g, 1.25 mmol), to give 4-(3,4-difluoro-phenyl)-2-methoxy-6-phenyl-nicotinonitrile (43 mg, 12%) as a white solid. MS: [M+H]⁺ 323. ¹H NMR (300 MHz, DMSO-d₆) δ 8.24-8.32 (m, 2H), 7.94 (dd, J=8.49, 10.25 Hz, 1H), 7.87 (s, 1H), 7.63-7.76 (m, 2H), 7.51-7.58 (m, 3H), 4.15 (s, 3H).

### 2′-Methoxy-6′-phenyl-[3,4′]bipyridinyl-3′-carbonitrile

### (i) 2-Pyridin-3-ylmethylene-malononitrile

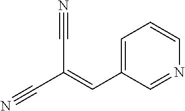 [SMILES: N#CC(C#N)=Cc1cccnc1]

A mixture of pyridine-3-carbaldehyde (1.92 g, 16.00 mmol), and malononitrile (1.06 g, 16.00 mmol) in water (8 mL) was treated with DABCO (1.80 g, 16.00 mmol). The reaction was stirred for 30 minutes, then was extracted with ethyl acetate (2×30 mL). The combined organic extracts was washed with brine (3 mL), dried (Na₂SO₄), and concentrated. The crude material was purified by column on silica (0-5% methanol:dichloromethane), followed by column on silica (0-75% ethyl acetate:hexane) to give 2-pyridin-3-ylmethylene-malononitrile (176 mg, 7%) as a white solid. ¹H NMR (300 MHz, CDCl₃) δ 8.89 (d, J=2.34 Hz, 1H), 8.83 (dd, J=1.46, 4.98 Hz, 1H), 8.48 (dd, J=1.76, 8.20 Hz, 1H), 7.83 (s, 1H), 7.52 (dd, J=4.69, 8.20 Hz, 1H).

### (ii) 2′-Methoxy-6′-phenyl-[3,4′]bipyridinyl-3′-carbonitrile

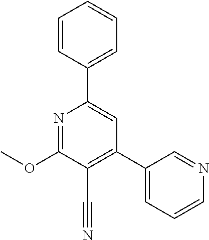 [SMILES: COc1nc(-c2ccccc2)cc(-c2cccnc2)c1C#N]

General Procedure D was followed, using 2-(4-fluoro-benzylidene)-malononitrile (170 mg, 1.02 mmol) and acetophenone (122 mg, 1.02 mmol), to give 2′-methoxy-6′-phenyl-[3,4′]bipyridinyl-3′-carbonitrile as the HCl salt (57 mg, 17%) as a white solid. MS: [M+H]⁺ 288. ¹H NMR (300 MHz, DMSO-d₆) δ 9.03 (s, 1H), 8.84 (d, J=4.69 Hz, 1H), 8.26-8.42 (m, 3H), 8.00 (s, 1H), 7.75 (dd, J=4.98, 7.91 Hz, 1H), 7.49-7.69 (m, 3H), 4.19 (s, 3H).

### 2′-Methoxy-6′-phenyl-[3,4′]bipyridinyl-3′-carbonitrile

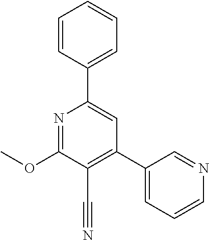 [SMILES: COc1nc(-c2ccccc2)cc(-c2cccnc2)c1C#N]

A mixture of 2′-methoxy-6′-phenyl-[3,4′]bipyridinyl-3′-carbonitrile HCl salt (50 mg, 0.15 mmol) in dilute aqueous sodium bicarbonate solution (30 mL) was extracted with ethyl acetate (2×20 mL). The combined organic extracts was washed with brine (1 mL), dried (Na₂SO₄), and concentrated, to give 2′-methoxy-6′-phenyl-[3,4′]bipyridinyl-3′-carbonitrile (22 mg, 51%) as a white solid. MS: [M+H]⁺ 288. ¹H NMR (300 MHz, DMSO-d₆) δ 9.03 (s, 1H), 8.83 (br. s., 1H), 8.31 (d, J=4.10 Hz, 3H), 7.99 (s, 1H), 7.69-7.82 (m, 1H), 7.56 (br. s., 3H), 4.18 (s, 3H).

### 4-(4-Fluoro-phenyl)-6-phenyl-pyridin-2-ol

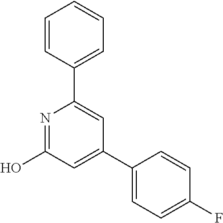 [SMILES: Oc1cc(-c2ccc(F)cc2)cc(-c2ccccc2)n1]

A mixture of 4-fluorochalcone (290 mg, 1.28 mmol), N-acetyl glycine amide (200 mg, 1.72 mmol), and cesium carbonate (550 mg, 1.69 mmol) in DMF (4 mL) was heated at 150° C. for 1.5 hours. The mixture was cooled to 0° C., and 1N HCl (4 mL) was added. The mixture was allowed to warm to 20° C., and was allowed to stand for 16 hours. The mixture was filtered, then the solid was washed with pentane (2×5 mL). The solid was dissolved in ethyl acetate (30 mL), then was washed with water (2×10 mL), and brine (1 mL), dried (Na₂SO₄), and concentrated. The crude material was purified by column on silica (0-20% ethyl acetate:hexane), to give 4-(4-fluoro-phenyl)-6-phenyl-pyridin-2-ol (16 mg, 5%) as an off-white solid. MS: [M+H]⁺ 266. ¹H NMR (300 MHz, CDCl₃) δ 7.82 (d, J=6.44 Hz, 2H), 7.63-7.71 (m, 2H), 7.50-7.62 (m, 3H), 7.29-7.33 (m, 1H), 7.16-7.27 (m, 2H), 6.75 (s, 2H).

### 2-Ethoxy-4-(4-fluoro-phenyl)-6-phenyl-pyridine

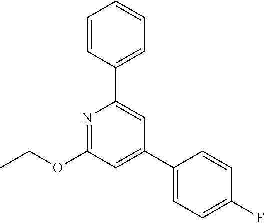 [SMILES: CCOc1cc(-c2ccc(F)cc2)cc(-c2ccccc2)n1]

A mixture of 4-(4-fluoro-phenyl)-6-phenyl-pyridin-2-ol (16 mg, 0.06 mmol), iodoethane (14 mg, 0.09 mmol), and silver (I) carbonate (17 mg, 0.06 mmol) in DMF (0.6 mL) was protected from light, and heated in a sealed vessel at 100° C. for 16 hours. The mixture was treated with water (10 mL), then was extracted with ethyl acetate (20 mL). The organic extract was washed with water (2×10 mL), and brine (1 mL), dried (Na₂SO₄), and concentrated. The crude material was purified by column on silica (0-10% ethyl acetate:hexane), to give 2-ethoxy-4-(4-fluoro-phenyl)-6-phenyl-pyridine (7 mg, 40%) as a clear gum. MS: [M+H]⁺ 294. ¹H NMR (300 MHz, CDCl₃) δ 8.03-8.18 (m, 2H), 7.60-7.75 (m, 2H), 7.38-7.57 (m, 4H), 7.18 (t, J=8.49 Hz, 2H), 6.85 (d, J=1.17 Hz, 1H), 4.56 (q, J=7.03 Hz, 2H), 1.48 (t, J=7.03 Hz, 3H).

### 4-(4-Fluoro-phenyl)-2-methoxy-6-pyrimidin-2-yl-nicotinonitrile

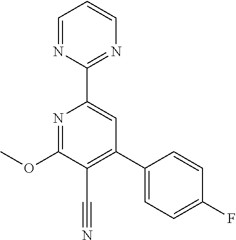 [SMILES: COc1nc(-c2ncccn2)cc(-c2ccc(F)cc2)c1C#N]

A mixture of 2-(4-fluoro-benzylidene)-malononitrile (0.22 g, 1.25 mmol) and 1-pyrimidin-2-yl-ethanone (151 mg, 1.25 mmol) in methanol (4 mL) was treated with crushed sodium hydroxide (100 mg, 2.50 mmol). The reaction was stirred for 5 days. The mixture was filtered, then the solid was washed with cold methanol (2×5 mL) and pentane (5 mL). The solid was recrystallized using ethanol. The filtrate was purified by column on silica (0-100% ethyl acetate:hexane). The product was dissolved in CH₂Cl₂ (5 mL) and methanol (1 mL), then was treated with 4N HCl in 1,4-dioxane (0.25 mL). The mixture was allowed to stand for 5 minutes, then was concentrated to give 4-(4-fluoro-phenyl)-2-methoxy-6-pyrimidin-2-yl-nicotinonitrile as the HCl salt (25 mg, 6%) as a pale yellow solid. MS: [M+H]⁺ 307. ¹H NMR (300 MHz, DMSO-d₆) δ 9.06 (d, J=4.69 Hz, 2H), 8.18 (s, 1H), 7.77-7.93 (m, 2H), 7.59-7.73 (m, 1H), 7.35-7.57 (m, 2H), 4.16 (s, 3H).

### 6-(2-Chloro-6-fluoro-phenyl)-4-(4-fluoro-phenyl)-2-methoxy-nicotinonitrile

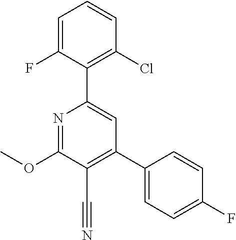 [SMILES: COc1nc(-c2c(F)cccc2Cl)cc(-c2ccc(F)cc2)c1C#N]

General Procedure F was followed, using 2-(4-fluoro-benzylidene)-malononitrile (0.22 g, 1.25 mmol) and 1-(2-chloro-6-fluoro-phenyl)-ethanone (216 mg, 1.25 mmol), to give 6-(2-chloro-6-fluoro-phenyl)-4-(4-fluoro-phenyl)-2-methoxy-nicotinonitrile (75 mg, 17%) as a white solid. MS: [M+H]⁺ 357. ¹H NMR (300 MHz, CDCl₃) δ 7.62-7.72 (m, 2H), 7.30-7.45 (m, 2H), 7.09-7.29 (m, 4H), 4.12 (s, 3H).

### 4-(4-Fluoro-phenyl)-6-methoxy-3′-methyl-[2,2′]bipyridinyl-5-carbonitrile

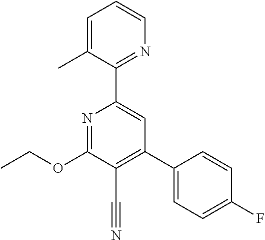 [SMILES: CCOc1nc(-c2ncccc2C)cc(-c2ccc(F)cc2)c1C#N]

General Procedure D was followed, using 2-(4-fluoro-benzylidene)-malononitrile (220 mg, 1.25 mmol) and 1-(3-methyl-pyridin-2-yl)-ethanone (169 mg, 1.25 mmol), to give 4-(4-fluoro-phenyl)-6-methoxy-3′-methyl-[2,2′]bipyridinyl-5-carbonitrile as the HCl salt (17 mg, 4%) as a white solid. MS: [M+H]⁺ 320. ¹H NMR (300 MHz, DMSO-d₆) δ 8.60 (d, J=4.10 Hz, 1H), 7.87-7.99 (m, 2H), 7.76-7.87 (m, 3H), 7.38-7.58 (m, 3H), 4.11 (s, 3H), 2.67 (s, 3H).

### 4-(4-Fluoro-phenyl)-6-methoxy-4′-methyl-[2,2′]bipyridinyl-5-carbonitrile

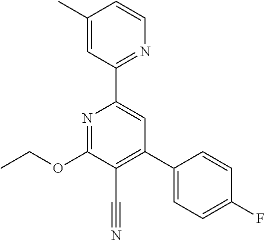 [SMILES: CCOc1nc(-c2cc(C)ccn2)cc(-c2ccc(F)cc2)c1C#N]

General Procedure D was followed, using 2-(4-fluoro-benzylidene)-malononitrile (220 mg, 1.25 mmol), and 1-(4-methyl-pyridin-2-yl)-ethanone (169 mg, 1.25 mmol), to give 4-(4-fluoro-phenyl)-6-methoxy-4′-methyl-[2,2′]bipyridinyl-5-carbonitrile as the HCl salt (25 mg, 7%) as a white solid. MS: [M+H]⁺ 320. ¹H NMR (300 MHz, DMSO-d₆) δ 8.61 (d, J=5.27 Hz, 1H), 8.35 (s, 1H), 8.15 (s, 1H), 7.74-7.86 (m, 2H), 7.38-7.51 (m, 3H), 4.20 (s, 3H), 2.47 (br. s., 3H).

### 2-Methoxy-6-phenyl-4-p-tolyl-nicotinonitrile

### (i) 4-(4-Fluoro-phenyl)-6-methoxy-4′-methyl-[2,2′]bipyridinyl-5-carbonitrile

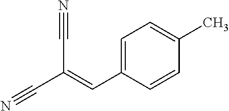

General Procedure A was followed, using malononitrile (0.53 g, 8.00 mmol), and 4-methyl-benzaldehyde (0.96 g 8.00 mmol), to give 2-(4-methyl-benzylidene)-malononitrile (1.21 g, 93%) as a white solid. MS: [M+H]⁺ 164; ¹H NMR (300 MHz, CDCl₃) δ 7.82 (d, J=8.20 Hz, 2H), 7.73 (s, 1H), 7.35 (d, J=8.20 Hz, 2H), 2.47 (s, 3H).

### (ii) 2-Methoxy-6-phenyl-4-p-tolylpyridine-3-carbonitrile

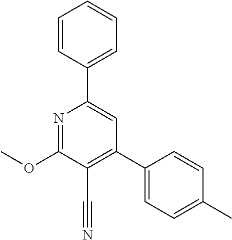

General Procedure E was followed, using 2-(4-methyl-benzylidene)-malononitrile (0.20 g, 1.25 mmol) and acetophenone (0.15 g, 1.25 mmol), to give 2-methoxy-6-phenyl-4-p-tolylpyridine-3-carbonitrile (17 mg, 5%) as a white solid. MS: [M+H]⁺ 301. ¹H NMR (300 MHz, DMSO-d₆) δ 8.22-8.32 (m, 2H), 7.80 (s, 1H), 7.66 (d, J=8.20 Hz, 2H), 7.50-7.59 (m, 3H), 7.40 (d, J=8.20 Hz, 2H), 4.15 (s, 3H), 2.41 (s, 3H).

### 4-(4-Fluoro-phenyl)-2-methoxy-5-methyl-6-phenyl-nicotinonitrile

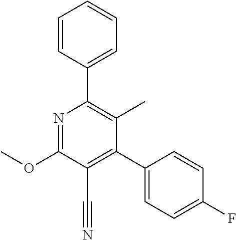 [SMILES: COc1nc(-c2ccccc2)c(C)c(-c2ccc(F)cc2)c1C#N]

General Procedure E was followed, using 2-(3,4-difluoro-benzylidene)-malononitrile (0.22 g, 1.25 mmol), and 1-phenyl-propan-1-one (0.17 g, 1.25 mmol), to give 4-(4-fluoro-phenyl)-2-methoxy-5-methyl-6-phenyl-nicotinonitrile (123 mg, 31%) as a white solid. MS: [M+H]⁺319; ¹H NMR (300 MHz, DMSO-d₆) δ 7.66 (d, J=6.44 Hz, 2H), 7.49-7.61 (m, 5H), 7.37-7.48 (m, 2H), 4.02 (s, 3H), 2.00 (s, 3H).

### 5′-Fluoro-4-(4-fluoro-phenyl)-6-methoxy-[2,2′]bipyridinyl-5-carbonitrile

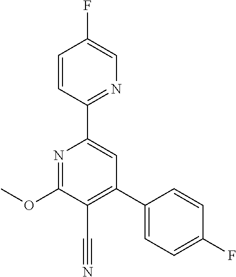 [SMILES: COc1nc(-c2ccc(F)cn2)cc(-c2ccc(F)cc2)c1C#N]

General Procedure D was followed, using 2-(4-fluoro-benzylidene)-malononitrile (110 mg, 0.63 mmol) and 1-(5-fluoro-pyridin-2-yl)-ethanone (89 mg, 0.64 mmol), to give 5′-fluoro-4-(4-fluoro-phenyl)-6-methoxy-[2,2′]bipyridinyl-5-carbonitrile as the HCl salt (17 mg, 4%) as a white solid. MS: [M+H]⁺ 324; ¹H NMR (300 MHz, DMSO-d₆) δ 8.74-8.79 (m, 1H), 8.53-8.63 (m, 1H), 8.08 (d, J=1.17 Hz, 1H), 7.93-8.05 (m, 1H), 7.77-7.88 (m, 2H), 7.40-7.52 (m, 2H), 4.12-4.27 (m, 3H).

### 2′-Methoxy-6′-phenyl-[2,4′]bipyridinyl-3′-carbonitrile

### (i) 1-Phenyl-3-pyridin-2-yl-propenone

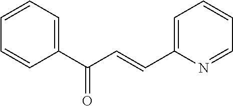 [SMILES: O=C(/C=C/c1ccccn1)c1ccccc1]

To a 0° C. mixture of pyridine-2-carbaldehyde (1.56 g, 13.00 mmol) in 10% aqueous potassium hydroxide (5 mL) and methanol (2.5 mL) was slowly added acetophenone (0.84 g, 7.00 mmol). The reaction was allowed to slowly warm up to 20° C., whilst stirring for 2 hours. The mixture was diluted with water (20 mL), then was extracted with diethyl ether (50 mL). The organic extract was washed with brine (1 mL), dried (Na₂SO₄), and concentrated. The crude material was purified by column on silica (0-25% ethyl acetate:hexane), to give 1-phenyl-3-pyridin-2-yl-propenone (1.05 g, 72%) as a pale yellow solid. MS: [M+H]⁺ 210. ¹H NMR (300 MHz, CDCl₃) δ 8.70 (d, J=4.69 Hz, 1H), 8.02-8.27 (m, 3H), 7.82 (s, 1H), 7.72-7.79 (m, 1H), 7.56-7.65 (m, 1H), 7.46-7.56 (m, 3H), 7.28-7.35 (m, 1H).

### (ii) 2′-Methoxy-6′-phenyl-[2,4′]bipyridinyl-3′-carbonitrile

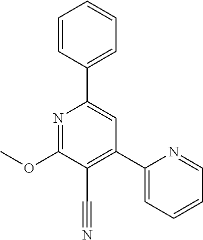 [SMILES: COc1nc(-c2ccccc2)cc(-c2ccccn2)c1C#N]

A mixture of 1-phenyl-3-pyridin-2-yl-propenone (200 mg, 0.96 mmol) and malononitrile (63 mg, 0.96 mmol) in methanol (3 mL) was treated with crushed sodium hydroxide (192 mg, 4.80 mmol). The reaction was stirred for 4 days. The mixture was filtered, then the filtrate was concentrated. The residue was partitioned between ethyl acetate (25 mL) and water (10 mL). The organic extract was washed with brine (1 mL), dried (Na₂SO₄), and concentrated. The crude material was purified by column on silica (0-25% ethyl acetate:hexane). The product was dissolved in CH₂Cl₂ (3 mL) and methanol (1 mL), then was treated with 4N HCl in 1,4-dioxane (0.25 mL). The mixture was allowed to stand for 5 minutes, then was concentrated to give 2′-methoxy-6′-phenyl-[2,4′]bipyridinyl-3′-carbonitrile as the HCl salt (27 mg, 7%) as a pale yellow solid. MS: [M+H]⁺ 288; ¹H NMR (300 MHz, DMSO-d₆) δ 8.66-8.88 (m, 1H), 8.12-8.20 (m, 2H), 7.85-7.93 (m, 2H), 7.80-7.85 (m, 1H), 7.44-7.59 (m, 5H), 4.04 (s, 3H).

### 6-(3-Chloropyridin-2-yl)-4-(4-fluorophenyl)-2-methoxypyridine-3-carbonitrile

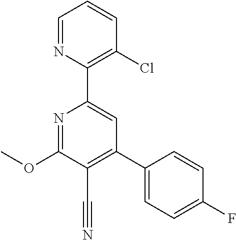 [SMILES: COc1nc(-c2ncccc2Cl)cc(-c2ccc(F)cc2)c1C#N]

General Procedure G was followed, using 2-(4-fluoro-benzylidene)-malononitrile (220 mg, 1.25 mmol) and 1-(3-chloro-pyridin-2-yl)-ethanone (195 mg, 1.25 mmol) to give 3′-chloro-4-(4-fluoro-phenyl)-6-methoxy-[2,2′]bipyridinyl-5-carbonitrile as the HCl salt (48 mg, 10%) as a white solid. MS: M+H⁺ 340. ¹H NMR (300 MHz, DMSO-d₆) δ 8.69 (dd, J=1.17, 4.69 Hz, 1H), 8.14 (dd, J=1.17, 8.20 Hz, 1H), 7.74-7.89 (m, 2H), 7.69 (s, 1H), 7.59 (dd, J=4.69, 8.20 Hz, 1H), 7.44 (t, J=8.79 Hz, 2H), 4.09 (s, 3H).

### 4-(4-Fluorophenyl)-6-(3-fluoropyridin-2-yl)-2-methoxypyridine-3-carbonitrile

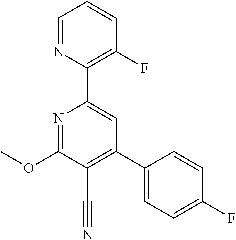 [SMILES: COc1nc(-c2ncccc2F)cc(-c2ccc(F)cc2)c1C#N]

General Procedure G was followed, using 2-(4-fluoro-benzylidene)-malononitrile (220 mg, 1.25 mmol) and 1-(3-fluoro-pyridin-2-yl)-ethanone (174 mg, 1.25 mmol), to give 4-(4-fluorophenyl)-6-(3-fluoropyridin-2-yl)-2-methoxypyridine-3-carbonitrile as the HCl salt (41 mg, 19%) as an off-white solid. MS: M+H⁺ 324. ¹H NMR (300 MHz, DMSO-d₆) δ 8.51-8.72 (m, 1H), 7.95 (ddd, J=1.17, 8.49, 11.42 Hz, 1H), 7.74-7.90 (m, 3H), 7.68 (td, J=4.32, 8.35 Hz, 1H), 7.35-7.54 (m, 2H), 4.11 (s, 3H).

### 4-(4-Fluorophenyl)-2-hydroxy-6-(pyridin-2-yl)pyridine-3-carbonitrile

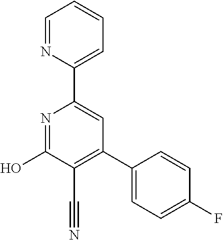 [SMILES: N#Cc1c(-c2ccc(F)cc2)cc(-c2ccccn2)nc1O]

A mixture of 4-fluorobenzaldehyde (1.00 g. 0.84 mL, 7.99 mmol), 1-(pyridin-2-yl)ethanone (0.96 g, 0.91 mL, 7.99 mmol), ethylcyanoacetate (0.90 g, 0.85 mL, 7.99 mmol), and ammonium acetate (4.93 g, 63.92 mmol) in 1,4-dioxane (32 mL) was heated at reflux for 16 hours. The mixture was cooled to 20° C., then was filtered, and the solid was washed with 1,4-dioxane (2×15 mL), and ethyl acetate (15 mL). The solid was treated with ethanol (30 mL), then was heated to boiling, and was allowed to cool to 20° C. The mixture was filtered, then the solid was washed with cold ethanol (10 mL), and pentane (10 mL). The solid was slurried in water (15 mL), and then sonicated for 5 minutes, and filtered. The solid was washed with isopropanol (15 mL), and pentane (10 mL), to give 4-(4-fluorophenyl)-2-hydroxy-6-(pyridin-2-yl)pyridine-3-carbonitrile (1.02 g, 44%) as a pale yellow solid. MS: M+H⁺ 292. ¹H NMR (300 MHz, DMSO-d₆) δ 8.77 (d, J=4.10 Hz, 1H), 8.26-8.39 (m, 1H), 7.98-8.12 (m, 1H), 7.75-7.89 (m, 2H), 7.54-7.67 (m, 1H), 7.39-7.52 (m, 2H), 7.15-7.39 (m, 1H).

### 4-(4-Fluorophenyl)-2-methoxy-6-(pyridin-2-yl)pyridine-3-carboxamide

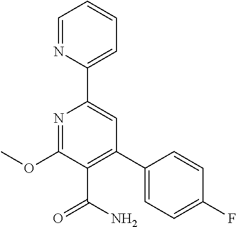 [SMILES: COc1nc(-c2ccccn2)cc(-c2ccc(F)cc2)c1C(N)=O]

A mixture of 4-(4-fluorophenyl)-2-methoxy-6-(pyridin-2-yl)pyridine-3-carbonitrile (50 mg, 0.16 mmol) in tert-butanol (0.5 mL) was treated with crushed potassium hydroxide (33 mg, 0.59 mmol). The mixture was heated in a sealed vessel at 95° C. for 2 hours. The reaction was treated with brine (2 mL), and water (10 mL). The mixture was extracted with 5% methanol in dichloromethane (3×15 mL). The combined organic extracts were washed with brine (5 mL), dried (Na₂SO₄), and concentrated. The crude material was purified by column on silica (0-5% methanol:dichloromethane). The product was dissolved in CH₂Cl₂ (3 mL) and methanol (1 mL), then was treated with 4N HCl in 1,4-dioxane (0.25 mL). The mixture was allowed to stand for 5 minutes, and then was concentrated to give 4-(4-fluorophenyl)-2-methoxy-6-(pyridin-2-yl)pyridine-3-carboxamide as the HCl salt (16 mg, 28%) as a pale yellow solid. MS: M+H⁺ 324. ¹H NMR (300 MHz, DMSO-d₆) δ 8.66-8.80 (m, 1H), 8.49 (d, J=7.62 Hz, 1H), 8.04-8.18 (m, 1H), 7.95-8.04 (m, 1H), 7.88 (br. s., 1H), 7.59-7.72 (m, 2H), 7.50-7.59 (m, 1H), 7.28-7.41 (m, 1H).

### 4-(4-Fluorophenyl)-2-methoxy-6-(3-methoxypyridin-2-yl)pyridine-3-carbonitrile

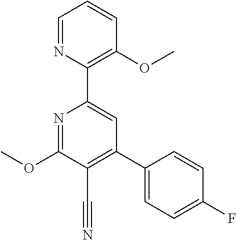 [SMILES: COc1cccnc1-c1cc(-c2ccc(F)cc2)c(C#N)c(OC)n1]

General Procedure G was followed, using 2-(4-fluoro-benzylidene)-malononitrile (220 mg, 1.25 mmol) and 1-(3-methoxypyridin-2-yl)ethanone (189 mg, 1.25 mmol), to give 4-(4-fluorophenyl)-2-methoxy-6-(3-methoxypyridin-2-yl)pyridine-3-carbonitrile as the HCl salt (38 mg, 8%) as a pale yellow solid. MS: M+H⁺ 336. ¹H NMR (300 MHz, DMSO-d₆) δ 8.30-8.45 (m, 1H), 7.73-7.89 (m, 3H), 7.57-7.72 (m, 2H), 7.37-7.53 (m, 2H), 4.10 (s, 3H), 3.91 (s, 3H).

### 2-(2-Hydroxyethoxy)-4-(4-fluorophenyl)-6-(pyridin-2-yl)pyridine-3-carbonitrile

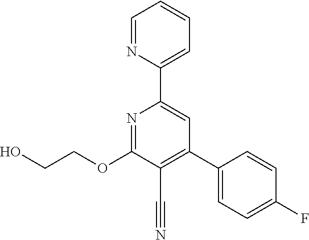 [SMILES: N#Cc1c(-c2ccc(F)cc2)cc(-c2ccccn2)nc1OCCO]

A mixture of 4-(4-fluorophenyl)-2-hydroxy-6-(pyridin-2-yl)pyridine-3-carbonitrile (50 mg, 0.17 mmol), and silver carbonate (69 mg, 0.25 mmol) in DMF (1 mL) was treated with 2-bromoethanol (31 mg, 18 μL, 0.25 mmol). The reaction was heated in a sealed vessel at 95° C. for 24 hours. The reaction was treated with water (15 mL), and then was extracted with ethyl acetate (2×20 mL). The combined organic extracts were washed with water (2×10 mL), and brine (1 mL), dried (Na₂SO₄), and concentrated. The crude material was purified by column on silica (0-5% methanol:dichloromethane). The product was dissolved in CH₂Cl₂ (3 mL) and methanol (1 mL), and then was treated with 4N HCl in 1,4-dioxane (0.25 mL). The mixture was allowed to stand for 5 minutes, and then was concentrated to give 2-(2-hydroxyethoxy)-4-(4-fluorophenyl)-6-(pyridin-2-yl)pyridine-3-carbonitrile as the HCl salt (8 mg, 13%) as a white solid. MS: M+H⁺ 336. ¹H NMR (300 MHz, DMSO-d₆) δ 8.69-8.83 (m, 1H), 8.47 (d, J=7.62 Hz, 1H), 8.14 (s, 1H), 8.00-8.11 (m, 1H), 7.76-7.91 (m, 2H), 7.53-7.66 (m, 1H), 7.46 (t, J=8.49 Hz, 2H), 4.58-4.73 (m, 2H), 3.78-3.93 (m, 2H).

### 2-Methoxy-6-phenyl-4-(pyridin-2-yl)pyridine-3-carbonitrile

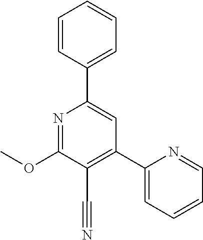 [SMILES: COc1nc(-c2ccccc2)cc(-c2ccccn2)c1C#N]

A mixture of 1-phenyl-3-pyridin-2-yl-propenone (200 mg, 0.96 mmol) in 0.5 M sodium methoxide in methanol (2.5 mL, 1.25 mmol) was treated with methanol (1 mL), followed by malononitrile (63 mg, 0.96 mmol) in methanol (3 mL) and crushed sodium hydroxide (192 mg, 4.80 mmol). The reaction mixture was stirred for 16 hours and concentrated. The residue was treated with ethyl acetate (20 mL), and water (10 mL). The aqueous layer was extracted with ethyl acetate (20 mL). The combined organic extracts was washed with brine (1 mL), dried (Na₂SO₄), and concentrated. The crude material was purified by column on silica (0-20% ethyl acetate:hexane) to give 2′-methoxy-6′-phenyl-[2,4′]bipyridinyl-3′-carbonitrile (23 mg, 8%) as a white solid. MS: M+H⁺ 288. ¹H NMR (300 MHz, CDCl₃) δ 8.77-8.94 (m, 1H), 8.10-8.24 (m, 2H), 7.83-8.03 (m, 3H), 7.40-7.61 (m, 4H), 4.24 (s, 3H).

### 2-(2-(2-Methoxyethoxy)ethoxy)-4-(4-fluorophenyl)-6-(pyridin-2-yl)pyridine-3-carbonitrile

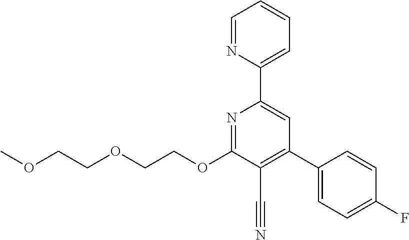 [SMILES: COCCOCCOc1nc(-c2ccccn2)cc(-c2ccc(F)cc2)c1C#N]

A mixture of 4-(4-fluorophenyl)-2-hydroxy-6-(pyridin-2-yl)pyridine-3-carbonitrile (50 mg, 0.17 mmol), 1-(2-methoxyethoxy)-2-bromoethane (62 mg, 46 μL, 0.34 mmol), and silver carbonate (69 mg, 0.25 mmol) in DMF (1 mL) was heated in a sealed vessel at 140° C. for 16 hours. The reaction was treated with water (15 mL), and then extracted with ethyl acetate (2×20 mL). The combined organic extracts were washed with water (2×10 mL), and brine (1 mL), dried (Na₂SO₄), and concentrated. The crude material was purified by column on silica (0-50% ethyl acetate:hexane). The product was dissolved in CH₂Cl₂ (3 mL) and methanol (1 mL), then was treated with 4N HCl in 1,4-dioxane (0.25 mL). The mixture was allowed to stand for 5 minutes, then was concentrated to give 2-(2-(2-methoxyethoxy)ethoxy)-4-(4-fluorophenyl)-6-(pyridin-2-yl)pyridine-3-carbonitrile as the HCl salt (55 mg, 75%) as an off-white solid. MS: M+H⁺ 394. ¹H NMR (300 MHz, DMSO-d₆) δ 8.69-8.87 (m, 1H), 8.49 (d, J=8.20 Hz, 1H), 8.00-8.24 (m, 2H), 7.81 (dd, J=5.57, 8.49 Hz, 2H), 7.60 (dd, J=4.39, 6.74 Hz, 1H), 7.33-7.52 (m, 2H), 4.74 (d, J=4.69 Hz, 2H), 4.14-4.33 (m, 4H), 3.86 (d, J=4.10 Hz, 2H), 3.58-3.71 (m, 2H), 3.36-3.51 (m, 2H), 3.22 (s, 3H).

### General Procedure H:

### 2-(2-(-(isoindoline-1,3-dione-2-yl)ethoxy)-4-(4-fluorophenyl)-6-(pyridin-2-yl)pyridine-3-carbonitrile

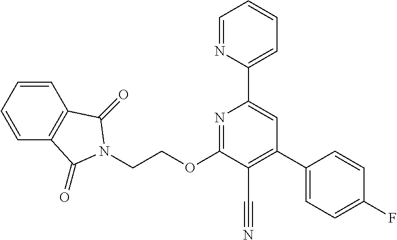 [SMILES: N#Cc1c(-c2ccc(F)cc2)cc(-c2ccccn2)nc1OCCN1C(=O)c2ccccc2C1=O]

A mixture of 4-(4-fluorophenyl)-2-hydroxy-6-(pyridin-2-yl)pyridine-3-carbonitrile (50 mg, 0.17 mmol), 2-(2-bromoethyl)isoindoline-1,3-dione (86 mg, 0.34 mmol), and silver carbonate (69 mg, 0.25 mmol) in DMF (1 mL) was heated in a sealed vessel at 140° C. for 16 hours. The reaction was treated with water (15 mL), and then was extracted with ethyl acetate (2×20 mL). The combined organic extracts was washed with water (2×10 mL), and brine (1 mL), dried (Na₂SO₄), and concentrated. The crude material was purified by column on silica (0-50% ethyl acetate:hexane), to give 2-(2-(-(isoindoline-1,3-dione-2-yl)ethoxy)-4-(4-fluorophenyl)-6-(pyridin-2-yl)pyridine-3-carbonitrile (55 mg, 70%) as a white solid. MS: M+H⁺ 465. ¹H NMR (300 MHz, CDCl₃) δ 8.58-8.77 (m, 1H), 8.35-8.53 (m, 1H), 8.13-8.25 (m, 1H), 7.58-7.99 (m, 9H), 7.32-7.45 (m, 1H), 7.10-7.32 (m, 4H), 4.79-4.99 (m, 2H), 4.14-4.34 (m, 2H).

### 2-(2-Aminoethoxy)-4-(4-fluorophenyl)-6-(pyridin-2-yl)pyridine-3-carbonitrile

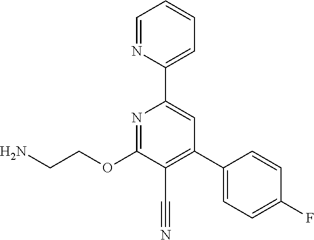 [SMILES: N#Cc1c(-c2ccc(F)cc2)cc(-c2ccccn2)nc1OCCN]

A mixture of 2-(2-(-(isoindoline-1,3-dione-2-yl)ethoxy)-4-(4-fluorophenyl)-6-(pyridin-2-yl)pyridine-3-carbonitrile (50 mg, 0.11 mmol) in THF (1 mL) and methanol (5 drops) was treated with hydrazine (17 mg, 0.54 mmol). The reaction mixture was stirred for 16 hours, and then concentrated. The residue was purified by column on silica (2-7% methanol:dichloromethane). The product was dissolved in CH₂Cl₂ (3 mL) and methanol (1 mL), then was treated with 4N HCl in 1,4-dioxane (0.25 mL). The mixture was allowed to stand for 5 minutes, then was concentrated to give 2-(2-aminoethoxy)-4-(4-fluorophenyl)-6-(pyridin-2-yl)pyridine-3-carbonitrile (13 mg, 29%) as a white solid. MS: M+H⁺ 335. ¹H NMR (300 MHz, DMSO-d₆) δ 8.69-8.83 (m, 1H), 8.49 (d, J=8.20 Hz, 1H), 7.97-8.34 (m, 5H), 7.76-7.93 (m, 2H), 7.53-7.64 (m, 1H), 7.42-7.52 (m, 1H), 4.79-4.93 (m, 2H), 2.54-2.43 (m, 2H).

### Methyl 2-(3-cyano-4-(4-fluorophenyl)-6-(pyridin-2-yl)pyridin-2-yloxy)acetate

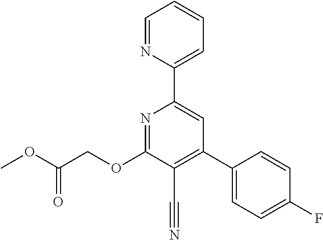 [SMILES: COC(=O)COc1nc(-c2ccccn2)cc(-c2ccc(F)cc2)c1C#N]

General Procedure H was followed, using 4-(4-fluorophenyl)-2-hydroxy-6-(pyridin-2-yl) pyridine-3-carbonitrile (100 mg, 0.34 mmol), methyl 2-bromoacetate (104 mg, 0.68 mmol), and silver carbonate (138 mg, 0.50 mmol), to give methyl 2-(3-cyano-4-(4-fluorophenyl)-6-(pyridin-2-yl)pyridin-2-yloxy)acetate (112 mg, 91%) as an off-white solid. MS: M+H⁺ 364. ¹H NMR (300 MHz, DMSO-d₆) δ 8.68-8.81 (m, 1H), 8.24 (d, J=8.20 Hz, 1H), 8.18 (s, 1H), 8.00-8.12 (m, 1H), 7.83 (dd, J=5.86, 8.20 Hz, 2H), 7.51-7.63 (m, 1H), 7.37-7.51 (m, 2H), 5.18-5.32 (m, 2H), 3.66-3.80 (m, 3H).

### 2-(3-Cyano-4-(4-fluorophenyl)-6-(pyridin-2-yl)pyridin-2-yloxy)acetic Acid

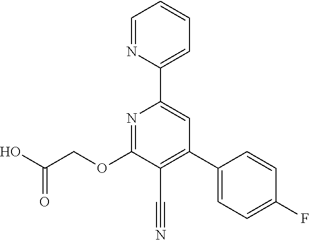 [SMILES: N#Cc1c(-c2ccc(F)cc2)cc(-c2ccccn2)nc1OCC(=O)O]

To a mixture of methyl 2-(3-cyano-4-(4-fluorophenyl)-6-(pyridin-2-yl)pyridin-2-yloxy)acetate (55 mg, 0.15 mmol) in THF (0.5 mL) and methanol (0.5 mL) was added 10N aqueous sodium hydroxide (0.1 mL). The reaction mixture was stirred for 24 hours, and then concentrated. The residue was partitioned between 10% aqueous acetic acid (10 mL) and ethyl acetate (30 mL). The organic layer was dried (Na₂SO₄), and concentrated. The crude material was purified by column on silica (0-100% ethyl acetate:hexane), to give 2-(3-cyano-4-(4-fluorophenyl)-6-(pyridin-2-yl)pyridin-2-yloxy)acetic acid (17 mg, 33%) as a white solid. MS: M+H⁺ 350. ¹H NMR (300 MHz, DMSO-d₆) δ 8.73 (d, J=4.10 Hz, 1H), 8.25-8.39 (m, 1H), 8.12-8.20 (m, 1H), 8.04 (t, J=7.62 Hz, 1H), 7.78-7.91 (m, 2H), 7.51-7.63 (m, 1H), 7.40-7.51 (m, 2H), 5.07-5.19 (m, 2H).

### 4-(Furan-2-yl)-2-methoxy-6-(pyridin-2-yl)pyridine-3-carbonitrile

### (i) (E)-1-(4-Fluorophenyl)-4,4-dimethylpent-1-en-3-one

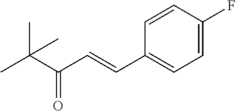

To a 0° C. mixture of 3,3-dimethylbutan-2-one (300 mg, 3.00 mmol) in methanol (15 mL) was added 10% aqueous potassium hydroxide solution (9 mL) followed by slow addition of 4-fluorobenzaldehyde (372 mg, 3.00 mmol). The reaction was allowed to slowly warm to 20° C., and was stirred for 3 days. The mixture was extracted with dichloromethane (2×20 mL). The combined organic extracts was washed with water (15 mL), and brine (3 mL), dried (Na₂SO₄), and concentrated. The crude material was purified by column on silica (0-5% ethyl acetate:hexane), to give (E)-1-(4-fluorophenyl)-4,4-dimethylpent-1-en-3-one (194 mg, 31%) as a white solid. MS: M+H⁺ 207. ¹H NMR (300 MHz, CDCl₃) δ 7.64 (d, J=15.82 Hz, 1H), 7.52-7.60 (m, 2H), 7.00-7.14 (m, 3H), 1.23 (s, 9H).

### (ii) 6-tert-Butyl-4-(4-fluorophenyl)-2-methoxypyridine-3-carbonitrile

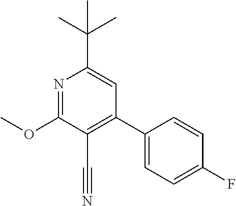 [SMILES: COc1nc(C(C)(C)C)cc(-c2ccc(F)cc2)c1C#N]

A mixture of (E)-1-(4-fluorophenyl)-4,4-dimethylpent-1-en-3-one (100 mg, 0.49 mmol) in 0.5M sodium methoxide in methanol (0.9 mL), and methanol (0.9 mL) was treated with malononitrile (36 mg, 0.54 mmol). The reaction was stirred for 16 hours, then was concentrated. The residue was partitioned between 5% methanol:dichloromethane (20 mL) and 5% aqueous acetic acid (10 mL). The organic layer was dried (Na₂SO₄), and concentrated. The crude material was purified by column on silica (0-10% ethyl acetate:hexane), to give 6-tert-butyl-4-(4-fluorophenyl)-2-methoxypyridine-3-carbonitrile (37 mg, 27%) as a white solid. MS: M+H⁺ 285. ¹H NMR (300 MHz, DMSO-d₆) δ 7.62-7.84 (m, 2H), 7.32-7.52 (m, 2H), 7.18 (s, 1H), 4.03 (s, 3H), 1.34 (s, 9H).

### (iii) (E)-3-(Furan-2-yl)-1-(pyridin-2-yl)prop-2-en-1-one

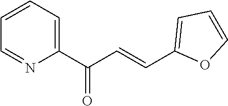 [SMILES: O=C(/C=C/c1ccco1)c1ccccn1]

To a mixture of 1-(pyridin-2-yl)ethanone (484 mg, 4.00 mmol) in methanol (20 mL) was added 10% aqueous potassium hydroxide (9 mL), followed by slow addition of furan-2-carbaldehyde (384 mg, 4.00 mmol). The reaction was stirred for 1.5 hours, then was filtered. The solid was washed with 5:3 methanol:water (2×10 mL), then was dried under high vacuum, to give (E)-3-(furan-2-yl)-1-(pyridin-2-yl)prop-2-en-1-one (207 mg, 26%) as a yellow solid. MS: M+H⁺ 200. ¹H NMR (300 MHz, CDCl₃) δ 8.75 (d, J=4.10 Hz, 1H), 8.13-8.22 (m, 2H), 8.08-8.13 (m, 1H), 8.07-8.24 (m, 1H), 7.87 (dt, J=1.46, 7.76 Hz, 1H), 7.55 (s, 1H), 7.48 (dd, J=4.98, 6.15 Hz, 1H), 6.78 (d, J=2.93 Hz, 1H).

### (iv) 4-(Furan-2-yl)-2-methoxy-6-(pyridin-2-yl)pyridine-3-carbonitrile

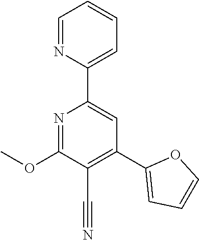 [SMILES: COc1nc(-c2ccccn2)cc(-c2ccco2)c1C#N]

A mixture of (E)-3-(furan-2-yl)-1-(pyridin-2-yl)prop-2-en-1-one (98 mg, 0.49 mmol) in methanol (0.9 mL) and 0.5 M sodium methoxide in methanol (0.9 mL) was treated with malononitrile (36 mg, 0.54 mmol). The reaction mixture was stirred for 3 days. The mixture was filtered, and then the solid was washed with cold methanol (2×5 mL). The crude material was purified by column on silica (0-20% ethyl acetate:hexane), to give 4-(furan-2-yl)-2-methoxy-6-(pyridin-2-yl)pyridine-3-carbonitrile (8 mg, 5%) as a white solid. MS: M+H⁺ 278. ¹H NMR (300 MHz, DMSO-d₆) δ 8.76 (d, J=4.10 Hz, 1H), 8.40-8.51 (m, 2H), 8.09 (s, 1H), 8.03 (dt, J=1.46, 7.76 Hz, 1H), 7.61 (d, J=3.51 Hz, 1H), 7.56 (dd, J=5.27, 6.44 Hz, 1H), 6.78-6.89 (m, 1H), 4.15 (s, 3H).

### 4-Ethoxy-2,6-diphenylpyrimidine-5-carbonitrile

### (i) (Z)—N-(Ethoxy(phenyl)methylene)benzamide

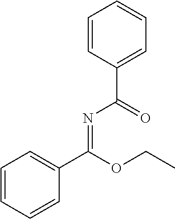 [SMILES: CCO/C(=N\C(=O)c1ccccc1)c1ccccc1]

A mixture of ethyl benzimidate.HCl (1.00 g, 5.39 mmol) in toluene (15 mL) was treated with trimethylamine (1.20 g, 1.64 mL, 11.86 mmol). The reaction was stirred for 5 minutes, then a solution of benzoyl chloride (0.76 g, 0.63 mL, 5.39 mmol) in toluene (4 mL) was added dropwise. The reaction was stirred for 40 hours, then was filtered. The solid was washed with toluene (10 mL), then the combined filtrates were concentrated, to give (Z)—N-(ethoxy(phenyl) methylene)benzamide (1.25 g, 92%) as a viscous liquid, which was used without further purification. MS: M+H⁺ 254. ¹H NMR (300 MHz, CDCl₃) δ 8.11-8.25 (m, 1H), 8.01 (d, J=7.03 Hz, 2H), 7.36-7.76 (m, 8H), 7.17-7.36 (m, 2H), 4.31-4.57 (m, 2H), 1.36-1.60 (m, 3H).

### (ii) 4-Hydroxy-2,6-diphenylpyrimidine-5-carbonitrile

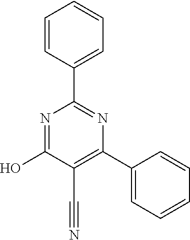 [SMILES: N#Cc1c(O)nc(-c2ccccc2)nc1-c1ccccc1]

To a mixture of 0.5M sodium methoxide in methanol (9.87 mL, 4.94 mmol) and methanol (7 mL) was added in one portion cyanoacetamide (0.42 g, 4.94 mmol). The mixture was stirred for 5 minutes, then a solution of (Z)—N-(ethoxy(phenyl)methylene)benzamide (1.25 g, 4.94 mmol) in methanol (3 mL) was added dropwise. The reaction was stirred for 4 days, then was treated with sulfuric acid (0.15 mL). The mixture was stirred for 10 minutes, then was filtered. The solid was washed with cold methanol (2×5 mL), and hexane (5 mL). The solid was treated with ethanol (50 mL), then was heated to boiling, and then was allowed to cool to 20° C. The mixture was filtered, then the solid was washed with cold ethanol (2×5 mL), and hexane (5 mL), to give 4-hydroxy-2,6-diphenylpyrimidine-5-carbonitrile (672 mg, 50%) as a white solid. MS: M+H⁺ 274. ¹H NMR (300 MHz, DMSO-d₆) δ 8.16-8.33 (m, 2H), 7.89-8.09 (m, 2H), 7.42-7.68 (m, 6H).

### (iii) 4-Ethoxy-2,6-diphenylpyrimidine-5-carbonitrile

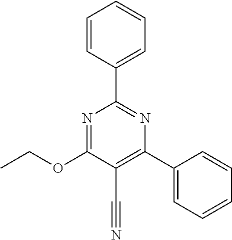 [SMILES: CCOc1nc(-c2ccccc2)nc(-c2ccccc2)c1C#N]

General Procedure H was followed, using 4-hydroxy-2,6-diphenylpyrimidine-5-carbonitrile (50 mg, 0.18 mmol), ethyl iodide (56 mg, 29 μL, 0.36 mmol), and silver carbonate (69 mg, 0.25 mmol), to give 4-ethoxy-2,6-diphenylpyrimidine-5-carbonitrile (18 mg, 33%) as a white solid. MS: M+H⁺ 302. ¹H NMR (300 MHz, DMSO-d₆) δ 8.42-8.59 (m, 2H), 7.95-8.15 (m, 2H), 7.44-7.76 (m, 6H), 4.63-4.82 (m, 2H), 1.35-1.56 (m, 3H).

### N-(2-(3-Cyano-4-(4-fluorophenyl)-6-(pyridin-2-yl)pyridin-2-yloxy)ethyl)acetamide

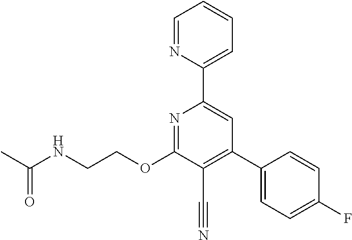 [SMILES: CC(=O)NCCOc1nc(-c2ccccn2)cc(-c2ccc(F)cc2)c1C#N]

A mixture of 4-(4-fluorophenyl)-2-hydroxy-6-(pyridin-2-yl)pyridine-3-carbonitrile (50 mg, 0.17 mmol), N-(2-chloroethyl)acetamide (41 mg, 0.34 mmol), sodium iodide (15 mg), and silver carbonate (69 mg, 0.25 mmol) in DMF (1 mL) was heated in a sealed vessel at 115° C. for 16 hours. The reaction was treated with water (15 mL), and then was extracted with ethyl acetate (2×15 mL). The combined organic extracts were washed with water (2×10 mL) and brine (1 mL), dried (Na₂SO₄), and concentrated. The crude material was purified by preparative TLC (5% MeOH:CH₂Cl₂), to give N-(2-(3-cyano-4-(4-fluorophenyl)-6-(pyridin-2-yl)pyridin-2-yloxy)ethyl/acetamide (4 mg, 6%) as a white solid. MS: M+H⁺ 377. ¹H NMR (300 MHz, CDCl₃) δ 8.73 (d, J=4.29 Hz, 1H), 8.46 (d, J=8.20 Hz, 1H), 8.29 (s, J=2.63 Hz, 1H), 7.91 (t, J=7.00 Hz, 1H), 7.75 (dd, J=4.98, 8.49 Hz, 2H), 5.91-6.21 (m, 1H), 4.73 (t, J=4.70 Hz, 2H), 3.79-3.87 (m, 2H), 2.06 (s, 3H).

### 2-(Cyanomethoxy)-4-(4-fluorophenyl)-6-(pyridin-2-yl)pyridine-3-carbonitrile

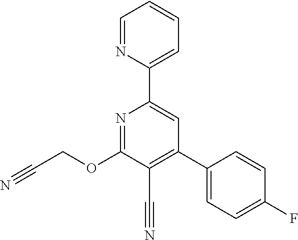 [SMILES: N#CCOc1nc(-c2ccccn2)cc(-c2ccc(F)cc2)c1C#N]

### General Procedure I:

A mixture of 4-(4-fluorophenyl)-2-hydroxy-6-(pyridin-2-yl)pyridine-3-carbonitrile (50 mg, 0.17 mmol), 2-bromoacetonitrile (41 mg, 23 μL, 0.34 mmol), and silver carbonate (69 mg, 0.25 mmol) in DMF (1 mL) was heated in a sealed vessel at 140° C. for 16 hours. The reaction was treated with water (15 mL), then was extracted with ethyl acetate (2×20 mL). The combined organic extracts were washed with water (2×10 mL), and brine (1 mL), dried (Na₂SO₄), and concentrated. The crude material was purified by column on silica (0-50% ethyl acetate:hexane). The product was dissolved in dichloromethane (3 mL) and methanol (1 mL), and was treated with 4N HCl in 1,4-dioxane (0.25 mL). The mixture was allowed to stand for 5 minutes, then was concentrated to give 2-(cyanomethoxy)-4-(4-fluorophenyl)-6-(pyridin-2-yl)pyridine-3-carbonitrile as the HCl salt (23 mg, 37%) as a white solid. MS: M+H⁺ 331. ¹H NMR (300 MHz, DMSO-d₆) δ 8.76 (d, J=4.69 Hz, 1H), 8.54 (d, J=8.20 Hz, 1H), 8.25 (s, 1H), 8.00-8.16 (m, 1H), 7.83 (dd, J=5.27, 8.20 Hz, 2H), 7.53-7.68 (m, 1H), 7.45 (t, J=8.49 Hz, 2H), 5.58 (s, 2H).

### 2-((S)-5-(Methoxy)pyrrolidin-2-one)-4-(4-fluorophenyl)-6-(pyridin-2-yl)pyridine-3-carbonitrile

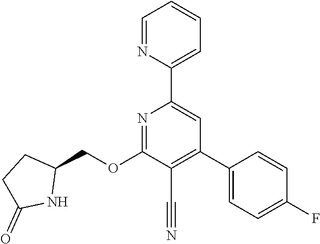 [SMILES: N#Cc1c(-c2ccc(F)cc2)cc(-c2ccccn2)nc1OC[C@@H]1CCC(=O)N1]

### General Procedure J:

A mixture of 4-(4-fluorophenyl)-2-hydroxy-6-(pyridin-2-yl)pyridine-3-carbonitrile (50 mg, 0.17 mmol), (S)-5-(bromomethyl)pyrrolidin-2-one (85 mg, 0.48 mmol), and silver carbonate (69 mg, 0.25 mmol) in DMF (1 mL) was heated in a sealed vessel at 140° C. for 16 hours. The reaction was treated with water (15 mL) and was extracted with ethyl acetate (2×20 mL). The combined organic extracts were washed with water (2×10 mL), and brine (1 mL), dried (Na₂SO₄), and concentrated. The crude material was purified by column on silica (0-5% methanol:dichloromethane). The product was dissolved in dichloromethane (3 mL) and methanol (1 mL), then was treated with 4N HCl in 1,4-dioxane (0.25 mL). The mixture was allowed to stand for 5 minutes, then was concentrated to give 2-((S)-5-(methoxy)pyrrolidin-2-one)-4-(4-fluorophenyl)-6-(pyridin-2-yl)pyridine-3-carbonitrile as the HCl salt (35 mg, 49%) as a pale yellow solid. MS: M+H⁺ 389. ¹H NMR (300 MHz, DMSO-d₆) δ 8.69-8.80 (m, 1H), 8.48 (d, J=7.62 Hz, 1H), 8.01-8.21 (m, 2H), 7.70-7.92 (m, 3H), 7.51-7.66 (m, 1H), 7.43 (t, J=8.20 Hz, 2H), 4.50-4.66 (m, 2H), 3.95-4.08 (m, 1H), 2.32-2.42 (m, 1H), 1.93-2.29 (m, 3H).

### 2-((R)-5-(Methoxy)pyrrolidin-2-one)-4-(4-fluorophenyl)-6-(pyridin-2-yl)pyridine-3-carbonitrile

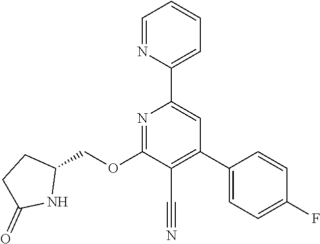 [SMILES: N#Cc1c(-c2ccc(F)cc2)cc(-c2ccccn2)nc1OC[C@H]1CCC(=O)N1]

Following General Procedure J, using 4-(4-fluorophenyl)-2-hydroxy-6-(pyridin-2-yl)pyridine-3-carbonitrile (50 mg, 0.17 mmol), (R)-5-(bromomethyl)pyrrolidin-2-one (85 mg, 0.48 mmol), and silver carbonate (69 mg, 0.25 mmol), to give 2-((R)-5-(methoxy)pyrrolidin-2-one)-4-(4-fluorophenyl)-6-(pyridin-2-yl)pyridine-3-carbonitrile as the HCl salt (39 mg, 54%) as a pale yellow solid. MS: M+H⁺ 389. ¹H NMR (300 MHz, DMSO-d₆) δ 8.75 (t, J=3.56 Hz, 1H), 8.49 (d, J=7.62 Hz, 1H), 8.03-8.20 (m, 2H), 7.73-7.90 (m, 3H), 7.52-7.66 (m, 1H), 7.44 (t, J=8.49 Hz, 2H), 4.58 (d, J=3.51 Hz, 2H), 3.97-4.07 (m, 1H), 2.31-2.43 (m, 1H), 1.93-2.30 (m, 3H).

### 2-(Allyloxy)-4-(4-fluorophenyl)-6-(pyridin-2-yl)pyridine-3-carbonitrile

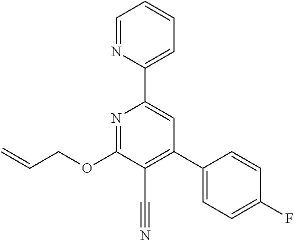 [SMILES: C=CCOc1nc(-c2ccccn2)cc(-c2ccc(F)cc2)c1C#N]

Following General Procedure J, using 4-(4-fluorophenyl)-2-hydroxy-6-(pyridin-2-yl)pyridine-3-carbonitrile (50 mg, 0.17 mmol), allylbromide (41 mg, 29 μL, 0.34 mmol), and silver carbonate (69 mg, 0.25 mmol), to give 2-(allyloxy)-4-(4-fluorophenyl)-6-(pyridin-2-yl)pyridine-3-carbonitrile as the HCl salt (55 mg, 88%) as a white solid. MS: M+H⁺ 332. ¹H NMR (300 MHz, DMSO-d₆) δ 8.69-8.81 (m, 1H), 8.40-8.55 (m, 1H), 7.98-8.21 (m, 2H), 7.80 (t, J=6.15 Hz, 2H), 7.50-7.63 (m, 1H), 7.39-7.49 (m, 2H), 6.09-6.24 (m, 1H), 5.51 (d, J=17.57 Hz, 1H), 5.32 (d, J=11.13 Hz, 1H), 5.10-5.19 (m, 2H).

### (Z)-1,3-Diphenyl-3-(prop-2-ynylamino)prop-2-en-1-one

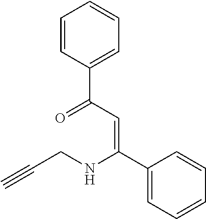 [SMILES: C#CCN/C(=C\C(=O)c1ccccc1)c1ccccc1]

A mixture of 1,3-diphenylprop-2-yn-1-one (200 mg, 0.97 mmol) and propargylamine (64 mg, 74 μL, 1.17 mmol) in methanol (2.6 mL) was heated at 60° C. for 6 hours. The mixture was concentrated, and the residue was partitioned between ethyl acetate (30 mL) and brine (3 mL). The organic layer was dried (Na₂SO₄) and concentrated. The crude material was purified by column on silica (0-20% ethyl acetate:hexane), to give (Z)-1,3-diphenyl-3-(prop-2-ynylamino)prop-2-en-1-one (143 mg, 57%) as a pale yellow solid. MS: M+H⁺ 262. ¹H NMR (300 MHz, CDCl₃) δ 11.05-11.50 (m, 1H), 7.90 (d, J=7.03 Hz, 2H), 7.37-7.53 (m, 8H), 5.84 (s, 1H), 3.90-3.98 (m, 2H).

### 2-Methoxy-3-methyl-4,6-diphenylpyridine

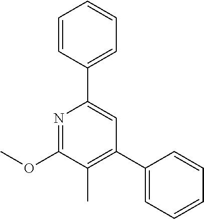 [SMILES: COc1nc(-c2ccccc2)cc(-c2ccccc2)c1C]

A mixture of (Z)-1,3-diphenyl-3-(prop-2-ynylamino)prop-2-en-1-one (50 mg, 0.19 mmol), methanol (12 mg, 15 μL, 0.38 mmol), and NaOH (8 mg, 0.19 mmol) in DMSO (0.14 mL) was stirred for 1.5 hours. The mixture was treated with water (10 mL), and was extracted with ethyl acetate (3×15 mL). The combined organic extracts were dried (Na₂SO₄), and concentrated. The crude material was purified by column on silica (0-5% ethyl acetate:hexane), to give 2-methoxy-3-methyl-4,6-diphenylpyridine (19 mg, 36%) as a clear gum. MS: M+H⁺ 276. ¹H NMR (300 MHz, CDCl₃) δ 8.08 (d, J=7.03 Hz, 2H), 7.34-7.51 (m, 8H), 7.29 (s, 1H), 4.12 (s, 3H), 2.17 (s, 3H).

### 4-(4-Fluorophenyl)-2-hydroxy-6-(3-methylpyridin-2-yl)pyridine-3-carbonitrile

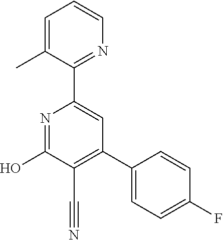 [SMILES: Cc1cccnc1-c1cc(-c2ccc(F)cc2)c(C#N)c(O)n1]

A mixture of 4-fluorobenzaldehyde (1.00 g, 0.84 mL, 7.99 mmol), l-(3-methylpyridin-2-yl)ethanone (1.08 g, 7.99 mmol), ethylcyanoacetate (0.90 g, 0.85 mL, 7.99 mmol), and ammonium acetate (4.93 g, 63.92 mmol) in 1,4-dioxane (32 mL) was heated at reflux for 16 hours. The mixture was concentrated, then the residue was treated with ethyl acetate (25 mL) and was sonicated for 5 minutes. The mixture was filtered, then the solid was washed with ethyl acetate (2×5 mL). The solid was slurried in water (25 mL), then was sonicated for 5 minutes. The mixture was filtered, then the solid was washed with water (5 mL), isopropanol (5 mL), and hexane (5 mL), to give 4-(4-fluorophenyl)-2-hydroxy-6-(3-methylpyridin-2-yl)pyridine-3-carbonitrile (833 mg, 34%) as a pale yellow solid. MS: M+H⁺ 306. ¹H NMR (300 MHz, DMSO-d₆) δ 12.29-12.91 (m, 1H), 8.28-8.70 (m, 1H), 7.64-7.91 (m, 3H), 7.32-7.55 (m, 3H), 6.42-6.89 (m, 1H), 2.35-2.43 (m, 3H).

### 2-(2-Morpholinoethoxy)-4-(4-fluorophenyl)-6-(3-methylpyridin-2-yl)pyridine-3-carbonitrile

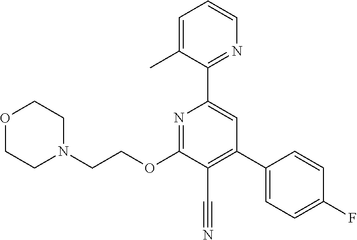 [SMILES: Cc1cccnc1-c1cc(-c2ccc(F)cc2)c(C#N)c(OCCN2CCOCC2)n1]

A mixture of 4-(4-fluorophenyl)-2-hydroxy-6-(3-methylpyridin-2-yl)pyridine-3-carbonitrile (50 mg, 0.16 mmol), 4-(2-chloroethyl)morpholine. HCl (61 mg, 0.32 mmol), sodium iodide (15 mg), and silver carbonate (133 mg, 0.48 mmol) in DMF (1 mL) was heated in a sealed vessel at 140° C. for 24 hours. The reaction was treated with water (15 mL), then was extracted with ethyl acetate (2×20 mL). The combined organic extracts were washed with water (2×10 mL), and brine (1 mL), dried (Na₂SO₄), and concentrated. The crude material was purified by column on silica (0-5% methanol:dichloromethane). Each product was dissolved in dichloromethane (3 mL) and methanol (1 mL), then was treated with 4N HCl in 1,4-dioxane (0.25 mL). The mixture was allowed to stand for 5 minutes, then was concentrated to give 2-(2-morpholinoethoxy)-4-(4-fluorophenyl)-6-(3-methylpyridin-2-yl)pyridine-3-carbonitrile as the bis-HCl salt (29 mg, 37%) as a pale yellow solid, and 4-(4-fluorophenyl)-1,2-dihydro-6-(3-methylpyridin-2-yl)-1-(2-morpholinoethyl)-2-oxopyridine-3-carbonitrile as the bis-HCl salt (35 mg, 45%) as a pale yellow solid.

For 2-(2-morpholinoethoxy)-4-(4-fluorophenyl)-6-(3-methylpyridin-2-yl)pyridine-3-carbonitrile: MS: M+H⁺ 419. ¹H NMR (300 MHz, DMSO-d₆) δ 11.01-11.48 (m, 1H), 8.57 (d, J=3.51 Hz, 1H), 7.75-7.96 (m, 4H), 7.40-7.55 (m, 3H), 4.82-5.04 (m, 2H), 3.89-4.22 (m, 2H), 3.74-3.85 (m, 2H), 3.62-3.71 (m, 2H), 3.45-3.61 (m, 2H), 3.14-3.42 (m, 2H), 2.63 (s, 3H).

For 4-(4-fluorophenyl)-1,2-dihydro-6-(3-methylpyridin-2-yl)-1-(2-morpholinoethyl)-2-oxopyridine-3-carbonitrile: MS: M+H⁺ 419. ¹H NMR (300 MHz, DMSO-d₆) δ 8.58 (t, J=3.71 Hz, 1H), 7.89 (br. s., 1H), 7.77 (t, J=5.86 Hz, 2H), 7.35-7.56 (m, 3H), 6.68 (s, J=3.24 Hz, 1H), 3.82-4.13 (m, 6H), 3.20-3.44 (m, 4H), 2.86-3.16 (m, 2H), 2.32 (s, 3H).

### Methyl 2-(3-cyano-4-(4-fluorophenyl)-6-(3-methylpyridin-2-yl)pyridin-2-yloxy) 2-(2-methoxyethoxy) acetate

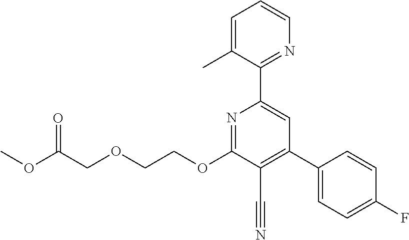 [SMILES: COC(=O)COCCOc1nc(-c2ncccc2C)cc(-c2ccc(F)cc2)c1C#N]

A mixture of 4-(4-fluorophenyl)-2-hydroxy-6-(3-methylpyridin-2-yl)pyridine-3-carbonitrile (50 mg, 0.16 mmol), methyl 2-(2-chloroethoxy)acetate (49 mg, 0.32 mmol), sodium iodide (15 mg), and silver carbonate (133 mg, 0.48 mmol) in DMF (1 mL) was heated in a sealed vessel at 140° C. for 16 hours. The reaction was treated with water (15 mL), then was extracted with ethyl acetate (2×20 mL). The combined organic extracts were washed with water (2×10 mL), and brine (1 mL), dried (Na₂SO₄), and concentrated. The crude material was purified by column on silica (0-50% ethyl acetate:hexane), to give methyl 2-(3-cyano-4-(4-fluorophenyl)-6-(3-methylpyridin-2-yl)pyridin-2-yloxy) 2-(2-methoxyethoxy) acetate (32 mg, 48%) as a white solid. MS: M+H⁺ 422. ¹H NMR (300 MHz, CDCl₃) δ 8.53 (d, J=3.51 Hz, 1H), 7.81 (s, 1H), 7.58-7.74 (m, 3H), 7.15-7.33 (m, 3H), 4.59-4.79 (m, 2H), 4.29 (s, 2H), 3.90-4.15 (m, 2H), 3.76 (s, 3H), 2.66 (s, 3H).

### 2-((Pyridin-3-yl)methoxy)-4-(4-fluorophenyl)-6-(3-methylpyridin-2-yl)pyridine-3-carbonitrile

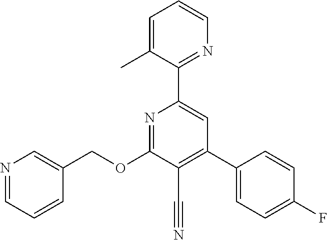 [SMILES: Cc1cccnc1-c1cc(-c2ccc(F)cc2)c(C#N)c(OCc2cccnc2)n1]

mixture of 4-(4-fluorophenyl)-2-hydroxy-6-(3-methylpyridin-2-yl)pyridine-3-carbonitrile (50 mg, 0.16 mmol), 3-(bromomethyl)pyridine. HBr (81 mg, 0.32 mmol), and silver carbonate (133 mg, 0.48 mmol) in DMF (1 mL) was heated in a sealed vessel at 140° C. for hours. The reaction was treated with water (15 mL), then was extracted with ethyl acetate (2×20 mL). The combined organic extracts were washed with water (2×10 mL) and brine (1 mL), dried (Na₂SO₄), and concentrated. The crude material was purified by column on silica (0-100% ethyl acetate:hexane) followed by column on silica (0-5% methanol:dichloromethane). Each product was dissolved in dichloromethane (3 mL) and methanol (1 mL), then was treated with 4N HCl in 1,4-dioxane (0.25 mL). The mixture was allowed to stand for 5 minutes, then was concentrated to give 2-((pyridin-3-yl)methoxy)-4-(4-fluorophenyl)-6-(3-methylpyridin-2-yl)pyridine-3-carbonitrile (25 mg, 33%) as a pale yellow solid, and 4-(4-fluorophenyl)-1,2-dihydro-6-(3-methylpyridin-2-yl)-2-oxo-1-((pyridin-3-yl)methyl)pyridine-3-carbonitrile (19 mg, 25%) as a white solid.

For 2-((pyridin-3-yl)methoxy)-4-(4-fluorophenyl)-6-(3-methylpyridin-2-yl)pyridine-3-carbonitrile: MS: M+H⁺ 397. ¹H NMR (300 MHz, CD₃OD) δ 9.03-9.21 (m, 1H), 8.79-9.00 (m, 3H), 8.61 (d, J=8.20 Hz, 1H), 8.11-8.28 (m, 1H), 7.99-8.12 (m, 1H), 7.74-7.97 (m, 3H), 7.23-7.51 (m, 2H), 5.95 (s, 2H), 2.73 (s, 3H).

For 4-(4-fluorophenyl)-1,2-dihydro-6-(3-methylpyridin-2-yl)-2-oxo-1-((pyridin-3-yl)methyl)pyridine-3-carbonitrile: MS: M+H⁺ 397. ¹H NMR (300 MHz, CD₃OD) δ 8.88 (s, 1H), 8.80 (d, J=5.86 Hz, 1H), 8.48-8.60 (m, 1H), 7.94-8.14 (m, 2H), 7.73-7.88 (m, 2H), 7.51-7.68 (m, 1H), 7.31 (s, 2H), 6.77 (s, J=3.02 Hz, 1H), 3.65 (s, 3H), 2.36 (s, 2H).

### tert-Butyl 4-(2-(3-cyano-4-(4-fluorophenyl)-6-(3-methylpyridin-2-yl)pyridin-2-yloxy)ethyl)piperazine-1-carboxylate

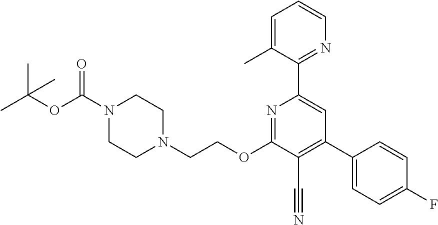 [SMILES: Cc1cccnc1-c1cc(-c2ccc(F)cc2)c(C#N)c(OCCN2CCN(C(=O)OC(C)(C)C)CC2)n1]

A mixture of 4-(4-fluorophenyl)-2-hydroxy-6-(3-methylpyridin-2-yl)pyridine-3-carbonitrile (50 mg, 0.16 mmol), tert-butyl 4-(2-chloroethyl)piperazine-1-carboxylate (80 mg, 0.32 mmol), sodium iodide (15 mg), and silver carbonate (133 mg, 0.48 mmol) in DMF (1 mL) was heated in a sealed vessel at 140° C. for 60 hours. The reaction was treated with water (15 mL), then was extracted with ethyl acetate (2×20 mL). The combined organic extracts were washed with water (2×10 mL) and brine (1 mL), dried (Na₂SO₄), and concentrated. The crude material was purified by column on silica (0-100% ethyl acetate:hexane) to give tert-butyl 4-(2-(3-cyano-4-(4-fluorophenyl)-6-(3-methylpyridin-2-yl)pyridin-2-yloxy)ethyl)piperazine-1-carboxylate (45 mg, 54%) as a clear gum. MS:M+H⁺ 518. ¹H NMR (300 MHz, CDCl₃) δ 8.46-8.58 (m, 1H), 7.76 (s, 1H), 7.55-7.71 (m, 3H), 7.10-7.32 (m, 3H), 4.46-4.70 (m, 2H), 3.33-3.49 (m, 4H), 2.77-2.98 (m, 2H), 2.64 (s, 3H), 2.40-2.60 (m, 4H), 1.43 (s, 9H).

### 2-(2-(Piperazin-1-yl)ethoxy)-4-(4-fluorophenyl)-6-(3-methylpyridin-2-yl)pyridine-3-carbonitrile

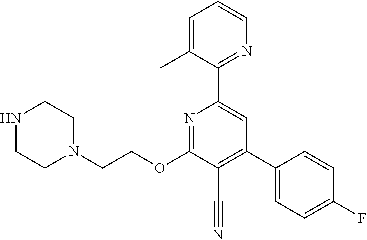 [SMILES: Cc1cccnc1-c1cc(-c2ccc(F)cc2)c(C#N)c(OCCN2CCNCC2)n1]

A mixture of tert-butyl 4-(2-(3-cyano-4-(4-fluorophenyl)-6-(3-methylpyridin-2-yl)pyridin-2-yloxy)ethyl)piperazine-1-carboxylate (109 mg, 0.21 mmol) in 1,4-dioxane (1.5 mL) was treated with 4N HCl in 1,4-dioxane (0.25 mL), then the reaction was stirred for 16 hours. The mixture was treated with water (1.5 mL), then the mixture was freeze dried to give 2-(2-(piperazin-1-yl)ethoxy)-4-(4-fluorophenyl)-6-(3-methylpyridin-2-yl)pyridine-3-carbonitrile as a yellow solid, which was used without further purification. MS: M+H⁺ 418.

### Methyl 4-(2-(3-cyano-4-(4-fluorophenyl)-6-(3-methylpyridin-2-yl)pyridin-2-yloxy)ethyl)piperazine-1-carboxylate

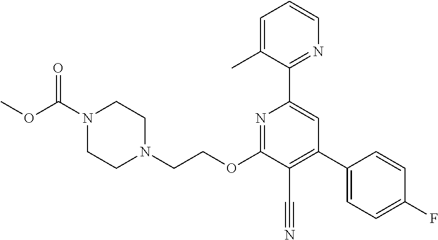 [SMILES: COC(=O)N1CCN(CCOc2nc(-c3ncccc3C)cc(-c3ccc(F)cc3)c2C#N)CC1]

A suspension of 2-(2-(piperazin-1-yl)ethoxy)-4-(4-fluorophenyl)-6-(3-methylpyridin-2-yl)pyridine-3-carbonitrile tris-trifluoro acetic acid salt (21 mg, 0.027 mmol) in dichloromethane (0.2 mL) was treated with trimethylamine (28 mg, 38 μL, 0.66 mmol), followed by methyl chloroformate (8 mg, 6 μL. 0.082 mmol). The reaction with stirred for 16 hours, then was quenched with saturated aqueous NaHCO₃. The mixture was treated with further dichloromethane, then the aqueous layer was extracted with dichloromethane (2×). The combined organic extracts were dried (Na₂SO₄), and concentrated. The crude material was purified by column on silica (0-7% methanol:dichloromethane), to give methyl 4-(2-(3-cyano-4-(4-fluorophenyl)-6-(3-methylpyridin-2-yl)pyridin-2-yloxy)ethyl)piperazine-1-carboxylate (9 mg, 68%) as an off-white solid. The solid was dissolved in 1,4-dioxane (0.5 mL), then was treated with 4N HCl in 1,4-dioxane (0.1 mL). The mixture was freeze dried to give 4-(2-(3-cyano-4-(4-fluorophenyl)-6-(3-methylpyridin-2-yl)pyri din-2-yloxy)ethyl)piperazine-1-carboxylate as the bis-HCl salt (12 mg, >100%) as a white solid. MS: M+H⁺ 477. ¹H NMR (300 MHz, CDCl₃) δ 8.54 (d, J=4.45 Hz, 1H), 7.78 (s, J=2.78 Hz, 1H), 7.62-7.72 (m, 3H), 7.17-7.32 (m, 4H), 4.64 (t, J=5.57 Hz, 2H), 3.69 (s, 3H), 3.44-3.54 (m, 4H), 2.91 (t, J=5.57 Hz, 2H), 2.57-2.67 (m, 7H).

### Ethyl 4-(2-(3-cyano-4-(4-fluorophenyl)-6-(3-methylpyridin-2-yl)pyridin-2-yloxy)ethyl)piperazine-1-carboxylate

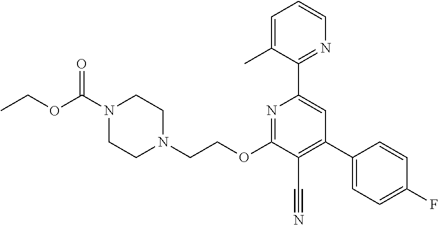 [SMILES: CCOC(=O)N1CCN(CCOc2nc(-c3ncccc3C)cc(-c3ccc(F)cc3)c2C#N)CC1]

A suspension of 2-(2-(piperazin-1-yl)ethoxy)-4-(4-fluorophenyl)-6-(3-methylpyridin-2-yl)pyridine-3-carbonitrile (35 mg, 0.066 mmol) in dichloromethane (0.2 mL) was treated with trimethylamine (67 mg, 93 μL, 0.66 mmol), followed by ethyl chloroformate (22 mg, 19 μL, 0.20 mmol). The reaction with stirred for 45 minutes, then was quenched with saturated aqueous NaHCO₃. The mixture was extracted with dichloromethane. The combined organic extracts were dried (Na₂SO₄), and concentrated. The crude material was purified by column on silica (0-7% methanol:dichloromethane), followed by purification by preparative HPLC. The desired product was free-based by partitioning between saturated aqueous NaHCO₃ and dichloromethane. The organic layer was dried (Na₂SO₄), and concentrated to give ethyl 4-(2-(3-cyano-4-(4-fluorophenyl)-6-(3-methylpyridin-2-yl)pyridin-2-yloxy)ethyl)piperazine-1-carboxylate (15 mg, 45%) as a white solid. The solid was dissolved in 1,4-dioxane (0.5 mL), then was treated with 4N HCl in 1,4-dioxane (0.1 mL). The mixture was freeze dried to give 4-(2-(3-cyano-4-(4-fluorophenyl)-6-(3-methylpyridin-2-yl)pyridin-2-yloxy)ethyl)piperazine-1-carboxylate as the bis-HCl salt (15 mg, 89%) as a white solid. MS: M+H⁺ 490. ¹H NMR (300 MHz, CDCl₃) δ 8.50-8.63 (m, 1H), 7.78 (s, 1H), 7.61-7.74 (m, 3H), 7.16-7.33 (m, 3H), 4.65 (t, J=4.98 Hz, 2H), 4.06-4.18 (m, 2H), 3.50 (br. s., 4H), 2.92 (br. s., 2H), 2.54-2.69 (m, 7H), 1.13-1.37 (m, 3H).

### Isopropyl 4-(2-(3-cyano-4-(4-fluorophenyl)-6-(3-methylpyridin-2-yl)pyridin-2-yloxy)ethyl)piperazine-1-carboxylate

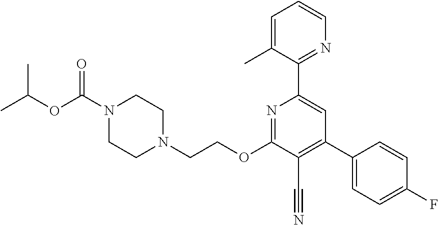 [SMILES: Cc1cccnc1-c1cc(-c2ccc(F)cc2)c(C#N)c(OCCN2CCN(C(=O)OC(C)C)CC2)n1]

A suspension of 2-(2-(piperazin-1-yl)ethoxy)-4-(4-fluorophenyl)-6-(3-methylpyridin-2-yl)pyridine-3-carbonitrile (35 mg, 0.066 mmol) in dichloromethane (0.2 mL) was treated with trimethylamine (67 mg, 93 μL, 0.66 mmol), followed by propan-2-yl chloroformate (22 mg, 24 μL, 0.20 mmol). The reaction with stirred for 30 minutes, then was quenched with saturated aqueous NaHCO₃. The mixture was extracted with dichloromethane. The combined organic extracts were dried (Na₂SO₄), and concentrated. The crude material was purified by column on silica (0-7% methanol:dichloromethane), to give isopropyl 4-(2-(3-cyano-4-(4-fluorophenyl)-6-(3-methylpyridin-2-yl)pyridin-2-yloxy)ethyl)piperazine-1-carboxylate (24 mg, 72%) as an off-white solid. The solid was dissolved in 1,4-dioxane (0.5 mL), then was treated with 4N HCl in 1,4-dioxane (0.1 mL). The mixture was freeze dried to give isopropyl 4-(2-(3-cyano-4-(4-fluorophenyl)-6-(3-methylpyridin-2-yl)pyridin-2-yloxy)ethyl)piperazine-1-carboxylate as the bis-HCl salt (26 mg, 93%) as a white solid. MS: M+H⁺ 505. ¹H NMR (300 MHz, CDCl₃) δ 8.50-8.57 (m, 1H), 7.78 (s, J=3.41, 3.41 Hz, 1H), 7.61-7.72 (m, 3H), 7.15-7.32 (m, 3H), 4.91 (br. s., 1H), 4.64 (t, J=4.98 Hz, 2H), 3.48 (br. s., 4H), 2.91 (t, J=4.69 Hz, 2H), 2.65 (s, 3H), 2.59 (br. s., 4H), 1.23 (d, J=5.86 Hz, 6H).

### 2-(3-(Dimethylamino)propoxy)-4-(4-fluorophenyl)-6-(pyridin-2-yl)pyridine-3-carbonitrile

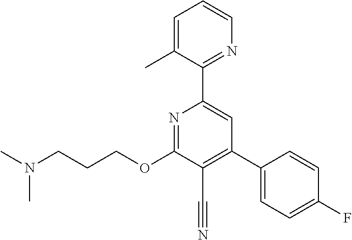 [SMILES: Cc1cccnc1-c1cc(-c2ccc(F)cc2)c(C#N)c(OCCCN(C)C)n1]

A mixture of 4-(4-fluorophenyl)-2-hydroxy-6-(3-methylpyridin-2-yl)pyridine-3-carbonitrile (50 mg, 0.16 mmol), 3-chloro-N,N-dimethylpropan-1-amine (76 mg, 0.48 mmol), sodium iodide (15 mg), and silver carbonate (133 mg, 0.48 mmol) in DMF (0.8 mL) was heated in a sealed vessel at 140° C. for 16 hours. The reaction was treated with water (15 mL), then was extracted with ethyl acetate (2×20 mL). The combined organic extracts were washed with water (2×10 mL), and brine (1 mL), dried (Na₂SO₄), and concentrated. The crude material was purified by preparative HPLC, to give 2-(3-(dimethylamino)propoxy)-4-(4-fluorophenyl)-6-(pyridin-2-yl)pyridine-3-carbonitrile as the bis-TFA salt (28 mg, 25%) as a white solid. MS: M+H⁺ 377. ¹H NMR (300 MHz, CD₃OD) δ 8.67-8.76 (m, 1H), 8.51 (d, J=7.62 Hz, 1H), 8.23 (s, 1H), 7.99-8.09 (m, 1H), 7.76-7.85 (m, 2H), 7.51-7.60 (m, 1H), 7.28-7.39 (m, 2H), 4.78 (t, J=5.86 Hz, 2H), 3.38-3.49 (m, 2H), 2.99 (s, 6H), 2.21-2.48 (m, 2H).

### 2-((Pyridin-2-yl)methoxy)-4-(4-fluorophenyl)-6-(3-methylpyridin-2-yl)pyridine-3-carbonitrile

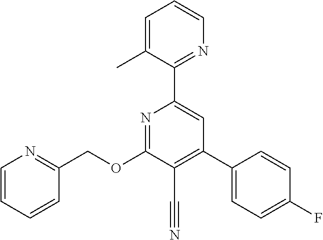 [SMILES: Cc1cccnc1-c1cc(-c2ccc(F)cc2)c(C#N)c(OCc2ccccn2)n1]

### General Procedure K

A mixture of 4-(4-fluorophenyl)-2-hydroxy-6-(3-methylpyridin-2-yl)pyridine-3-carbonitrile (50 mg, 0.16 mmol), 2-(bromomethyl)pyridine. HBr (81 mg, 0.32 mmol), and silver carbonate (133 mg, 0.48 mmol) in DMF (1 mL) was heated in a sealed vessel at 140° C. for 16 hours. The reaction was treated with water (15 mL), then was extracted with ethyl acetate (2×20 mL). The combined organic extracts were washed with water (2×10 mL), and brine (1 mL), dried (Na₂SO₄), and concentrated. The crude material was purified by column on silica (0-5% methanol:dichloromethane). The product was dissolved in dichloromethane (4 mL) and methanol (1 mL), then was treated with 4N HCl in 1,4-dioxane (0.25 mL). The mixture was allowed to stand for 5 minutes, then was concentrated to give 2-((pyri din-2-yl)methoxy)-4-(4-fluorophenyl)-6-(3-methylpyridin-2-yl)pyridine-3-carbonitrile as the bis-HCl salt (35 mg, 47%) was a pale yellow solid. MS: M+H⁺ 397. ¹H NMR (300 MHz, CD₃OD) δ 8.92 (t, J=6.44 Hz, 2H), 8.62-8.79 (m, 2H), 8.35 (d, J=8.20 Hz, 1H), 8.04-8.19 (m, 2H), 7.78-8.01 (m, 3H), 7.38 (t, J=1.00 Hz, 2H), 6.14 (s, 2H), 2.70 (s, 3H).

### 2-((Pyridin-4-yl)methoxy)-4-(4-fluorophenyl)-6-(3-methylpyridin-2-yl)pyridine-3-carbonitrile

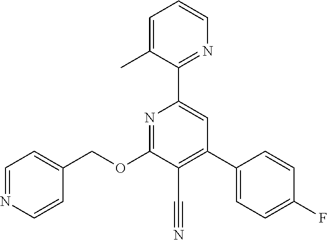 [SMILES: Cc1cccnc1-c1cc(-c2ccc(F)cc2)c(C#N)c(OCc2ccncc2)n1]

Following General Procedure K, using 4-(4-fluorophenyl)-2-hydroxy-6-(3-methylpyridin-2-yl)pyridine-3-carbonitrile (50 mg, 0.16 mmol), 4-(bromomethyl)pyridine. HBr (81 mg, 0.32 mmol), and silver carbonate (133 mg, 0.48 mmol), to give 2-((pyridin-4-yl)methoxy)-4-(4-fluorophenyl)-6-(3-methylpyridin-2-yl)pyridine-3-carbonitrile as the bis-HCl salt (30 mg, 40%) as a pale yellow solid. MS: M+H⁺ 397. ¹H NMR (300 MHz, CD₃OD) δ 8.52-8.74 (m, 2H), 8.03-8.23 (m, 2H), 7.95 (d, J=7.03 Hz, 1H), 7.74-7.90 (m, 4H), 7.71 (s, 1H), 7.52-7.70 (m, 2H), 7.24-7.41 (m, 3H), 4.23 (s, 2H), 2.66 (s, 3H).

### 2-Methoxy-4-(4-methoxyphenyl)-6-(3-methylpyridin-2-yl)pyridine-3-carbonitrile

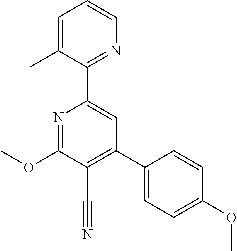

### (i) 2-(4-Methoxybenzylidene)malononitrile

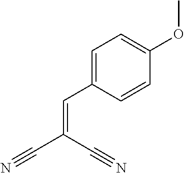

A mixture of 4-methoxybenzaldehyde (1.00 g, 7.35 mmol) and malononitrile (0.149 g, 7.35 mmol) in ethanol (7 mL) was treated with a 10% aqueous KOH solution (0.15 mL). The mixture was stirred for 15 minutes, then was allowed to stand for 30 minutes. The mixture was treated with ethanol (3 mL), then was filtered. The solid was washed with cold ethanol (10 mL), and hexane (10 ml), to give 2-(4-methoxybenzylidene)malononitrile (1.12 g, 83%) as a pale yellow solid. MS: M+H⁺ 185. ¹H NMR (300 MHz, CDCl₃) δ 7.91 (t, J=4.22 Hz, 2H), 7.65 (s, J=3.85 Hz, 1H), 7.01 (d, J=9.37 Hz, 2H), 3.91 (s, 3H).

### (ii) 2-methoxy-4-(4-methoxyphenyl)-6-(3-methylpyridin-2-yl)pyridine-3-carbonitrile

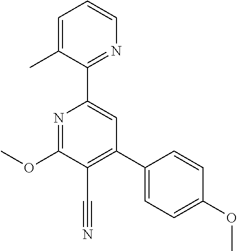

A mixture of 2-(4-methoxybenzylidene)malononitrile (230 mg, 1.25 mmol) and 1-(3-methylpyridin-2-yl)ethanone (169 mg, 1.25 mmol) in methanol (4 mL) was treated with crushed NaOH (100 mg, 2.50 mmol). The reaction was stirred for 60 hours. The mixture was filtered, then the solid was washed with void methanol (3 mL). The crude material was purified by column on silica (0-50% ethyl acetate:hexane). The product was dissolved in dichloromethane (3 mL) and methanol (1 mL), then was treated with 4N HCl in 1,4-dioxane (0.25 mL). The mixture was allowed to stand for 5 minutes, then was concentrated to give 2-methoxy-4-(4-methoxyphenyl)-6-(3-methylpyridin-2-yl)pyridine-3-carbonitrile as the HCl salt (37 mg, 8%) as a pale yellow solid. MS: M+H⁺ 332. ¹H NMR (300 MHz, DMSO-dg) δ 8.50-8.67 (m, 1H), 7.92 (d, J=1.00 Hz, 1H), 7.77-7.81 (m, 1H), 7.63-7.75 (m, 2H), 7.51 (dd, J=4.69, 7.62 Hz, 1H), 7.03-7.23 (m, 2H), 3.75-3.90 (m, 3H), 3.49-3.62 (m, 3H), 2.64 (s, 3H).

### 2-Methoxy-4-(3-nitrophenyl)-6-phenylpyridine-3-carbonitrile

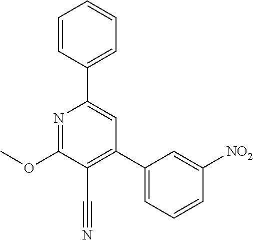 [SMILES: COc1nc(-c2ccccc2)cc(-c2cccc([N+](=O)[O-])c2)c1C#N]

A mixture of (E)-3-(3-nitrophenyl)-1-phenylprop-2-en-1-one (1.00 g, 3.95 mmol) in methanol (13 mL) and 5.4 M NaOMe in methanol (0.7 mL) was treated with malononitrile (263 mg, 3.95 mmol). The reaction was stirred for 16 hours. The mixture was treated with methanol (5 mL), then was filtered, and the solid was washed with cold methanol (2×5 mL). The solid was recrystallized from ethanol, to give 2-methoxy-4-(3-nitrophenyl)-6-phenylpyridine-3-carbonitrile (0.49 g, 37%) as a pale yellow solid. MS: M+H⁺ 332. ¹H NMR (300 MHz, DMSO-d₆) δ 8.59 (t, J=2.05 Hz, 1H), 8.41 (td, J=1.17, 8.20 Hz, 1H), 8.26-8.31 (m, 2H), 8.20 (s, J=5.26, 5.26 Hz, 1H), 7.83-8.03 (m, 2H), 7.47-7.61 (m, 3H), 4.16 (s, 3H).

### 4-(3-Aminophenyl)-2-methoxy-6-phenylpyridine-3-carbonitrile

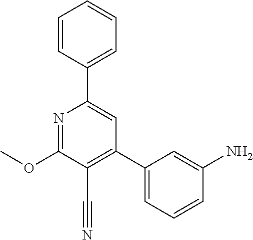 [SMILES: COc1nc(-c2ccccc2)cc(-c2cccc(N)c2)c1C#N]

### General Procedure L:

A mixture of 2-methoxy-4-(3-nitrophenyl)-6-phenylpyridine-3-carbonitrile (50 mg, 0.15 mmol), and SnCl₂.2 H₂O (170 mg. 0.75 mmol) in ethanol (1.5 mL) was heated in a sealed vessel at 80° C. for 3 hours. The reaction was cooled to 20° C., then was poured onto ice/water (40 mL). The mixture was adjusted to pH7 using saturated aqueous NaHCO₃, then was extracted with ethyl acetate (2×25 mL). The combined organic extracts were filtered through Celite, then were washed with brine (1 mL), dried (Na₂SO₄), and concentrated. The crude material was purified by column on silica (0-5% methanol:dichloromethane), to give 4-(3-aminophenyl)-2-methoxy-6-phenylpyridine-3-carbonitrile (29 mg, 64%) as a pale yellow solid. MS: M+H⁺ 302. ¹H NMR (300 MHz, CDCl₃) δ 8.04-8.14 (m, 2H), 7.43-7.54 (m, 5H), 7.23-7.32 (m, 1H), 7.00 (d, J=7.62 Hz, 1H), 6.89-6.94 (m, 1H), 6.80 (dd, J=1.76, 7.62 Hz, 1H), 4.18 (s, 3H).

### 2-(3-(Dimethylamino)propoxy)-4-(4-fluorophenyl)-6-(3-methylpyridin-2-yl)pyridine-3-carbonitrile

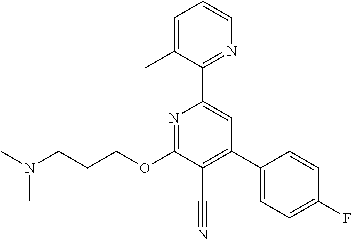 [SMILES: Cc1cccnc1-c1cc(-c2ccc(F)cc2)c(C#N)c(OCCCN(C)C)n1]

A mixture of 4-(4-fluorophenyl)-2-hydroxy-6-(3-methylpyridin-2-yl)pyridine-3-carbonitrile (50 mg, 0.16 mmol), 23-chloro-N,N-dimethylpropan-1-amine. C1 (51 mg, 0.32 mmol), sodium iodide (15 mg), and silver carbonate (133 mg, 0.48 mmol) in DMF (1 mL) was heated in a sealed vessel at 140° C. for 16 hours. The reaction was treated with water (15 mL), then was extracted with ethyl acetate (2×15 mL). The combined organic extracts were washed with water (2×10 mL), and brine (1 mL), dried (Na₂SO₄), and concentrated. The crude material was purified by column on silica (0-5% methanol:dichloromethane), to give 2-(3-(dimethylamino)propoxy)-4-(4-fluorophenyl)-6-(3-methylpyridin-2-yl)pyridine-3-carbonitrile (12 mg, 19%) as a white solid. MS: M+H⁺ 391. ¹H NMR (300 MHz, CDCl₃) δ 8.38-8.60 (m, 1H), 7.79 (s, 1H), 7.54-7.72 (m, 3H), 7.10-7.34 (m, 3H), 4.57 (t, J=5.57 Hz, 2H), 3.03-3.19 (m, 2H), 2.70 (s, 6H), 2.63 (s, 3H), 2.22-2.44 (m, 2H).

### 2-Methoxy-4-(4-nitrophenyl)-6-phenylpyridine-3-carbonitrile

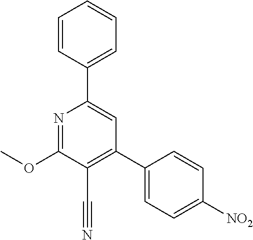 [SMILES: COc1nc(-c2ccccc2)cc(-c2ccc([N+](=O)[O-])cc2)c1C#N]

### (i) (E)-3-(4-Nitrophenyl)-1-phenylprop-2-en-1-one

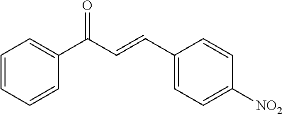 [SMILES: O=C(/C=C/c1ccc([N+](=O)[O-])cc1)c1ccccc1]

A mixture of acetophenone (0.50 g, 4.16 mmol) and 4-nitrobenzaldehyde (0.63 g, 4.16 mmol) in methanol (14 mL) was treated with sodium hydroxide (333 mg, 8.32 mmol), The reaction was stirred for 16 hours. The mixture was poured into cold water (75 mL), then was filtered. The solid was treated with hot ethanol (20 mL), then was allowed to cool to 20° C. The mixture was filtered, then the solid was washed with ethanol (5 mL), and hexane (5 mL), to give (E)-3-(4-nitrophenyl)-1-phenylprop-2-en-1-one (670 mg, 64%) as a yellow solid. MS: M+H⁺ 254. ¹H NMR (300 MHz, CDCl₃) δ 8.22-8.32 (m, 2H), 7.98-8.07 (m, 2H), 7.73-7.86 (m, 3H), 7.46-7.71 (m, 4H).

### (ii) 2-Methoxy-4-(4-nitrophenyl)-6-phenylpyridine-3-carbonitrile

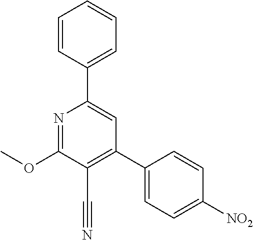 [SMILES: COc1nc(-c2ccccc2)cc(-c2ccc([N+](=O)[O-])cc2)c1C#N]

A mixture of (E)-3-(4-nitrophenyl)-1-phenylprop-2-en-1-one (250 mg, 0.99 mmol) in methanol (3 mL) and 5.4 M sodium methoxide in methanol (0.108 mL) was treated with malononitrile (66 mg, 0.99 mmol). The reaction was stirred for 16 hours. The mixture was filtered, then the solid was washed with cold methanol (2×2 mL). The solid was treated with hot ethanol (25 mL), then was allowed to cool. The mixture was filtered, then the solid was washed with cold ethanol (3 mL). The solid was treated with hot isopropanol (25 mL), then was allowed to cool. The mixture was filtered, then the solid was washed with ispopropanol (5 mL), and hexane (5 mL), to give 2-methoxy-4-(4-nitrophenyl)-6-phenylpyridine-3-carbonitrile (137 mg, 42%) as a pale yellow solid. MS: M+H⁺ 332. ¹H NMR (300 MHz, DMSO-d₆) δ 8.41 (d, J=8.79 Hz, 2H), 8.24-8.35 (m, 2H), 8.02 (d, J=8.79 Hz, 2H), 7.88-7.94 (m, 1H), 7.49-7.59 (m, 3H), 4.15 (s, 3H).

### 4-(4-Aminophenyl)-2-methoxy-6-phenylpyridine-3-carbonitrile

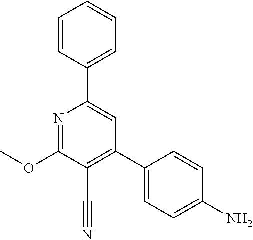 [SMILES: COc1nc(-c2ccccc2)cc(-c2ccc(N)cc2)c1C#N]

Following General Procedure L, using 2-methoxy-4-(4-nitrophenyl)-6-phenylpyridine-3-carbonitrile (50 mg, 0.15 mmol) and SnCl₂.2 H₂O (170 mg, 0.75 mmol), to give 4-(4-aminophenyl)-2-methoxy-6-phenylpyridine-3-carbonitrile (25 mg, 55%) as a pale yellow solid. MS: M+H⁺ 302. ¹H NMR (300 MHz, CDCl₃) δ 8.01-8.18 (m, 2H), 7.43-7.56 (m, 6H), 6.78 (t, J=5.61 Hz, 2H), 4.16-4.20 (m, 3H).

### 4-(4-Hydroxyphenyl)-2-methoxy-6-phenylpyridine-3-carbonitrile

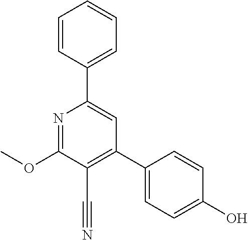 [SMILES: COc1nc(-c2ccccc2)cc(-c2ccc(O)cc2)c1C#N]

A mixture of (E)-3-(4-hydroxyphenyl)-1-phenylprop-2-en-1-one (0.25 g, 1.12 mmol) in methanol (3 mL) and 5.4 M sodium methoxide in methanol (0.20 mL) was treated with malononitrile (74 mg, 1.12 mmol). The reaction was stirred for 16 hours, and was then filtered. The solid was washed with cold methanol (3 mL). The solid was dissolved in hot ethanol, then was cooled at 0° C. for 16 hours. The mixture was filtered, then the solid was washed with cold ethanol (2 mL), to give 4-(4-hydroxyphenyl)-2-methoxy-6-phenylpyridine-3-carbonitrile (43 mg, 13%) as a pale orange solid. MS: M+H⁺ 303. ¹H NMR (300 MHz, DMSO-d₆) δ 10.05 (s, 1H), 8.16-8.30 (m, 2H), 7.74 (s, J=3.75 Hz, 1H), 7.62 (d, J=8.20 Hz, 2H), 7.51 (d, J=1.76 Hz, 3H), 6.93 (d, J=8.79 Hz, 2H), 4.11 (s, 3H), 3.32 (s, 5H).

### 4-(3-Hydroxyphenyl)-2-methoxy-6-phenylpyridine-3-carbonitrile

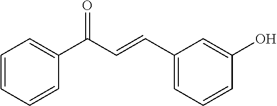 [SMILES: O=C(/C=C/c1cccc(O)c1)c1ccccc1]

### (i) (E)-3-(3-Hydroxyphenyl)-1-phenylprop-2-en-1-one

A mixture of sodium hydroxide (0.44 g, 11 mmol) in water (4 mL) and ethanol (2 mL) was cooled to 0° C., then acetophenone (1.03 g, 1.00 mL, 8.60 mmol), followed by 3-hydroxybenzaldehyde (1.05 g, 8.60 mmol) were added. The reaction was stirred for 16 hours. The mixture was treated with 1N HCl (10 mL), then was filtered. The solid was washed with water (10 mL), isopropanol (10 mL), and hexane (10 mL), to give (E)-3-(3-hydroxyphenyl)-1-phenylprop-2-en-1-one (1.24 g, 64%) as a white solid. MS: M+H⁺ 225. ¹H NMR (300 MHz, DMSO-d₆) δ 9.62 (s, J=2.39 Hz, 1H), 8.12 (d, J=7.03 Hz, 2H), 7.79 (d, J=1.00 Hz, 1H), 7.65 (s, 2H), 7.55 (s, 2H), 7.17-7.35 (m, 3H), 6.79-6.91 (m, 1H).

### (ii) 4-(3-Hydroxyphenyl)-2-methoxy-6-phenylpyridine-3-carbonitrile

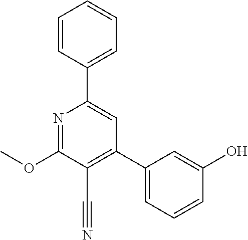 [SMILES: COc1nc(-c2ccccc2)cc(-c2cccc(O)c2)c1C#N]

A mixture of (E)-3-(3-hydroxyphenyl)-1-phenylprop-2-en-1-one (0.25 g, 1.12 mmol) in methanol (3 mL) and 5.4 M sodium methoxide in methanol (0.20 mL) was treated with malononitrile (74 mg, 1.12 mmol). The reaction was stirred for 3 days. The mixture was treated with 1 N HCl (3 mL), then was concentrated. The residue was partitioned between ethyl acetate (20 mL) and water (3 mL). The organic layer was washed with brine (3 mL), dried (Na₂SO₄), and concentrated. The crude material was purified by column on silica (0-30% ethyl acetate:hexane), to give 4-(3-hydroxyphenyl)-2-methoxy-6-phenylpyridine-3-carbonitrile (59 mg, 17%) as a white solid. MS: M+H⁺ 303. ¹H NMR (300 MHz, DMSO-d₆) δ 9.83 (s, 1H), 8.17-8.33 (m, 2H), 7.76 (s, 1H), 7.46-7.57 (m, 3H), 7.35 (t, J=7.91 Hz, 1H), 7.02-7.17 (m, 2H), 6.85-7.01 (m, 1H), 4.12 (s, 3H).

### 4-(4-Fluorophenyl)-2-methoxy-6-(2-nitrophenyl)pyridine-3-carbonitrile

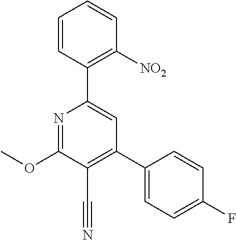 [SMILES: COc1nc(-c2ccccc2[N+](=O)[O-])cc(-c2ccc(F)cc2)c1C#N]

### (i) (E)-3-(4-Fluorophenyl)-1-(2-nitrophenyl)prop-2-en-1-one

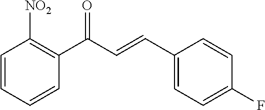 [SMILES: O=C(/C=C/c1ccc(F)cc1)c1ccccc1[N+](=O)[O-]]

A mixture of 1-(2-nitrophenyl)ethanone (500 mg, 3.03 mmol) and 4-fluorobenzaldehyde (413 mg, 353 μL, 3.33 mmol) in methanol (15 mL) was treated with 10% aqueous sodium hydroxide (1.6 mL). The reaction was stirred for 16 hours. The mixture was filtered, then the solid was washed with cold methanol (5 mL), and hexane (5 mL), to give (E)-3-(4-fluorophenyl)-1-(2-nitrophenyl)prop-2-en-1-one (518 mg, 63%) as a white solid. MS: M+H⁺272. ¹H NMR (300 MHz, DMSO-d₆) δ 8.13-8.22 (m, 1H), 7.75-7.94 (m, 4H), 7.67-7.74 (m, 1H), 7.37 (d, J=16.40 Hz, 1H), 7.27 (t, J=8.80 Hz, 3H).

### (ii) 4-(4-Fluorophenyl)-2-methoxy-6-(2-nitrophenyl)pyridine-3-carbonitrile

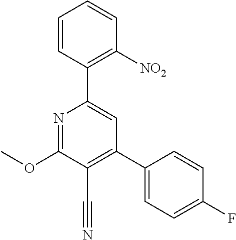 [SMILES: COc1nc(-c2ccccc2[N+](=O)[O-])cc(-c2ccc(F)cc2)c1C#N]

A mixture of (E)-3-(4-fluorophenyl)-1-(2-nitrophenyl)prop-2-en-1-one (250 mg, 0.92 mmol) in methanol (3 mL) and 5.4 M sodium methoxide in methanol (0.20 mL) was treated with malononitrile (61 mg, 0.92 mmol). The reaction was stirred for 16 hours. The mixture was filtered, then the solid was washed with cold methanol (2 mL). The crude material was purified by column on silica (0-20% ethyl acetate:hexane), to give 4-(4-fluorophenyl)-2-methoxy-6-(2-nitrophenyl)pyridine-3-carbonitrile (126 mg, 39%) as a yellow solid. MS: M+H⁺ 350. ¹H NMR (300 MHz, DMSO-d₆) δ 8.07 (dd, J=1.46, 7.32 Hz, 1H), 7.97 (dd, J=1.17, 8.20 Hz, 1H), 7.74 (s, 5H), 7.44 (s, 2H), 3.31 (s, 4H).

### 6-(2-Aminophenyl)-4-(4-fluorophenyl)-2-methoxypyridine-3-carbonitrile

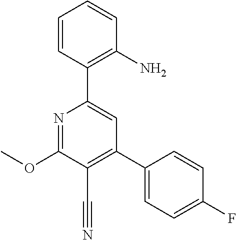 [SMILES: COc1nc(-c2ccccc2N)cc(-c2ccc(F)cc2)c1C#N]

A mixture of 4-(4-fluorophenyl)-2-methoxy-6-(2-nitrophenyl)pyridine-3-carbonitrile (50 mg, 0.104 mmol) and SnCl₂.2 H₂O (162 mg, 0.70 mmol) in ethanol (1.5 mL) was heated in a sealed vessel at 80° C. for 3 hours. The mixture was poured in to ice/water (40 mL), then was extracted with ethyl acetate (2×25 mL). The combined organic extracts were filtered through Celite, then the organic layer was washed with brine (5 mL) dried (Na₂SO₄), and concentrated. The crude material was purified by column on silica (0-30% ethyl acetate:hexane), to give 6-(2-aminophenyl)-4-(4-fluorophenyl)-2-methoxypyridine-3-carbonitrile (31 mg, 69%) as a yellow solid. MS: M+H⁺ 320. ¹H NMR (300 MHz, CDCl₃) δ 7.53-7.68 (m, 3H), 7.31-7.40 (m, 1H), 7.16-7.27 (m, 3H), 6.76 (t, J=7.41 Hz, 2H), 5.55-5.80 (m, 2H), 4.10 (s, 3H).

### 4-(4-fluorophenyl)-6-(3-hydroxyphenyl)-2-methoxypyridine-3-carbonitrile

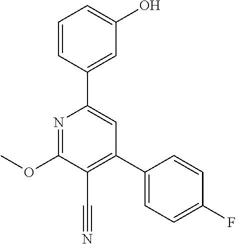 [SMILES: COc1nc(-c2cccc(O)c2)cc(-c2ccc(F)cc2)c1C#N]

### (i) (E)-3-(4-fluorophenyl)-1-(3-hydroxyphenyl)prop-2-en-1-one

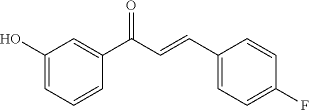 [SMILES: O=C(/C=C/c1ccc(F)cc1)c1cccc(O)c1]

To a 0° C. mixture of sodium hydroxide (0.22 g, 5.50 mmol) in water (2 mL) and ethanol (1 mL) was added 1-(3-hydroxyphenyl)ethanone (0.59 g, 4.30 mmol) and 4-fluorobenzaldehyde (0.53 g, 0.46 mL, 4.30 mmol). The reaction was allowed to warm to 20° C., and was stirred for 16 hours. The solution was treated with 1N HCl (5 mL0, then was filtered, to give (E)-3-(4-fluorophenyl)-1-(3-hydroxyphenyl)prop-2-en-1-one (1.02 g, 98%) as a yellow solid. MS: M+H⁺ 243. ¹H NMR (300 MHz, DMSO-d₆) δ 7.84-7.99 (m, 2H), 7.61-7.82 (m, 2H), 7.51 (d, J=8.20 Hz, 1H), 7.40 (d, J=1.76 Hz, 1H), 7.19-7.35 (m, 3H), 6.99 (dd, J=2.05, 7.91 Hz, 1H).

### (ii) 4-(4-fluorophenyl)-6-(3-hydroxyphenyl)-2-methoxypyridine-3-carbonitrile

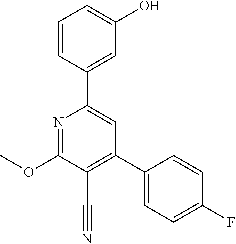 [SMILES: COc1nc(-c2cccc(O)c2)cc(-c2ccc(F)cc2)c1C#N]

A mixture of (E)-3-(4-fluorophenyl)-1-(3-hydroxyphenyl)prop-2-en-1-one (271 mg, 1.12 mmol) in methanol (3 mL) and 5.4 M sodium methoxide in methanol (0.20 mL) was treated with malononitrile (74 mg, 1.12 mmol). The reaction was stirred for 16 hours. The reaction was concentrated, then the residue was partitioned between ethyl acetate (25 mL) and 0.5 N HCl (20 mL). The aqueous layer was extracted with ethyl acetate (25 mL). The combined organic extracts were washed with brine (2 mL), dried (Na₂SO₄), and concentrated. The crude material was purified by column on silica (0-30% ethyl acetate:hexane), followed by column on silica (0-3% methanol:dichloromethane), to give 4-(4-fluorophenyl)-6-(3-hydroxyphenyl)-2-methoxypyridine-3-carbonitrile (98 mg, 27%) as a white solid. MS: M+H⁺ 321. ¹H NMR (300 MHz, CDCl₃) δ 7.57-7.71 (m, 5H), 7.36 (t, J=8.20 Hz, 1H), 7.16-7.29 (m, 3H), 6.91-7.02 (m, 1H), 4.20 (s, 3H).

### 4-(4-Fluorophenyl)-6-(4-hydroxyphenyl)-2-methoxypyridine-3-carbonitrile

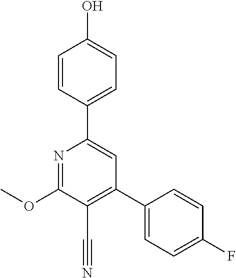 [SMILES: COc1nc(-c2ccc(O)cc2)cc(-c2ccc(F)cc2)c1C#N]

### (i) (E)-3-(4-fluorophenyl)-1-(4-hydroxyphenyl)prop-2-en-1-one

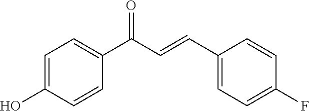 [SMILES: O=C(/C=C/c1ccc(F)cc1)c1ccc(O)cc1]

To a mixture of sodium hydroxide (0.22 g, 5.50 mmol) in water (2 mL) and ethanol (1 mL) at 0° C. was added 1-(4-hydroxyphenyl)ethanone (0.59 g, 4.30 mmol) and 4-fluorobenzaldehyde (0.53 g, 0.46 mL, 4.30 mmol). The reaction was allowed to warm to 20° C., and was stirred for 60 hours. The mixture was treated with 1 N HCl (5 mL), then was filtered, and the solid was washed with water (5 mL), to give (E)-3-(4-fluorophenyl)-1-(4-hydroxyphenyl)prop-2-en-1-one (0.91 g, 87%) as a pale yellow solid. MS: M+H⁺ 243. ¹H NMR (300 MHz, DMSO-d₆) δ 10.38 (br. s, 1H), 8.03 (d, J=8.79 Hz, 2H), 7.80-7.96 (m, 3H), 7.59-7.68 (m, 1H), 7.25 (t, J=8.79 Hz, 2H), 6.85 (d, J=8.79 Hz, 2H).

### (ii) 4-(4-Fluorophenyl)-6-(4-hydroxyphenyl)-2-methoxypyridine-3-carbonitrile

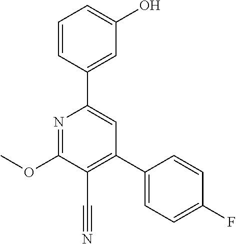 [SMILES: COc1nc(-c2cccc(O)c2)cc(-c2ccc(F)cc2)c1C#N]

A mixture of (E)-3-(4-fluorophenyl)-1-(4-hydroxyphenyl)prop-2-en-1-one (271 mg, 1.12 mmol) in methanol (3 mL) and 5.4 M NaOMe in methanol (0.20 mL) was treated with malononitrile (74 mg, 1.12 mmol). The reaction was stirred for 40 hours. The mixture was concentrated, then the residue was partitioned between ethyl acetate (40 mL) and 0.5 N HCl (20 mL). The organic layer was washed with brine (3 mL), dried (Na₂SO₄), and concentrated. The crude material was purified by column on silica (0-3% methanol:dichloromethane), to give 4-(4-fluorophenyl)-6-(4-hydroxyphenyl)-2-methoxypyridine-3-carbonitrile (68 mg, 19%) as a yellow solid. MS: M+H⁺ 321. ¹H NMR (300 MHz, DMSO-d₆) δ 9.99-10.13 (m, 1H), 8.08-8.19 (m, 2H), 7.71-7.84 (m, 2H), 7.60-7.71 (m, 1H), 7.31-7.49 (m, 2H), 6.78-6.97 (m, 2H), 4.11 (s, 3H).

### 2-Methoxy-6-(3-methylpyridin-2-yl)-4-(4-nitrophenyl)pyridine-3-carbonitrile

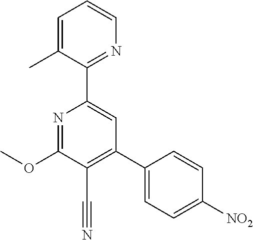 [SMILES: COc1nc(-c2ncccc2C)cc(-c2ccc([N+](=O)[O-])cc2)c1C#N]

### (i) (E)-1-(3-Methylpyridin-2-yl)-3-(4-nitrophenyl)prop-2-en-1-one

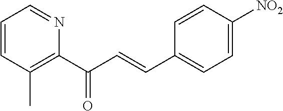 [SMILES: Cc1cccnc1C(=O)/C=C/c1ccc([N+](=O)[O-])cc1]

A mixture of 1-(3-methylpyridin-2-yl)ethanone (562 mg, 4.16 mmol) and 4-nitrobenzaldehyde (630 mg, 4.16 mmol) in methanol (14 mL) was treated with crushed NaOH (333 mg, 8.32 mmol). The reaction was stirred for 16 hours, then was filtered. The solid was washed with cold methanol (5 mL), to give (E)-1-(3-methylpyridin-2-yl)-3-(4-nitrophenyl)prop-2-en-1-one (1.04 g, 93%) as a pale yellow solid. MS: M+H⁺ 269. ¹H NMR (300 MHz, DMSO-d₆) δ 8.57-8.65 (m, 1H), 8.25 (d, J=8.20 Hz, 2H), 8.14 (s, 1H), 7.99-8.12 (m, 3H), 7.83 (d, J=7.62 Hz, 1H), 7.70-7.79 (m, 1H), 7.50-7.59 (m, 1H), 2.53 (s, 3H).

### (ii) 2-Methoxy-6-(3-methylpyridin-2-yl)-4-(4-nitrophenyl)pyridine-3-carbonitrile

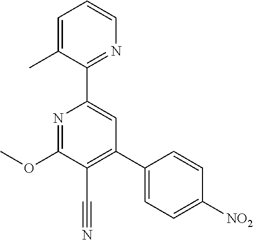 [SMILES: COc1nc(-c2ncccc2C)cc(-c2ccc([N+](=O)[O-])cc2)c1C#N]

A mixture of (E)-1-(3-methylpyridin-2-yl)-3-(4-nitrophenyl)prop-2-en-1-one (300 mg, 1.12 mmol) in methanol (3 mL) and 5.4 M NaOMe in methanol (0.20 mL) was treated with malononitrile (74 mg, 1.12 mmol). The reaction was stirred for 16 hours. The mixture was treated with methanol (5 mL), then was filtered, and the solid was washed with cold methanol (5 mL). The solid was treated with hot ethanol (10 mL), then was allowed to cool to 20° C. The mixture was filtered, then the solid was washed with cold ethanol (10 mL), to give 2-methoxy-6-(3-methylpyridin-2-yl)-4-(4-nitrophenyl)pyridine-3-carbonitrile (139 mg, 36%) as a beige solid. MS: M+H⁺ 347. ¹H NMR (300 MHz, DMSO-d₆) δ 8.53-8.59 (m, 1H), 8.41 (d, J=8.79 Hz, 2H), 7.96-8.03 (m, 2H), 7.89 (s, 1H), 7.82 (d, J=7.62 Hz, 1H), 7.41-7.49 (m, 1H), 4.11 (s, 3H), 2.67 (s, 3H).

### 4-(4-Aminophenyl)-2-methoxy-6-(3-methylpyridin-2-yl)pyridine-3-carbonitrile

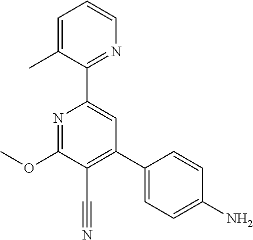 [SMILES: COc1nc(-c2ncccc2C)cc(-c2ccc(N)cc2)c1C#N]

### iii) tert-Butyl 4-((E)-3-(3-methylpyridin-2-yl)-3-oxoprop-1-enyl)phenylcarbamate

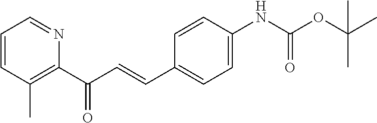 [SMILES: Cc1cccnc1C(=O)/C=C/c1ccc(NC(=O)OC(C)(C)C)cc1]

A mixture of tert-butyl 4-formylphenylcarbamate (1.20 g, 5.42 mmol) in methanol (30 mL) was treated with 1-(3-methylpyridin-2-yl)ethanone (1.10 g, 8.14 mmol), and a solution of KOH (457 mg, 8.14 mmol) in water (0.3 mL). The reaction was stirred for 3 days. The mixture was treated with water (50 mL), then was concentrated to remove the methanol. The residue was extracted with ethyl acetate (2×50 mL). The combined organic extracts were washed with brine (10 mL), dried (Na₂SO₄), and concentrated. The crude material was purified by column on silica (0-20% ethyl acetate/hexanes), to give tert-butyl 4-((E)-3-(3-methylpyridin-2-yl)-3-oxoprop-1-enyl)phenylcarbamate (1.15 g, 63%) as a yellow solid. MS: [M+H⁺] 339. ¹H NMR (300 MHz, CDCl₃) δ 8.48-8.60 (m, 1H), 7.51-7.85 (m, 5H), 7.40 (d, J=8.79 Hz, 2H), 7.31 (dd, J=4.39, 7.91 Hz, 1H), 6.84 (s, 1H), 2.58 (s, 3H), 1.50 (s, 9H).

### iv) tert-Butyl 4-(3-cyano-2-methoxy-6-(3-methylpyridin-2-yl)pyridin-4-yl)phenylcarbamate

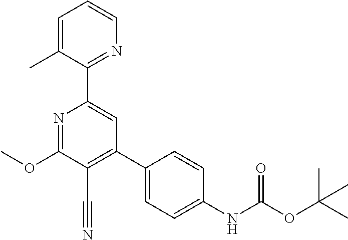 [SMILES: COc1nc(-c2ncccc2C)cc(-c2ccc(NC(=O)OC(C)(C)C)cc2)c1C#N]

A mixture of tert-butyl 4-((E)-3-(3-methylpyridin-2-yl)-3-oxoprop-1-enyl)phenylcarbamate (429 mg, 1.27 mmol) in methanol (3.4 mL) and 5.4M sodium methoxide in methanol (0.24 mL) was treated with malononitrile (83 mg, 1.27 mmol). The reaction was stirred for 16 hours, then was filtered. The combined solids were washed with cold methanol (10 mL). The crude solid was purified by column on silica (0-2% methanol:dichloromethane), to give tert-butyl 4-(3-cyano-2-methoxy-6-(3-methylpyridin-2-yl)pyridin-4-yl)phenylcarbamate (623 mg, 38%) as a pale yellow solid. MS: [M+H⁺] 417. ¹H NMR (300 MHz, cdcl₃) δ 8.50-8.58 (m, 1H), 7.78 (s, 1H), 7.60-7.70 (m, 2H), 7.51 (d, J=8.79 Hz, 1H), 7.26-7.32 (m, 1H), 6.56-6.66 (m, 1H), 4.13 (s, 3H), 2.67 (s, 3H), 1.36-1.66 (m, 9H).

### v) 4-(4-Aminophenyl)-2-methoxy-6-(3-methylpyridin-2-yl)pyridine-3-carbonitrile

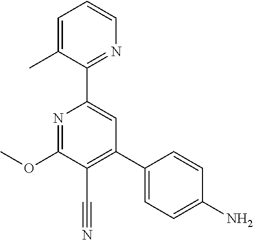 [SMILES: COc1nc(-c2ncccc2C)cc(-c2ccc(N)cc2)c1C#N]

To a mixture of tert-butyl 4-(3-cyano-2-methoxy-6-(3-methylpyridin-2-yl)pyridin-4-yl)phenylcarbamate (615 mg, 1.48 mmol) in dichloromethane (7 mL) and methanol (3 mL) was added a 4N solution of HCl in 1,4-dioxane (3.75 mL). The reaction was stirred for 3 days. The mixture was filtered, then the solid was washed with dichloromethane (10 mL), to give 4-(4-aminophenyl)-2-methoxy-6-(3-methylpyridin-2-yl)pyridine-3-carbonitrile (572 mg, 99%) as a white solid. MS: [M+H⁺] 317. ¹H NMR (300 MHz, dmso) δ 8.62-8.69 (m, 1H), 8.09 (d, J=7.62 Hz, 1H), 7.82 (s, 1H), 7.75 (d, J=8.20 Hz, 2H), 7.66 (dd, J=4.98, 7.91 Hz, 1H), 7.28 (d, J=8.20 Hz, 2H), 4.09 (s, 3H), 2.65 (s, 3H).

### 4-tert-Butyl-2-methoxy-6-(pyridin-2-yl)pyridine-3-carbonitrile

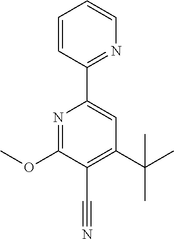 [SMILES: COc1nc(-c2ccccn2)cc(C(C)(C)C)c1C#N]

### (i) (E)-4,4-Dimethyl-1-(pyridin-2-yl)pent-2-en-1-one

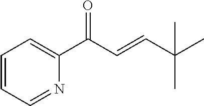

To a solution of 2-acetylpyridine (0.50 g, 0.47 mL, 4.15 mmol) and NaOH (166 mg, 4.15 mmol) in methanol (5 mL) was added pivaldehyde (0.36 g, 0.46 mL, 4.15 mmol). The reaction was stirred for 40 hours. The mixture was diluted with ethyl acetate (100 mL), then was washed with saturated aqueous NaHCO₃ (15 mL), water (15 mL), and brine (10 mL), dried (Na₂SO₄), and concentrated. The crude material was purified by column on silica (0-10% ethyl acetate/hexane) to give (E)-4,4-dimethyl-1-(pyridin-2-yl)pent-2-en-1-one (115 mg, 15%) as a clear liquid. MS: [M+H⁺] 190. ¹H NMR (300 MHz, CDCl₃) δ 8.63-8.80 (m, 1H), 8.12 (d, J=7.62 Hz, 1H), 7.75-7.94 (m, 1H), 7.38-7.63 (m, 2H), 7.16-7.32 (m, 1H), 1.17 (s, 9H).

### (ii) 4-tert-Butyl-2-methoxy-6-(pyridin-2-yl)pyridine-3-carbonitrile

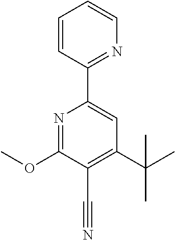 [SMILES: COc1nc(-c2ccccn2)cc(C(C)(C)C)c1C#N]

A mixture of (E)-4,4-dimethyl-1-(pyridin-2-yl)pent-2-en-1-one (102 mg, 0.54 mol) in 0.5M NaOMe in methanol (1 ml) and methanol (1 mL) was treated with malononitrile (40 mg, 0.59 mmol). The reaction mixture was stirred for 24 hours, then concentrated. The residue was partitioned between ethyl acetate (20 mL) and water (10 mL). The organic layer was washed with brine (1 mL), dried (Na₂SO₄), and concentrated. The crude material was purified by column on silica (0-15% ethyl acetate/hexane). The product was dissolved in dichloromethane (3 mL) and methanol (1 mL), then was treated with a 4N solution of HCl in 1,4-dioxane (0.25 mL). The mixture was allowed to stand for 5 minutes, then was concentrated, to give 4-tert-butyl-2-methoxy-6-(pyridin-2-yl)pyridine-3-carbonitrile as the HCl salt (22 mg, 14%) as a white solid. [M+H⁺] 268. ¹H NMR (300 MHz, dmso) δ 8.68-8.82 (m, 1H), 8.35-8.52 (m, 1H), 8.12-8.25 (m, 1H), 7.95-8.10 (m, 1H), 7.46-7.62 (m, 1H), 4.10 (s, 3H), 1.48 (s, 9H).

The disclosures of each and every patent, patent application, and publication cited herein are hereby incorporated herein by reference in their entirety. While this invention has been disclosed with reference to specific embodiments, it is apparent that other embodiments and variations of this invention can be devised by others skilled in the art without departing from the true spirit and scope of the invention. The appended claims are intended to be construed to include all such embodiments and equivalent variations.

1. A compound of formula (IA), or a salt, solvate, stereoisomer, geometric isomer, and/or tautomer thereof: 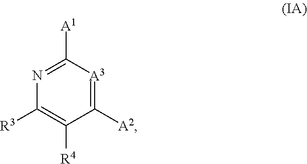
wherein in (IA):
A¹ is —C(R⁷)(R⁸)(R⁹), 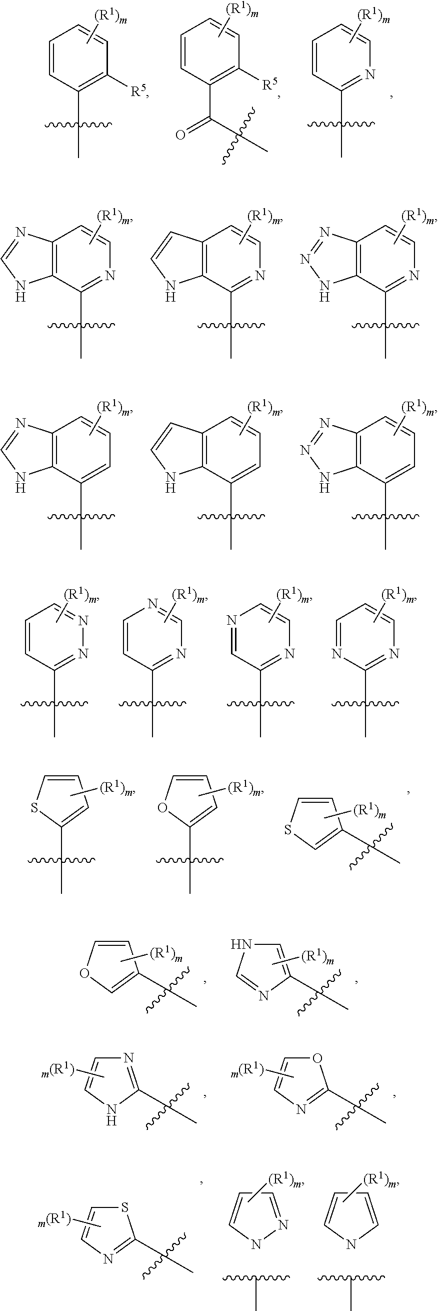 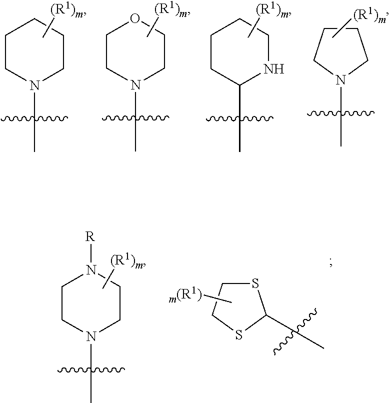
A² is —C(R¹⁰)(R¹¹)(R¹²), 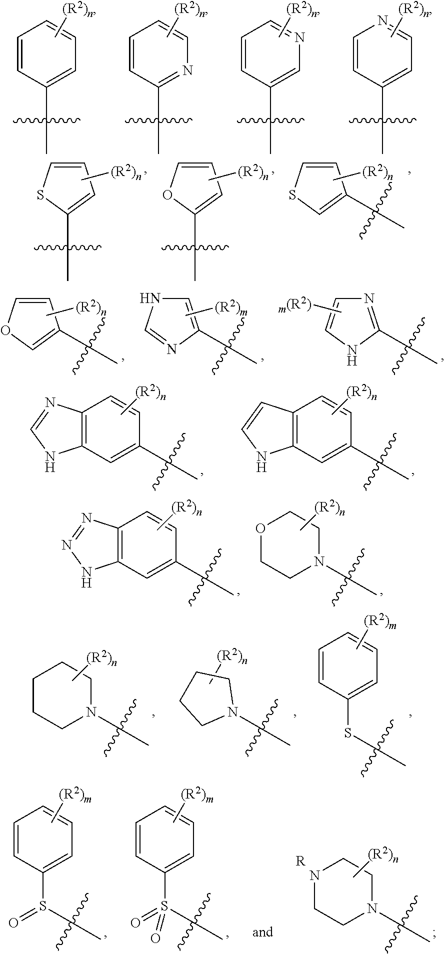
A³ is N or C—R⁶;
m is 1, 2 or 3;
n is 1, 2 or 3;
each occurrence of R¹ is independently selected from the group consisting of H, F, Cl, Br, I, —OR, —SR, —S(O)(C₁-C₆ alkyl), —S(O)₂C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R¹ groups are present in neighboring carbons, they optionally combine to form the divalent group —O(CH₂)₁₋₃O—;
each occurrence of R² is independently selected from the group consisting of H, F, Cl, Br, I, —OR, —SR, —S(O)(C₁-C₆ alkyl), —S(O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R² groups are present in neighboring carbons, they optionally combine to form the divalent group —O(CH₂)₁₋₃O—;
with the proviso that R² is not positioned ortho to the bond between the central heteroaryl ring and A²;
R⁴ is independently selected from the group consisting of —CH₃, —CN, —C≡CR, —C(═O)OR, and —C(═O)NR₂; or R⁴ can combine with R³ to form 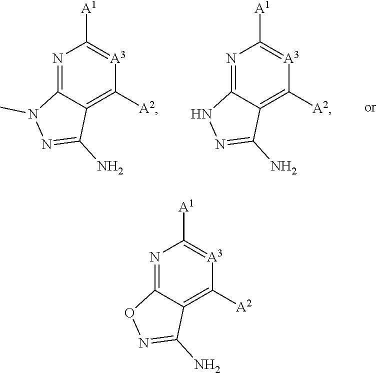
R³, R⁵ and R⁶ are selected such that:
(a) R⁵ and R⁶ combine to form the divalent group —O—, —CH₂—, —CH(C₁-C₆ alkyl) or —C(C₁-C₆ alkyl)(C₁-C₆ alkyl)-, and
R³ is selected from the group consisting of C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆ alkyl), —S(O)(C₁-C₆ alkyl), —S(O)₂(C₁-C₆ alkyl), N¹-β-propiolactam, N¹-γ-butyrolactam, N¹-δ-valerolactam, oxetanoyl, oxetanoxyl N¹-ε-caprolactam, (C₂-C₆)alkenyloxy, (C₂-C₆)alkynyloxy, —O(CH₂)₂₋₄OR, —O(CH₂)₂₋₄O(CH₂)₁₋₄C(═O)OR, —O(CH₂)₂₋₄NRR, —O(CH₂)₁₋₄(aryl), O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, N¹-pyrrolidinyl, —O(CH₂)₂₋₄(N¹-pyrrolidinyl), —N¹-piperidinyl, —O(CH₂)₂₋₄(N⁴-morpholinyl), —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;
(b) R⁵ and R⁶ combine to form the divalent group —CH₂CH₂—, and
R³ is selected from the group consisting of C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆ alkyl), —S(O)(C₁-C₆ alkyl), —S(O)₂(C₁-C₆ alkyl), N¹-β-propiolactam, N¹-γ-butyrolactam, N¹-δ-valerolactam, oxetanoyl, oxetanoxyl, N¹-δ-caprolactam, (C₂-C₆)alkenyloxy, (C₂-C₆)alkynyloxy, —O(CH₂)₂₋₄OR, —O(CH₂)₂₋₄O(CH₂)₂₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl, —O(CH₂)₂₋₄(N¹-pyrrolidinyl), —N¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR,
(c) R⁵ and R⁶ combine to form the divalent group —CR₂CR₂—, wherein at least one R is not H;
R³ is selected from the group consisting of C₁-C₆ alkyl, (C₁-C₆)alkoxy, (C₁-C₆)haloalkyl, (C₁-C₆)haloalkoxy, dialkylamino(C₁-C₆)alkoxy, —NR₂, N¹-β-propiolactam, N¹-γ-butyrolactam, N¹-δ-valerolactam, oxetanoyl, oxetanoxyl N¹-ε-caprolactam, (C₂-C₆)alkenyloxy, (C₂-C₆)alkynyloxy, —O(CH₂)₂₋₄OR, —O(CH₂)₂₋₄O(CH₂)₁₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl, —O(CH₂)₂₋₄(N¹-pyrrolidinyl), N¹-piperidinyl, —O(CH₂)₂₋₄(N¹-δ-piperidinyl), —O(CH₂)₂₋₄(N¹-δ-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR, and
(d) R⁵ is R₁, and R⁶ is H or C₁-C₆ alkyl, and
R³ is selected from the group consisting of C₁-C₆ alkyl, (C₁-C₆)alkoxy, (C₁-C₆)haloalkyl, (C₁-C₆)haloalkoxy, dialkylamino(C₁-C₆)alkoxy, —NR₂, N¹-β-propiolactam, N¹-γ-butyrolactam, N¹-δ-valerolactam, oxetanoyl, oxetanoxyl, N-ε-caprolactam, (C₂-C₆)alkenyloxy, —(C₂-C₆)alkynyloxy, —O(CH₂)₂₋₄OR, —O(CH₂)₂₋₄O(CH₂)₁₋₄C(═O)OR, —O(CH₂)₁₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl, —O(CH₂)₂₋₄(N¹-pyrrolidinyl), N¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam) N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;
each of R⁷, R⁸, R⁹, R¹⁰, R¹¹, and R¹² is independently selected from optionally substituted C₁-C₁₀ alkyl, wherein two or three of R⁷, R⁸, R⁹ or of R¹⁰, R¹¹, R¹² can optionally combine to form monocyclic or polycyclic groups (such as, for example, cyclohexyl or adamantyl);
each occurrence of R is independently H or C₁-C₆ alkyl,
with the provisos that
i) when R³ is (C₁-C₆)alkoxy, R⁴ is CN and A¹ is 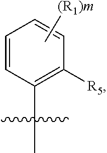
then A² is not 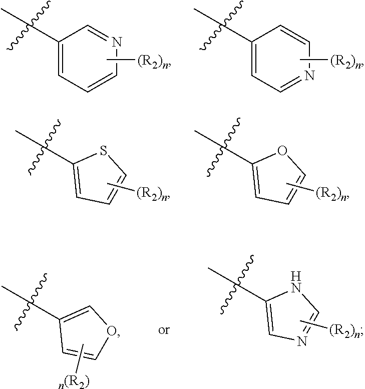
ii) when R³ is (C₁-C₆)alkoxy, R⁴ is CN and A¹ is 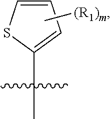
then A² is not 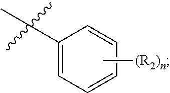
iii) when R³ is (C₁-C₆)alkoxy, R⁴ is CN and A¹ is 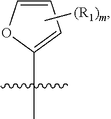
then A² is not; 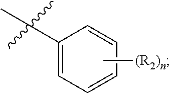
iv) when R³ is (C₁-C₆)alkoxy, R⁴ is CN and A¹ is 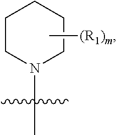
then A² is not 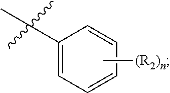
v) when R³ is (C₁-C₆)alkoxy, R⁴ is CN and A¹ is 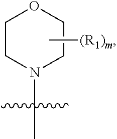
then A is not; 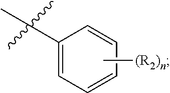
vi) when R³ is (C₁-C₆)alkoxy, R⁴ is CN, A¹ is 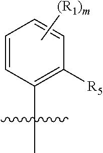
and R⁵ and R⁶ combine to form the divalent group —CH₂CH₂—,
then A² is not 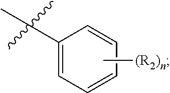
vii) when R³ is (C₁-C₆)alkoxy, R⁴ is C(═O)OR and A¹ is 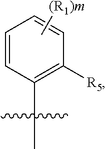
then A² is not 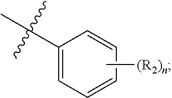
vii) when R³ is (C₁-C₆)alkoxy, R⁴ is CN and A¹ is 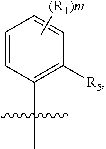
then A² is not 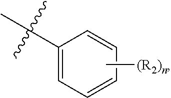
except for the compounds selected from the group consisting of:

| | |
|---|---|
| 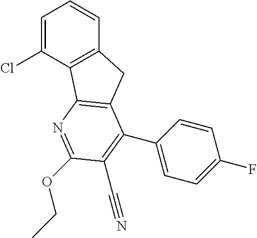 [SMILES: CCOc1nc2c(c(-c3ccc(F)cc3)c1C#N)Cc1cccc(Cl)c1-2] | 9-Chloro-2-ethoxy-4-(4-fluorophenyl)-5H- indeno[1,2-b]pyridine-3-carbonitrile; |
| 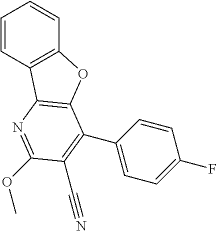 [SMILES: COc1nc2c(oc3ccccc32)c(-c2ccc(F)cc2)c1C#N] | 4-(4-Fluorophenyl)-2-methoxybenzofuro[3,2- b]pyridine-3-carbonitrile; |
| 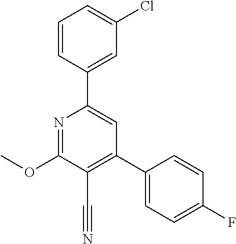 [SMILES: COc1nc(-c2cccc(Cl)c2)cc(-c2ccc(F)cc2)c1C#N] | 6-(3-Chlorophenyl)-4-(4-fluorophenyl)-2- methoxypyridine-3-carbonitrile; |
| 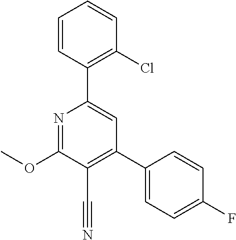 [SMILES: COc1nc(-c2ccccc2Cl)cc(-c2ccc(F)cc2)c1C#N] | 6-(2-Chlorophenyl)-4-(4-fluorophenyl)-2- methoxypyridine-3-carbonitrile; |
| 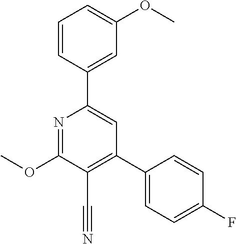 [SMILES: COc1cccc(-c2cc(-c3ccc(F)cc3)c(C#N)c(OC)n2)c1] | 4-(4-Fluorophenyl)-2-methoxy-6-(3- methoxyphenyl)pyridine-3-carbonitrile; |
| 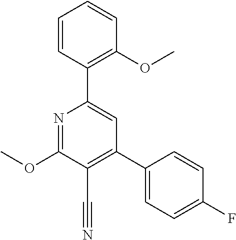 [SMILES: COc1ccccc1-c1cc(-c2ccc(F)cc2)c(C#N)c(OC)n1] | 4-(4-Fluorophenyl)-2-methoxy-6-(2- methoxyphenyl)pyridine-3-carbonitrile; |
| 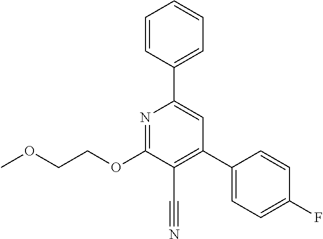 [SMILES: COCCOc1nc(-c2ccccc2)cc(-c2ccc(F)cc2)c1C#N] | 2-(2-Methoxyethoxy)-4-(4-fluorophenyl)-6- phenylpyridine-3-carbonitrile; |
| 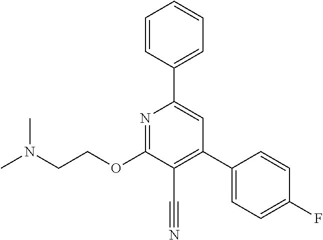 [SMILES: CN(C)CCOc1nc(-c2ccccc2)cc(-c2ccc(F)cc2)c1C#N] | 2-(2-(Dimethylamino)ethoxy)-4-(4- fluorophenyl)-6-phenylpyridine-3-carbonitrile; |
| 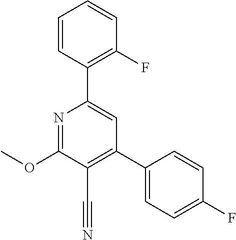 [SMILES: COc1nc(-c2ccccc2F)cc(-c2ccc(F)cc2)c1C#N] | 6-(2-Fluorophenyl)-4-(4-fluorophenyl)-2- methoxypyridine-3-carbonitrile; |
| 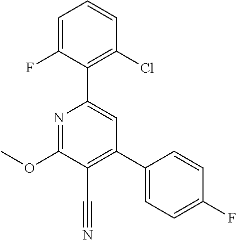 [SMILES: COc1nc(-c2c(F)cccc2Cl)cc(-c2ccc(F)cc2)c1C#N] | 6-(2-Chloro-6-fluoro-phenyl)-4-(4-fluoro- phenyl)-2-methoxy-nicotinonitrile; |
| 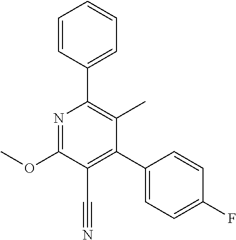 [SMILES: COc1nc(-c2ccccc2)c(C)c(-c2ccc(F)cc2)c1C#N] | 4-(4-Fluoro-phenyl)-2-methoxy-5-methyl-6- phenyl-nicotinonitrile; |
| 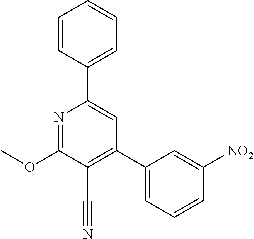 [SMILES: COc1nc(-c2ccccc2)cc(-c2cccc([N+](=O)[O-])c2)c1C#N] | 2-Methoxy-4-(3-nitrophenyl)-6-phenylpyridine- 3-carbonitrile; |
| 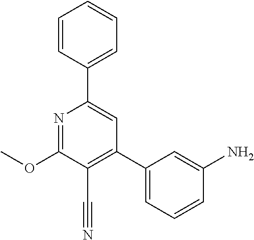 [SMILES: COc1nc(-c2ccccc2)cc(-c2cccc(N)c2)c1C#N] | 4-(3-Aminophenyl)-2-methoxy-6-phenylpyridine- 3-carbonitrile; |
| 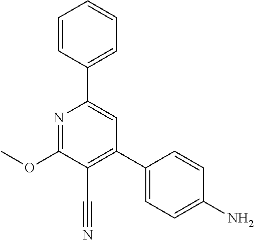 [SMILES: COc1nc(-c2ccccc2)cc(-c2ccc(N)cc2)c1C#N] | 4-(4-Aminophenyl)-2-methoxy-6-phenylpyridine- 3-carbonitrile; |
| 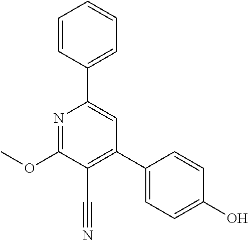 [SMILES: COc1nc(-c2ccccc2)cc(-c2ccc(O)cc2)c1C#N] | 4-(4-Hydroxyphenyl)-2-methoxy-6- phenylpyridine-3-carbonitrile; |
| 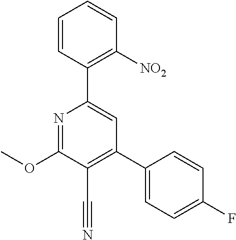 [SMILES: COc1nc(-c2ccccc2[N+](=O)[O-])cc(-c2ccc(F)cc2)c1C#N] | 4-(4-Fluorophenyl)-2-methoxy-6-(2- nitrophenyl)pyridine-3-carbonitrile; |
| 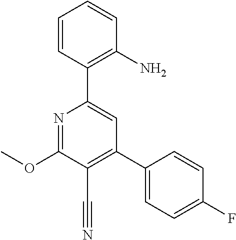 [SMILES: COc1nc(-c2ccccc2N)cc(-c2ccc(F)cc2)c1C#N] | 6-(2-Aminophenyl)-4-(4-fluorophenyl)-2- methoxypyridine-3-carbonitrile; |
| 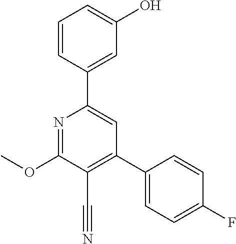 [SMILES: COc1nc(-c2cccc(O)c2)cc(-c2ccc(F)cc2)c1C#N] | 4-(4-fluorophenyl)-6-(3-hydroxyphenyl)-2- methoxypyridine-3-carbonitrile; |
| 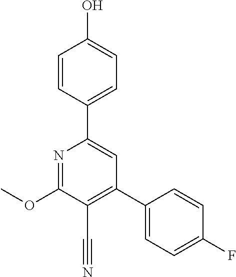 [SMILES: COc1nc(-c2ccc(O)cc2)cc(-c2ccc(F)cc2)c1C#N] | 4-(4-Fluorophenyl)-6-(4-hydroxyphenyl)-2- methoxypyridine-3-carbonitrile; |

viii) when R³ is (C₁-C₆)alkoxy, R⁴ is CN and A¹ is 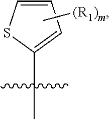
then A is not 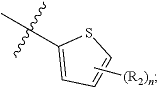
ix) when R³ is (C₁-C₆)alkoxy, R⁴ is CN and A¹ is 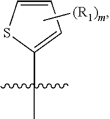
then A is not 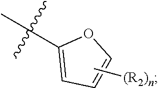
x) when R³ is (C₁-C₆)alkoxy, R⁴ is CN and A¹ is 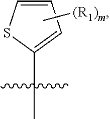
then A² is not 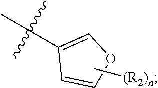
xi) when R³ is (C₁-C₆)alkoxy, R⁴ is CN and A¹ is 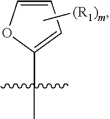
then A² is not; 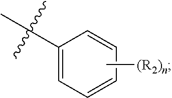
xii) when R³ is (C₁-C₆)alkoxy, R⁴ is CN and A¹ is 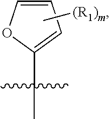
then A is not 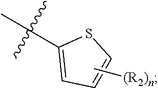
xiii) when R³ is (C₁-C₆)alkoxy, R⁴ is CN and A¹ is 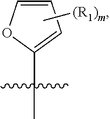
then A² is not 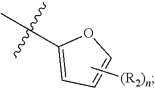
xiv) when R³ is (C₁-C₆)alkoxy, R⁴ is CN and A¹ is 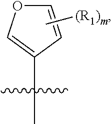
then A² is not, 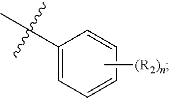
xv) when R³ is (C₁-C₆)alkoxy, R⁴ is CN and A¹ is 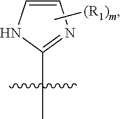
then A² is not; 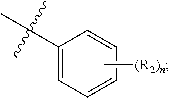
xvi) when R³ is (C₁-C₆)alkoxy, R⁴ is CN and A¹ is 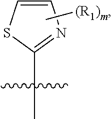
then A² is not 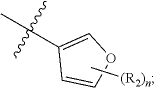
and with the proviso that the compound is not any of the following compounds:

| | |
|---|---|
| 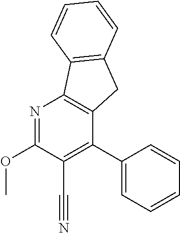 [SMILES: COc1nc2c(c(-c3ccccc3)c1C#N)Cc1ccccc1-2] | 2-Methoxy-4-phenyl-5H-indeno[1,2-b]pyridine-3- carbonitrile; |
| 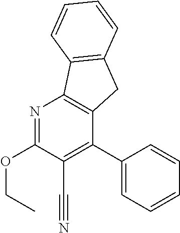 [SMILES: CCOc1nc2c(c(-c3ccccc3)c1C#N)Cc1ccccc1-2] | 2-Ethoxy-4-phenyl-5H-indeno[1,2-b]pyridine-3- carbonitrile; |
| 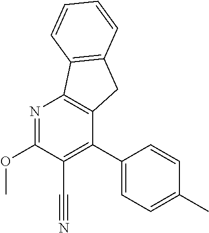 [SMILES: COc1nc2c(c(-c3ccc(C)cc3)c1C#N)Cc1ccccc1-2] | 2-Methoxy-4-p-tolyl-5H-indeno[1,2-b]pyridine-3- carbonitrile; |
| 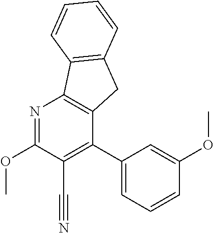 [SMILES: COc1cccc(-c2c(C#N)c(OC)nc3c2Cc2ccccc2-3)c1] | 2-Methoxy-4-(3-methoxy-phenyl)-5H-indeno[1,2- b]pyridine-3-carbonitrile; |
| 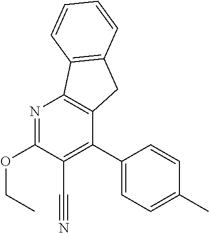 [SMILES: CCOc1nc2c(c(-c3ccc(C)cc3)c1C#N)Cc1ccccc1-2] | 2-Ethoxy-4-p-tolyl-5H-indeno[1,2-b]pyridine-3- carbonitrile; |
| 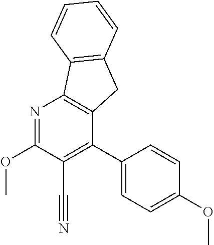 [SMILES: COc1ccc(-c2c(C#N)c(OC)nc3c2Cc2ccccc2-3)cc1] | 2-Methoxy-4-(4-methoxy-phenyl)-5H-indeno[1,2- b]pyridine-3-carbonitrile; |
| 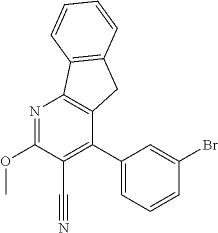 [SMILES: COc1nc2c(c(-c3cccc(Br)c3)c1C#N)Cc1ccccc1-2] | 4-(3-Bromo-phenyl)-2-methoxy-5H-indeno[1,2- b]pyridine-3-carbonitrile; |
| 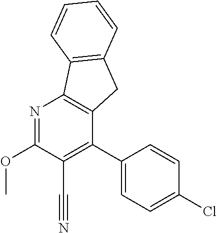 [SMILES: COc1nc2c(c(-c3ccc(Cl)cc3)c1C#N)Cc1ccccc1-2] | 4-(4-Chloro-phenyl)-2-methoxy-5H-indeno[1,2- b]pyridine-3-carbonitrile; |
| 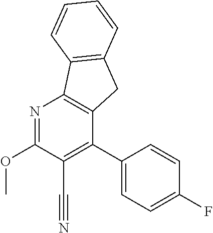 [SMILES: COc1nc2c(c(-c3ccc(F)cc3)c1C#N)Cc1ccccc1-2] | 4-(4-Fluoro-phenyl)-2-methoxy-5H-indeno[1,2- b]pyridine-3-carbonitrile; |
| 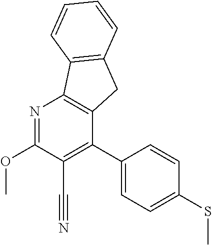 [SMILES: COc1nc2c(c(-c3ccc(SC)cc3)c1C#N)Cc1ccccc1-2] | 2-Methoxy-4-(4-methylsulfanyl-phenyl)-5H- indeno[1,2-b]pyridine-3-carbonitrile; |
| 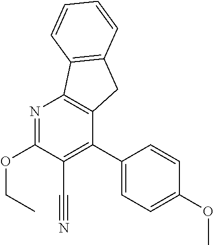 [SMILES: CCOc1nc2c(c(-c3ccc(OC)cc3)c1C#N)Cc1ccccc1-2] | 2-Ethoxy-4-(4-methoxy-phenyl)-5H-indeno[1,2- b]pyridine-3-carbonitrile; |
| 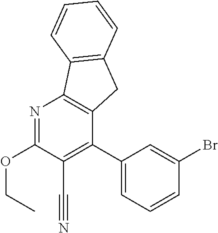 [SMILES: CCOc1nc2c(c(-c3cccc(Br)c3)c1C#N)Cc1ccccc1-2] | 4-(3-Bromo-phenyl)-2-ethoxy-5H-indeno[1,2- b]pyridine-3-carbonitrile; |
| 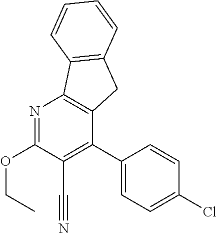 [SMILES: CCOc1nc2c(c(-c3ccc(Cl)cc3)c1C#N)Cc1ccccc1-2] | 4-(4-Chloro-phenyl)-2-ethoxy-5H-indeno[1,2- b]pyridine-3-carbonitrile; |
| 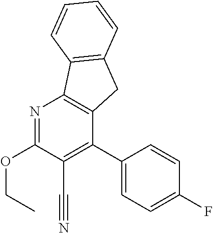 [SMILES: CCOc1nc2c(c(-c3ccc(F)cc3)c1C#N)Cc1ccccc1-2] | 2-Ethoxy-4-(4-fluoro-phenyl)-5H-indeno[1,2- b]pyridine-3-carbonitrile; |
| 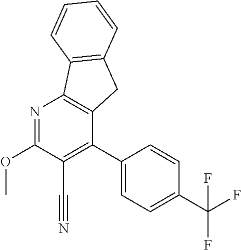 [SMILES: COc1nc2c(c(-c3ccc(C(F)(F)F)cc3)c1C#N)Cc1ccccc1-2] | 2-Methoxy-4-(4-trifluoromethyl-phenyl)-5H- indeno[1,2-b]pyridine-3-carbonitrile; |
| 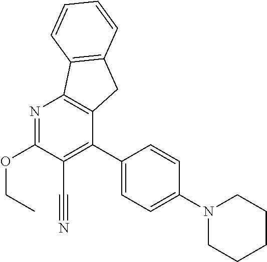 [SMILES: CCOc1nc2c(c(-c3ccc(N4CCCCC4)cc3)c1C#N)Cc1ccccc1-2] | 2-Ethoxy-4-(4-piperidin-1-yl-phenyl)-5H- indeno[1,2-b]pyridine-3-carbonitrile; |
| 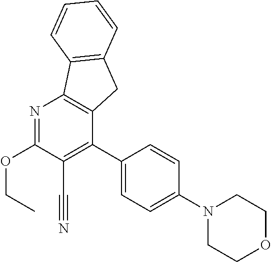 [SMILES: CCOc1nc2c(c(-c3ccc(N4CCOCC4)cc3)c1C#N)Cc1ccccc1-2] | 2-Ethoxy-4-(4-morpholin-4-yl-phenyl)-5H- indeno[1,2-b]pyridine-3-carbonitrile; |
| 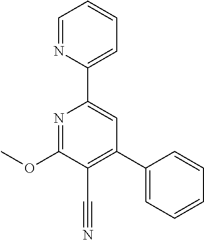 [SMILES: COc1nc(-c2ccccn2)cc(-c2ccccc2)c1C#N] | 6-Methoxy-4-phenyl-[2,2′]bipyridinyl- 5-carbonitrile; |
| 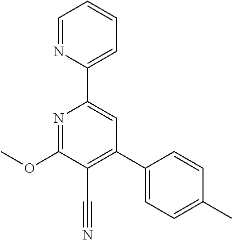 | 6-Methoxy-4-p-tolyl-[2,2′]bipyridinyl- 5-carbonitrile; |
| 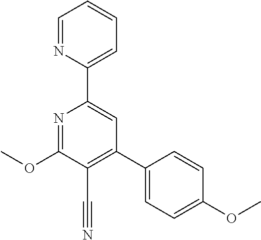 | 6-Methoxy-4-(4-methoxy-phenyl)- [2,2′]bipyridinyl-5-carbonitrile; |
| 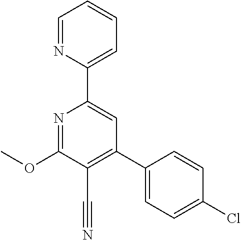 [SMILES: COc1nc(-c2ccccn2)cc(-c2ccc(Cl)cc2)c1C#N] | 4-(4-Chloro-phenyl)-6-methoxy- [2,2′]bipyridinyl-5-carbonitrile; |
| 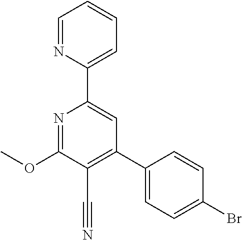 [SMILES: COc1nc(-c2ccccn2)cc(-c2ccc(Br)cc2)c1C#N] | 4-(4-Brorno-phenyl)-6-methoxy- [2,2′]bipyridinyl-5-carbonitrile; |
| 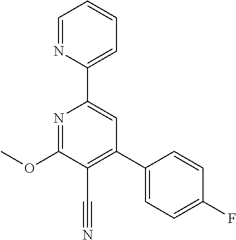 [SMILES: COc1nc(-c2ccccn2)cc(-c2ccc(F)cc2)c1C#N] | 4-(4-Fluoro-phenyl)-6-methoxy- [2,2′]bipyridinyl-5-carbonitrile; |
| 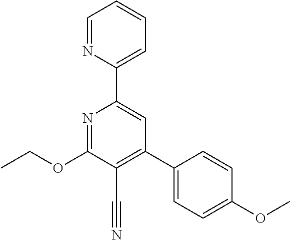 [SMILES: CCOc1nc(-c2ccccn2)cc(-c2ccc(OC)cc2)c1C#N] | 6-Ethoxy-4-(4-methoxy-phenyl)- [2,2′]bipyridinyl-5-carbonitrile; |
| 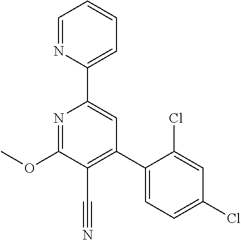 [SMILES: COc1nc(-c2ccccn2)cc(-c2ccc(Cl)cc2Cl)c1C#N] | 4-(2,4-Dichloro-phenyl)-6-methoxy- [2,2′]bipyridinyl-5-carbonitrile; |
| 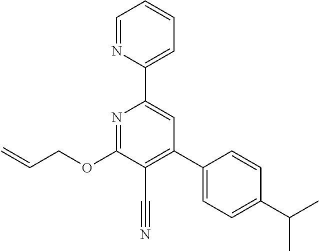 [SMILES: C=CCOc1nc(-c2ccccn2)cc(-c2ccc(C(C)C)cc2)c1C#N] | 6-Allyloxy-4-(4-isopropyl-phenyl)- [2,2′]bipyridinyl-5-carbonitrile; |
| 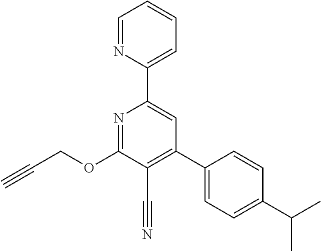 [SMILES: C#CCOc1nc(-c2ccccn2)cc(-c2ccc(C(C)C)cc2)c1C#N] | 4-(4-Isopropyl-phenyl)-6-prop-2- ynyloxy-[2,2′]bipyridinyl-5- carbonitrile; |
| 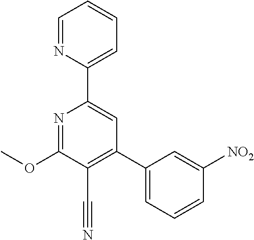 [SMILES: COc1nc(-c2ccccn2)cc(-c2cccc([N+](=O)[O-])c2)c1C#N] | 6-Methoxy-4-(3-nitro-phenyl)- [2,2′]bipyridinyl-5-carbonitrile; |
| 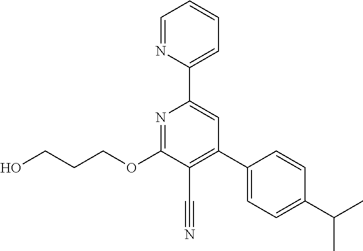 [SMILES: CC(C)c1ccc(-c2cc(-c3ccccn3)nc(OCCCO)c2C#N)cc1] | 6-(3-Hydroxy-propoxy)-4- (4-isopropyl-phenyl)- [2,2′]bipyridinyl-5- carbonitrile; |
| 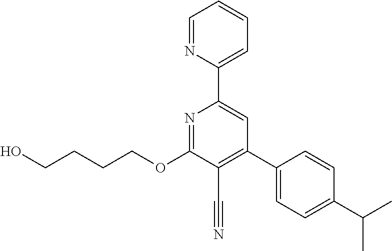 [SMILES: CC(C)c1ccc(-c2cc(-c3ccccn3)nc(OCCCCO)c2C#N)cc1] | 6-(4-Hydroxy-butoxy)-4- (4-isopropyl-phenyl)- [2,2′]bipyridinyl-5- carbonitrile; |
| 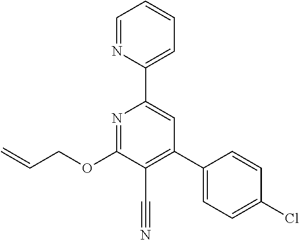 [SMILES: C=CCOc1nc(-c2ccccn2)cc(-c2ccc(Cl)cc2)c1C#N] | 6-Allyloxy-4-(4-chloro- phenyl)-[2,2′]bipyridinyl- 5-carbonitrile; |
| 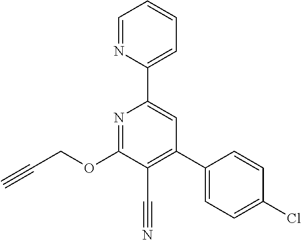 [SMILES: C#CCOc1nc(-c2ccccn2)cc(-c2ccc(Cl)cc2)c1C#N] | 4-(4-Chloro-phenyl)-6- prop-2-ynyloxy- [2,2′]bipyridinyl-5- carbonitrile; |
| 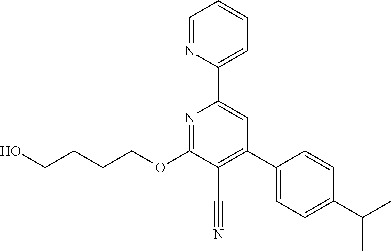 [SMILES: CC(C)c1ccc(-c2cc(-c3ccccn3)nc(OCCCCO)c2C#N)cc1] | 6-(4-Hydroxy-butoxy)-4- (4-isopropyl-phenyl)- [2,2′]bipyridinyl-5- carbonitrile; |
| 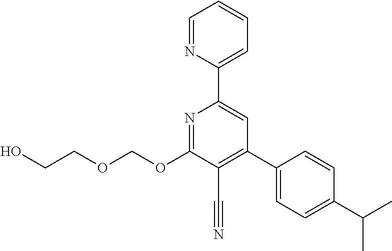 [SMILES: CC(C)c1ccc(-c2cc(-c3ccccn3)nc(OCOCCO)c2C#N)cc1] | 6-(2-Hydroxy- ethoxymethoxy)-4-(4- isopropyl-phenyl)- [2,2′]bipyridinyl-5- carbonitrile; |
| 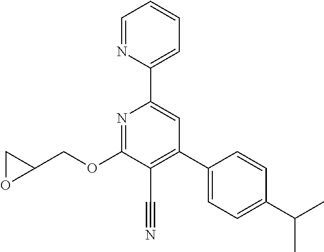 [SMILES: CC(C)c1ccc(-c2cc(-c3ccccn3)nc(OCC3CO3)c2C#N)cc1] | 4-(4-Isopropyl-phenyl)-6- oxiranylmethoxy- [2,2′]bipyridinyl-5- carbonitrile; |
| 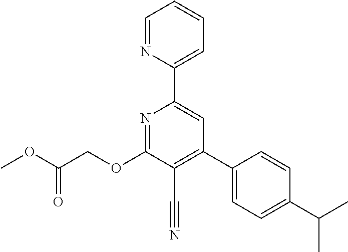 [SMILES: COC(=O)COc1nc(-c2ccccn2)cc(-c2ccc(C(C)C)cc2)c1C#N] | [5-Cyano-4-(4-isopropyl- phenyl)-[2,2′]bipyridinyl- 6-yloxy]-acetic acid methyl ester; |
| 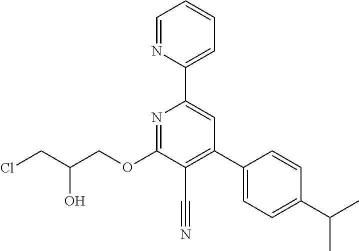 [SMILES: CC(C)c1ccc(-c2cc(-c3ccccn3)nc(OCC(O)CCl)c2C#N)cc1] | 6-(3-Chloro-2-hydroxy- propoxy)-4-(4-isopropyl- phenyl)-[2,2′]bipyridinyl- 5-carbonitrile; |
| 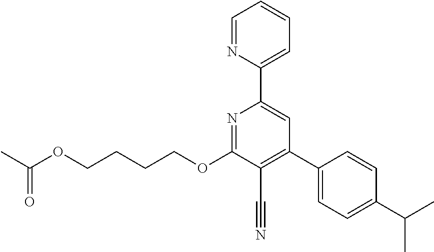 [SMILES: CC(=O)OCCCCOc1nc(-c2ccccn2)cc(-c2ccc(C(C)C)cc2)c1C#N] | Acetic acid 4-[5-cyano-4- (4-isopropyl-phenyl)- [2,2′]bipyridinyl-6- yloxy]-butyl ester; |
| 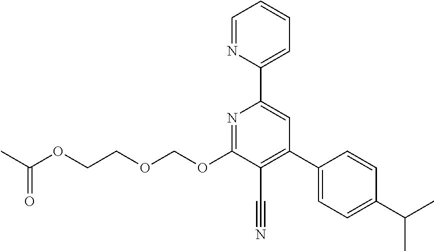 [SMILES: CC(=O)OCCOCOc1nc(-c2ccccn2)cc(-c2ccc(C(C)C)cc2)c1C#N] | Acetic acid 2-[5-cyano-4- (4-isopropyl-phenyl)- [2,2′]bipyridinyl-6- yloxymethoxy]-ethyl ester; |
| 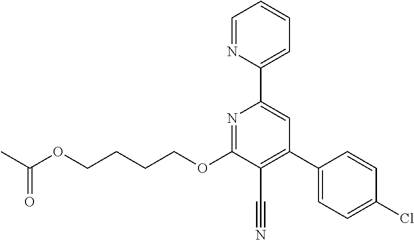 [SMILES: CC(=O)OCCCCOc1nc(-c2ccccn2)cc(-c2ccc(Cl)cc2)c1C#N] | Acetic acid 4-[4-(4- chloro-phenyl)-5-cyano- [2,2′]bipyridinyl-6- yloxy]-butyl ester; |
| 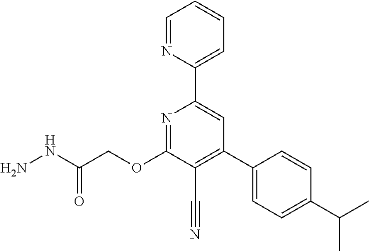 [SMILES: CC(C)c1ccc(-c2cc(-c3ccccn3)nc(OCC(=O)NN)c2C#N)cc1] | [5-Cyano-4-(4-isopropyl-phenyl)- [2,2′]bipyridinyl-6-yloxy]-acetic acid hydrazide; |
| 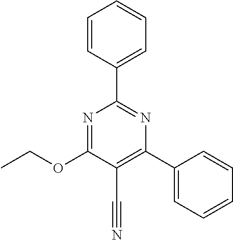 [SMILES: CCOc1nc(-c2ccccc2)nc(-c2ccccc2)c1C#N] | 4-Ethoxy-2,6-diphenyl-pyrimidine- 5-carbonitrile; |
| 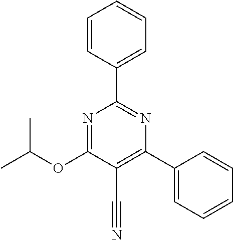 [SMILES: CC(C)Oc1nc(-c2ccccc2)nc(-c2ccccc2)c1C#N] | 4-Isopropoxy-2,6-diphenyl- pyrimidine-5-carbonitrile; |
| 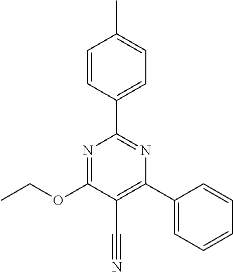 [SMILES: CCOc1nc(-c2ccc(C)cc2)nc(-c2ccccc2)c1C#N] | 4-Ethoxy-2,6-di-p-tolyl- pyrimidine-5-carbonitrile; |
| 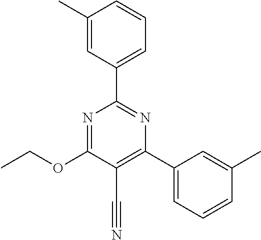 [SMILES: CCOc1nc(-c2cccc(C)c2)nc(-c2cccc(C)c2)c1C#N] | 4-Ethoxy-2,6-di-m-tolyl- pyrimidine-5-carbonitrile; |
| 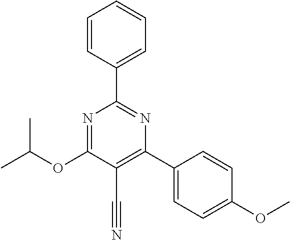 [SMILES: COc1ccc(-c2nc(-c3ccccc3)nc(OC(C)C)c2C#N)cc1] | 4-Isopropoxy-6-(4-methoxy- phenyl)-2-phenyl-pyrimidine-5- carbonitrile; |
| 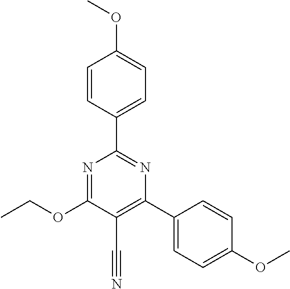 [SMILES: CCOc1nc(-c2ccc(OC)cc2)nc(-c2ccc(OC)cc2)c1C#N] | 4-Ethoxy-2,6-bis-(4-methoxy- phenyl)-pyrimidine-5-carbonitrile; |
| 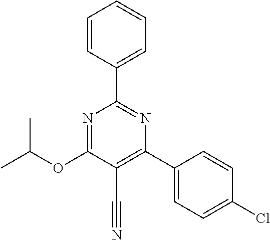 [SMILES: CC(C)Oc1nc(-c2ccccc2)nc(-c2ccc(Cl)cc2)c1C#N] | 4-(4-Chloro-phenyl)-6-isopropoxy- 2-phenyl-pyrimidine-5-carbonitrile; |
| 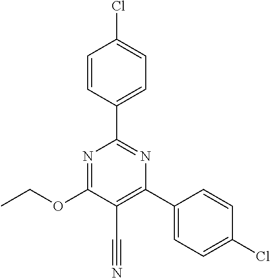 [SMILES: CCOc1nc(-c2ccc(Cl)cc2)nc(-c2ccc(Cl)cc2)c1C#N] | 2,4-Bis-(4-chloro-phenyl)-6- ethoxy-pyrimidine-5-carbonitrile; |
| 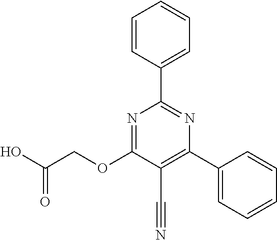 [SMILES: N#Cc1c(OCC(=O)O)nc(-c2ccccc2)nc1-c1ccccc1] | (5-Cyano-2,6-diphenyl-pyrimidin- 4-yloxy)-acetic acid; |
| 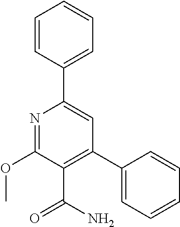 [SMILES: COc1nc(-c2ccccc2)cc(-c2ccccc2)c1C(N)=O] | 2-Methoxy-4,6-diphenyl- nicotinamide; |
| 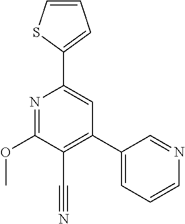 [SMILES: COc1nc(-c2cccs2)cc(-c2cccnc2)c1C#N] | 2′-Methoxy-6′-thiophen-2-yl- [3,4′]bipyridinyl-3′-carbonitrile; |
| 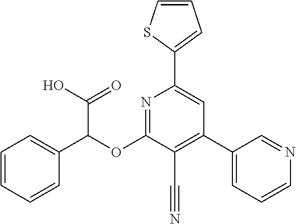 [SMILES: N#Cc1c(-c2cccnc2)cc(-c2cccs2)nc1OC(C(=O)O)c1ccccc1] | (3′-Cyano-6′-thiophen-2-yl-[3,4′]bipyridinyl-2′- yloxy)-phenyl-acetic acid; |
| 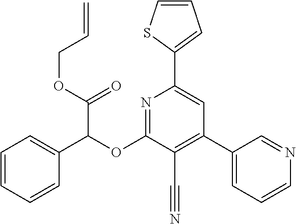 [SMILES: C=CCOC(=O)C(Oc1nc(-c2cccs2)cc(-c2cccnc2)c1C#N)c1ccccc1] | (3′-Cyano-6′-thiophen-2-yl-[3,4′]bipyridinyl-2′- yloxy)-phenyl-acetic acid allyl ester; |
| 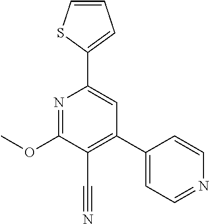 [SMILES: COc1nc(-c2cccs2)cc(-c2ccncc2)c1C#N] | 2-Methoxy-6-thiophen-2-yl-[4,4′]bipyridinyl-3- carbonitrile; |
| 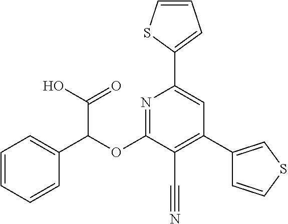 [SMILES: N#Cc1c(-c2ccsc2)cc(-c2cccs2)nc1OC(C(=O)O)c1ccccc1] | (3-Cyano-6-thiophen-2-yl-4-thiophen-3-yl- pyridin-2-yloxy)-phenyl-acetic acid; |
| 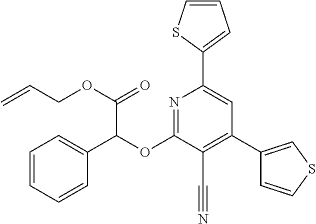 [SMILES: C=CCOC(=O)C(Oc1nc(-c2cccs2)cc(-c2ccsc2)c1C#N)c1ccccc1] | (3-Cyano-6-thiophen-2-yl-4-thiophen-3-yl- pyridin-2-yloxy)-phenyl-acetic acid allyl ester; |
| 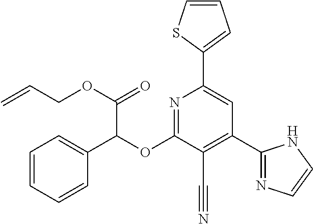 [SMILES: C=CCOC(=O)C(Oc1nc(-c2cccs2)cc(-c2ncc[nH]2)c1C#N)c1ccccc1] | [3-Cyano-4-(1H-imidazol-2-yl)-6-thiophen-2- yl-pyridin-2-yloxy]-phenyl-acetic acid allyl ester; |
| 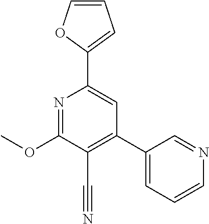 [SMILES: COc1nc(-c2ccco2)cc(-c2cccnc2)c1C#N] | 6′-Furan-2-yl-2′-methoxy-[3,4′]bipyridinyl-3′- carbonitrile; |
| 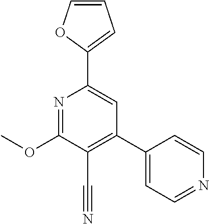 [SMILES: COc1nc(-c2ccco2)cc(-c2ccncc2)c1C#N] | 6-Furan-2-yl-2-methoxy-[4,4′]bipyridinyl-3- carbonitrile; |
| 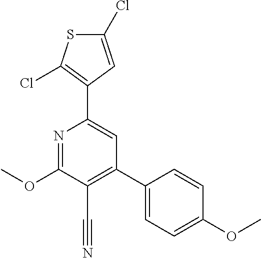 | 6-(2,5-Dichloro-thiophen-3-yl)-2-methoxy- 4-(4-methoxy-phenyl)-nicotinonitrile; |
| 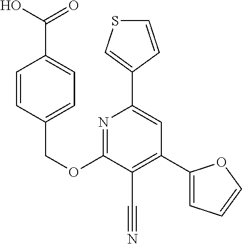 [SMILES: N#Cc1c(-c2ccco2)cc(-c2ccsc2)nc1OCc1ccc(C(=O)O)cc1] | 4-(3-Cyano-4-furan-2-yl-6-thiophen-3-yl- pyridin-2-yloxymethyl)-benzoic acid; |
| 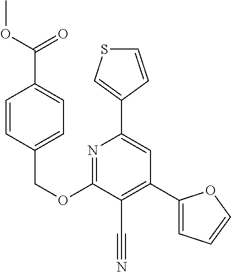 [SMILES: COC(=O)c1ccc(COc2nc(-c3ccsc3)cc(-c3ccco3)c2C#N)cc1] | 4-(3-Cyano-4-furan-2-yl-6-thiophen-3-yl- pyridin-2-yloxymethyl)-benzoic acid methyl ester; |
| 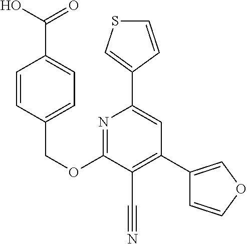 [SMILES: N#Cc1c(-c2ccoc2)cc(-c2ccsc2)nc1OCc1ccc(C(=O)O)cc1] | 4-(3-Cyano-4-furan-3-yl-6-thiophen-3-yl- pyridin-2-yloxymethyl)-benzoic acid; |
| 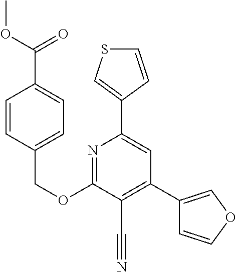 [SMILES: COC(=O)c1ccc(COc2nc(-c3ccsc3)cc(-c3ccoc3)c2C#N)cc1] | 4-(3-Cyano-4-furan-3-yl-6-thiophen-3-yl- pyridin-2-yloxymethyl)-benzoic acid methyl ester; |
| 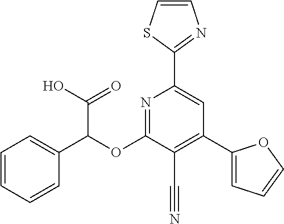 [SMILES: N#Cc1c(-c2ccco2)cc(-c2nccs2)nc1OC(C(=O)O)c1ccccc1] | (3-Cyano-4-furan-2-yl-6-thiazol-2-yl- pyridin-2-yloxy)-phenyl-acetic acid; |
| 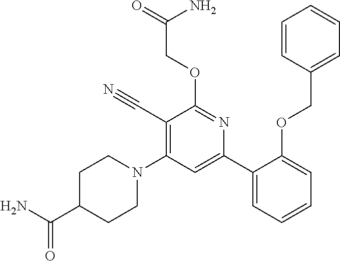 [SMILES: N#Cc1c(N2CCC(C(N)=O)CC2)cc(-c2ccccc2OCc2ccccc2)nc1OCC(N)=O] | 6′-(2-Benzyloxy-phenyl)-2′- carbamoylmethoxy-3′-cyano-3,4,5,6- tetrahydro-2H-[1,4′]bipyridinyl-4- carboxylic acid amide; |
| 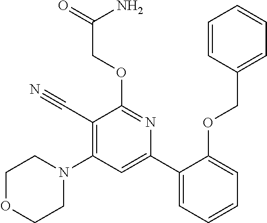 [SMILES: N#Cc1c(N2CCOCC2)cc(-c2ccccc2OCc2ccccc2)nc1OCC(N)=O] | 2-[6-(2-Benzyloxy-phenyl)-3-cyano-4- morpholin-4-yl-pyridin-2-yloxy]-acetamide; |
| 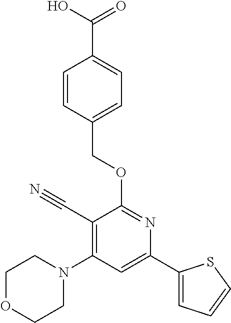 [SMILES: N#Cc1c(N2CCOCC2)cc(-c2cccs2)nc1OCc1ccc(C(=O)O)cc1] | 4-(3-Cyano-4-morpholin-4-yl-6-thiophen-2- yl-pyridin-2-yloxymethyl)-benzoic acid; |
| 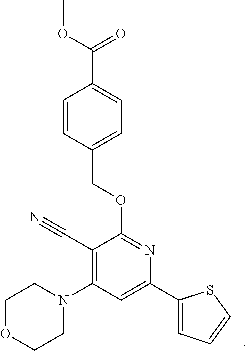 [SMILES: COC(=O)c1ccc(COc2nc(-c3cccs3)cc(N3CCOCC3)c2C#N)cc1] | 4-(3-Cyano-4-morpholin-4-yl-6-thiophen-2- yl-pyridin-2-yloxymethyl)-benzoic acid methyl ester; |

2. The compound of claim 1, which is selected from the group consisting of:

| | |
|---|---|
| 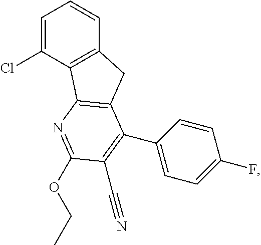 [SMILES: CCOc1nc2c(c(-c3ccc(F)cc3)c1C#N)Cc1cccc(Cl)c1-2] | 9-Chloro-2-ethoxy-4-(4-fluorophenyl)-5H- indeno[1,2-b]pyridine-3-carbonitrile; |
| 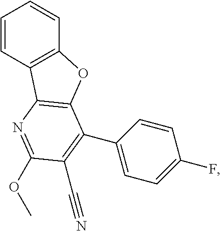 [SMILES: COc1nc2c(oc3ccccc32)c(-c2ccc(F)cc2)c1C#N] | 4-(4-Fluorophenyl)-2-methoxybenzofuro[3,2- b]pyridine-3-carbonitrile; |
| 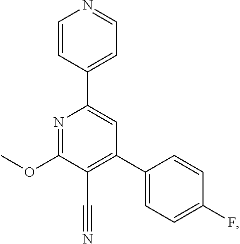 [SMILES: COc1nc(-c2ccncc2)cc(-c2ccc(F)cc2)c1C#N] | 4-(4-Fluorophenyl)-2-methoxy-6-(pyridin-4- yl)pyridine-3-carbonitrile; |
| 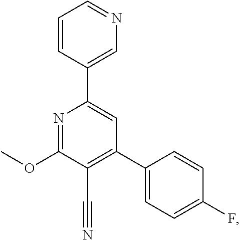 [SMILES: COc1nc(-c2cccnc2)cc(-c2ccc(F)cc2)c1C#N] | 4-(4-Fluorophenyl)-2-methoxy-6-(pyridin-3- yl)pyridine-3-carbonitrile; |
| 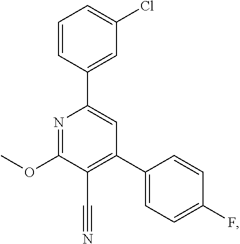 [SMILES: COc1nc(-c2cccc(Cl)c2)cc(-c2ccc(F)cc2)c1C#N] | 6-(3-Chlorophenyl)-4-(4-fluorophenyl)-2- methoxypyridine-3-carbonitrile; |
| 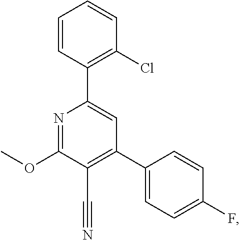 [SMILES: COc1nc(-c2ccccc2Cl)cc(-c2ccc(F)cc2)c1C#N] | 6-(2-Chlorophenyl)-4-(4-fluorophenyl)-2- methoxypyridine-3-carbonitrile; |
| 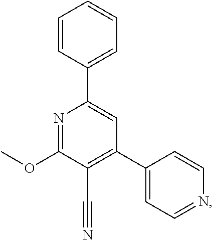 [SMILES: COc1nc(-c2ccccc2)cc(-c2ccncc2)c1C#N] | 2-Methoxy-6-phenyl-4-(pyridin-4-yl)pyridine-3- carbonitrile; |
| 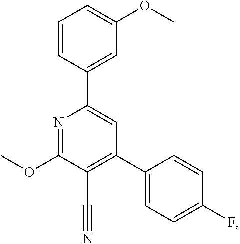 [SMILES: COc1cccc(-c2cc(-c3ccc(F)cc3)c(C#N)c(OC)n2)c1] | 4-(4-Fluorophenyl)-2-methoxy-6-(3- methoxyphenyl)pyridine-3-carbonitrile; |
| 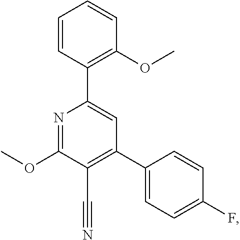 [SMILES: COc1ccccc1-c1cc(-c2ccc(F)cc2)c(C#N)c(OC)n1] | 4-(4-Fluorophenyl)-2-methoxy-6-(2- methoxyphenyl)pyridine-3-carbonitrile; |
| 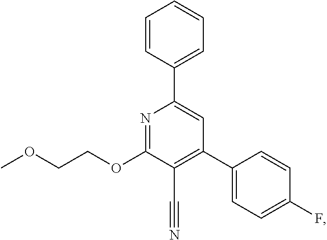 [SMILES: COCCOc1nc(-c2ccccc2)cc(-c2ccc(F)cc2)c1C#N] | 2-(2-Methoxyethoxy)-4-(4-fluorophenyl)-6- phenylpyridine-3-carbonitrile; |
| 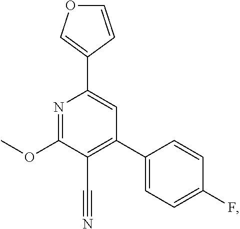 [SMILES: COc1nc(-c2ccoc2)cc(-c2ccc(F)cc2)c1C#N] | 4-(4-Fluoro-phenyl)-6-furan-3-yl-2-methoxy- nicotinonitrile; |
| 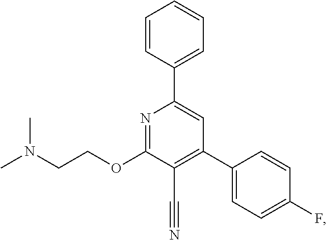 [SMILES: CN(C)CCOc1nc(-c2ccccc2)cc(-c2ccc(F)cc2)c1C#N] | 2-(2-(Dimethylamino)ethoxy)-4-(4- fluorophenyl)-6-phenylpyridine-3-carbonitrile; |
| 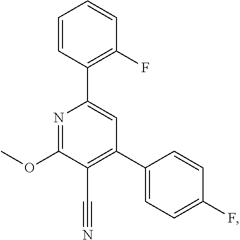 [SMILES: COc1nc(-c2ccccc2F)cc(-c2ccc(F)cc2)c1C#N] | 6-(2-Fluorophenyl)-4-(4-fluorophenyl)-2- methoxypyridine-3-carbonitrile; |
| 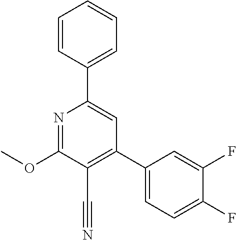 [SMILES: COc1nc(-c2ccccc2)cc(-c2ccc(F)c(F)c2)c1C#N] | 4-(3,4-Difluoro-phenyl)-2-methoxy-6-phenyl- nicotinonitrile; |
| 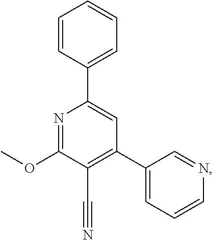 [SMILES: COc1nc(-c2ccccc2)cc(-c2cccnc2)c1C#N] | 2′-Methoxy-6′-phenyl-[3,4′]bipyridinyl-3′- carbonitrile; |
| 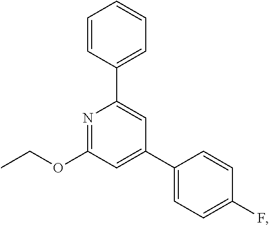 [SMILES: CCOc1cc(-c2ccc(F)cc2)cc(-c2ccccc2)n1] | 2-Ethoxy-4-(4-fluoro-phenyl)-6-phenyl-pyridine; |
| 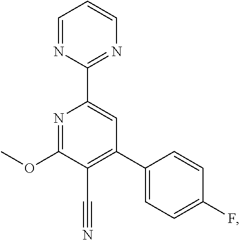 [SMILES: COc1nc(-c2ncccn2)cc(-c2ccc(F)cc2)c1C#N] | 4-(4-Fluoro-phenyl)-2-methoxy-6-pyrimidin-2- yl-nicotinonitrile; |
| 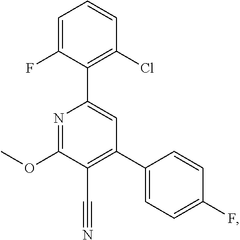 [SMILES: COc1nc(-c2c(F)cccc2Cl)cc(-c2ccc(F)cc2)c1C#N] | 6-(2-Chloro-6-fluoro-phenyl)-4-(4-fluoro- phenyl)-2-methoxy-nicotinonitrile; |
| 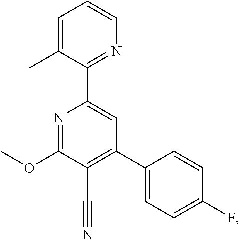 [SMILES: COc1nc(-c2ncccc2C)cc(-c2ccc(F)cc2)c1C#N] | 4-(4-Fluoro-phenyl)-6-methoxy-3′-methyl- [2,2′]bipyridinyl-5-carbonitrile; |
| 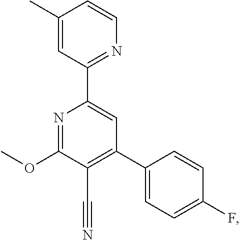 [SMILES: COc1nc(-c2cc(C)ccn2)cc(-c2ccc(F)cc2)c1C#N] | 4-(4-Fluoro-phenyl)-6-methoxy-4′-methyl- [2,2′]bipyridinyl-5-carbonitrile; |
| 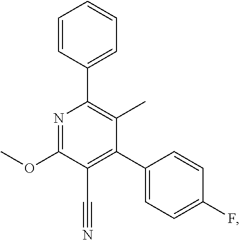 [SMILES: COc1nc(-c2ccccc2)c(C)c(-c2ccc(F)cc2)c1C#N] | 4-(4-Fluoro-phenyl)-2-methoxy-5-methyl-6- phenyl-nicotinonitrile; |
| 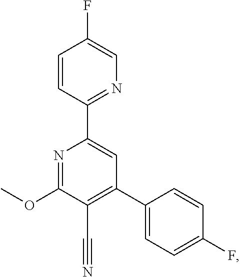 [SMILES: COc1nc(-c2ccc(F)cn2)cc(-c2ccc(F)cc2)c1C#N] | 5′-Fluoro-4-(4-fluoro-phenyl)-6-methoxy- [2,2′]bipyridinyl-5-carbonitrile; |
| 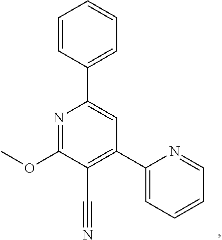 [SMILES: COc1nc(-c2ccccc2)cc(-c2ccccn2)c1C#N] | 2′-Methoxy-6′-phenyl-[2,4′]bipyridinyl-3′- carbonitrile; |
| 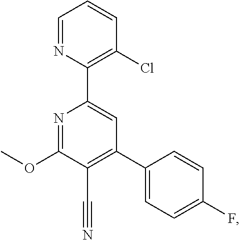 [SMILES: COc1nc(-c2ncccc2Cl)cc(-c2ccc(F)cc2)c1C#N] | 6-(3-Chloropyridin-2-yl)-4-(4-fluorophenyl)-2- methoxypyridine-3-carbonitrile; |
| 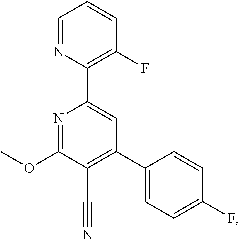 [SMILES: COc1nc(-c2ncccc2F)cc(-c2ccc(F)cc2)c1C#N] | 4-(4-Fluorophenyl)-6-(3-fluoropyridin-2-yl)-2- methoxypyridine-3-carbonitrile; |
| 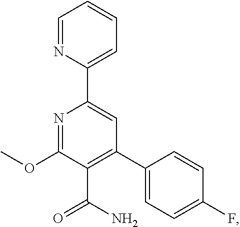 [SMILES: COc1nc(-c2ccccn2)cc(-c2ccc(F)cc2)c1C(N)=O] | 4-(4-Fluorophenyl)-2-methoxy-6-(pyridin-2- yl)pyridine-3-carboxamide; |
| 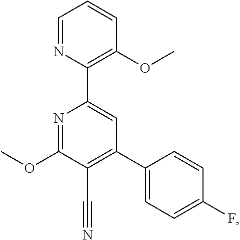 [SMILES: COc1cccnc1-c1cc(-c2ccc(F)cc2)c(C#N)c(OC)n1] | 4-(4-Fluorophenyl)-2-methoxy-6-(3- methoxypyridin-2-yl)pyridine-3-carbonitrile; |
| 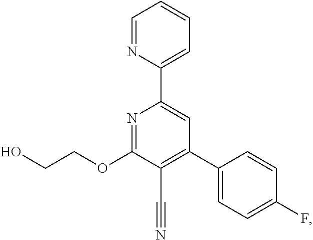 [SMILES: N#Cc1c(-c2ccc(F)cc2)cc(-c2ccccn2)nc1OCCO] | 2-(2-Hydroxyethoxy)-4-(4-fluorophenyl)-6- (pyridin-2-yl)pyridine-3-carbonitrile; |
| 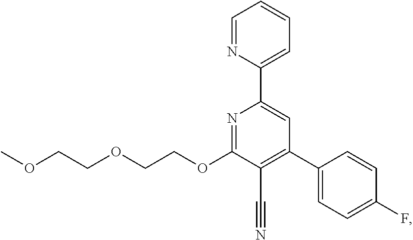 [SMILES: COCCOCCOc1nc(-c2ccccn2)cc(-c2ccc(F)cc2)c1C#N] | 2-(2-(2-Methoxyethoxy)ethoxy)-4-(4- fluorophenyl)-6-(pyridin-2-yl)pyridine-3- carbonitrile; |
| 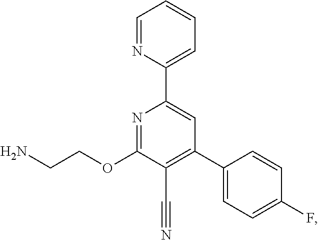 [SMILES: N#Cc1c(-c2ccc(F)cc2)cc(-c2ccccn2)nc1OCCN] | 2-(2-Aminoethoxy)-4-(4-fluorophenyl)-6- (pyridin-2-yl)pyridine-3-carbonitrile; |
| 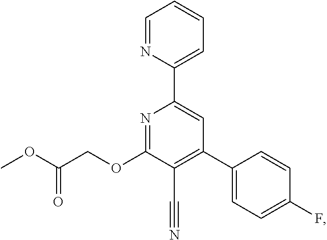 [SMILES: COC(=O)COc1nc(-c2ccccn2)cc(-c2ccc(F)cc2)c1C#N] | Methyl 2-(3-cyano-4-(4-fluorophenyl)-6-(pyridin- 2-yl)pyridin-2-yloxy)acetate; |
| 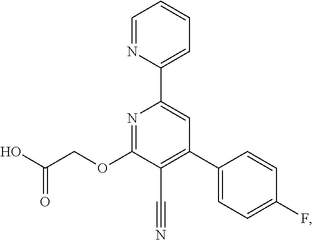 [SMILES: N#Cc1c(-c2ccc(F)cc2)cc(-c2ccccn2)nc1OCC(=O)O] | 2-(3-Cyano-4-(4-fluorophenyl)-6-(pyridin-2- yl)pyridin-2-yloxy)acetic acid; |
| 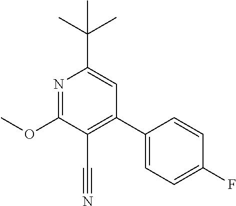 [SMILES: COc1nc(C(C)(C)C)cc(-c2ccc(F)cc2)c1C#N] | 6-tert-Butyl-4-(4-fluoro-phenyl)-2-methoxy- nicotinonitrile; |
| 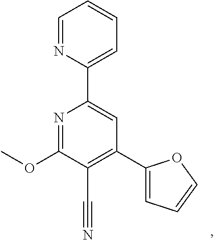 [SMILES: COc1nc(-c2ccccn2)cc(-c2ccco2)c1C#N] | 4-(Furan-2-yl)-2-methoxy-6-(pyridin-2- yl)pyridine-3-carbonitrile; |
| 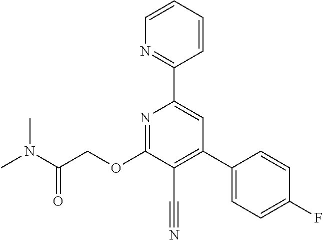 [SMILES: CN(C)C(=O)COc1nc(-c2ccccn2)cc(-c2ccc(F)cc2)c1C#N] | Cyano-4-(4-fluoro-phenyl)-[2,2′]bipyridinyl-6- yloxy]-N,N-dimethyl-acetamide; |
| 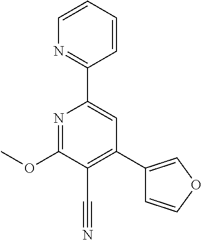 [SMILES: COc1nc(-c2ccccn2)cc(-c2ccoc2)c1C#N] | 4-Furan-3-yl-6-methoxy-[2,2′]bipyridinyl-5- carbonitrile; |
| 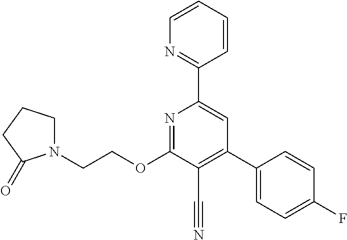 [SMILES: N#Cc1c(-c2ccc(F)cc2)cc(-c2ccccn2)nc1OCCN1CCCC1=O] | Fluoro-phenyl)-6-[2-(2-oxo-pyrrolidin-1-yl)- ethoxy]-[2,2′]bipyridinyl-5-carbonitrile; |
| 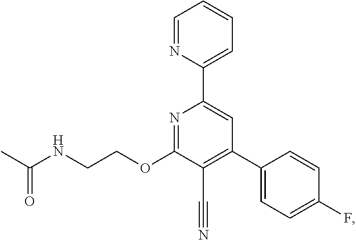 [SMILES: CC(=O)NCCOc1nc(-c2ccccn2)cc(-c2ccc(F)cc2)c1C#N] | N-(2-(3-Cyano-4-(4-fluorophenyl)-6-(pyridin-2- yl)pyridin-2-yloxy)ethyl)acetamide; |
| 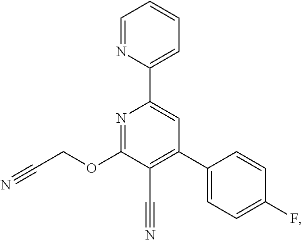 [SMILES: N#CCOc1nc(-c2ccccn2)cc(-c2ccc(F)cc2)c1C#N] | 2-(Cyanomethoxy)-4-(4-fluorophenyl)-6- (pyridin-2-yl)pyridine-3-carbonitrile; |
| 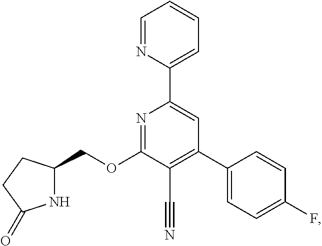 [SMILES: N#Cc1c(-c2ccc(F)cc2)cc(-c2ccccn2)nc1OC[C@@H]1CCC(=O)N1] | 2-((S)-5-(Methoxy)pyrrolidin-2-one)-4-(4- fluorophenyl)-6-(pyridin-2-yl)pyridine-3- carbonitrile; |
| 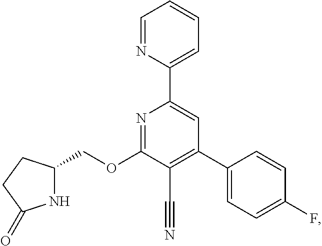 [SMILES: N#Cc1c(-c2ccc(F)cc2)cc(-c2ccccn2)nc1OC[C@H]1CCC(=O)N1] | 2-((R)-5-(Methoxy)pyrrolidin-2-one)-4-(4- fluorophenyl)-6-(pyridin-2-yl)pyridine-3- carbonitrile; |
| 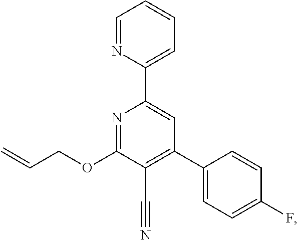 [SMILES: C=CCOc1nc(-c2ccccn2)cc(-c2ccc(F)cc2)c1C#N] | 2-(Allyloxy)-4-(4-fluorophenyl)-6-(pyridin-2- yl)pyridine-3-carbonitrile; |
| 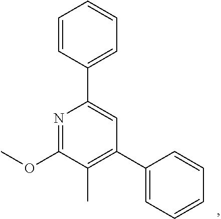 [SMILES: COc1nc(-c2ccccc2)cc(-c2ccccc2)c1C] | 2-Methoxy-3-methyl-4,6-diphenylpyridine; |
| 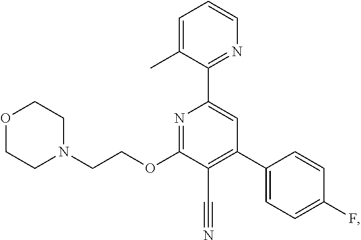 [SMILES: Cc1cccnc1-c1cc(-c2ccc(F)cc2)c(C#N)c(OCCN2CCOCC2)n1] | 2-(2-Morpholinoethoxy)-4-(4-fluorophenyl)-6-(3- methylpyridin-2-yl)pyridine-3-carbonitrile; |
| 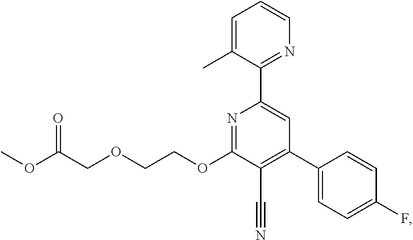 [SMILES: COC(=O)COCCOc1nc(-c2ncccc2C)cc(-c2ccc(F)cc2)c1C#N] | Methyl 2-(3-cyano-4-(4-fluorophenyl)-6-(3- methylpyridin-2-yl)pyridin-2-yloxy) 2-(2- methoxyethoxy)acetate; |
| 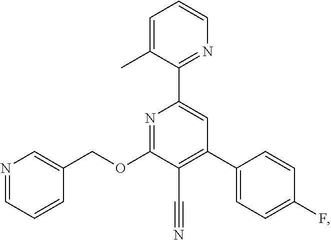 [SMILES: Cc1cccnc1-c1cc(-c2ccc(F)cc2)c(C#N)c(OCc2cccnc2)n1] | 2-((Pyridin-3-yl)methoxy)-4-(4-fluorophenyl)-6- (3-methylpyridin-2-yl)pyridine-3-carbonitrile; |
| 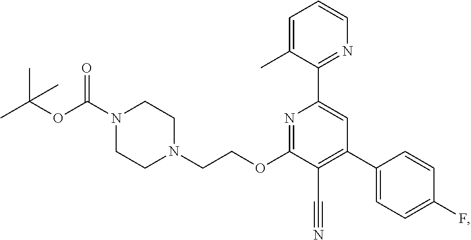 [SMILES: Cc1cccnc1-c1cc(-c2ccc(F)cc2)c(C#N)c(OCCN2CCN(C(=O)OC(C)(C)C)CC2)n1] | tert-Butyl 4-(2-(3-cyano-4-(4-fluorophenyl)-6-(3- methylpyridin-2-yl)pyridin-2- yloxy)ethyl)piperazine-1-carboxylate; |
| 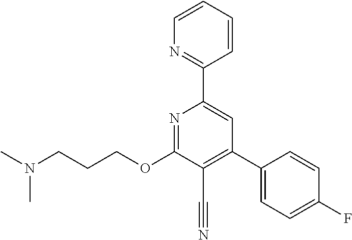 [SMILES: CN(C)CCCOc1nc(-c2ccccn2)cc(-c2ccc(F)cc2)c1C#N] | 6-(3-Dimethylamino-propoxy)-4-(4-fluoro- phenyl)-[2,2′]bipyridinyl-5-carbonitrile; |
| 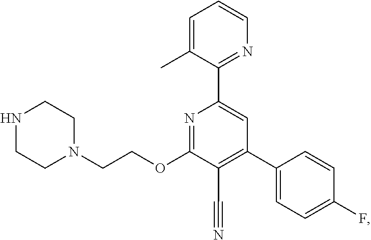 [SMILES: Cc1cccnc1-c1cc(-c2ccc(F)cc2)c(C#N)c(OCCN2CCNCC2)n1] | 2-(2-(Piperazin-1-yl)ethoxy)-4-(4-fluorophenyl)- 6-(3-methylpyridin-2-yl)pyridine-3-carbonitrile |
| 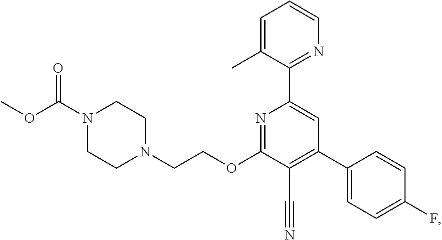 [SMILES: COC(=O)N1CCN(CCOc2nc(-c3ncccc3C)cc(-c3ccc(F)cc3)c2C#N)CC1] | Methyl 4-(2-(3-cyano-4-(4-fluorophenyl)-6-(3- methylpyridin-2-yl)pyridin-2- yloxy)ethyl)piperazine-1-carboxylate; |
| 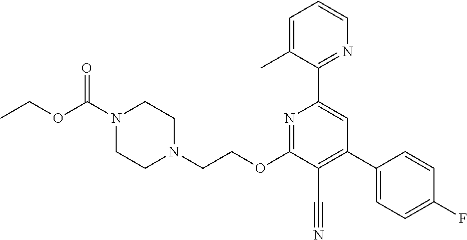 [SMILES: CCOC(=O)N1CCN(CCOc2nc(-c3ncccc3C)cc(-c3ccc(F)cc3)c2C#N)CC1] | Ethyl 4-(2-(3-cyano-4-(4-fluorophenyl)-6-(3- methylpyridin-2-yl)pyridin-2- yloxy)ethyl)piperazine-1-carboxylate; |
| 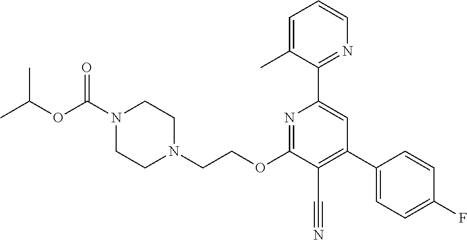 [SMILES: Cc1cccnc1-c1cc(-c2ccc(F)cc2)c(C#N)c(OCCN2CCN(C(=O)OC(C)C)CC2)n1] | Isopropyl 4-(2-(3-cyano-4-(4-fluorophenyl)-6-(3- methylpyridin-2-yl)pyridin-2- yloxy)ethyl)piperazine-1-carboxylate; |
| 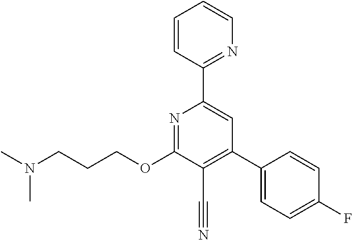 [SMILES: CN(C)CCCOc1nc(-c2ccccn2)cc(-c2ccc(F)cc2)c1C#N] | 2-(3-(Dimethylamino)propoxy)-4-(4- fluorophenyl)-6-(pyridin-2-yl)pyridine-3- carbonitrile; |
| 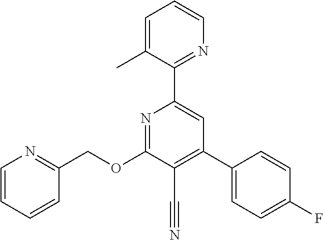 [SMILES: Cc1cccnc1-c1cc(-c2ccc(F)cc2)c(C#N)c(OCc2ccccn2)n1] | 2-((Pyridin-2-yl)methoxy)-4-(4-fluorophenyl)-6- (3-methylpyridin-2-yl)pyridine-3-carbonitrile; |
| 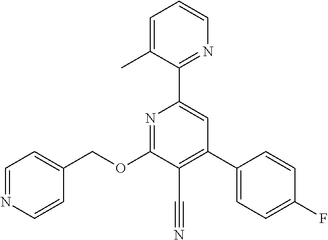 [SMILES: Cc1cccnc1-c1cc(-c2ccc(F)cc2)c(C#N)c(OCc2ccncc2)n1] | 2-((Pyridin-4-yl)methoxy)-4-(4-fluorophenyl)-6- (3-methylpyridin-2-yl)pyridine-3-carbonitrile; |
| 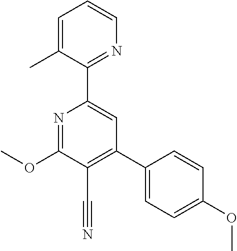 | 2-Methoxy-4-(4-methoxyphenyl)-6-(3- methylpyridin-2-yl)pyridine-3-carbonitrile; |
| 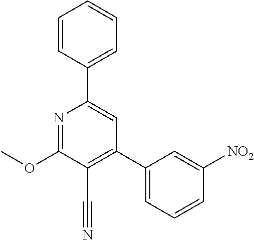 [SMILES: COc1nc(-c2ccccc2)cc(-c2cccc([N+](=O)[O-])c2)c1C#N] | 2-Methoxy-4-(3-nitrophenyl)-6-phenylpyridine- 3-carbonitrile; |
| 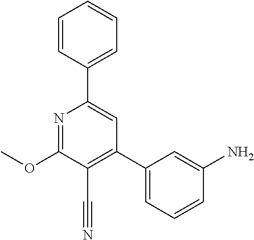 [SMILES: COc1nc(-c2ccccc2)cc(-c2cccc(N)c2)c1C#N] | 4-(3-Aminophenyl)-2-methoxy-6-phenylpyridine- 3-carbonitrile; |
| 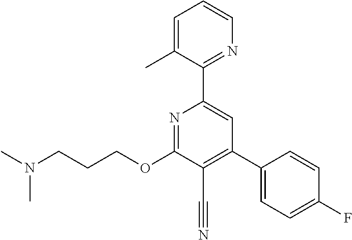 [SMILES: Cc1cccnc1-c1cc(-c2ccc(F)cc2)c(C#N)c(OCCCN(C)C)n1] | 2-(3-(Dimethylamino)propoxy)-4-(4- fluorophenyl)-6-(3-methylpyridin-2-yl)pyridine- 3-carbonitrile; |
| 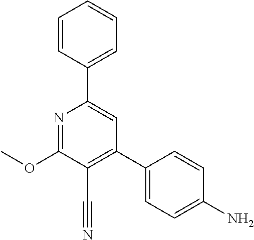 [SMILES: COc1nc(-c2ccccc2)cc(-c2ccc(N)cc2)c1C#N] | 4-(4-Aminophenyl)-2-methoxy-6-phenylpyridine- 3-carbonitrile; |
| 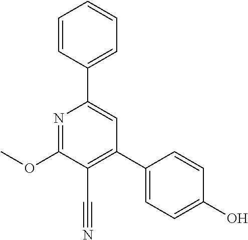 [SMILES: COc1nc(-c2ccccc2)cc(-c2ccc(O)cc2)c1C#N] | 4-(4-Hydroxyphenyl)-2-methoxy-6- phenylpyridine-3-carbonitrile; |
| 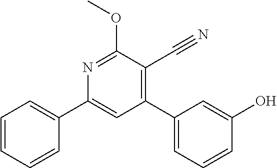 [SMILES: COc1nc(-c2ccccc2)cc(-c2cccc(O)c2)c1C#N] | 4-(3-Hydroxyphenyl)-2-methoxy-6- phenylpyridine-3-carbonitrile; |
| 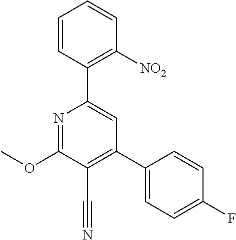 [SMILES: COc1nc(-c2ccccc2[N+](=O)[O-])cc(-c2ccc(F)cc2)c1C#N] | 4-(4-Fluorophenyl)-2-methoxy-6-(2- nitrophenyl)pyridine-3-carbonitrile; |
| 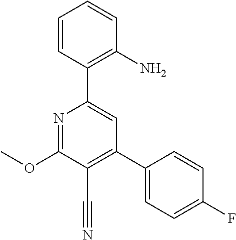 [SMILES: COc1nc(-c2ccccc2N)cc(-c2ccc(F)cc2)c1C#N] | 6-(2-Aminophenyl)-4-(4-fluorophenyl)-2- methoxypyridine-3-carbonitrile; |
| 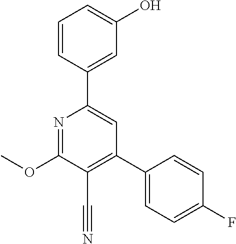 [SMILES: COc1nc(-c2cccc(O)c2)cc(-c2ccc(F)cc2)c1C#N] | 4-(4-fluorophenyl)-6-(3-hydroxyphenyl)-2- methoxypyridine-3-carbonitrile; |
| 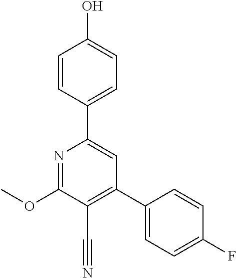 [SMILES: COc1nc(-c2ccc(O)cc2)cc(-c2ccc(F)cc2)c1C#N] | 4-(4-Fluorophenyl)-6-(4-hydroxyphenyl)-2- methoxypyridine-3-carbonitrile; |
| 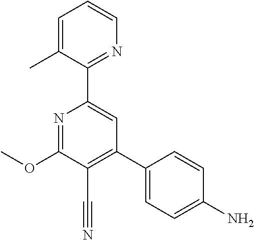 [SMILES: COc1nc(-c2ncccc2C)cc(-c2ccc(N)cc2)c1C#N] | 4-(4-Aminophenyl)-2-methoxy-6-(3- methylpyridin-2-yl)pyridine-3-carbonitrile, and |
| 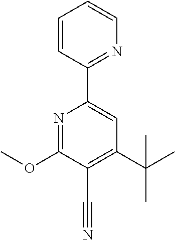 [SMILES: COc1nc(-c2ccccn2)cc(C(C)(C)C)c1C#N] | 4-tert-Butyl-2-methoxy-6-(pyridin-2-yl)pyridine- 3-carbonitrile. |

3. A pharmaceutical composition comprising the compound of claim 1 and at least one pharmaceutically acceptable carrier.

4. The composition of claim 3, further comprising at least one additional agent that treats or prevents heart failure, high or elevated blood pressure, atherosclerotic plaque formation, or cardiac remodeling.

5. The composition of claim 4, wherein the at least one additional agent is selected from the group consisting of ACE inhibitors, beta blockers, neprilysin inhibitors, diuretics, aldosterone antagonists, vasodilators, and nitrates.

6. A method of treating or ameliorating heart failure in a subject in need thereof, the method comprising administering to the subject a therapeutically effective amount of at least one compound selected from the group consisting of:
(a) 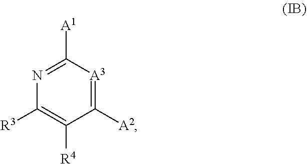
wherein in (IB):
A¹ is —C(R⁷)(R⁸)(R⁹), 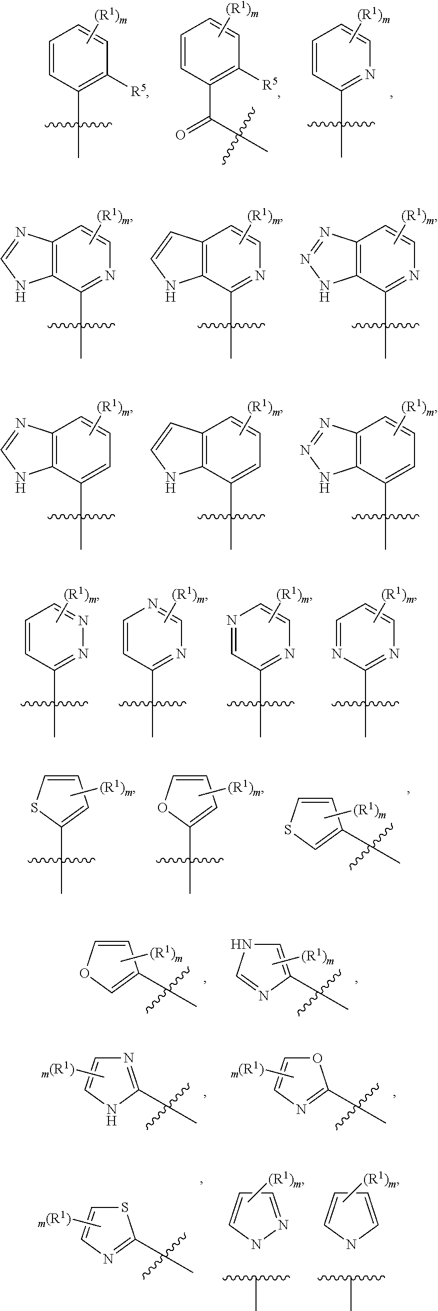 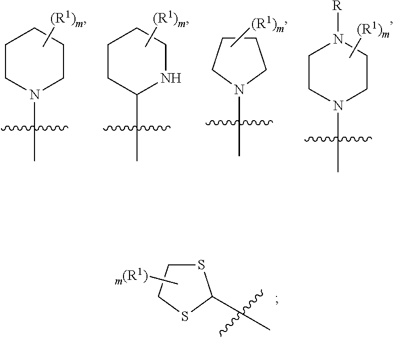
A² is —C(R¹⁰)(R^{U})(R¹²), 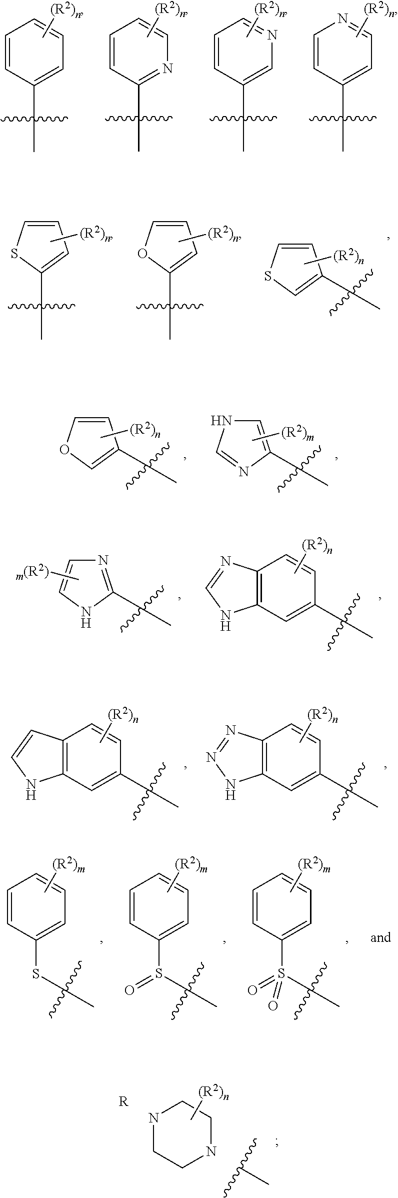
A³ is N or C—R⁶;
m is 1, 2 or 3;
n is 1, 2 or 3;
each occurrence of R¹ is independently selected from the group consisting of H, F, Cl, Br, I, —OR, —NR₂, —SR, —S(O)(C₁-C₆ alkyl), —S(O)₂(C₁-C₆ alkyl), —(CH₂)₀₋₅NR₂, —CN, —NO₂, C₁-C₅ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R¹ groups are present in neighboring carbons, they optionally combine to form the divalent group —O(CH₂)₁₋₃O—;
each occurrence of R² is independently selected from the group consisting of H, F, Cl, Br, I, —OR, —SR, —S(O)(C₁-C₆ alkyl), —S(O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R² groups are present in neighboring carbons, they optionally combine to form the divalent group —O(CH₂)₁₋₃O—;
with the proviso that R² is not positioned ortho to the bond between the central heteroaryl ring and A²;
R³ is selected from the group consisting of H, C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆ alkyl), —S(O)(C₁-C₆ alkyl), —S(O)₂(C₁-C₆ alkyl), N¹-β-propiolactam, N¹-γ-butyrolactam, N¹-δ-valerolactam, oxetanoyl, oxetanoxyl, N¹-ε-caprolactam, (C₂-C₆)alkenyloxy, (C₂-C₆)alkynyloxy, —O(CH₂)₂₋₄OR, —O(CH₂)₂₋₄O(CH₂)₂₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl, —O(CH₂)₂₋₄(N¹-pyrrolidinyl), —N¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;
R⁴ is independently selected from the group consisting of H, —CH₃, —CN, —C≡CR, —C(═O)OR, and —C(═O)NHR, or R⁴ can combine with R³ to form: 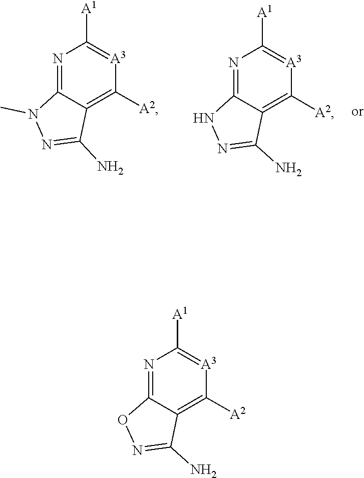
R⁵ is R¹ and R⁶ is H or C₁-C₆, alkyl, or R⁵ and R⁶ combine to form a divalent group selected from the group consisting of —O—, —CRR—, or —CRR—CRR—,
each of R⁷, R⁸, R⁹, R¹⁰, R¹¹, and R¹² is independently selected from optionally substituted C₁-C₁₀ alkyl, wherein two or three of R⁷, R⁸, R⁹ or of R¹⁰, R¹¹, R¹² can optionally combine to form monocyclic or polycyclic groups; and
each occurrence of R is independently H or C₁-C₆ alkyl;
(b) 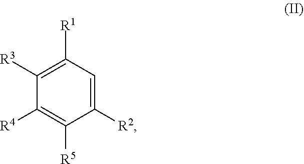
wherein in (II):
R¹ is selected from the group consisting of phenyl, N¹-pyrrolyl, N¹-imidazolyl, N¹-pyrazolyl, N¹-triazolyl, N²-1,2,3-triazolyl, N¹-triazolyl, N⁴-1,2,3-triazolyl, N¹-tetrazolyl, N¹-isoxazolyl, N¹-pyrrolidinyl, N¹-piperidinyl, N¹-morpholinyl, piperizin-1-yl, and N⁴—(C₁-C₆ alkyl)-piperizin-1-yl,
wherein the phenyl group is optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, C(═O)NR₂, N¹-β-propiolactam, N¹-γ-butyrolactam, N¹-δ-valerolactam, and N¹-ε-caprolactam; wherein, if two substituents are present in neighboring carbons, they optionally combine to form the divalent group —O(CH₂)₁₋₃O—;
R² is independently selected from the group consisting of H, piperizin-1-yl, N⁴—(C₁-C₆ alkyl)-piperizin-1-yl, phenyl, pyridin-2-yl, pyridin-3-yl, and pyridin-4-yl;
wherein the phenyl group is optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, C(═O)NR₂, N¹-β-propiolactam, N¹-γ-butyrolactam, N¹-δ-valerolactam, and N¹-ε-caprolactam; wherein, if two substituents are present in neighboring carbons, they optionally combine to form the divalent group —O(CH₂)₁₋₃O—;
R³ is selected from the group consisting of H and Cl;
R⁴ is selected from the group consisting of H, —OH, (C₁-C₆)alkyl, (C₁-C₆)alkoxy, (C₁-C₆)haloalkyl, (C₁-C₆)haloalkoxy, and benzyloxy;
R⁵ is independently selected from the group consisting of H, —NO₂, —CN, —C≡CH, —C(═O)OH, —SO₂(C₁-C₆ alkyl), —SO₂NHAc, and tetrazol-1-yl;
(c) 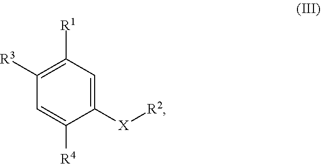
wherein in (III):
X is selected from the group consisting of O, S, S(═O), and S(═O)₂;
R¹ is selected from the group consisting of H, phenyl, —C(═O)phenyl, 1,3-dithiol-2-yl, pyrrol-1-yl, imidazole-1-yl, pyrazol-1-yl, 1,2,3-triazol-1-yl, 1,2,3-triazol-2-yl, 1,2,4-triazol-1-yl, 1,2,3-triazol-4-yl, tetrazol-1-yl, isoxazol-1-yl, pyrrolidin-1-yl, piperidin-1-yl, morpholin-1-yl, piperizin-1-yl, and N⁴—(C₁-C₆ alkyl)-piperizin-1-yl;
wherein each phenyl group is independently optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, —C(═O)NR₂, N¹-β-propiolactam, N¹-γ-butyrolactam, N¹-δ-valerolactam, and N¹-ε-caprolactam;
wherein, if two substituents are present in neighboring carbons, they optionally combine to form the divalent group —O(CH₂)₁₋₃O—;
R² is selected from the group consisting of phenyl, pyridin-2-yl, pyridin-3-yl, and pyridin-4-yl;
wherein each phenyl group is independently optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, —C(═O)NR₂, N¹-β-propiolactam, N¹-γ-butyrolactam, N¹-δ-valerolactam, and N¹-ε-caprolactam;
wherein, if two substituents are present in neighboring carbons, they optionally combine to form the divalent group —O(CH₂)₁₋₃O—;
R³ is selected from the group consisting of H, F, Cl, Br, I, C₁-C₆ alkyl, and C₁-C₆, alkoxy; and
R⁴ is independently selected from the group consisting of H, —NO₂, —CN, —C≡CH, —C(═O)OH, —SO₂(C₁-C₆ alkyl), —SO₂NHAC, and N¹-tetrazolyl.

7. The method of claim 6, wherein the compound is selected from the group consisting of:

| Structure | Name |
|---|---|
| 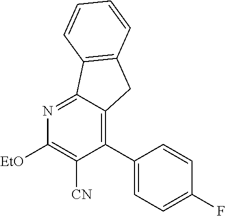 [SMILES: CCOc1nc2c(c(-c3ccc(F)cc3)c1C#N)Cc1ccccc1-2] | 2-ethoxy-4-(4-fluorophenyl)-5H- indeno[1,2-b]pyridine-3-carbonitrile; |
| 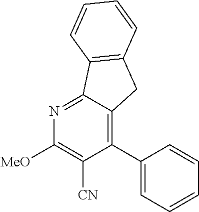 [SMILES: COc1nc2c(c(-c3ccccc3)c1C#N)Cc1ccccc1-2] | 2-methoxy-4-phenyl-5H-indeno[1,2- b]pyridine-3-carbonitrile; |
| 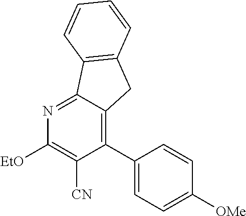 [SMILES: CCOc1nc2c(c(-c3ccc(OC)cc3)c1C#N)Cc1ccccc1-2] | 2-ethoxy-4-(4-methoxyphenyl)-5H- indeno[1,2-b]pyridine-3-carbonitrile; |
| 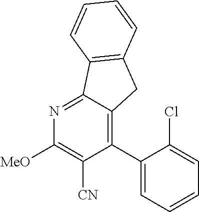 [SMILES: COc1nc2c(c(-c3ccccc3Cl)c1C#N)Cc1ccccc1-2] | 4-(2-chlorophenyl)-2-methoxy-5H- indeno[1,2-b]pyridine-3-carbonitrile; |
| 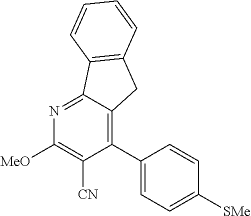 [SMILES: COc1nc2c(c(-c3ccc(SC)cc3)c1C#N)Cc1ccccc1-2] | 2-methoxy-4-(4-(methylthio)phenyl)-5H- indeno[1,2-b]pyridine-3-carbonitrile; |
| 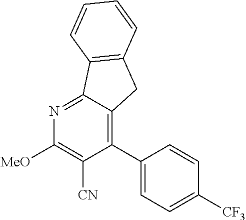 [SMILES: COc1nc2c(c(-c3ccc(C(F)(F)F)cc3)c1C#N)Cc1ccccc1-2] | 2-methoxy-4-(4-(trifluoromethyl)phenyl)- 5H-indeno[1,2-b]pyridine-3-carbonitrile; |
| 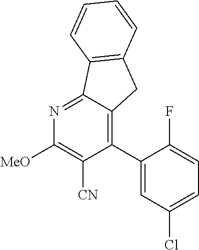 [SMILES: COc1nc2c(c(-c3cc(Cl)ccc3F)c1C#N)Cc1ccccc1-2] | 4-(5-chloro-2-fluorophenyl)-2-methoxy-5H- indeno[1,2-b]pyridine-3-carbonitrile; |
| 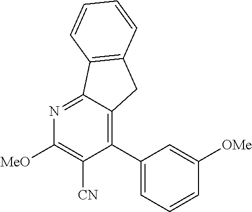 [SMILES: COc1cccc(-c2c(C#N)c(OC)nc3c2Cc2ccccc2-3)c1] | 2-methoxy-4-(3-methoxyphenyl)-5H- indeno[1,2-b]pyridine-3-carbonitrile; |
| 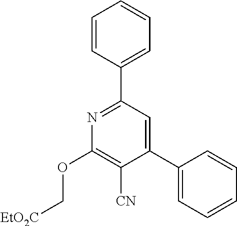 [SMILES: CCOC(=O)COc1nc(-c2ccccc2)cc(-c2ccccc2)c1C#N] | ethyl 2-((3-cyano-4,6-diphenylpyridin-2- yl)oxy)acetate; |
| 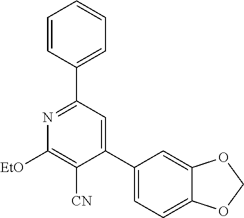 [SMILES: CCOc1nc(-c2ccccc2)cc(-c2ccc3c(c2)OCO3)c1C#N] | 4-(benzo[d][1,3]dioxol-5-yl)-2-ethoxy-6- phenylnicotinonitrile; |
| 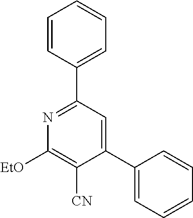 [SMILES: CCOc1nc(-c2ccccc2)cc(-c2ccccc2)c1C#N] | 2-ethoxy-4,6-diphenylnicotinonitrile; |
| 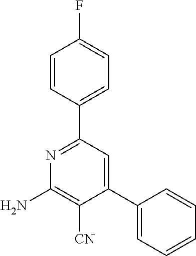 [SMILES: N#Cc1c(-c2ccccc2)cc(-c2ccc(F)cc2)nc1N] | 2-amino-6-(4-fluorophenyl)-4- phenylnicotinonitrile; |
| 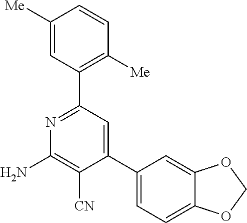 [SMILES: Cc1ccc(C)c(-c2cc(-c3ccc4c(c3)OCO4)c(C#N)c(N)n2)c1] | 2-amino-4-(benzo[d][1,3]dioxol-5-yl)-6- (2,5-dimethylphenyl)nicotinonitrile; |
| 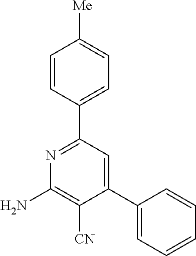 [SMILES: Cc1ccc(-c2cc(-c3ccccc3)c(C#N)c(N)n2)cc1] | 2-amino-4-phenyl-6-(p-tolyl)nicotinonitrile; |
| 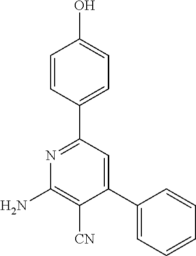 [SMILES: N#Cc1c(-c2ccccc2)cc(-c2ccc(O)cc2)nc1N] | 2-amino-6-(4-hydroxyphenyl)-4- phenylnicotinonitrile; |
| 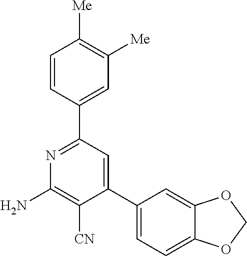 [SMILES: Cc1ccc(-c2cc(-c3ccc4c(c3)OCO4)c(C#N)c(N)n2)cc1C] | 2-amino-4-(benzo[d][1,3]dioxol-5-yl)-6- (3,4-dimethylphenyl)nicotinonitrile; |
| 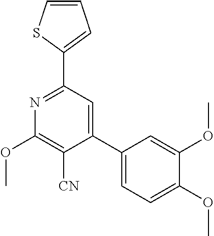 [SMILES: COc1ccc(-c2cc(-c3cccs3)nc(OC)c2C#N)cc1OC] | 4-(3,4-Dimethoxy-phenyl)-2-methoxy-6- thiophen-2-yl-nicotinonitrile; |
| 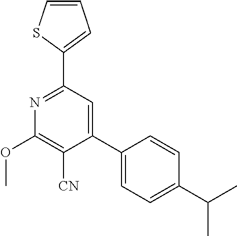 [SMILES: COc1nc(-c2cccs2)cc(-c2ccc(C(C)C)cc2)c1C#N] | 4-(4-Isopropyl-phenyl)-2-methoxy-6- thiophen-2-yl-nicotinonitrile; |
| 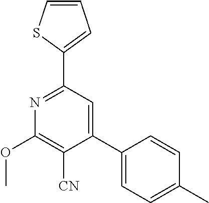 [SMILES: COc1nc(-c2cccs2)cc(-c2ccc(C)cc2)c1C#N] | 2-Methoxy-6-thiophen-2-yl-4-p-tolyl- nicotinonitrile; |
| 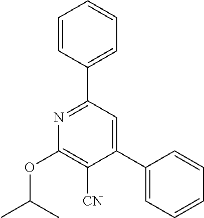 [SMILES: CC(C)Oc1nc(-c2ccccc2)cc(-c2ccccc2)c1C#N] | 2-Isopropoxy-4,6-diphenyl-nicotinonitrile; |
| 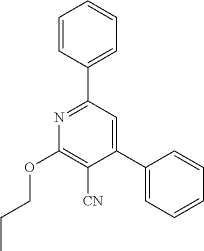 [SMILES: CCCOc1nc(-c2ccccc2)cc(-c2ccccc2)c1C#N] | 4,6-Diphenyl-2-propoxy-nicotinonitrile; |
| 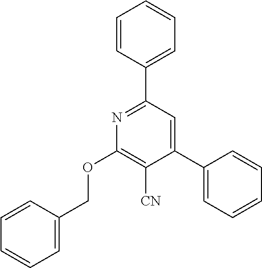 [SMILES: N#Cc1c(-c2ccccc2)cc(-c2ccccc2)nc1OCc1ccccc1] | 2-Benzyloxy-4,6-diphenyl-nicotinonitrile; |
| 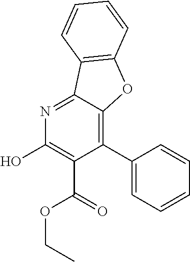 [SMILES: CCOC(=O)c1c(O)nc2c(oc3ccccc32)c1-c1ccccc1] | 2-Hydroyx-4-phenyl-benzo[4,5]furo[3,2- b]pyridine-3-carboxylic acid ethyl ester; |
| 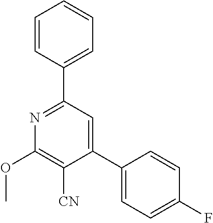 [SMILES: COc1nc(-c2ccccc2)cc(-c2ccc(F)cc2)c1C#N] | 4-(4-Fluoro-phenyl)-2-methoxy-6-phenyl- nicotinonitrile; |
| 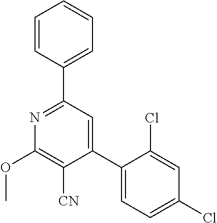 [SMILES: COc1nc(-c2ccccc2)cc(-c2ccc(Cl)cc2Cl)c1C#N] | 4-(2,4-Dichloro-phenyl)-2-methoxy-6- phenyl-nicotinonitrile; |
| 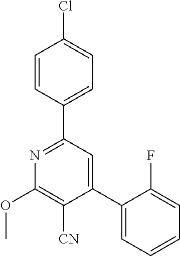 [SMILES: COc1nc(-c2ccc(Cl)cc2)cc(-c2ccccc2F)c1C#N] | 6-(4-Chloro-phenyl)-4-(2-fluoro-phenyl)-2- methoxy-nicotinonitrile; |
| 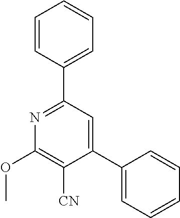 [SMILES: COc1nc(-c2ccccc2)cc(-c2ccccc2)c1C#N] | 2-Methoxy-4,6-diphenyl-nicotinonitrile; |
| 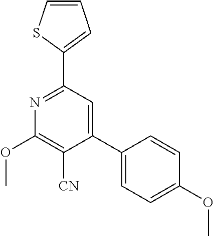 [SMILES: COc1ccc(-c2cc(-c3cccs3)nc(OC)c2C#N)cc1] | 2-Methoxy-4-(4-methoxy-phenyl)-6- thiophen-2-yl-nicotinonitile; |
| 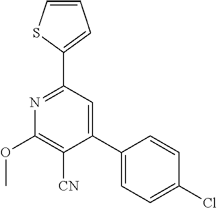 [SMILES: COc1nc(-c2cccs2)cc(-c2ccc(Cl)cc2)c1C#N] | 4-(4-Chloro-phenyl)-2-methoxy-6-thiophen- 2-yl-nicotinonitrile; |
| 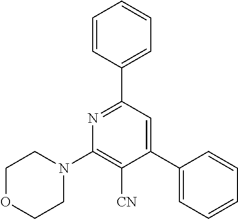 [SMILES: N#Cc1c(-c2ccccc2)cc(-c2ccccc2)nc1N1CCOCC1] | 2-Morpholin-4-yl-4,6-diphenyl- nicotinonitrile; |
| 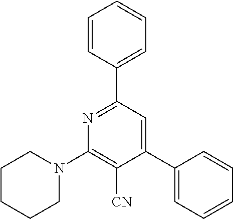 [SMILES: N#Cc1c(-c2ccccc2)cc(-c2ccccc2)nc1N1CCCCC1] | 4,6-diphenyl-2-(piperidin-1-yl)pyridine-3- carbonitrile; |
| 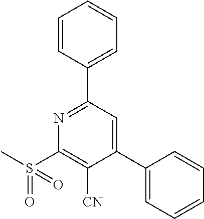 [SMILES: CS(=O)(=O)c1nc(-c2ccccc2)cc(-c2ccccc2)c1C#N] | 2-Methanesulfonyl-4,6-diphenyl- nicotinonitrile; |
| 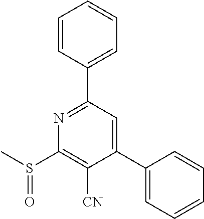 [SMILES: CS(=O)c1nc(-c2ccccc2)cc(-c2ccccc2)c1C#N] | 2-Methanesulfinyl-4,6-diphenyl- nicotinonitrile; |
| 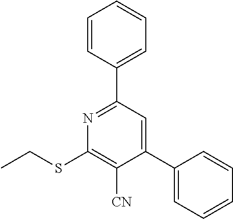 [SMILES: CCSc1nc(-c2ccccc2)cc(-c2ccccc2)c1C#N] | 2-Ethylsulfanyl-4,6-diphenyl- nicotinonitrile; |
| 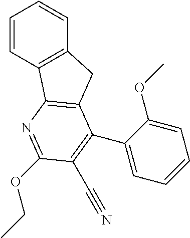 [SMILES: CCOc1nc2c(c(-c3ccccc3OC)c1C#N)Cc1ccccc1-2] | 2-Ethoxy-4-(2-methoxyphenyl)-5H- indeno[1,2-b]pyridine-3-carbonitrile; |
| 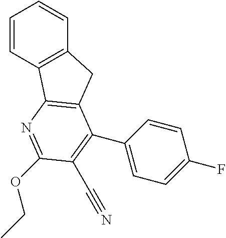 [SMILES: CCOc1nc2c(c(-c3ccc(F)cc3)c1C#N)Cc1ccccc1-2] | 4-(4-Fluorophenyl)-2-ethoxy-5H- indeno[1,2-b]pyridine-3-carbonitrile; |
| 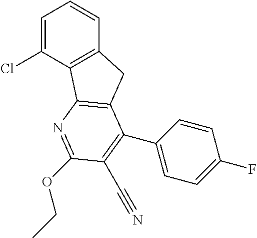 [SMILES: CCOc1nc2c(c(-c3ccc(F)cc3)c1C#N)Cc1cccc(Cl)c1-2] | 9-Chloro-2-ethoxy-4-(4-fluorophenyl)-5H- indeno[1,2-b]pyridine-3-carbonitrile; |
| 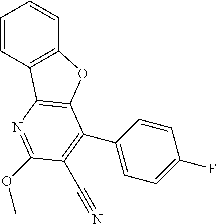 [SMILES: COc1nc2c(oc3ccccc32)c(-c2ccc(F)cc2)c1C#N] | 4-(4-Fluorophenyl)-2- methoxybenzofuro[3,2-b]pyridine-3- carbonitrile; |
| 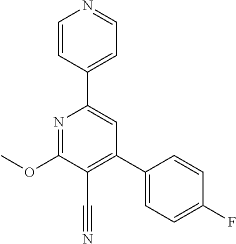 [SMILES: COc1nc(-c2ccncc2)cc(-c2ccc(F)cc2)c1C#N] | 4-(4-Fluorophenyl)-2-methoxy-6-(pyridin- 4-yl)pyridine-3-carbonitrile; |
| 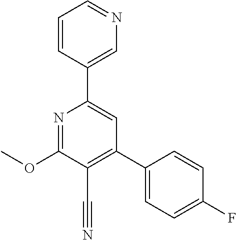 [SMILES: COc1nc(-c2cccnc2)cc(-c2ccc(F)cc2)c1C#N] | 4-(4-Fluorophenyl)-2-methoxy-6-(pyridin- 3-yl)pyridine-3-carbonitrile; |
| 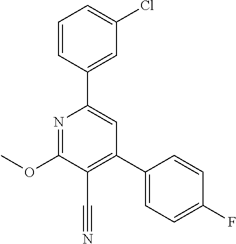 [SMILES: COc1nc(-c2cccc(Cl)c2)cc(-c2ccc(F)cc2)c1C#N] | 6-(3-Chlorophenyl)-4-(4-fluorophenyl)-2- methoxypyridine-3-carbonitrile; |
| 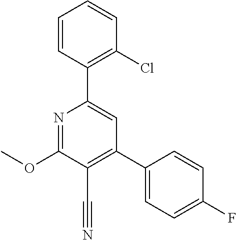 [SMILES: COc1nc(-c2ccccc2Cl)cc(-c2ccc(F)cc2)c1C#N] | 6-(2-Chlorophenyl)-4-(4-fluorophenyl)-2- methoxypyridine-3-carbonitrile; |
| 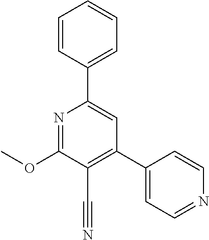 [SMILES: COc1nc(-c2ccccc2)cc(-c2ccncc2)c1C#N] | 2-Methoxy-6-phenyl-4-(pyridin-4- yl)pyridine-3-carbonitrile; |
| 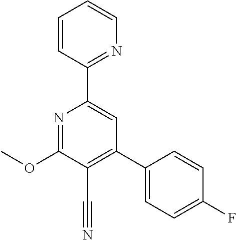 [SMILES: COc1nc(-c2ccccn2)cc(-c2ccc(F)cc2)c1C#N] | 4-(4-Fluorophenyl)-2-methoxy-6-(pyridin- 2-yl)pyridine-3-carbonitrile; |
| 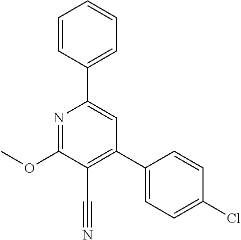 [SMILES: COc1nc(-c2ccccc2)cc(-c2ccc(Cl)cc2)c1C#N] | 4-(4-Chlorophenyl)-2-methoxy-6- phenylpyridine-3-carbonitrile; |
| 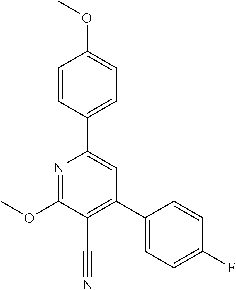 [SMILES: COc1ccc(-c2cc(-c3ccc(F)cc3)c(C#N)c(OC)n2)cc1] | 4-(4-Fluorophenyl)-2-methoxy-6-(4- methoxyphenyl)pyridine-3-carbonitrile; |
| 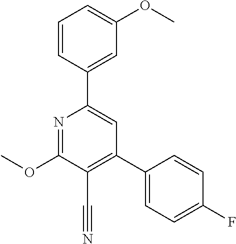 [SMILES: COc1cccc(-c2cc(-c3ccc(F)cc3)c(C#N)c(OC)n2)c1] | 4-(4-Fluorophenyl)-2-methoxy-6-(3- methoxyphenyl)pyridine-3-carbonitrile; |
| 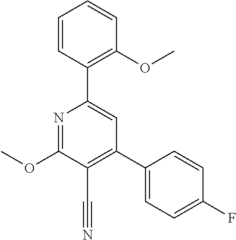 [SMILES: COc1ccccc1-c1cc(-c2ccc(F)cc2)c(C#N)c(OC)n1] | 4-(4-Fluorophenyl)-2-methoxy-6-(2- methoxyphenyl)pyridine-3-carbonitrile; |
| 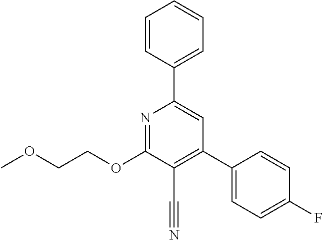 [SMILES: COCCOc1nc(-c2ccccc2)cc(-c2ccc(F)cc2)c1C#N] | 2-(2-Methoxyethoxy)-4-(4-fluorophenyl)-6- phenylpyridine-3-carbonitrile; |
| 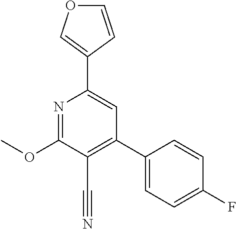 [SMILES: COc1nc(-c2ccoc2)cc(-c2ccc(F)cc2)c1C#N] | 4-(4-Fluoro-phenyl)-6-furan-3-yl-2- methoxy-nicotinonitrile; |
| 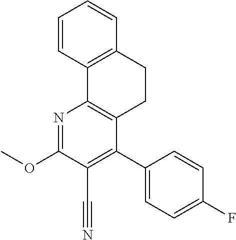 [SMILES: COc1nc2c(c(-c3ccc(F)cc3)c1C#N)CCc1ccccc1-2] | 4-(4-Fluorophenyl)-5,6-dihydro-2- methoxybenzo[h]quinoline-3-carbonitrile; |
| 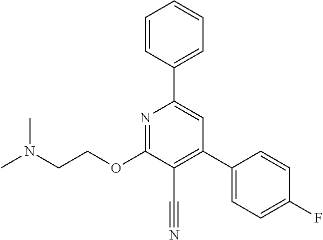 [SMILES: CN(C)CCOc1nc(-c2ccccc2)cc(-c2ccc(F)cc2)c1C#N] | 2-(2-(Dimethylamino)ethoxy)-4-(4- fluorophenyl)-6-phenylpyridine-3- carbonitrile; |
| 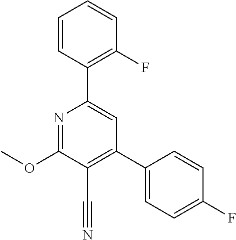 [SMILES: COc1nc(-c2ccccc2F)cc(-c2ccc(F)cc2)c1C#N] | 6-(2-Fluorophenyl)-4-(4-fluorophenyl)-2- methoxypyridine-3-carbonitrile; |
| 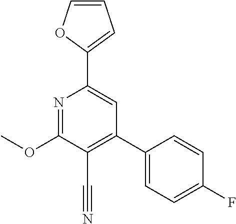 [SMILES: COc1nc(-c2ccco2)cc(-c2ccc(F)cc2)c1C#N] | 4-(4-Fluoro-phenyl)-6-furan-2-yl-2- methoxy-nicotinonitrile; |
| 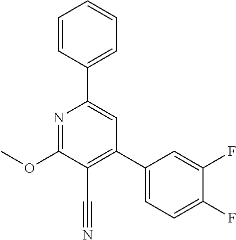 [SMILES: COc1nc(-c2ccccc2)cc(-c2ccc(F)c(F)c2)c1C#N] | 4-(3,4-Difluoro-phenyl)-2-methoxy-6- phenyl-nicotinonitrile; |
| 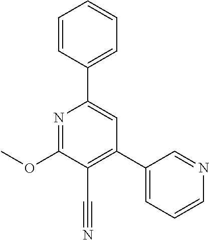 [SMILES: COc1nc(-c2ccccc2)cc(-c2cccnc2)c1C#N] | 2′-Methoxy-6′-phenyl-[3,4′]bipyridinyl-3′- carbonitrile; |
| 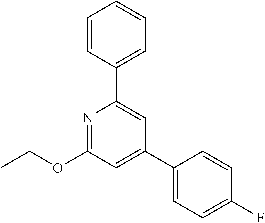 [SMILES: CCOc1cc(-c2ccc(F)cc2)cc(-c2ccccc2)n1] | 2-Ethoxy-4-(4-fluoro-phenyl)-6-phenyl- pyridine; |
| 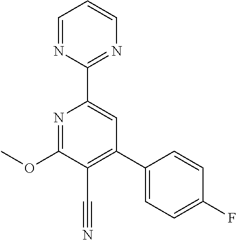 [SMILES: COc1nc(-c2ncccn2)cc(-c2ccc(F)cc2)c1C#N] | 4-(4-Fluoro-phenyl)-2-methoxy-6- pyrimidin-2-yl-nicotinonitrile; |
| 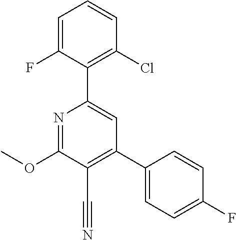 [SMILES: COc1nc(-c2c(F)cccc2Cl)cc(-c2ccc(F)cc2)c1C#N] | 6-(2-Chloro-6-fluoro-phenyl)-4-(4-fluoro- phenyl)-2-methoxy-nicotinonitrile; |
| 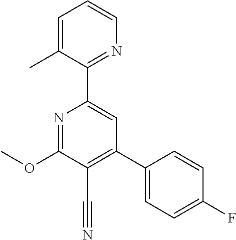 [SMILES: COc1nc(-c2ncccc2C)cc(-c2ccc(F)cc2)c1C#N] | 4-(4-Fluoro-phenyl)-6-methoxy-3′-methyl- [2,2′]bipyridinyl-5-carbonitrile; |
| 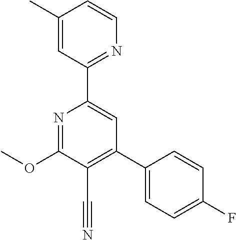 [SMILES: COc1nc(-c2cc(C)ccn2)cc(-c2ccc(F)cc2)c1C#N] | 4-(4-Fluoro-phenyl)-6-methoxy-4′-methyl- [2,2′]bipyridinyl-5-carbonitrile; |
| 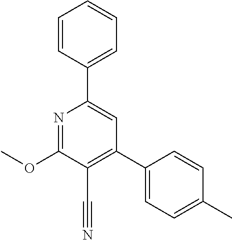 | 2-Methoxy-6-phenyl-4-p-tolyl- nicotinonitrile; |
| 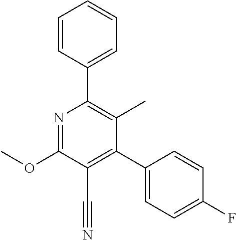 [SMILES: COc1nc(-c2ccccc2)c(C)c(-c2ccc(F)cc2)c1C#N] | 4-(4-Fluoro-phenyl)-2-methoxy-5-methyl-6- phenyl-nicotinonitrile; |
| 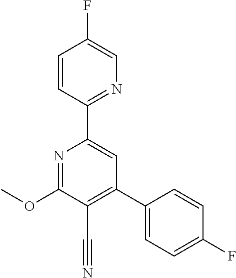 [SMILES: COc1nc(-c2ccc(F)cn2)cc(-c2ccc(F)cc2)c1C#N] | 5′-Fluoro-4-(4-fluoro-phenyl)-6-methoxy- [2,2′]bipyridinyl-5-carbonitrile; |
| 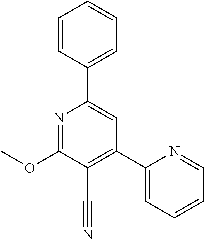 [SMILES: COc1nc(-c2ccccc2)cc(-c2ccccn2)c1C#N] | 2′-Methoxy-6′-phenyl-[2,4′]bipyridinyl-3′- carbonitrile; |
| 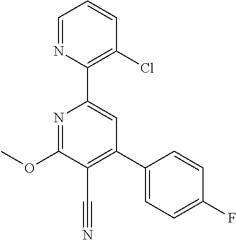 [SMILES: COc1nc(-c2ncccc2Cl)cc(-c2ccc(F)cc2)c1C#N] | 6-(3-Chloropyridin-2-yl)-4-(4- fluorophenyl)-2-methoxypyridine-3- carbonitrile; |
| 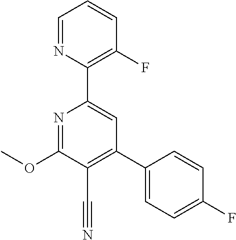 [SMILES: COc1nc(-c2ncccc2F)cc(-c2ccc(F)cc2)c1C#N] | 4-(4-Fluorophenyl)-6-(3-fluoropyridin-2- yl)-2-methoxypyridine-3-carbonitrile; |
| 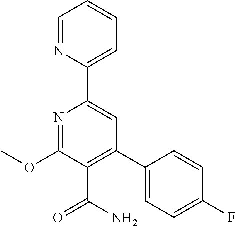 [SMILES: COc1nc(-c2ccccn2)cc(-c2ccc(F)cc2)c1C(N)=O] | 4-(4-Fluorophenyl)-2-methoxy-6-(pyridin- 2-yl)pyridine-3-carboxamide; |
| 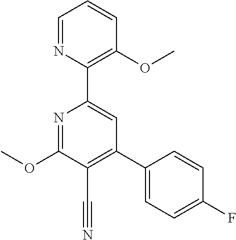 [SMILES: COc1cccnc1-c1cc(-c2ccc(F)cc2)c(C#N)c(OC)n1] | 4-(4-Fluorophenyl)-2-methoxy-6-(3- methoxypyridin-2-yl)pyridin-3- carbonitrile; |
| 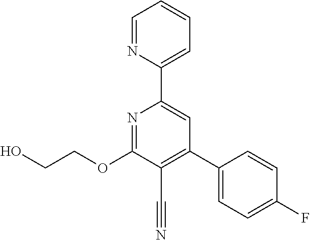 [SMILES: N#Cc1c(-c2ccc(F)cc2)cc(-c2ccccn2)nc1OCCO] | 2-(2-Hydroxyethoxy)-4-(4-fluorophenyl)-6- (pyridin-2-yl)pyridine-3-carbonitrile; |
| 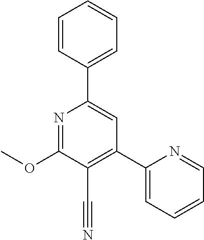 [SMILES: COc1nc(-c2ccccc2)cc(-c2ccccn2)c1C#N] | 2-Methoxy-6-phenyl-4-(pyridin-2- yl)pyridine-3-carbonitrile; |
| 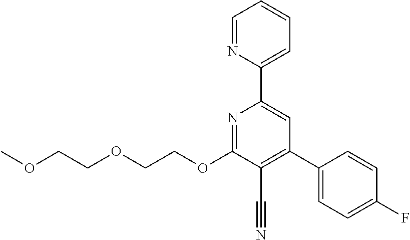 [SMILES: COCCOCCOc1nc(-c2ccccn2)cc(-c2ccc(F)cc2)c1C#N] | 2-(2-(2-Methoxyethoxy)ethoxy)-4-(4- fluorophenyl)-6-(pyridin-2-yl)pyridine-3- carbonitrile; |
| 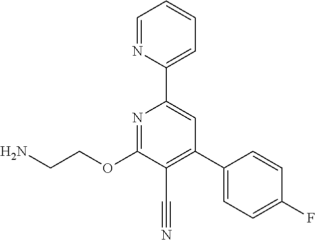 [SMILES: N#Cc1c(-c2ccc(F)cc2)cc(-c2ccccn2)nc1OCCN] | 2-(2-Aminoethoxy)-4-(4-fluorophenyl)-6- (pyridin-2-yl)pyridine-3-carbonitrile; |
| 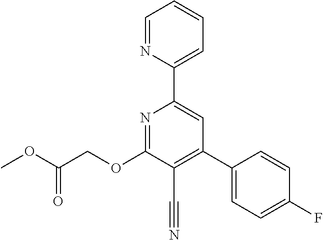 [SMILES: COC(=O)COc1nc(-c2ccccn2)cc(-c2ccc(F)cc2)c1C#N] | Methyl 2-(3-cyano-4-(4-fluorophenyl)-6- (pyridin-2-yl)pyridin-2-yloxy)acetate; |
| 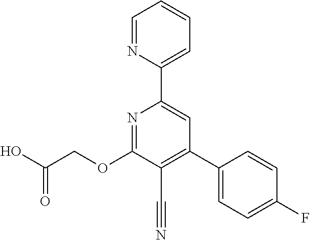 [SMILES: N#Cc1c(-c2ccc(F)cc2)cc(-c2ccccn2)nc1OCC(=O)O] | 2-(3-Cyano-4-(4-fluorophenyl)-6-(pyridin- 2-yl)pyridin-2-yloxy)acetic acid; |
| 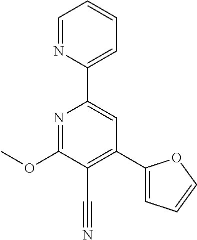 [SMILES: COc1nc(-c2ccccn2)cc(-c2ccco2)c1C#N] | 4-(Furan-2-yl)-2-methoxy-6-(pyridin-2- yl)pyridine-3-carbonitrile; |
| 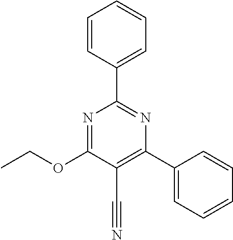 [SMILES: CCOc1nc(-c2ccccc2)nc(-c2ccccc2)c1C#N] | 4-ethoxy-2,6-diphenylpyrimidine-5- carbonitrile; |
| 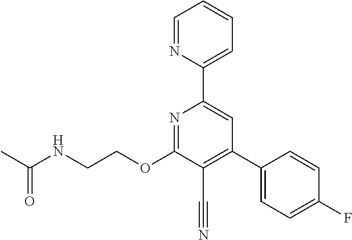 [SMILES: CC(=O)NCCOc1nc(-c2ccccn2)cc(-c2ccc(F)cc2)c1C#N] | N-(2-(3-Cyano-4-(4-fluorophenyl)-6- (pyridin-2-yl)pyridin-2- yloxy)ethyl)acetamide; |
| 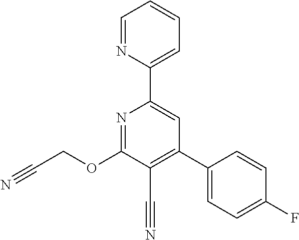 [SMILES: N#CCOc1nc(-c2ccccn2)cc(-c2ccc(F)cc2)c1C#N] | 2-(Cyanomethoxy)-4-(4-fluorophenyl)-6- (pyridin-2-yl)pyridine-3-carbonitrile; |
| 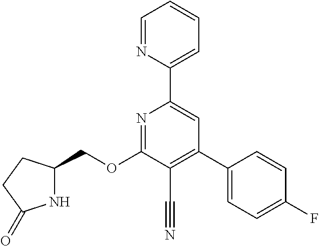 [SMILES: N#Cc1c(-c2ccc(F)cc2)cc(-c2ccccn2)nc1OC[C@@H]1CCC(=O)N1] | 2-((S)-5-(Methoxy)pyrrolidin-2-one)-4-(4- fluorophenyl)-6-(pyridin-2-yl)pyridine-3- carbonitrile; |
| 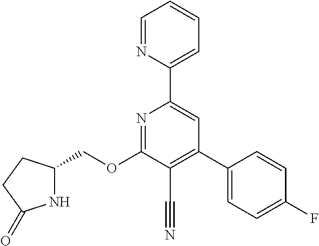 [SMILES: N#Cc1c(-c2ccc(F)cc2)cc(-c2ccccn2)nc1OC[C@H]1CCC(=O)N1] | 2-((R)-5-(Methoxy)pyrrolidin-2-one)-4-(4- fluorophenyl)-6-(pyridin-2-yl)pyridine-3- carbonitrile; |
| 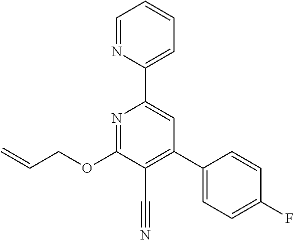 [SMILES: C=CCOc1nc(-c2ccccn2)cc(-c2ccc(F)cc2)c1C#N] | 2-(Allyloxy)-4-(4-fluorophenyl)-6-(pyridin- 2-yl)pyridine-3-carbonitrile; |
| 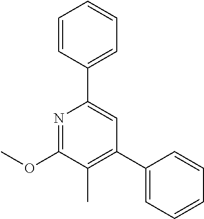 [SMILES: COc1nc(-c2ccccc2)cc(-c2ccccc2)c1C] | 2-Methoxy-3-methyl-4,6-diphenylpyridine; |
| 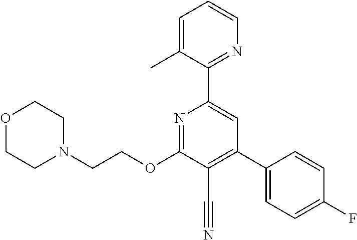 [SMILES: Cc1cccnc1-c1cc(-c2ccc(F)cc2)c(C#N)c(OCCN2CCOCC2)n1] | 2-(2-Morpholinoethoxy)-4-(4- fluorophenyl)-6-(3-methylpyridin-2- yl)pyridine-3-carbonitrile; |
| 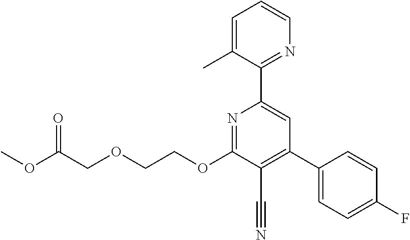 [SMILES: COC(=O)COCCOc1nc(-c2ncccc2C)cc(-c2ccc(F)cc2)c1C#N] | Methyl 2-(3-cyano-4-(4-fluorophenyl)-6-(3- methylpyridin-2-yl)pyridin-2-yloxy)2-(2- methoxyethoxy)acetate; |
| 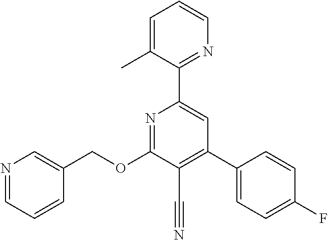 [SMILES: Cc1cccnc1-c1cc(-c2ccc(F)cc2)c(C#N)c(OCc2cccnc2)n1] | 2-((Pyridin-3-yl)methoxy)-4-(4- fluorophenyl)-6-(3-methylpyridin-2- yl)pyridine-3-carbonitrile; |
| 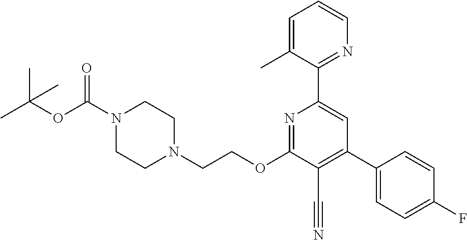 [SMILES: Cc1cccnc1-c1cc(-c2ccc(F)cc2)c(C#N)c(OCCN2CCN(C(=O)OC(C)(C)C)CC2)n1] | tert-Butyl 4-(2-(3-cyano-4-(4- fluorophenyl)-6-(3-methylpyridin-2- yl)pyridin-2-yloxy)ethyl)piperazine-1- carboxylate; |
| 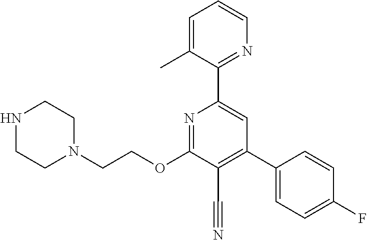 [SMILES: Cc1cccnc1-c1cc(-c2ccc(F)cc2)c(C#N)c(OCCN2CCNCC2)n1] | 2-(2-(Piperazin-1-yl)ethoxy)-4-(4- fluorophenyl)-6-(3-methylpyridin-2- yl)pyridine-3-carbonitrile; |
| 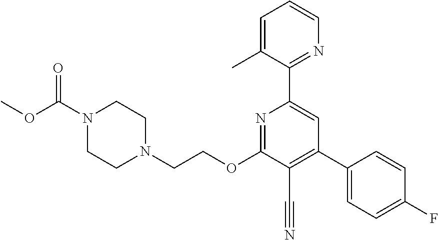 [SMILES: COC(=O)N1CCN(CCOc2nc(-c3ncccc3C)cc(-c3ccc(F)cc3)c2C#N)CC1] | Methyl 4-(2-(3-cyano-4-(4-fluorophenyl)-6- (3-methylpyridin-2-yl)pyridin-2- yloxy)ethyl)piperazine-1-carboxylate; |
| 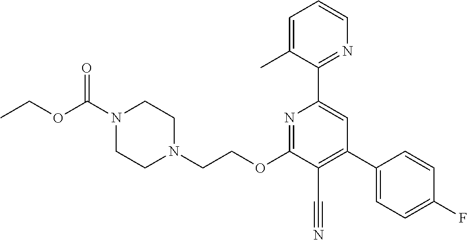 [SMILES: CCOC(=O)N1CCN(CCOc2nc(-c3ncccc3C)cc(-c3ccc(F)cc3)c2C#N)CC1] | Ethyl 4-(2-(3-cyano-4-(4-fluorophenyl)-6- (3-methylpyridin-2-yl)pyridin-2- yloxy)ethyl)piperazine-1-carboxylate; |
| 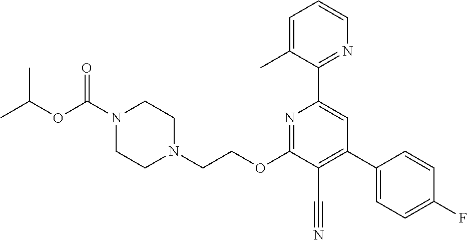 [SMILES: Cc1cccnc1-c1cc(-c2ccc(F)cc2)c(C#N)c(OCCN2CCN(C(=O)OC(C)C)CC2)n1] | Isopropyl 4-(2-(3-cyano-4-(4-fluorophenyl)- 6-(3-methylpyridin-2-yl)pyridin-2- yloxy)ethyl)piperazine-1-carboxylate; |
| 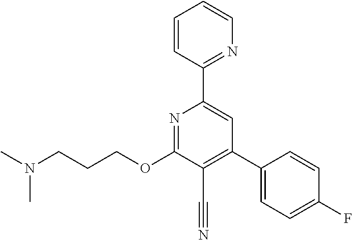 [SMILES: CN(C)CCCOc1nc(-c2ccccn2)cc(-c2ccc(F)cc2)c1C#N] | 2-(3-(Dimethylamino)propoxy)-4-(4- fluorophenyl)-6-(pyridin-2-yl)pyridine-3- carbonitrile; |
| 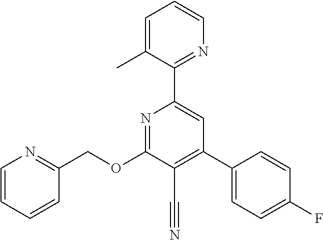 [SMILES: Cc1cccnc1-c1cc(-c2ccc(F)cc2)c(C#N)c(OCc2ccccn2)n1] | 2-((Pyridin-2-yl)methoxy)-4-(4- fluorophenyl)-6-(3-methylpyridin-2- yl)pyridine-3-carbonitrile; |
| 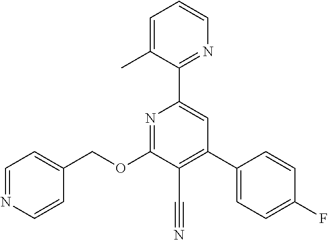 [SMILES: Cc1cccnc1-c1cc(-c2ccc(F)cc2)c(C#N)c(OCc2ccncc2)n1] | 2-((Pyridin-4-yl)methoxy)-4-(4- fluorophenyl)-6-(3-methylpyridin-2- yl)pyridine-3-carbonitrile; |
| 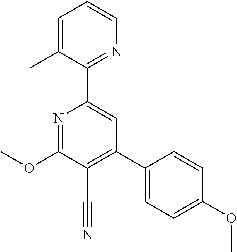 | 2-Methoxy-4-(4-methoxyphenyl)-6-(3- methylpyridin-2-yl)pyridine-3-carbonitrile; |
| 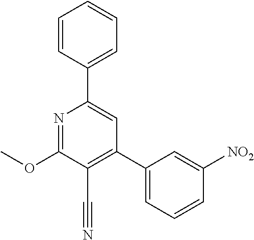 [SMILES: COc1nc(-c2ccccc2)cc(-c2cccc([N+](=O)[O-])c2)c1C#N] | 2-Methoxy-4-(3-nitrophenyl)-6- phenylpyridine-3-carbonitrile; |
| 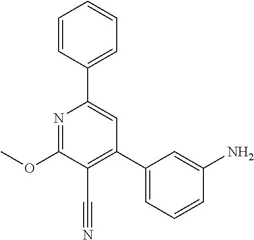 [SMILES: COc1nc(-c2ccccc2)cc(-c2cccc(N)c2)c1C#N] | 4-(3-Aminophenyl)-2-methoxy-6- phenylpyridine-3-carbonitrile; |
| 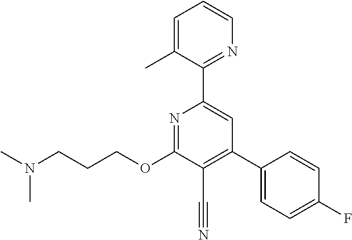 [SMILES: Cc1cccnc1-c1cc(-c2ccc(F)cc2)c(C#N)c(OCCCN(C)C)n1] | 2-(3-(Dimethylamino)propoxy)-4-(4- fluorophenyl)-6-(3-methylpyridin-2- yl)pyridine-3-carbonitrile; |
| 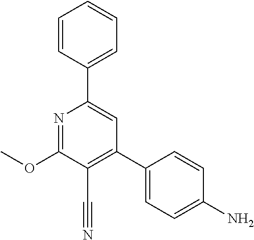 [SMILES: COc1nc(-c2ccccc2)cc(-c2ccc(N)cc2)c1C#N] | 4-(4-Aminophenyl)-2-methoxy-6- phenylpyridine-3-carbonitrile; |
| 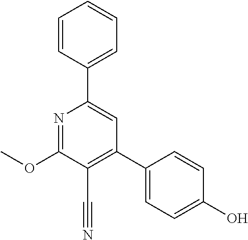 [SMILES: COc1nc(-c2ccccc2)cc(-c2ccc(O)cc2)c1C#N] | 4-(4-Hydroxyphenyl)-2-methoxy-6- phenylpyridine-3-carbonitrile; |
| 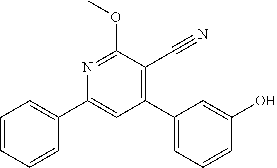 [SMILES: COc1nc(-c2ccccc2)cc(-c2cccc(O)c2)c1C#N] | 4-(3-Hydroxyphenyl)-2-methoxy-6- phenylpyridine-3-carbonitrile; |
| 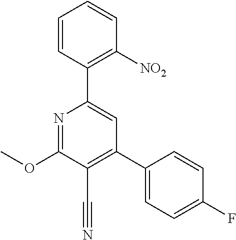 [SMILES: COc1nc(-c2ccccc2[N+](=O)[O-])cc(-c2ccc(F)cc2)c1C#N] | 4-(4-Fluorophenyl)-2-methoxy-6-(2- nitrophenyl)pyridine-3-carbonitrile; |
| 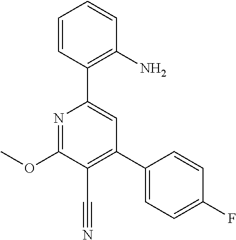 [SMILES: COc1nc(-c2ccccc2N)cc(-c2ccc(F)cc2)c1C#N] | 6-(2-Aminophenyl)-4-(4-fluorophenyl)-2- methoxypyridine-3-carbonitrile; |
| 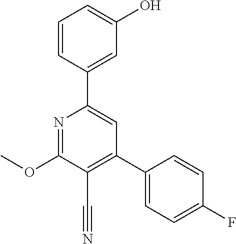 [SMILES: COc1nc(-c2cccc(O)c2)cc(-c2ccc(F)cc2)c1C#N] | 4-(4-fluorophenyl)-6-(3-hydroxyphenyl)-2- methoxypyridine-3-carbonitrile; |
| 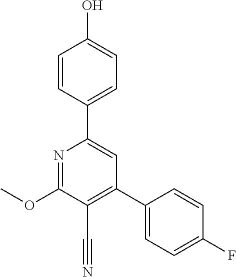 [SMILES: COc1nc(-c2ccc(O)cc2)cc(-c2ccc(F)cc2)c1C#N] | 4-(4-Fluorophenyl)-6-(4-hydroxyphenyl)-2- methoxypyridine-3-carbonitrile; |
| 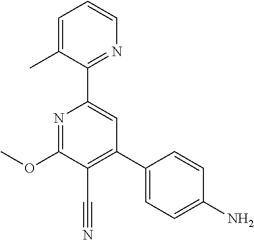 [SMILES: COc1nc(-c2ncccc2C)cc(-c2ccc(N)cc2)c1C#N] | 4-(4-aminophenyl)-2-methoxy-6-(3- methylpyridin-2-yl)pyridine-3-carbonitrile; and |
| 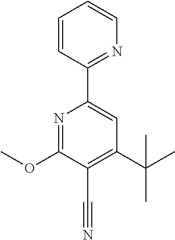 [SMILES: COc1nc(-c2ccccn2)cc(C(C)(C)C)c1C#N] | 4-(tert-Butyl-2-methoxy-6-(pyridin-2- yl)pyridine-3-carbonitrile. |

8. The method of claim 6, wherein the compound is selected from the group consisting of:

| Structure | Name |
|---|---|
| 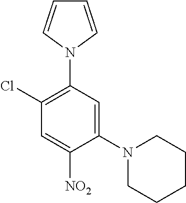 [SMILES: O=[N+]([O-])c1cc(Cl)c(-n2cccc2)cc1N1CCCCC1] | 1-(4-chloro-2-nitro-5-(1H-pyrrol-1- yl)phenyl)piperidine; |
| 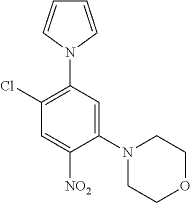 [SMILES: O=[N+]([O-])c1cc(Cl)c(-n2cccc2)cc1N1CCOCC1] | 4-(4-chloro-2-nitro-5-(1H-pyrrol-1- yl)phenyl)morpholine; and |
| 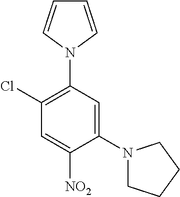 [SMILES: O=[N+]([O-])c1cc(Cl)c(-n2cccc2)cc1N1CCCC1] | 1-(2-chloro-4-nitro-5-(pyrrolidin-1- yl)phenyl)-1H-pyrrole; |

9. The method of claim 6, wherein the compound is selected from the group consisting of:

| | |
|---|---|
| 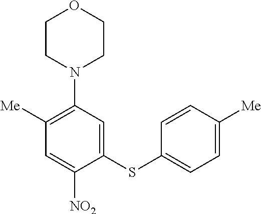 [SMILES: Cc1ccc(Sc2cc(N3CCOCC3)c(C)cc2[N+](=O)[O-])cc1] | 4-(2-methyl-4-nitro-5-(p- tolylthio)phenyl)morpholine; |
| 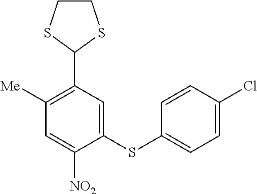 [SMILES: Cc1cc([N+](=O)[O-])c(Sc2ccc(Cl)cc2)cc1C1SCCS1] | 2-(5-((4-chlorophenyl)thio)-2-methyl-4- nitrophenyl)-1,3-dithiolane; |
| 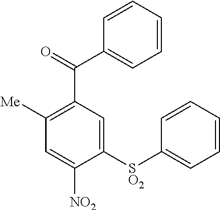 [SMILES: Cc1cc([N+](=O)[O-])c(S(=O)(=O)c2ccccc2)cc1C(=O)c1ccccc1] | (2-methyl-4-nitro-5-(phenylsulfonyl)phenyl) (phenyl)methanone; and |
| 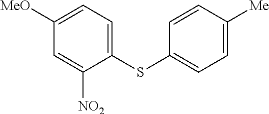 [SMILES: COc1ccc(Sc2ccc(C)cc2)c([N+](=O)[O-])c1] | (4-methoxy-2-nitrophenyl)(p-tolyl)sulfane. |

10. The method of claim 6, wherein treating or ameliorating heart failure comprises at least one from the group consisting of: regulating or lowering blood pressure, inhibiting or minimizing atherosclerotic plaque formation, and reducing or reversing cardiac remodeling.

11. The method of claim 6, wherein the subject is further administered at least one additional agent that treats or ameliorates heart failure, high or elevated blood pressure, atherosclerotic plaque formation, or cardiac remodeling.

12. The method of claim 11, wherein the at least one additional agent is selected from the group consisting of ACE inhibitors, beta blockers, neprilysin inhibitors, diuretics, aldosterone antagonists, vasodilators, and nitrates.

13. The method of claim 11, wherein the compound and the at least one additional agent are co-administered to the subject.

14. The method of claim 13, wherein the compound and the at least one additional agent are co-formulated.

15. The method of claim 6, wherein the subject is a mammal.

16. The method of claim 15, wherein the mammal is a human.

17. A method of increasing, or reversing loss of, physiological levels of H₂S in a tissue from a subject, the method comprising administering to the subject a therapeutically effective amount of at least one compound selected from the group consisting of:
(a) 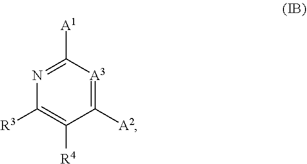
wherein in (IB):
A¹ is —C(R⁷)(R⁸)(R⁹), 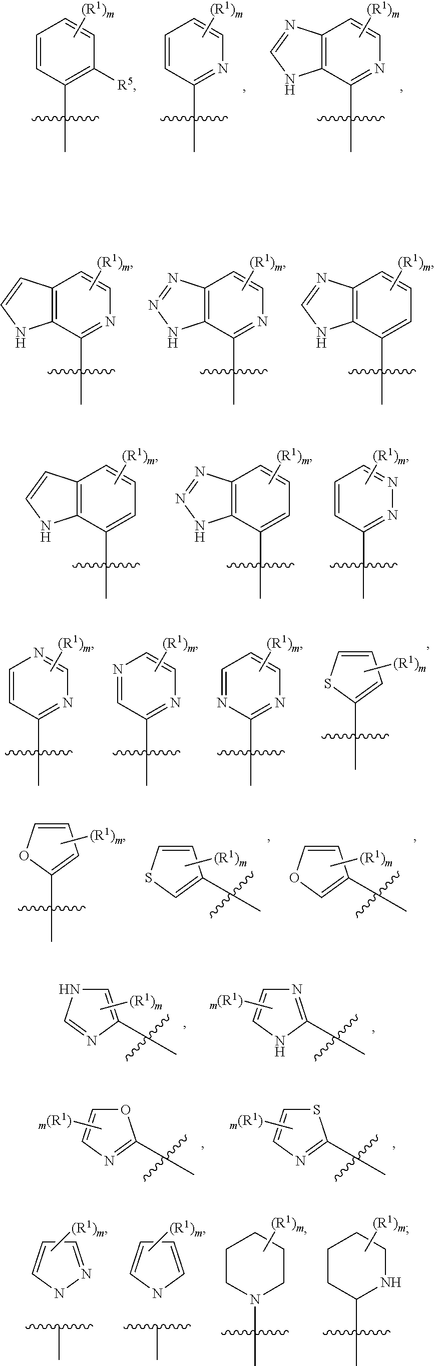
A² is —C(R¹⁰)(R¹¹)(R¹²), 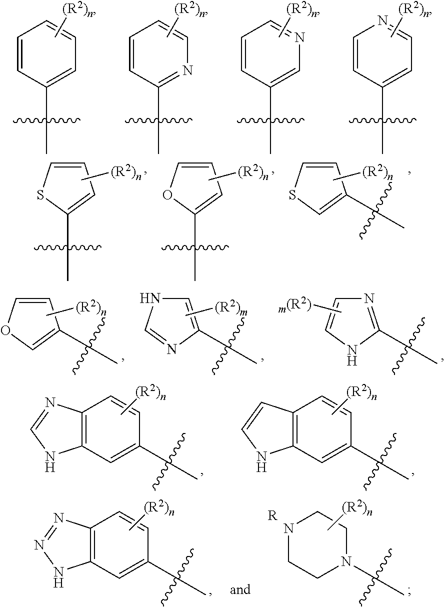
A³ is N or C—R⁶;
m is 1, 2 or 3;
n is 1, 2 or 3;
each occurrence of R¹ is independently selected from the group consisting of H, F, Cl, Br, I, —OR, —SR, —S(═O)(C₁-C₆ alkyl), —S(═O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, N¹-β-propiolactam, N¹-γ-butyrolactam, N¹-δ-valerolactam, and N¹-ε-caprolactam;
wherein, if two R¹ groups are present in neighboring carbons, they optionally combine to form the divalent group —O(CH₂)₁₋₃O—;
each occurrence of R² is independently selected from the group consisting of H, F, Cl, Br, I, —OR, —SR, —S(O)(C₁-C₆ alkyl), —S(O)₂(C₁-C₆ alkyl), —(CH₂)₀₋₅NR₂, —CN, —NO₂, C₁-C₆ alkyl, (C₁-C₆)haloalkyl, (C₁-C₆)haloalkoxy, —C(═O)NR₂, —SO₂NR₂, N¹-β-propiolactam, N¹-γ-butyrolactam, N¹-δ-valerolactam, and N¹-ε-caprolactam;
wherein, if two R² groups are present in neighboring carbons, they optionally combine to form the divalent group —O(CH₂)₁₋₃O—;
with the proviso that R² is not positioned ortho to the bond between the central heteroaryl ring and A²;
R³ is selected from the group consisting of H, C₁-C₆ alkyl, (C₁-C₆)alkoxy, (C₁-C₆)haloalkyl, (C₁-C₆)haloalkoxy, dialkylamino(C₁-C₆)alkoxy, —NR₂, —S(C₁-C₆ alkyl), —S(O)(C₁-C₆ alkyl), —S(O)₂(C₁-C₆ alkyl), N¹-β-propiolactam, N¹-γ-butyrolactam, N¹-δ-valerolactam, oxetanoyl, oxetanoxyl, N¹-ε-caprolactam, (C₂-C₆)alkenyloxy, (C₂-C₆)alkynyloxy, —O(CH₂)₂₋₄OR, —O(CH₂)₂₋₄O(CH₂)₂₋₄C(═O)OR, —O(CH₂)₂₋₄NRR, —O(CH₂)₁₋₄(aryl), —O(CH₂)₁₋₄(heteroaryl), —O(CH₂)₀₋₄CN, —O(CH₂)₂₋₄(pyrrolidin-2-one), —O(CH₂)₁₋₄C(═O)OR, —O(CH₂)₁₋₄C(═O)NRR, —O(CH₂)₂₋₄NRC(═O)R, —O(CH₂)₂₋₄NRC(═O)NRR, —O(CH₂)₂₋₄NRS(O)₂R, —O(CH₂)₂₋₄S(O)₂NRR, N⁴-morpholinyl, —O(CH₂)₂₋₄(N⁴-morpholinyl), N¹-pyrrolidinyl, —O(CH₂)₂₋₄(N¹-pyrrolidinyl), —N¹-piperidinyl, —O(CH₂)₂₋₄(N¹-piperidinyl), —O(CH₂)₂₋₄(N¹-β-propiolactam), —O(CH₂)₂₋₄(N¹-γ-butyrolactam), —O(CH₂)₂₋₄(N¹-δ-valerolactam), N¹-piperazinyl, and —O(CH₂)₂₋₄(N¹-piperazinyl); wherein the 4-position of the piperazinyl group is optionally substituted with a group selected from R, —C(═O)R, —S(O)₂R, and —C(═O)OR;
R⁴ is independently selected from the group consisting of H, —CH₃, —CN, —C≡CR, —C(═O)OR, and —C(═O)NR₂, or R⁴ can combine with R³ to form: 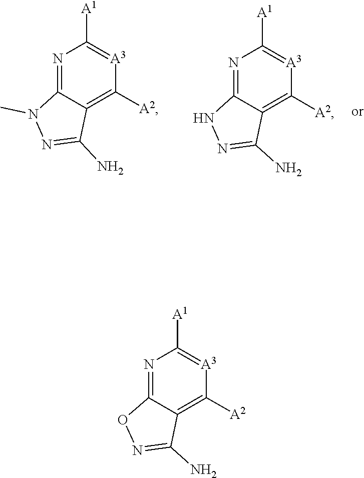
R⁵ is R¹ and R⁶ is H or C₁-C₆ alkyl, or R⁵ and R⁶ combine to form a divalent group selected from the group consisting of —O—, —CRR—, or —CRR—CRR—,
each of R⁷, R⁸, R⁹, R¹⁰, R¹¹, and R¹² is independently selected from optionally substituted C₁-C₁₀ alkyl, wherein two or three of R⁷, R⁸, R⁹ or of R¹⁰, R¹¹, R¹² can optionally combine to form monocyclic or polycyclic groups; and
each occurrence of R is independently H or C₁-C₆ alkyl; 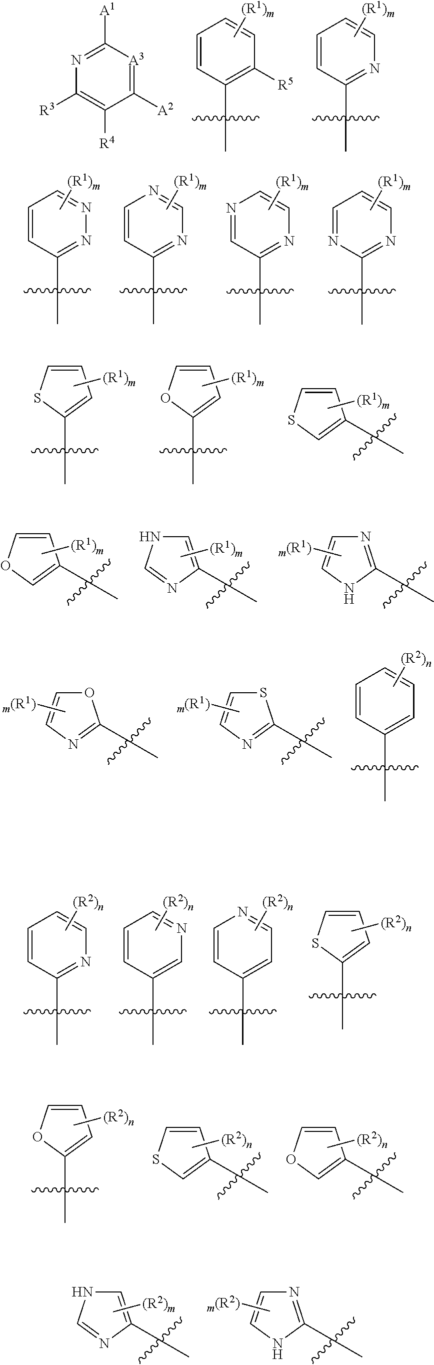
(b) 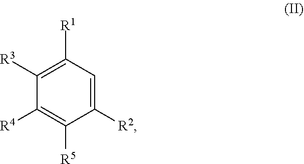
wherein in (II):
R¹ is selected from the group consisting of phenyl, N¹-pyrrolyl, N¹-imidazolyl, N¹-pyrazolyl, N¹-triazolyl, N²-1,2,3-triazolyl, N¹-triazolyl, N⁴-1,2,3-triazolyl, N¹-tetrazolyl, N¹-isoxazolyl, N¹-pyrrolidinyl, N¹-piperidinyl, N¹-morpholinyl, piperizin-1-yl, and N⁴—(C₁-C₆ alkyl)-piperizin-1-yl,
wherein the phenyl group is optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, C(═O)NR₂, N¹-β-propiolactam, N⁴-γ-butyrolactam, N¹-δ-valerolactam, and N¹-ε-caprolactam; wherein, if two substituents are present in neighboring carbons, they optionally combine to form the divalent group —O(CH₂)₁₋₃O—;
R² is independently selected from the group consisting of H, piperizin-1-yl, N⁴—(C₁-C₆ alkyl)-piperizin-1-yl, phenyl, pyridin-2-yl, pyridin-3-yl, and pyridin-4-yl;
wherein the phenyl group is optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, C(═O)NR₂, N¹-β-propiolactam, N¹-γ-butyrolactam, N¹-δ-valerolactam, and N¹-ε-caprolactam; wherein, if two substituents are present in neighboring carbons, they optionally combine to form the divalent group —O(CH₂)₁₋₃O—;
R³ is selected from the group consisting of H and Cl;
R⁴ is selected from the group consisting of H, —OH, (C₁-C₆)alkyl, (C₁-C₆)alkoxy, (C₁-C₆)haloalkyl, (C₁-C₆)haloalkoxy, and benzyloxy;
R⁵ is independently selected from the group consisting of H, —NO₂, —CN, —C≡CH, —C(═O)OH, —SO₂(C₁-C₆ alkyl), —SO₂NHAC, and tetrazol-1-yl;
(c) 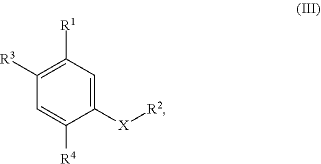
wherein in (III):
X is selected from the group consisting of O, S, S(═O), and S(═O)₂;
R¹ is selected from the group consisting of H, phenyl, —C(═O)phenyl, 1,3-dithiol-2-yl, pyrrol-1-yl, imidazole-1-yl, pyrazol-1-yl, 1,2,3-triazol-1-yl, 1,2,3-triazol-2-yl, 1,2,4-triazol-1-yl, 1,2,3-triazol-4-yl, tetrazol-1-yl, isoxazol-1-yl, pyrrolidin-1-yl, piperidin-1-yl, morpholin-1-yl, piperizin-1-yl, and N⁴—(C₁-C₆ alkyl)-piperizin-1-yl;
wherein each phenyl group is independently optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, —C(═O)NR₂, N¹-β-propiolactam, N¹-γ-butyrolactam, N¹-δ-valerolactam, and N¹-ε-caprolactam;
wherein, if two substituents are present in neighboring carbons, they optionally combine to form the divalent group —O(CH₂)₁₋₃O—;
R² is selected from the group consisting of phenyl, pyridin-2-yl, pyridin-3-yl, and pyridin-4-yl;
wherein each phenyl group is independently optionally substituted with 1-2 substituents independently selected from the group consisting of H, F, Cl, Br, I, —OR, —SR, —(CH₂)₀₋₅NR₂, —CN, —NO₂, (C₁-C₆)alkyl, (C₁-C₆)haloalkyl, (C₁-C₆)haloalkoxy, —S(═O)(C₁-C₆)alkyl, —S(═O)₂(C₁-C₆)alkyl, —C(═O)NR₂, N¹-β-propiolactam, N¹-γ-butyrolactam, N¹-δ-valerolactam, and N¹-ε-caprolactam;
wherein, if two substituents are present in neighboring carbons, they optionally combine to form the divalent group —O(CH₂)₁₋₃O—;
R³ is selected from the group consisting of H, F, Cl, Br, I, C₁-C₆ alkyl, and C₁-C₆ alkoxy; and
R⁴ is independently selected from the group consisting of H, —NO₂, —CN, —C≡CH, —C(═O)OH, —SO₂(C₁-C₆ alkyl), —SO₂NHAC, and N¹-tetrazolyl.

18. The method of claim 17, wherein the tissue comprises heart tissue.

19. The method of claim 17, wherein the subject is further administered at least one additional agent selected from the group consisting of ACE inhibitors, beta blockers, neprilysin inhibitors, diuretics, aldosterone antagonists, vasodilators, and nitrates.

20. The method of claim 19, wherein the compound and the at least one additional agent are co-administered to the subject.

21. The method of claim 20, wherein the compound and the at least one additional agent are co-formulated.

22. The method of claim 17, wherein the subject is a mammal.

23. The method of claim 22, wherein the mammal is a human.
